(19) United States
(12) Patent Application Publication
(10) Pub. No.: US 2026/0324283 A1
(43) Pub. Date: Oct. 15, 2026

(54) ORGANIC ELECTROLUMINESCENT MATERIAL AND DEVICE THEREOF

(71) Applicant: BEIJING SUMMER SPROUT TECHNOLOGY CO., LTD., Beijing (CN)

(72) Inventors: Feng Li, Beijing (CN); Yang Wang, Beijing (CN); Chi Yuen Raymond Kwong, Beijing (CN); Chuanjun Xia, Beijing (CN)

(73) Assignee: BEIJING SUMMER SPROUT TECHNOLOGY CO., LTD., Beijing (CN)

(21) Appl. No.: 18/497,316

(22) Filed: Oct. 30, 2023

(30) Foreign Application Priority Data
Oct. 31, 2022 (CN) 202211333457.5

(51) Int. Cl. H10K 85/60; C07D 403/10; C09K 11/06; H10K 50/12

(52) U.S. Cl. CPC H10K 85/6572; C07D 403/10; C09K 11/06; H10K 85/615; H10K 85/654; C09K 2211/1018; H10K 50/12

(57) ABSTRACT

Provided are an organic electroluminescent material and a device comprising the same. The organic electroluminescent material is a compound having a structure of Formula 1. The compound can be used as a host material, an electron transporting material or a hole blocking material in the organic electroluminescent device and can significantly improve the overall performance of the device, for example, high device efficiency, a low driving voltage and/or a significantly improved lifetime of the device. Further provided is a compound composition comprising a structure of Formula 1.

### CROSS-REFERENCE TO RELATED APPLICATION(S)

This application claims priority to Chinese Patent Application No. 202211333457.5 filed on Oct. 31, 2022, the disclosure of which is incorporated herein by reference in its entirety.

### TECHNICAL FIELD

The present disclosure relates to compounds for organic electronic devices such as organic light-emitting devices. In particular, the present disclosure relates to a compound having a structure of Formula 1, an organic electroluminescent device comprising the compound and a compound composition comprising the compound.

### BACKGROUND

Organic electronic devices include, but are not limited to, the following types: organic light-emitting diodes (OLEDs), organic field-effect transistors (O-FETs), organic light-emitting transistors (OLETs), organic photovoltaic devices (OPVs), dye-sensitized solar cells (DSSCs), organic optical detectors, organic photoreceptors, organic field-quench devices (OFQDs), light-emitting electrochemical cells (LECs), organic laser diodes and organic plasmon emitting devices.

In 1987, Tang and Van Slyke of Eastman Kodak reported a bilayer organic electroluminescent device, which comprises an arylamine hole transporting layer and a tris-8-hydroxyquinolato-aluminum layer as the electron and emitting layer (Applied Physics Letters, 1987, 51 (12): 913-915). Once a bias is applied to the device, green light was emitted from the device. This device laid the foundation for the development of modern organic light-emitting diodes (OLEDs). State-of-the-art OLEDs may comprise multiple layers such as charge injection and transporting layers, charge and exciton blocking layers, and one or multiple emissive layers between the cathode and anode. Since the OLED is a self-emitting solid state device, it offers tremendous potential for display and lighting applications. In addition, the inherent properties of organic materials, such as their flexibility, may make them well suited for particular applications such as fabrication on flexible substrates.

The OLED can be categorized as three different types according to its emitting mechanism. The OLED invented by Tang and van Slyke is a fluorescent OLED. It only utilizes singlet emission. The triplets generated in the device are wasted through nonradiative decay channels. Therefore, the internal quantum efficiency (IQE) of the fluorescent OLED is only 25%. This limitation hindered the commercialization of OLED. In 1997, Forrest and Thompson reported phosphorescent OLED, which uses triplet emission from heavy metal containing complexes as the emitter. As a result, both singlet and triplets can be harvested, achieving 100% IQE. The discovery and development of phosphorescent OLED contributed directly to the commercialization of active-matrix OLED (AMOLED) due to its high efficiency. Recently, Adachi achieved high efficiency through thermally activated delayed fluorescence (TADF) of organic compounds. These emitters have small singlet-triplet gap that makes the transition from triplet back to singlet possible. In the TADF device, the triplet excitons can go through reverse intersystem crossing to generate singlet excitons, resulting in high IQE.

OLEDs can also be classified as small molecule and polymer OLEDs according to the forms of the materials used. A small molecule refers to any organic or organometallic material that is not a polymer. The molecular weight of the small molecule can be large as long as it has well defined structure. Dendrimers with well-defined structures are considered as small molecules.

Polymer OLEDs include conjugated polymers and non-conjugated polymers with pendant emitting groups. Small molecule OLED can become the polymer OLED if post polymerization occurred during the fabrication process.

There are various methods for OLED fabrication. Small molecule OLEDs are generally fabricated by vacuum thermal evaporation. Polymer OLEDs are fabricated by solution process such as spin-coating, inkjet printing, and slit printing. If the material can be dissolved or dispersed in a solvent, the small molecule OLED can also be produced by solution process.

The emitting color of the OLED can be achieved by emitter structural design. An OLED may comprise one emitting layer or a plurality of emitting layers to achieve desired spectrum. In the case of green, yellow, and red OLEDs, phosphorescent emitters have successfully reached commercialization. Blue phosphorescent device still suffers from non-saturated blue color, short device lifetime, and high operating voltage. Commercial full-color OLED displays normally adopt a hybrid strategy, using fluorescent blue and phosphorescent yellow, or red and green. At present, efficiency roll-off of phosphorescent OLEDs at high brightness remains a problem. In addition, it is desirable to have more saturated emitting color, higher efficiency, and longer device lifetime. Triazine-based organic semiconductor materials are widely applied to OLEDs due to their superior photoelectric performance, redox performance and stability.

US20190214570A1 discloses an organic compound having the following structure and an organic light-emitting device comprising the compound:

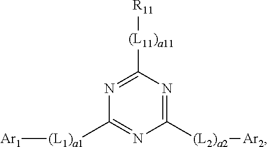

wherein Ar₁ is selected from

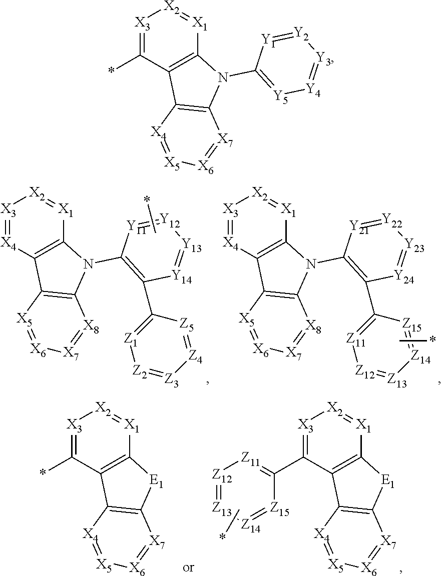

and Ar₂ is selected from

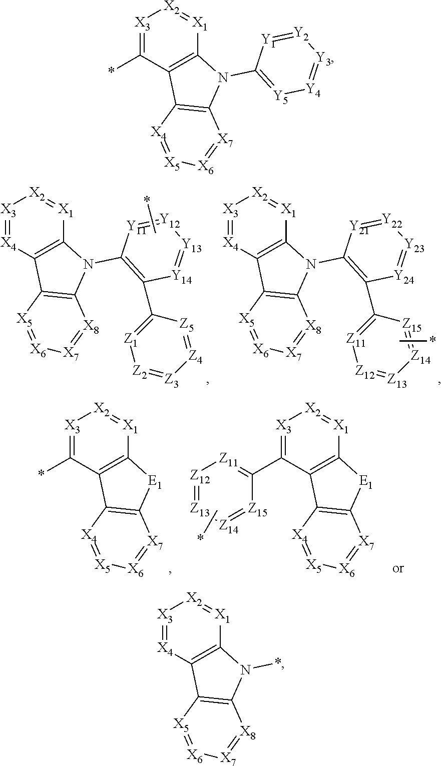

wherein E₁ is selected from C(R₂₁)(R₂₂), Si(R₂₃)(R₂₄), N(R₂₅), O or S. E₁ related to the Ar₁ and Ar₂ structures in the specific compound disclosed in this application is selected from N(R₂₅), and the specific compound comprises at least two carbazolyl groups. In addition, this application discloses the following specific compound comprising fluorenyl:

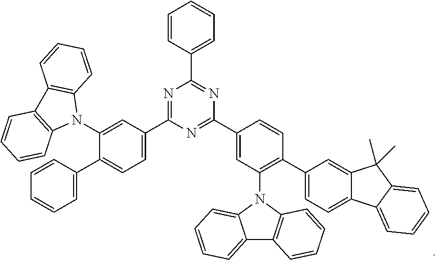 [SMILES: CC1(C)c2ccccc2-c2ccc(-c3ccc(-c4nc(-c5ccccc5)nc(-c5ccc(-c6ccccc6)c(-n6c7ccccc7c7ccccc76)c5)n4)cc3-n3c4ccccc4c4ccccc43)cc21]

However, the compound also comprises two carbazolyl groups, and the triazine fragment is joined to the 2 position of fluorene by phenylene. Therefore, this application has studied a compound where triazine is joined to a plurality of carbazole fragments and has neither focused on a compound where triazine (or by a linkage) is joined to a fluorenyl fragment nor taught a compound where triazine (or by a linkage) is joined to fluorenyl at a particular position.

WO2021040467A1 discloses an organic compound having the following structure and an organic light-emitting device comprising the compound:

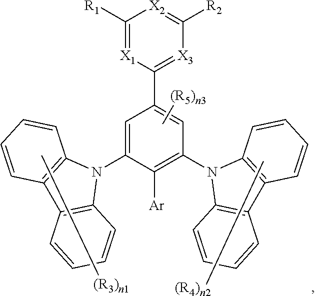

wherein Ar is substituted or unsubstituted C₆₋₆₀ aryl. This application discloses a compound having the following specific structure:

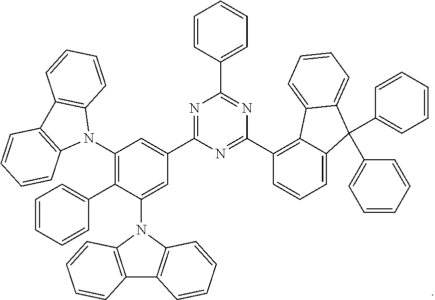 [SMILES: c1ccc(-c2nc(-c3cc(-n4c5ccccc5c5ccccc54)c(-c4ccccc4)c(-n4c5ccccc5c5ccccc54)c3)nc(-c3cccc4c3-c3ccccc3C4(c3ccccc3)c3ccccc3)n2)cc1]

The compound disclosed in this application must have at least two carbazolyl groups on phenylene joined to triazine, and this application has neither disclosed nor taught a compound with only one carbazolyl group on phenylene joined to triazine and a use of the compound in an organic electroluminescent device.

WO2020262861A1 discloses an organic compound having the following structure and an organic light-emitting device comprising the compound:

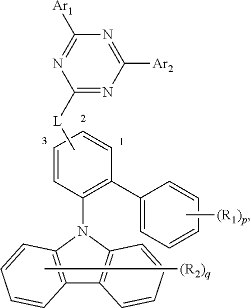

wherein L is joined to No. 1 carbon, No. 2 carbon or No. 3 carbon, L is single bond or substituted or unsubstituted C₆₋₆₀ arylene, Ar₁ and Ar₂ are each independently substituted or unsubstituted C₆₋₆₀ aryl or substituted or unsubstituted C₂₋₆₀ heteroaryl comprising any one or more of N, O and S, R₂ is each independently selected from substituted or unsubstituted C₆₋₆₀ aryl, benzoxazolyl, benzothiazolyl, dibenzofuranyl or benzothiazolyl substituted with phenyl, and q is an integer from 1 to 8. This application discloses a compound having the following specific structure:

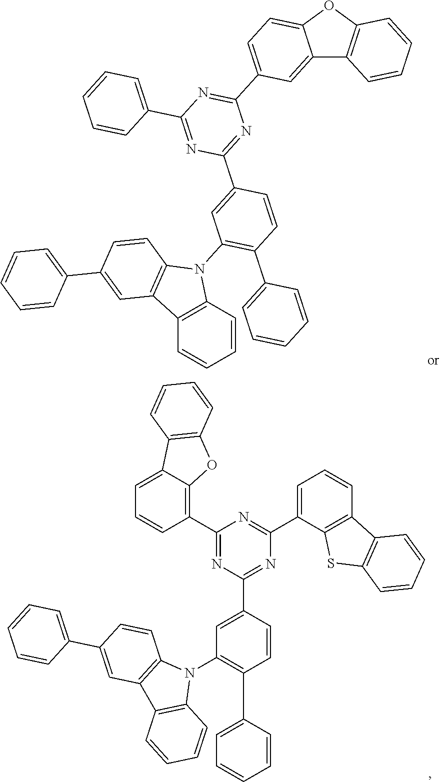

and does not disclose any compound comprising fluorene/silafluorene. Therefore, this application has neither disclosed nor taught a compound where a triazine fragment is joined to fluorene/silafluorene and has not taught a compound where triazine (or by a linkage) is joined to fluorene/silafluorene at a particular position.

However, currently reported triazine-based organic semiconductor materials have certain limitations in terms of carrier transporting ability and lifetime in optoelectronic devices. Therefore, the application potential of such materials is worthy of further research and development.

### SUMMARY

The present disclosure aims to provide a series of compounds each having a structure of Formula 1 to solve at least part of the above-mentioned problems. The compound can be applied to an organic electroluminescent device and can significantly improve the overall performance of the device, for example, high device efficiency, a low driving voltage and/or a significantly improved lifetime of the device.

According to an embodiment of the present disclosure, disclosed is a compound having a structure of Formula 1:

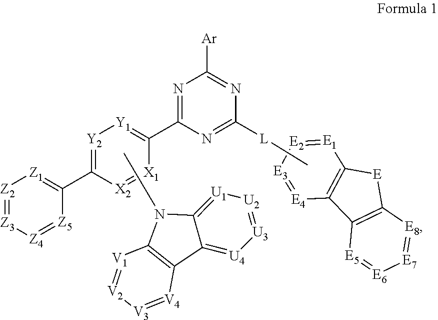

wherein in Formula 1,
one of X₁ and X₂ is selected from C, and the other is selected from CRₓ or N;
Y₁ and Y₂ are, at each occurrence identically or differently, selected from CR_{y} or N;
Z₁ to Z₅ are, at each occurrence identically or differently, selected from CR_{z} or N;
U₁ to U₄ are, at each occurrence identically or differently, selected from CRᵤ or N;
V₁ to V₄ are, at each occurrence identically or differently, selected from CR, or N;
E is, at each occurrence identically or differently, selected from CRR or SiRR;
one of E₁, E₃ and E₄ is selected from C and joined to L in Formula 1, and the other two are, at each occurrence identically or differently, selected from CRₑ or N;
E₂ and E₅ to E₈ are, at each occurrence identically or differently, selected from CRₑ or N;
Ar is, at each occurrence identically or differently, selected from substituted or unsubstituted aryl having 6 to 30 carbon atoms, substituted or unsubstituted heteroaryl having 3 to 30 carbon atoms or a combination thereof,
L is selected from a single bond, substituted or unsubstituted arylene having 6 to 30 carbon atoms, substituted or unsubstituted heteroarylene having 3 to 30 carbon atoms or a combination thereof,
Rₓ, R_{z}, Rᵤ, Rᵥ, R_{c} and R are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents R_{y} can be optionally joined to form a ring; adjacent substituents R_{z} can be optionally joined to form a ring; adjacent substituents Rᵤ can be optionally joined to form a ring; adjacent substituents Rᵥ can be optionally joined to form a ring; adjacent substituents Rₑ can be optionally joined to form a ring; adjacent substituents R can be optionally joined to form a ring.

According to another embodiment of the present disclosure, further disclosed is an electroluminescent device comprising an anode, a cathode and an organic layer disposed between the anode and the cathode, wherein the organic layer comprises the compound of Formula 1 in the preceding embodiment.

According to another embodiment of the present disclosure, further disclosed is a compound composition comprising the compound of Formula 1 in the preceding embodiment.

The present disclosure discloses the series of compounds each having the structure of Formula 1. The compound can be used as a host material, an electron transporting material or a hole blocking material in the organic electroluminescent device and can significantly improve the overall performance of the device, for example, the high device efficiency, the low driving voltage and/or the significantly improved lifetime of the device.

### BRIEF DESCRIPTION OF DRAWINGS

FIG. 1 is a schematic diagram of an organic light-emitting apparatus that may contain a compound and a compound composition disclosed herein.

FIG. 2 is a schematic diagram of another organic light-emitting apparatus that may contain a compound and a compound composition disclosed herein.

### DETAILED DESCRIPTION

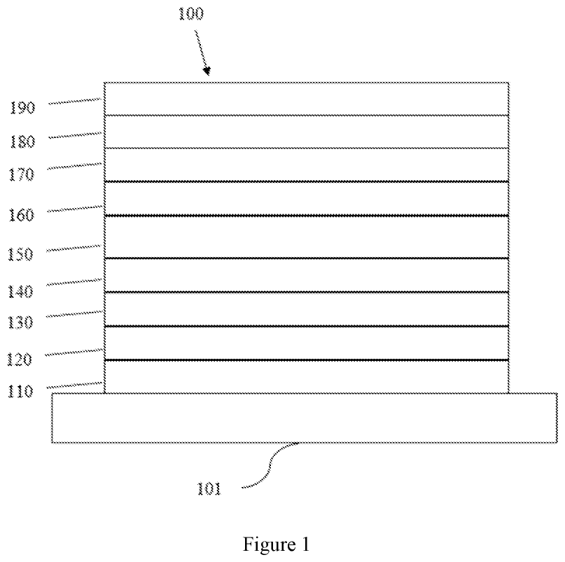

OLEDs can be fabricated on various types of substrates such as glass, plastic, and metal foil. FIG. 1 schematically shows an organic light-emitting device 100 without limitation. The figures are not necessarily drawn to scale. Some of the layers in the figures can also be omitted as needed. Device 100 may include a substrate 101, an anode 110, a hole injection layer 120, a hole transport layer 130, an electron blocking layer 140, an emissive layer 150, a hole blocking layer 160, an electron transport layer 170, an electron injection layer 180 and a cathode 190. Device 100 may be fabricated by depositing the layers described in order. The properties and functions of these various layers, as well as example materials, are described in more detail in U.S. Pat. No. 7,279,704 at cols. 6-10, the contents of which are incorporated by reference herein in its entirety.

More examples for each of these layers are available. For example, a flexible and transparent substrate-anode combination is disclosed in U.S. Pat. No. 5,844,363, which is incorporated by reference herein in its entirety. An example of a p-doped hole transport layer is m-MTDATA doped with F4-TCNQ at a molar ratio of 50:1, as disclosed in U.S. Patent Application Publication No. 2003/0230980, which is incorporated by reference herein in its entirety. Examples of host materials are disclosed in U.S. Pat. No. 6,303,238 to Thompson et al., which is incorporated by reference herein in its entirety. An example of an n-doped electron transport layer is BPhen doped with Li at a molar ratio of 1:1, as disclosed in U.S. Patent Application Publication No. 2003/0230980, which is incorporated by reference herein in its entirety. U.S. Pat. Nos. 5,703,436 and 5,707,745, which are incorporated by reference herein in their entireties, disclose examples of cathodes including composite cathodes having a thin layer of metal such as Mg:Ag with an overlying transparent, electrically-conductive, sputter-deposited ITO layer. The theory and use of blocking layers are described in more detail in U.S. Pat. No. 6,097,147 and U.S. Patent Application Publication No. 2003/0230980, which are incorporated by reference herein in their entireties. Examples of injection layers are provided in U.S. Patent Application Publication No. 2004/0174116, which is incorporated by reference herein in its entirety. A description of protective layers may be found in U.S. Patent Application Publication No. 2004/0174116, which is incorporated by reference herein in its entirety.

The layered structure described above is provided by way of non-limiting examples. Functional OLEDs may be achieved by combining the various layers described in different ways, or layers may be omitted entirely. It may also include other layers not specifically described. Within each layer, a single material or a mixture of multiple materials can be used to achieve optimum performance. Any functional layer may include several sublayers. For example, the emissive layer may have two layers of different emitting materials to achieve desired emission spectrum.

In one embodiment, an OLED may be described as having an “organic layer” disposed between a cathode and an anode. This organic layer may include a single layer or multiple layers.

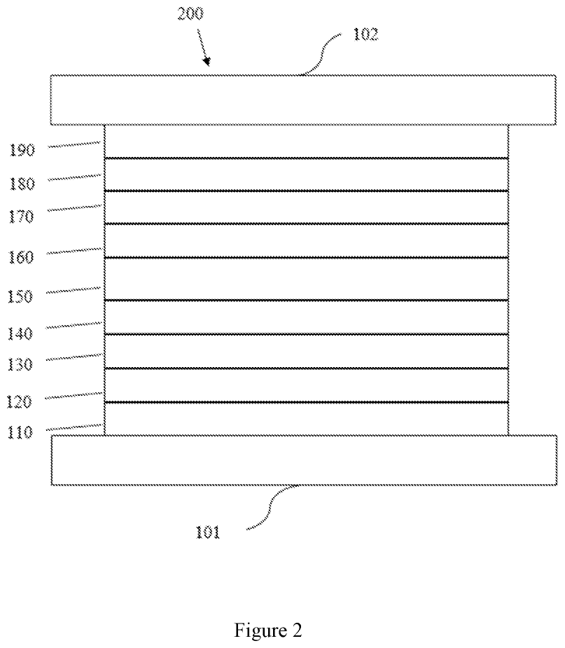

An OLED can be encapsulated by a barrier layer. FIG. 2 schematically shows an organic light emitting device 200 without limitation. FIG. 2 differs from FIG. 1 in that the organic light emitting device include a barrier layer 102, which is above the cathode 190, to protect it from harmful species from the environment such as moisture and oxygen. Any material that can provide the barrier function can be used as the barrier layer such as glass or organic-inorganic hybrid layers. The barrier layer should be placed directly or indirectly outside of the OLED device. Multilayer thin film encapsulation was described in U.S. Pat. No. 7,968,146, which is incorporated by reference herein in its entirety.

Devices fabricated in accordance with embodiments of the present disclosure can be incorporated into a wide variety of consumer products that have one or more of the electronic component modules (or units) incorporated therein. Some examples of such consumer products include flat panel displays, monitors, medical monitors, televisions, billboards, lights for interior or exterior illumination and/or signaling, heads-up displays, fully or partially transparent displays, flexible displays, smart phones, tablets, phablets, wearable devices, smart watches, laptop computers, digital cameras, camcorders, viewfinders, micro-displays, 3-D displays, vehicles displays, and vehicle tail lights.

The materials and structures described herein may be used in other organic electronic devices listed above.

As used herein, “top” means furthest away from the substrate, while “bottom” means closest to the substrate. Where a first layer is described as “disposed over” a second layer, the first layer is disposed further away from the substrate. There may be other layers between the first and second layers, unless it is specified that the first layer is “in contact with” the second layer. For example, a cathode may be described as “disposed over” an anode, even though there are various organic layers in between.

As used herein, “solution processible” means capable of being dissolved, dispersed, or transported in and/or deposited from a liquid medium, either in solution or suspension form.

A ligand may be referred to as “photoactive” when it is believed that the ligand directly contributes to the photoactive properties of an emissive material. A ligand may be referred to as “ancillary” when it is believed that the ligand does not contribute to the photoactive properties of an emissive material, although an ancillary ligand may alter the properties of a photoactive ligand.

It is believed that the internal quantum efficiency (IQE) of fluorescent OLEDs can exceed the 25% spin statistics limit through delayed fluorescence. As used herein, there are two types of delayed fluorescence, i.e. P-type delayed fluorescence and E-type delayed fluorescence. P-type delayed fluorescence is generated from triplet-triplet annihilation (TTA).

On the other hand, E-type delayed fluorescence does not rely on the collision of two triplets, but rather on the transition between the triplet states and the singlet excited states. Compounds that are capable of generating E-type delayed fluorescence are required to have very small singlet-triplet gaps to convert between energy states. Thermal energy can activate the transition from the triplet state back to the singlet state. This type of delayed fluorescence is also known as thermally activated delayed fluorescence (TADF). A distinctive feature of TADF is that the delayed component increases as temperature rises. If the reverse intersystem crossing (RISC) rate is fast enough to minimize the non-radiative decay from the triplet state, the fraction of back populated singlet excited states can potentially reach 75%. The total singlet fraction can be 100%, far exceeding 25% of the spin statistics limit for electrically generated excitons.

E-type delayed fluorescence characteristics can be found in an exciplex system or in a single compound. Without being bound by theory, it is believed that E-type delayed fluorescence requires the luminescent material to have a small singlet-triplet energy gap (ΔE_{S-T}). Organic, non-metal containing, donor-acceptor luminescent materials may be able to achieve this. The emission in these materials is generally characterized as a donor-acceptor charge-transfer (CT) type emission. The spatial separation of the HOMO and LUMO in these donor-acceptor type compounds generally results in small ΔE_{S-T}. These states may involve CT states. Generally, donor-acceptor luminescent materials are constructed by connecting an electron donor moiety such as amino- or carbazole-derivatives and an electron acceptor moiety such as N-containing six-membered aromatic rings.

### Definition of Terms of Substituents

Halogen or halide—as used herein includes fluorine, chlorine, bromine, and iodine.

Alkyl—as used herein includes both straight and branched chain alkyl groups. Alkyl may be alkyl having 1 to 20 carbon atoms, preferably alkyl having 1 to 12 carbon atoms, and more preferably alkyl having 1 to 6 carbon atoms. Examples of alkyl groups include a methyl group, an ethyl group, a propyl group, an isopropyl group, a n-butyl group, an s-butyl group, an isobutyl group, a t-butyl group, an n-pentyl group, an n-hexyl group, an n-heptyl group, an n-octyl group, an n-nonyl group, an n-decyl group, an n-undecyl group, an n-dodecyl group, an n-tridecyl group, an n-tetradecyl group, an n-pentadecyl group, an n-hexadecyl group, an n-heptadecyl group, an n-octadecyl group, a neopentyl group, a 1-methylpentyl group, a 2-methylpentyl group, a 1-pentylhexyl group, a 1-butylpentyl group, a 1-heptyloctyl group, and a 3-methylpentyl group. Of the above, preferred are a methyl group, an ethyl group, a propyl group, an isopropyl group, a n-butyl group, an s-butyl group, an isobutyl group, a t-butyl group, an n-pentyl group, a neopentyl group, and an n-hexyl group. Additionally, the alkyl group may be optionally substituted.

Cycloalkyl—as used herein includes cyclic alkyl groups. The cycloalkyl groups may be those having 3 to 20 ring carbon atoms, preferably those having 4 to 10 carbon atoms. Examples of cycloalkyl include cyclobutyl, cyclopentyl, cyclohexyl, 4-methylcyclohexyl, 4,4-dimethylcylcohexyl, 1-adamantyl, 2-adamantyl, 1-norbornyl, 2-norbornyl, and the like. Of the above, preferred are cyclopentyl, cyclohexyl, 4-methylcyclohexyl, and 4,4-dimethylcylcohexyl. Additionally, the cycloalkyl group may be optionally substituted.

Heteroalkyl—as used herein, includes a group formed by replacing one or more carbons in an alkyl chain with a hetero-atom(s) selected from the group consisting of a nitrogen atom, an oxygen atom, a sulfur atom, a selenium atom, a phosphorus atom, a silicon atom, a germanium atom, and a boron atom. Heteroalkyl may be those having 1 to 20 carbon atoms, preferably those having 1 to 10 carbon atoms, and more preferably those having 1 to 6 carbon atoms. Examples of heteroalkyl include methoxymethyl, ethoxymethyl, ethoxyethyl, methylthiomethyl, ethylthiomethyl, ethylthioethyl, methoxymethoxymethyl, ethoxymethoxymethyl, ethoxyethoxyethyl, hydroxymethyl, hydroxyethyl, hydroxypropyl, mercaptomethyl, mercaptoethyl, mercaptopropyl, aminomethyl, aminoethyl, aminopropyl, dimethylaminomethyl, trimethylgermanylmethyl, trimethylgermanylethyl, trimethylgermanylisopropyl, dimethylethylgermanylmethyl, dimethylisopropylgermanylmethyl, tert-butyldimethylgermanylmethyl, triethylgermanylmethyl, triethylgermanylethyl, triisopropylgermanylmethyl, triisopropylgermanylethyl, trimethylsilylmethyl, trimethylsilylethyl, trimethylsilylisopropyl, triisopropylsilylmethyl, and triisopropylsilylethyl. Additionally, the heteroalkyl group may be optionally substituted.

Alkenyl—as used herein includes straight chain, branched chain, and cyclic alkene groups. Alkenyl may be those having 2 to 20 carbon atoms, preferably those having 2 to 10 carbon atoms. Examples of alkenyl include vinyl, 1-propenyl group, 1-butenyl, 2-butenyl, 3-butenyl, 1,3-butandienyl, 1-methylvinyl, styryl, 2,2-diphenylvinyl, 1,2-diphenylvinyl, 1-methylallyl, 1,1-dimethylallyl, 2-methylallyl, 1-phenylallyl, 2-phenylallyl, 3-phenylallyl, 3,3-diphenylallyl, 1,2-dimethylallyl, 1-phenyl-1-butenyl, 3-phenyl-1-butenyl, cyclopentenyl, cyclopentadienyl, cyclohexenyl, cycloheptenyl, cycloheptatrienyl, cyclooctenyl, cyclooctatetraenyl, and norbornenyl. Additionally, the alkenyl group may be optionally substituted.

Alkynyl—as used herein includes straight chain alkynyl groups. Alkynyl may be those having 2 to 20 carbon atoms, preferably those having 2 to 10 carbon atoms. Examples of alkynyl groups include ethynyl, propynyl, propargyl, 1-butynyl, 2-butynyl, 3-butynyl, 1-pentynyl, 2-pentynyl, 3,3-dimethyl-1-butynyl, 3-ethyl-3-methyl-1-pentynyl, 3,3-diisopropyl-1-pentynyl, phenylethynyl, phenylpropynyl, etc. Of the above, preferred are ethynyl, propynyl, propargyl, 1-butynyl, 2-butynyl, 3-butynyl, 1-pentynyl, and phenylethynyl. Additionally, the alkynyl group may be optionally substituted.

Aryl or an aromatic group—as used herein includes non-condensed and condensed systems. Aryl may be those having 6 to 30 carbon atoms, preferably those having 6 to 20 carbon atoms, and more preferably those having 6 to 12 carbon atoms. Examples of aryl groups include phenyl, biphenyl, terphenyl, triphenylene, tetraphenylene, naphthalene, anthracene, phenalene, phenanthrene, fluorene, pyrene, chrysene, perylene, and azulene, preferably phenyl, biphenyl, terphenyl, triphenylene, fluorene, and naphthalene. Examples of non-condensed aryl groups include phenyl, biphenyl-2-yl, biphenyl-3-yl, biphenyl-4-yl, p-terphenyl-4-yl, p-terphenyl-3-yl, p-terphenyl-2-yl, m-terphenyl-4-yl, m-terphenyl-3-yl, m-terphenyl-2-yl, o-tolyl, m-tolyl, p-tolyl, p-(2-phenylpropyl)phenyl, 4′-methylbiphenylyl, 4″-t-butyl-p-terphenyl-4-yl, o-cumenyl, m-cumenyl, p-cumenyl, 2,3-xylyl, 3,4-xylyl, 2,5-xylyl, mesityl, and m-quarterphenyl. Additionally, the aryl group may be optionally substituted.

Heterocyclic groups or heterocycle—as used herein include non-aromatic cyclic groups. Non-aromatic heterocyclic groups include saturated heterocyclic groups having 3 to 20 ring atoms and unsaturated non-aromatic heterocyclic groups having 3 to 20 ring atoms, where at least one ring atom is selected from the group consisting of a nitrogen atom, an oxygen atom, a sulfur atom, a selenium atom, a silicon atom, a phosphorus atom, a germanium atom, and a boron atom. Preferred non-aromatic heterocyclic groups are those having 3 to 7 ring atoms, each of which includes at least one hetero-atom such as nitrogen, oxygen, silicon, or sulfur. Examples of non-aromatic heterocyclic groups include oxiranyl, oxetanyl, tetrahydrofuranyl, tetrahydropyranyl, dioxolanyl, dioxanyl, aziridinyl, dihydropyrrolyl, tetrahydropyrrolyl, piperidinyl, oxazolidinyl, morpholinyl, piperazinyl, oxepinyl, thiepinyl, azepinyl, and tetrahydrosilolyl. Additionally, the heterocyclic group may be optionally substituted.

Heteroaryl—as used herein, includes non-condensed and condensed hetero-aromatic groups having 1 to 5 hetero-atoms, where at least one hetero-atom is selected from the group consisting of a nitrogen atom, an oxygen atom, a sulfur atom, a selenium atom, a silicon atom, a phosphorus atom, a germanium atom, and a boron atom. A hetero-aromatic group is also referred to as heteroaryl. Heteroaryl may be those having 3 to 30 carbon atoms, preferably those having 3 to 20 carbon atoms, and more preferably those having 3 to 12 carbon atoms. Suitable heteroaryl groups include dibenzothiophene, dibenzofuran, dibenzoselenophene, furan, thiophene, benzofuran, benzothiophene, benzoselenophene, carbazole, indolocarbazole, pyridoindole, pyrrolodipyridine, pyrazole, imidazole, triazole, oxazole, thiazole, oxadiazole, oxatriazole, dioxazole, thiadiazole, pyridine, pyridazine, pyrimidine, pyrazine, triazine, oxazine, oxathiazine, oxadiazine, indole, benzimidazole, indazole, indoxazine, benzoxazole, benzisoxazole, benzothiazole, quinoline, isoquinoline, cinnoline, quinazoline, quinoxaline, naphthyridine, phthalazine, pteridine, xanthene, acridine, phenazine, phenothiazine, benzofuropyridine, furodipyridine, benzothienopyridine, thienodipyridine, benzoselenophenopyridine, and selenophenodipyridine, preferably dibenzothiophene, dibenzofuran, dibenzoselenophene, carbazole, indolocarbazole, imidazole, pyridine, triazine, benzimidazole, 1,2-azaborine, 1,3-azaborine, 1,4-azaborine, borazine, and aza-analogs thereof. Additionally, the heteroaryl group may be optionally substituted.

Alkoxy—as used herein, is represented by —O-alkyl, —O-cycloalkyl, —O-heteroalkyl, or —O-heterocyclic group. Examples and preferred examples of alkyl, cycloalkyl, heteroalkyl, and heterocyclic groups are the same as those described above. Alkoxy groups may be those having 1 to 20 carbon atoms, preferably those having 1 to 6 carbon atoms. Examples of alkoxy groups include methoxy, ethoxy, propoxy, butoxy, pentyloxy, hexyloxy, cyclopropyloxy, cyclobutyloxy, cyclopentyloxy, cyclohexyloxy, tetrahydrofuranyloxy, tetrahydropyranyloxy, methoxypropyloxy, ethoxyethyloxy, methoxymethyloxy, and ethoxymethyloxy. Additionally, the alkoxy group may be optionally substituted.

Aryloxy—as used herein, is represented by —O-aryl or —O-heteroaryl. Examples and preferred examples of aryl and heteroaryl are the same as those described above. Aryloxy groups may be those having 6 to 30 carbon atoms, preferably those having 6 to 20 carbon atoms. Examples of aryloxy groups include phenoxy and biphenyloxy. Additionally, the aryloxy group may be optionally substituted.

Arylalkyl—as used herein, contemplates alkyl substituted with an aryl group. Arylalkyl may be those having 7 to 30 carbon atoms, preferably those having 7 to 20 carbon atoms, and more preferably those having 7 to 13 carbon atoms. Examples of arylalkyl groups include benzyl, 1-phenylethyl, 2-phenylethyl, 1-phenylisopropyl, 2-phenylisopropyl, phenyl-t-butyl, alpha-naphthylmethyl, 1-alpha-naphthylethyl, 2-alpha-naphthylethyl, 1-alpha-naphthylisopropyl, 2-alpha-naphthylisopropyl, beta-naphthylmethyl, 1-beta-naphthylethyl, 2-beta-naphthylethyl, 1-beta-naphthylisopropyl, 2-beta-naphthylisopropyl, p-methylbenzyl, m-methylbenzyl, o-methylbenzyl, p-chlorobenzyl, m-chlorobenzyl, o-chlorobenzyl, p-bromobenzyl, m-bromobenzyl, o-bromobenzyl, p-iodobenzyl, m-iodobenzyl, o-iodobenzyl, p-hydroxybenzyl, m-hydroxybenzyl, o-hydroxybenzyl, p-aminobenzyl, m-aminobenzyl, o-aminobenzyl, p-nitrobenzyl, m-nitrobenzyl, o-nitrobenzyl, p-cyanobenzyl, m-cyanobenzyl, o-cyanobenzyl, 1-hydroxy-2-phenylisopropyl, and 1-chloro-2-phenylisopropyl. Of the above, preferred are benzyl, p-cyanobenzyl, m-cyanobenzyl, o-cyanobenzyl, 1-phenylethyl, 2-phenylethyl, 1-phenylisopropyl, and 2-phenylisopropyl. Additionally, the arylalkyl group may be optionally substituted.

Alkylsilyl—as used herein, contemplates a silyl group substituted with an alkyl group. Alkylsilyl groups may be those having 3 to 20 carbon atoms, preferably those having 3 to 10 carbon atoms. Examples of alkylsilyl groups include trimethylsilyl, triethylsilyl, methyldiethylsilyl, ethyldimethylsilyl, tripropylsilyl, tributylsilyl, triisopropylsilyl, methyldiisopropylsilyl, dimethylisopropylsilyl, tri-t-butylsilyl, triisobutylsilyl, dimethyl t-butylsilyl, and methyldi-t-butylsilyl. Additionally, the alkylsilyl group may be optionally substituted.

Arylsilyl—as used herein, contemplates a silyl group substituted with an aryl group. Arylsilyl groups may be those having 6 to 30 carbon atoms, preferably those having 8 to 20 carbon atoms. Examples of arylsilyl groups include triphenylsilyl, phenyldibiphenylylsilyl, diphenylbiphenylsilyl, phenyldiethylsilyl, diphenylethylsilyl, phenyldimethylsilyl, diphenylmethylsilyl, phenyldiisopropylsilyl, diphenylisopropylsilyl, diphenylbutylsilyl, diphenylisobutylsilyl, diphenyl t-butylsilyl. Additionally, the arylsilyl group may be optionally substituted.

Alkylgermanyl—as used herein contemplates a germanyl substituted with an alkyl group. The alkylgermanyl may be those having 3 to 20 carbon atoms, preferably those having 3 to 10 carbon atoms. Examples of alkylgermanyl include trimethylgermanyl, triethylgermanyl, methyldiethylgermanyl, ethyldimethylgermanyl, tripropylgermanyl, tributylgermanyl, triisopropylgermanyl, methyldiisopropylgermanyl, dimethylisopropylgermanyl, tri-t-butylgermanyl, triisobutylgermanyl, dimethyl-t-butylgermanyl, and methyldi-t-butylgermanyl. Additionally, the alkylgermanyl may be optionally substituted.

Arylgermanyl—as used herein contemplates a germanyl substituted with at least one aryl group or heteroaryl group. Arylgermanyl may be those having 6 to 30 carbon atoms, preferably those having 8 to 20 carbon atoms. Examples of arylgermanyl include triphenylgermanyl, phenyldibiphenylylgermanyl, diphenylbiphenylgermanyl, phenyldiethylgermanyl, diphenylethylgermanyl, phenyldimethylgermanyl, diphenylmethylgermanyl, phenyldiisopropylgermanyl, diphenylisopropylgermanyl, diphenylbutylgermanyl, diphenylisobutylgermanyl, and diphenyl-t-butylgermanyl. Additionally, the arylgermanyl may be optionally substituted.

The term “aza” in azadibenzofuran, azadibenzothiophene, etc. means that one or more of C—H groups in the respective aromatic fragment are replaced by a nitrogen atom. For example, azatriphenylene encompasses dibenzo[f,h]quinoxaline, dibenzo[f,h]quinoline and other analogs with two or more nitrogens in the ring system. One of ordinary skill in the art can readily envision other nitrogen analogs of the aza-derivatives described above, and all such analogs are intended to be encompassed by the terms as set forth herein.

In the present disclosure, unless otherwise defined, when any term of the group consisting of substituted alkyl, substituted cycloalkyl, substituted heteroalkyl, substituted heterocyclic group, substituted arylalkyl, substituted alkoxy, substituted aryloxy, substituted alkenyl, substituted alkynyl, substituted aryl, substituted heteroaryl, substituted alkylsilyl, substituted arylsilyl, substituted alkylgermanyl, substituted arylgermanyl, substituted amino, substituted acyl, substituted carbonyl, a substituted carboxylic acid group, a substituted ester group, substituted sulfinyl, substituted sulfonyl, and substituted phosphino is used, it means that any group of alkyl, cycloalkyl, heteroalkyl, heterocyclic group, arylalkyl, alkoxy, aryloxy, alkenyl, alkynyl, aryl, heteroaryl, alkylsilyl, arylsilyl, alkylgermanyl, arylgermanyl, amino, acyl, carbonyl, a carboxylic acid group, an ester group, sulfinyl, sulfonyl, and phosphino may be substituted with one or more groups selected from the group consisting of deuterium, halogen, unsubstituted alkyl having 1 to 20 carbon atoms, unsubstituted cycloalkyl having 3 to 20 ring carbon atoms, unsubstituted heteroalkyl having 1 to 20 carbon atoms, an unsubstituted heterocyclic group having 3 to 20 ring atoms, unsubstituted arylalkyl having 7 to 30 carbon atoms, unsubstituted alkoxy having 1 to 20 carbon atoms, unsubstituted aryloxy having 6 to 30 carbon atoms, unsubstituted alkenyl having 2 to 20 carbon atoms, unsubstituted alkynyl having 2 to 20 carbon atoms, unsubstituted aryl having 6 to 30 carbon atoms, unsubstituted heteroaryl having 3 to 30 carbon atoms, unsubstituted alkylsilyl having 3 to 20 carbon atoms, unsubstituted arylsilyl group having 6 to 20 carbon atoms, unsubstituted alkylgermanyl group having 3 to 20 carbon atoms, unsubstituted arylgermanyl group having 6 to 20 carbon atoms, unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group, and combinations thereof.

It is to be understood that when a molecular fragment is described as being a substituent or otherwise attached to another moiety, its name may be written as if it were a fragment (e.g. phenyl, phenylene, naphthyl, dibenzofuryl) or as if it were the whole molecule (e.g. benzene, naphthalene, dibenzofuran). As used herein, these different ways of designating a substituent or an attached fragment are considered to be equivalent.

In the compounds mentioned in the present disclosure, hydrogen atoms may be partially or fully replaced by deuterium. Other atoms such as carbon and nitrogen may also be replaced by their other stable isotopes. The replacement by other stable isotopes in the compounds may be preferred due to its enhancements of device efficiency and stability.

In the compounds mentioned in the present disclosure, multiple substitutions refer to a range that includes di-substitutions, up to the maximum available substitutions. When substitution in the compounds mentioned in the present disclosure represents multiple substitutions (including di-, tri-, and tetra-substitutions etc.), that means the substituent may exist at a plurality of available substitution positions on its linking structure, the substituents present at a plurality of available substitution positions may have the same structure or different structures.

In the compounds mentioned in the present disclosure, adjacent substituents in the compounds cannot be joined to form a ring unless otherwise explicitly defined, for example, adjacent substituents can be optionally joined to form a ring. In the compounds mentioned in the present disclosure, the expression that adjacent substituents can be optionally joined to form a ring includes a case where adjacent substituents may be joined to form a ring and a case where adjacent substituents are not joined to form a ring. When adjacent substituents can be optionally joined to form a ring, the ring formed may be monocyclic or polycyclic (including spirocyclic, endocyclic, fusedcyclic, and etc.), as well as alicyclic, heteroalicyclic, aromatic, or heteroaromatic. In such expression, adjacent substituents may refer to substituents bonded to the same atom, substituents bonded to carbon atoms which are directly bonded to each other, or substituents bonded to carbon atoms which are more distant from each other. Preferably, adjacent substituents refer to substituents bonded to the same carbon atom and substituents bonded to carbon atoms which are directly bonded to each other.

The expression that adjacent substituents can be optionally joined to form a ring is also intended to mean that two substituents bonded to the same carbon atom are joined to each other via a chemical bond to form a ring, which can be exemplified by the following formula:

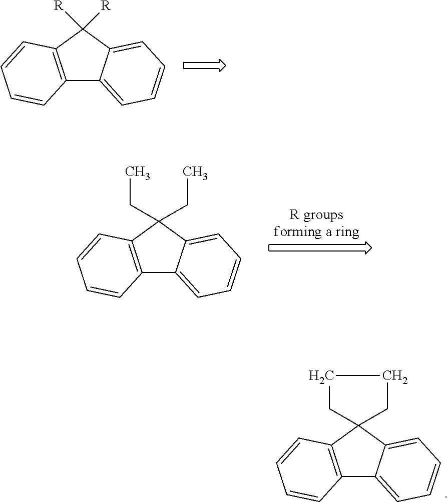

The expression that adjacent substituents can be optionally joined to form a ring is also intended to mean that two substituents bonded to carbon atoms which are directly bonded to each other are joined to each other via a chemical bond to form a ring, which can be exemplified by the following formula

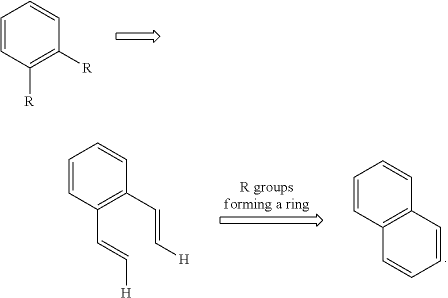

The expression that adjacent substituents can be optionally joined to form a ring is also intended to mean that two substituents bonded to further distant carbon atoms are joined to each other via a chemical bond to form a ring, which can be exemplified by the following formula:

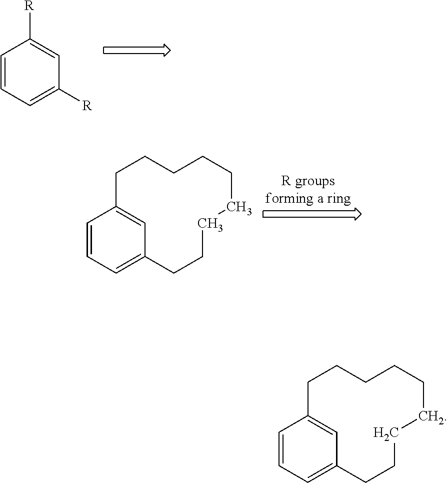

Furthermore, the expression that adjacent substituents can be optionally joined to form a ring is also intended to mean that, in the case where one of the two substituents bonded to carbon atoms which are directly bonded to each other represents hydrogen, the second substituent is bonded at a position at which the hydrogen atom is bonded, thereby forming a ring. This is exemplified by the following formula:

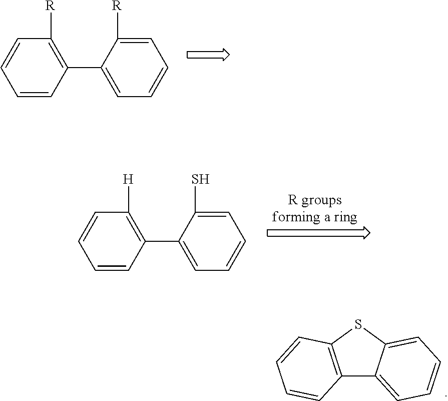

According to an embodiment of the present disclosure, disclosed is a compound having a structure of Formula 1:

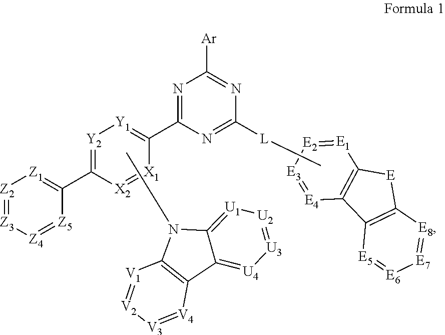

wherein in Formula 1,
one of X₁ and X₂ is selected from C, and the other is selected from CRₓ or N;
Y₁ and Y₂ are, at each occurrence identically or differently, selected from CR_{y} or N;
Z₁ to Z₅ are, at each occurrence identically or differently, selected from CR_{z} or N;
U₁ to U₄ are, at each occurrence identically or differently, selected from CRᵤ or N;
V₁ to V₄ are, at each occurrence identically or differently, selected from CR, or N;
E is, at each occurrence identically or differently, selected from CRR or SiRR;
one of E₁, E₃ and E₄ is selected from C and joined to L in Formula 1, and the other two are, at each occurrence identically or differently, selected from CRₑ or N;
E₂ and E₅ to E₅ are, at each occurrence identically or differently, selected from CRₑ or N;
Ar is, at each occurrence identically or differently, selected from substituted or unsubstituted aryl having 6 to 30 carbon atoms, substituted or unsubstituted heteroaryl having 3 to 30 carbon atoms or a combination thereof,
L is selected from a single bond, substituted or unsubstituted arylene having 6 to 30 carbon atoms, substituted or unsubstituted heteroarylene having 3 to 30 carbon atoms or a combination thereof,
Rₓ, R_{z}, Rᵤ, Rᵥ, Rₑ and R are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents R_{y} can be optionally joined to form a ring; adjacent substituents R_{z} can be optionally joined to form a ring; adjacent substituents Rᵤ can be optionally joined to form a ring; adjacent substituents Rᵥ can be optionally joined to form a ring; adjacent substituents Rₑ can be optionally joined to form a ring; adjacent substituents R can be optionally joined to form a ring.

Herein, the expression that “one of X₁ and X₂ is selected from C, and the other is selected from CRₓ or N” is intended to mean that one of X₁ and X₂ is selected from C and joined to the structure

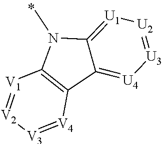

and the other is selected from CRₓ or N. Two cases are as follows: when X₁ is selected from C, X₁ is joined to the structure

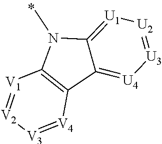

(“*” represents a position where the structure is joined to X₁), and X₂ is selected from CRₓ or N; when X₂ is selected from C, X₂ is joined to the structure

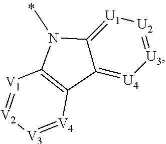

and X₁ is selected from CRₓ or N.

Herein, the expression that “adjacent substituents R_{y} can be optionally joined to form a ring” is intended to mean that any two adjacent substituents R_{y} can be joined to form a ring. Obviously, it is also possible that any adjacent R_{y} are not joined to form a ring.

Herein, the expression that “adjacent substituents R_{z} can be optionally joined to form a ring” is intended to mean that any two adjacent substituents R_{z} can be joined to form a ring. Obviously, it is also possible that any adjacent R_{z} are not joined to form a ring.

Herein, the expression that “adjacent substituents Rᵤ can be optionally joined to form a ring” is intended to mean that any two adjacent substituents Rᵤ can be joined to form a ring. Obviously, it is also possible that any adjacent Rᵤ are not joined to form a ring.

Herein, the expression that “adjacent substituents Rᵥ can be optionally joined to form a ring” is intended to mean that any two adjacent substituents Rᵥ can be joined to form a ring. Obviously, it is also possible that any adjacent Rᵥ are not joined to form a ring.

Herein, the expression that “adjacent substituents Rₑ can be optionally joined to form a ring” is intended to mean that any two adjacent substituents Rₑ can be joined to form a ring. Obviously, it is also possible that any adjacent Rₑ are not joined to form a ring.

Herein, the expression that “adjacent substituents R can be optionally joined to form a ring” is intended to mean that any two adjacent substituents R can be joined to form a ring. Obviously, it is also possible that any adjacent R are not joined to form a ring.

According to an embodiment of the present disclosure, adjacent substituents R_{y} and R_{z} in the compound having the structure of Formula 1 are not joined to form a ring.

According to an embodiment of the present disclosure, the compound having the structure of Formula 1 comprises only one carbazole structure or azacarbazole structure or fused carbazole ring structure or fused azacarbazole ring structure. The carbazole structure or azacarbazole structure or fused carbazole ring structure or fused azacarbazole ring structure is shown in the structure of Formula 1, that is, the fused ring structure where U₁ to U₄ and V₁ to V₄ are located.

According to an embodiment of the present disclosure, Rᵤ does not comprise a carbazole structure or an azacarbazole structure or a fused carbazole ring structure or a fused azacarbazole ring structure.

According to an embodiment of the present disclosure, Rᵥ does not comprise a carbazole structure or an azacarbazole structure or a fused carbazole ring structure or a fused azacarbazole ring structure.

According to an embodiment of the present disclosure, E is selected from CRR.

According to an embodiment of the present disclosure, R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group and combinations thereof.

According to an embodiment of the present disclosure, R is, at each occurrence identically or differently, selected from the group consisting of: hydrogen, deuterium, fluorine, substituted or unsubstituted methyl, substituted or unsubstituted ethyl, substituted or unsubstituted n-propyl, substituted or unsubstituted isopropyl, substituted or unsubstituted cyclopropyl, substituted or unsubstituted n-butyl, substituted or unsubstituted isobutyl, substituted or unsubstituted t-butyl, substituted or unsubstituted cyclopentyl, substituted or unsubstituted neopentyl, substituted or unsubstituted cyclohexyl, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

According to an embodiment of the present disclosure, one of X₁ and X₂ is selected from C and joined to the structure

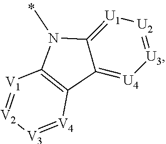

and the other is selected from CRₓ.

According to an embodiment of the present disclosure, Y₁ and Y₂ are, at each occurrence identically or differently, selected from CR_{y}.

According to an embodiment of the present disclosure, Z₁ and Z₅ are, at each occurrence identically or differently, selected from CR_{z}.

According to an embodiment of the present disclosure, U₁ to U₄ are, at each occurrence identically or differently, selected from CRᵤ.

According to an embodiment of the present disclosure, V₁ to V₄ are, at each occurrence identically or differently, selected from CRᵥ.

According to an embodiment of the present disclosure, one of E₁, E₃ and E₄ is selected from C and joined to L in Formula 1, the other two are selected from CRₑ, and E₂ and E₅ to E₈ are, at each occurrence identically or differently, selected from CRₑ.

According to an embodiment of the present disclosure, E₃ is selected from C and joined to L in Formula 1, and E₁ and E₄ are selected from CRₑ.

According to an embodiment of the present disclosure, E₄ is selected from C and joined to L in Formula 1, and E₁ and E₃ are selected from CRₑ.

According to an embodiment of the present disclosure, Ar is, at each occurrence identically or differently, selected from substituted or unsubstituted aryl having 6 to 20 carbon atoms, substituted or unsubstituted heteroaryl having 3 to 20 carbon atoms or a combination thereof.

According to an embodiment of the present disclosure, Ar is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

According to an embodiment of the present disclosure, L is, at each occurrence identically or differently, selected from a single bond, substituted or unsubstituted arylene having 6 to 20 carbon atoms, substituted or unsubstituted heteroarylene having 3 to 20 carbon atoms or a combination thereof.

According to an embodiment of the present disclosure, L is, at each occurrence identically or differently, selected from a single bond, substituted or unsubstituted phenylene, substituted or unsubstituted biphenylylene, substituted or unsubstituted naphthylene, substituted or unsubstituted pyridylene or a combination thereof.

According to an embodiment of the present disclosure, L is, at each occurrence identically or differently, selected from a single bond or substituted or unsubstituted phenylene.

According to an embodiment of the present disclosure, Rₓ, R_{z}, Rᵤ, Rᵥ and Rₑ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group and combinations thereof.

According to an embodiment of the present disclosure, Rₓ, R_{z}, Rᵤ, Rᵥ and Rₑ are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

According to an embodiment of the present disclosure, R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, a cyano group and combinations thereof.

According to an embodiment of the present disclosure, R_{y} is, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl and combinations thereof.

According to an embodiment of the present disclosure, the compound is selected from the group consisting of Compound A-1 to Compound A-460, wherein the specific structures of Compound A-1 to Compound A-460 are referred to claim 9.

According to an embodiment of the present disclosure, hydrogens in Compound A-1 to Compound A-460 can be partially or fully substituted with deuterium.

According to an embodiment of the present disclosure, further disclosed is an organic electroluminescent device. The organic electroluminescent device comprises:
an anode,
a cathode, and
an organic layer disposed between the anode and the cathode, wherein the organic layer comprises a compound having a structure of Formula 1, wherein the compound having the structure of Formula 1 is any one of the preceding embodiments.

According to an embodiment of the present disclosure, the organic layer is a light-emitting layer, and the compound is a host compound.

According to an embodiment of the present disclosure, the organic layer is an electron transporting layer, and the compound is an electron transporting compound.

According to an embodiment of the present disclosure, the organic layer is a hole blocking layer, and the compound is a hole blocking compound.

According to an embodiment of the present disclosure, the organic layer is a light-emitting layer, and the compound is a host compound; the light-emitting layer comprises a first metal complex, wherein the first metal complex has a general formula of M(Lₐ)ₘ(L_{b})ₙ(L_{c})_{q};
the metal M is selected from a metal with a relative atomic mass greater than 40;
Lₐ, L_{b} and L, are a first ligand, a second ligand, and a third ligand coordinated to the metal M, respectively; the ligands Lₐ, L_{b} and L, may be identical or different;
the ligands Lₐ, L_{b} and L, can be optionally joined to form a multidentate ligand;
m is 1, 2 or 3, n is 0, 1 or 2, q is 0, 1 or 2, and m+n+q is equal to an oxidation state of the metal M; when m is greater than or equal to 2, a plurality of Lₐ may be identical or different; when n is 2, two L_{b} may be identical or different; when q is 2, two L, may be identical or different;
the ligand Lₐ has a structure represented by Formula 2:

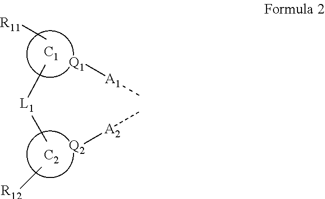

wherein the ring C₁ and the ring C₂ are, at each occurrence identically or differently, selected from an aromatic ring having 5 to 30 ring atoms, a heteroaromatic ring having 5 to 30 ring atoms or a combination thereof,
Q₁ and Q₂ are, at each occurrence identically or differently, selected from C or N;
A₁ and A₂ are, at each occurrence identically or differently, selected from a single bond, O or S;
L₁ is, at each occurrence identically or differently, selected from the group consisting of: a single bond, BR′, CR′R′, NR′, O, SiR′R′, PR′, S, GeR′R′, Se, substituted or unsubstituted vinylene, ethynylene, substituted or unsubstituted arylene having 6 to 30 carbon atoms, substituted or unsubstituted heteroarylene having 5 to 30 carbon atoms and combinations thereof, when two R′ are present at the same time, the two R′ are identical or different;
R₁₁ and R₁₂ represent, at each occurrence identically or differently, mono-substitution, multiple substitutions or non-substitution;
R′, R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
adjacent substituents R′, R₁₁ and R₁₂ can be optionally joined to form a ring; and
the ligands L_{b} and L_{c} are, at each occurrence identically or differently, selected from a monoanionic bidentate ligand.

In this embodiment, the expression that “adjacent substituents R′, R₁₁ and R₁₂ can be optionally joined to form a ring” is intended to mean that any one or more of groups of adjacent substituents, such as adjacent substituents R′, adjacent substituents Rn, adjacent substituents R₁₂, and adjacent substituents R₁₁ and R₁₂, can be joined to form a ring. Obviously, it is also possible that these substituents are not joined to form a ring.

According to an embodiment of the present disclosure, the ligands L_{b} and L_{c} are, at each occurrence identically or differently, selected from the group consisting of the following structures:

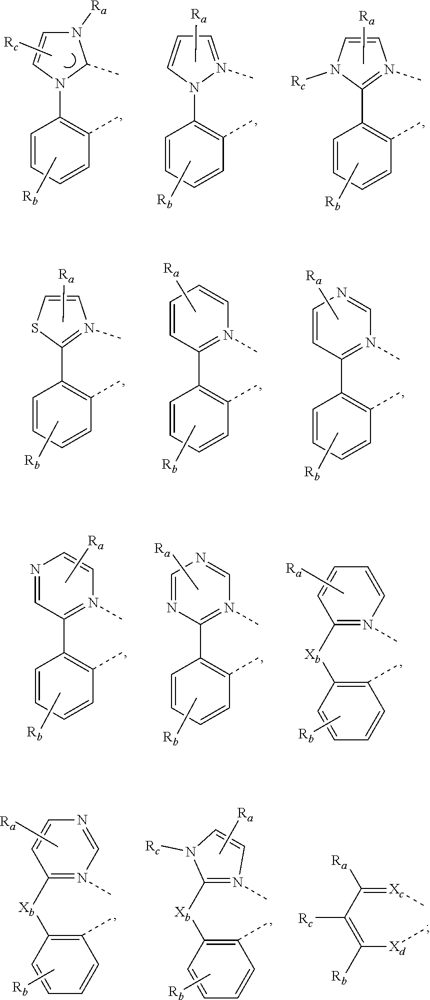

wherein
Rₐ, R_{b} and R_{c} represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
X, and X_{d} are, at each occurrence identically or differently, selected from the group consisting of: O, S, Se and NR_{N2},
Rₐ, R_{b}, Rₑ, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents Rₐ, R_{b}, Rₑ, R_{N1}, R_{N2}, R_{C1} and R_{C2} can be optionally joined to form a ring.

In this embodiment, the expression that adjacent substituents Rₐ, R_{b}, Rₑ, R_{N1}, R_{N2}, R_{C1} and R_{C2} can be optionally joined to form a ring is intended to mean that any one or more of groups of adjacent substituents, such as two substituents Rₐ, two substituents R_{b}, two substituents Rₑ, substituents Rₐ and R_{b}, substituents Rₐ and Rₑ, substituents R_{b} and Rₑ, substituents Rₐ and R_{N1}, substituents R_{b} and R_{N1}, substituents Rₐ and R_{C1}, substituents Rₐ and R_{C2}, substituents R_{b} and R_{C1}, substituents R_{b} and R_{C2}, substituents Rₐ and R_{N2}, substituents R_{b} and R_{N2}, and substituents R_{C1} and R_{C2}, can be joined to form a ring. For example, adjacent substituents Rₐ and R_{b} in

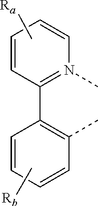

can be optionally joined to form a ring, which can form one or more of structures including but not limited to the following structures:

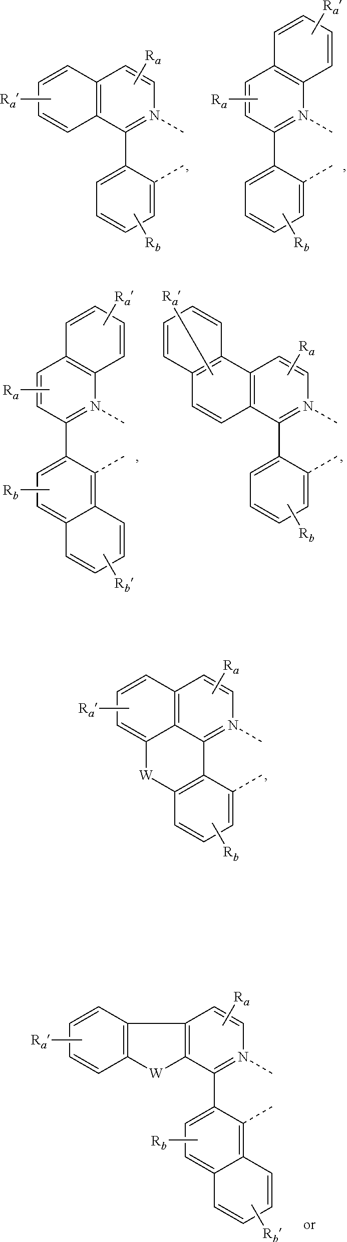

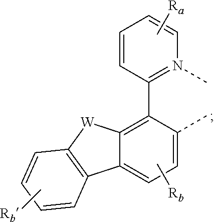

wherein W is selected from O, S, Se, NR_{w} or CR_{w}R_{w}, wherein the definition of each of R_{w}, Rₐ′ and R_{b}′ is the same as that of Rₐ. Obviously, it is also possible that these substituents are not joined to form a ring.

According to an embodiment of the present disclosure, the first metal complex has a general formula of Ir(Lₐ)ₘ(L_{b})₃₋ₘ and has a structure represented by Formula 2-1:

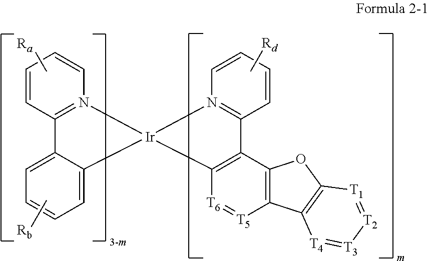

wherein
m is 0, 1, 2 or 3; when m is 2 or 3, a plurality of Lₐ are identical or different; when m is 0 or 1, a plurality of L_{b} are identical or different;
T₁ to T₆ are, at each occurrence identically or differently, selected from CR_{T} or N;
Rₐ, R_{b} and R_{d} represent, at each occurrence identically or differently, mono-substitution, multiple substitutions or non-substitution;
Rₐ, R_{b}, R_{d} and R_{T} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
adjacent substituents Rₐ and R_{b} can be optionally joined to form a ring; and
adjacent substituents R_{d} and R_{T} can be optionally joined to form a ring.

In this embodiment, the expression that “adjacent substituents Rₐ and R_{b} can be optionally joined to form a ring” is intended to mean that any one or more of groups of adjacent substituents, such as two substituents Rₐ, two substituents R_{b}, and substituents Rₐ and R_{b}, can be joined to form a ring. Obviously, it is also possible that these substituents are not joined to form a ring.

In this embodiment, the expression that “adjacent substituents R_{d} and R_{T} can be optionally joined to form a ring” is intended to mean that any one or more of groups of adjacent substituents, such as two substituents R_{T}, and two substituents R_{d}, can be joined to form a ring. Obviously, it is also possible that these substituents are not joined to form a ring.

According to an embodiment of the present disclosure, at least one of T₁ to T₆ is selected from CR_{T}, and R_{T} is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, at least one of T₁ to T₆ is selected from CR_{T}, and R_{T} is selected from fluorine or a cyano group.

According to an embodiment of the present disclosure, at least two of T₁ to T₆ are selected from CR_{T}, wherein one R_{T} is selected from fluorine or a cyano group, and another R_{T} is selected from substituted or unsubstituted alkyl having 1 to 20 carbon atoms, substituted or unsubstituted cycloalkyl having 3 to 20 ring carbon atoms, substituted or unsubstituted aryl having 6 to 30 carbon atoms or substituted or unsubstituted heteroaryl having 3 to 30 carbon atoms.

According to an embodiment of the present disclosure, T₁ to T₆ are, at each occurrence identically or differently, selected from CR_{T}.

According to an embodiment of the present disclosure, T₁ to T₆ are, at each occurrence identically or differently, selected from CR_{T} or N, and at least one of T₁ to T₆ is selected from N; for example, one of T₁ to T₆ is selected from N or two of T₁ to T₆ are selected from N.

According to an embodiment of the present disclosure, the first metal complex is selected from the group consisting of Compound GD1 to Compound GD76, wherein the specific structures of Compound GD1 to Compound GD76 are referred to claim 13.

According to an embodiment of the present disclosure, hydrogens in Compound GD1 to Compound GD76 can be partially or fully substituted with deuterium.

According to an embodiment of the present disclosure, the organic layer is the light-emitting layer, the compound is the host compound, and the light-emitting layer further comprises a second host compound, wherein the second host compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof.

According to an embodiment of the present disclosure, the second host compound comprises at least one chemical group selected from the group consisting of: benzene, carbazole, indolocarbazole, fluorene, silafluorene and combinations thereof.

According to an embodiment of the present disclosure, the second host compound has a structure represented by Formula 3 or Formula 4:

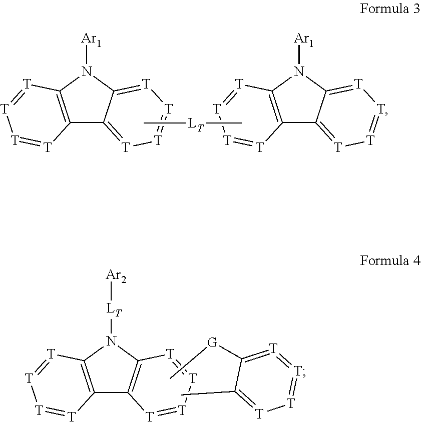

wherein
G is, at each occurrence identically or differently, selected from C(R_{g})₂, NR_{g}, O or S;
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof,
T is, at each occurrence identically or differently, selected from C, CRₜ or N;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof,
Ar₁ and Ar₂ are, at each occurrence identically or differently, selected from substituted or unsubstituted aryl having 6 to 30 carbon atoms, substituted or unsubstituted heteroaryl having 3 to 30 carbon atoms and combinations thereof, and
adjacent substituents Rₜ and R_{g} can be optionally joined to form a ring.

Herein, the expression that “adjacent substituents Rₜ and R_{g} can be optionally joined to form a ring” is intended to mean that any one or more of groups of adjacent substituents, such as two substituents Rₜ, two substituents R_{g}, and substituents Rₜ and R_{g}, can be joined to form a ring. Obviously, it is also possible that these substituents are not joined to form a ring.

According to an embodiment of the present disclosure, the second host compound has a structure represented by one of Formula 3-a to Formula 3-j and Formula 4-a to Formula 4-f:

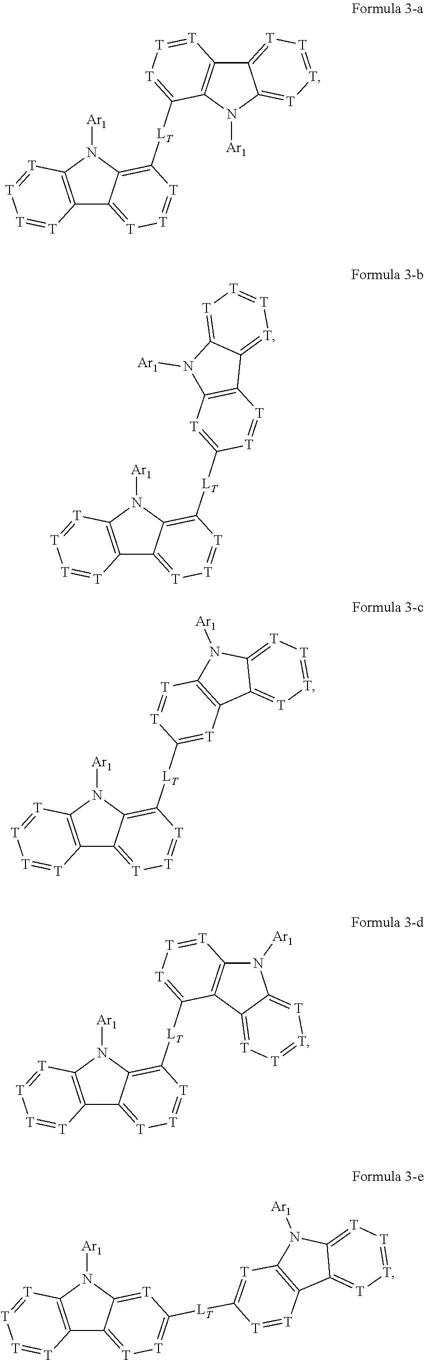

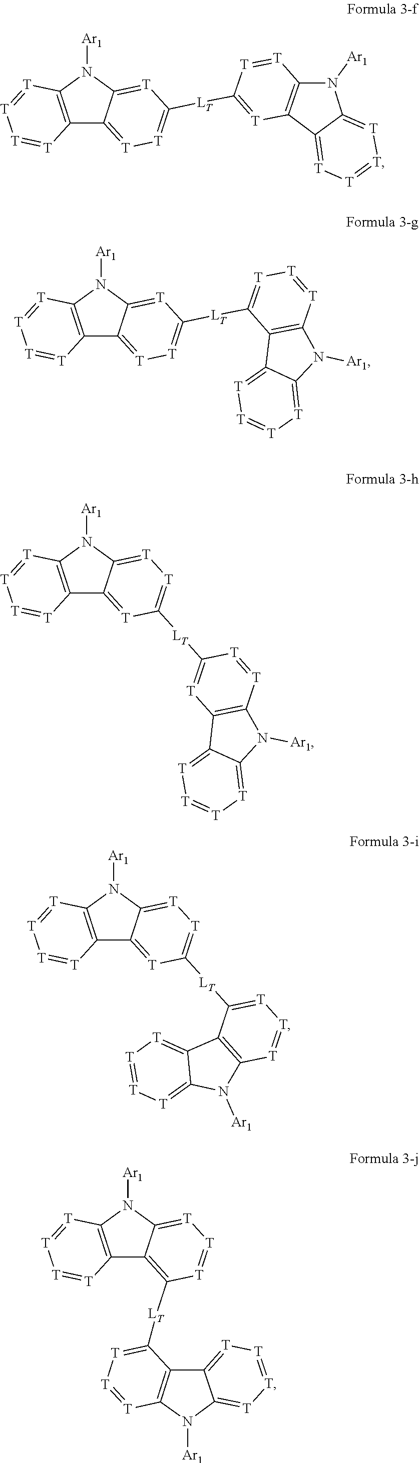

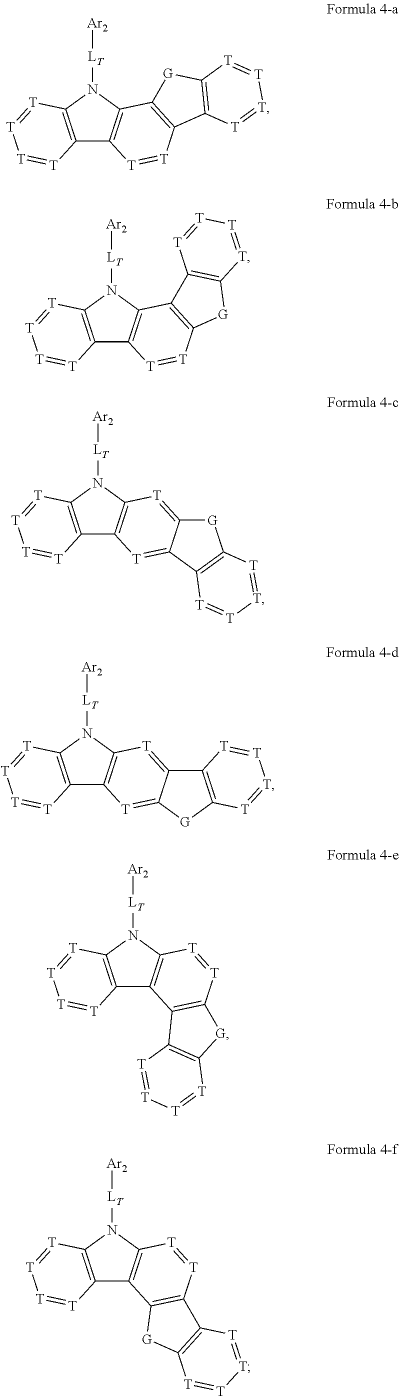

wherein
L_{T} is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof,
T is, at each occurrence identically or differently, selected from CRₜ or N;
G is, at each occurrence identically or differently, selected from C(R_{g})₂, NR_{g}, O or S;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms or a combination thereof,
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof, and
adjacent substituents Rₜ and R_{g} can be optionally joined to form a ring.

According to an embodiment of the present disclosure, in Formula 3-a to Formula 3-j and Formula 4-a to Formula 4-f, T is, at each occurrence identically or differently, selected from CRₜ.

According to an embodiment of the present disclosure, in Formula 3-a to Formula 3-j and Formula 4-a to Formula 4-f, T is, at each occurrence identically or differently, selected from CRₜ or N, and at least one of T is selected from N, for example, one of T is selected from N or two of T are selected from N.

According to an embodiment of the present disclosure, the second host compound is selected from the group consisting of Compound PH-1 to Compound PH-50, wherein the specific structures of Compound PH-1 to Compound PH-50 are referred to claim 16.

According to an embodiment of the present disclosure, the second host compound is selected from the group consisting of Compound PH-1 to Compound PH-106, wherein the specific structures of Compound PH-1 to Compound PH-50 are referred to claim 16, and the specific structures of Compound PH-51 to Compound PH-106 are as follows:

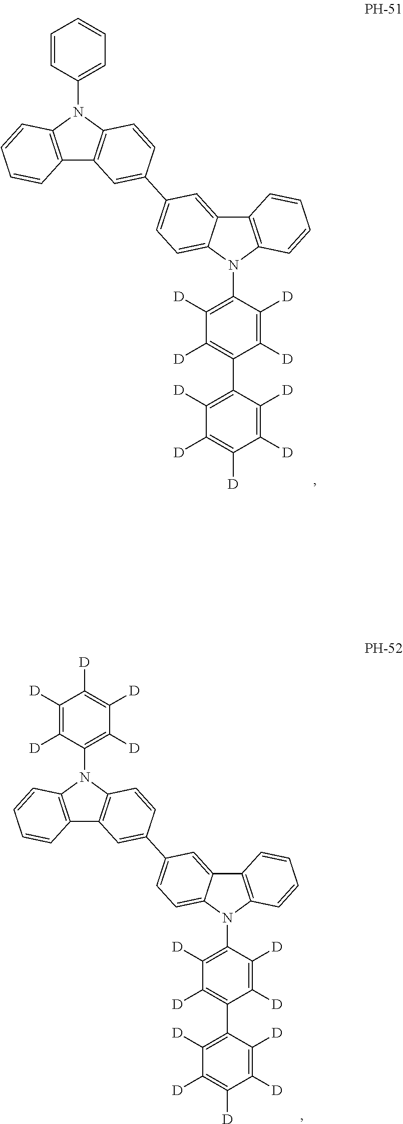 [SMILES: [2H]c1c([2H])c([2H])c(-c2c([2H])c([2H])c(-n3c4ccccc4c4cc(-c5ccc6c(c5)c5ccccc5n6-c5ccccc5)ccc43)c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c([2H])c([2H])c5[2H])ccc43)c([2H])c2[2H])c([2H])c1[2H]]

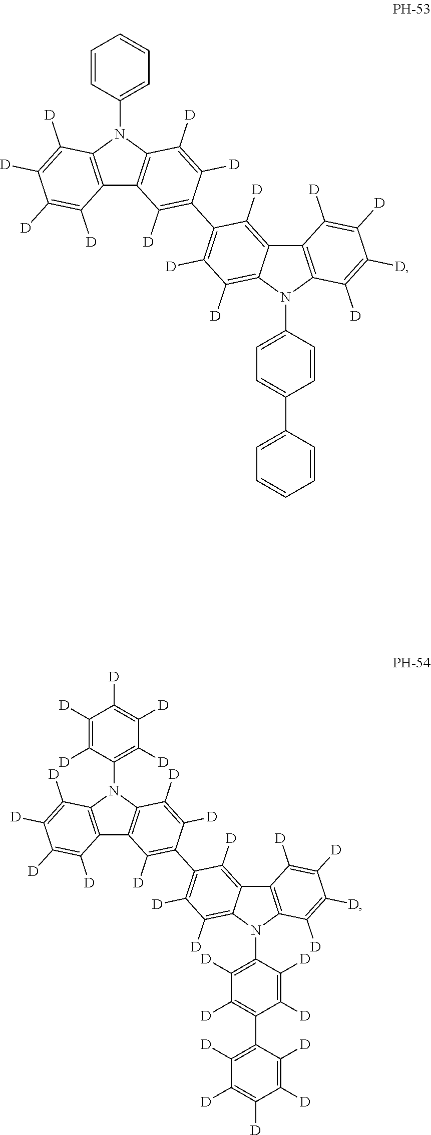

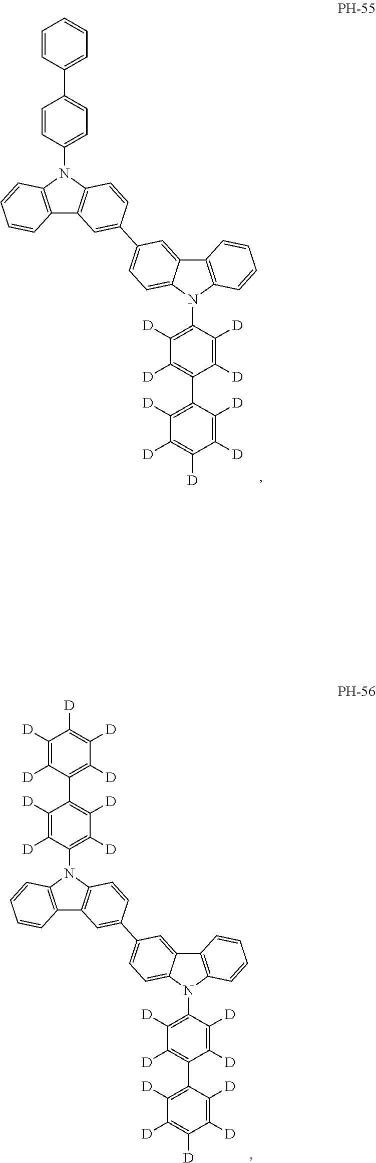 [SMILES: [2H]c1c([2H])c([2H])c(-c2c([2H])c([2H])c(-n3c4ccccc4c4cc(-c5ccc6c(c5)c5ccccc5n6-c5ccc(-c6ccccc6)cc5)ccc43)c([2H])c2[2H])c([2H])c1[2H] ; [2H]c1c([2H])c([2H])c(-c2c([2H])c([2H])c(-n3c4ccccc4c4cc(-c5ccc6c(c5)c5ccccc5n6-c5c([2H])c([2H])c(-c6c([2H])c([2H])c([2H])c([2H])c6[2H])c([2H])c5[2H])ccc43)c([2H])c2[2H])c([2H])c1[2H]]

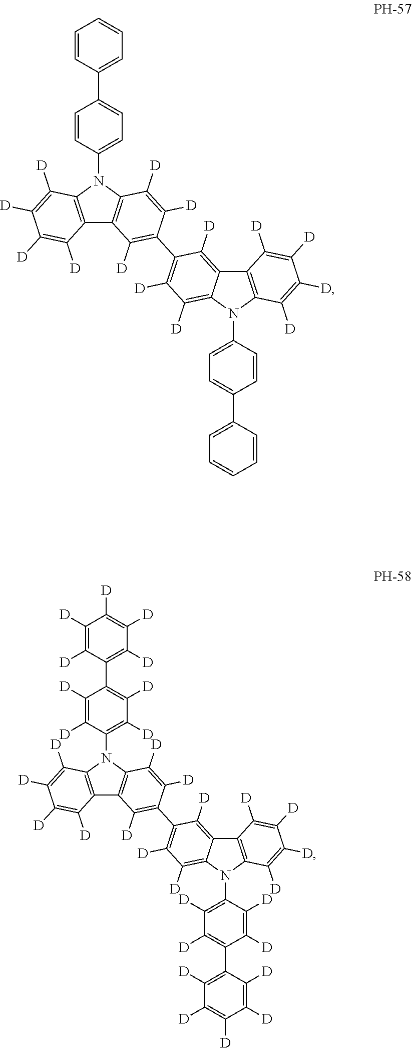

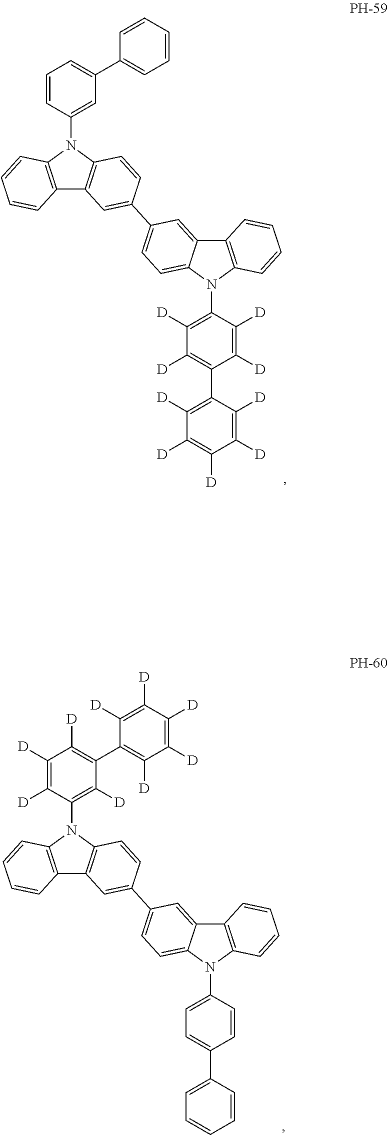 [SMILES: [2H]c1c([2H])c([2H])c(-c2c([2H])c([2H])c(-n3c4ccccc4c4cc(-c5ccc6c(c5)c5ccccc5n6-c5cccc(-c6ccccc6)c5)ccc43)c([2H])c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5ccc(-c6ccccc6)cc5)ccc43)c2[2H])c([2H])c1[2H]]

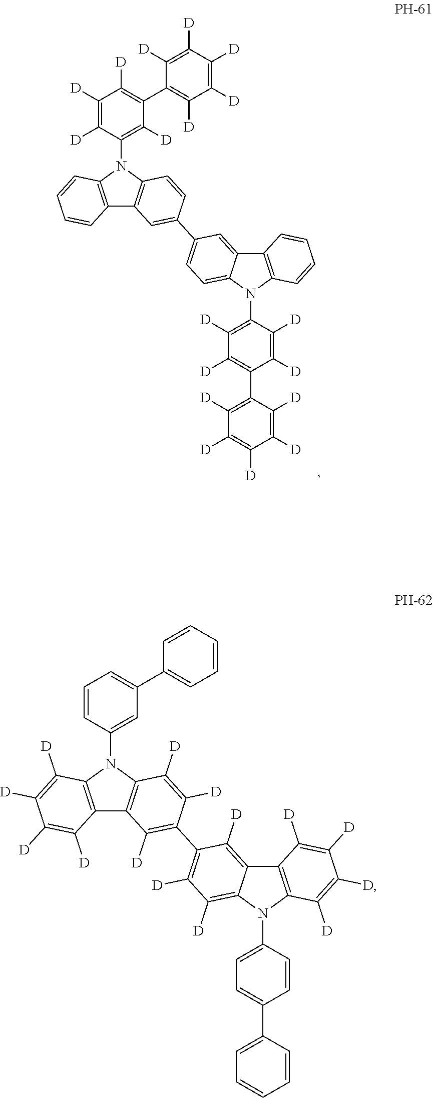

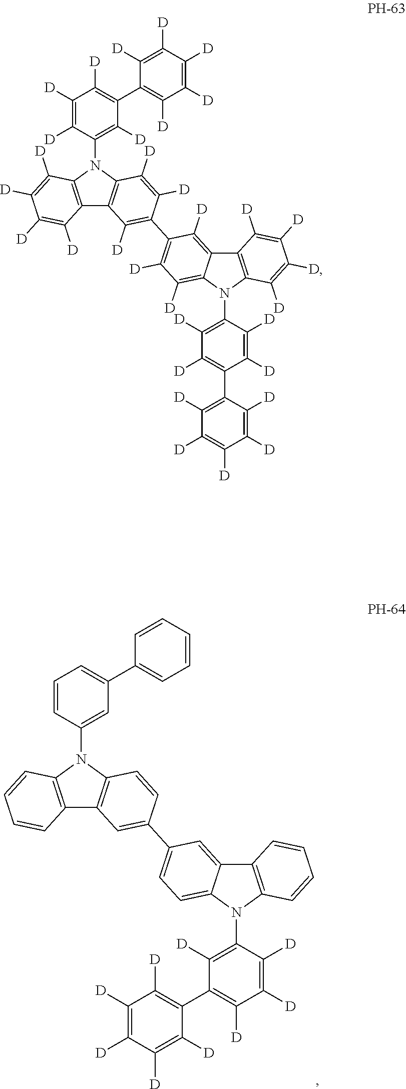

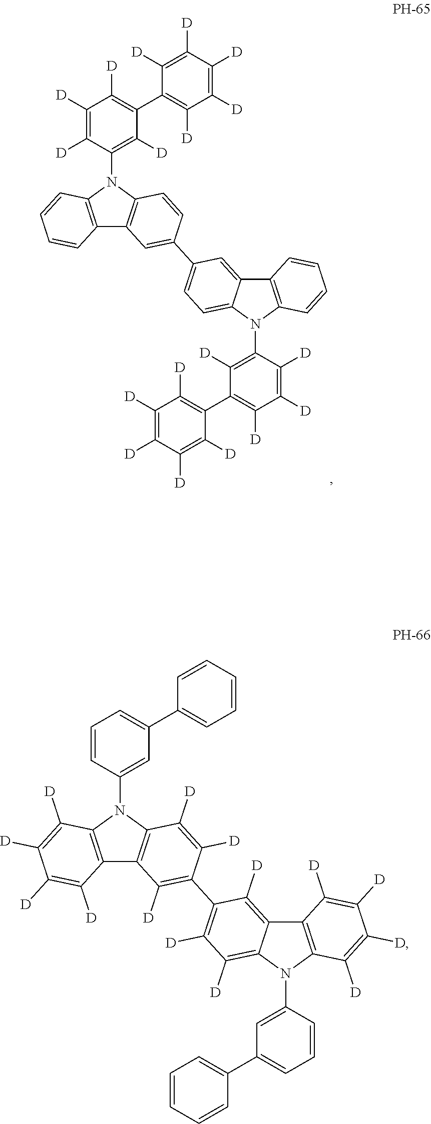

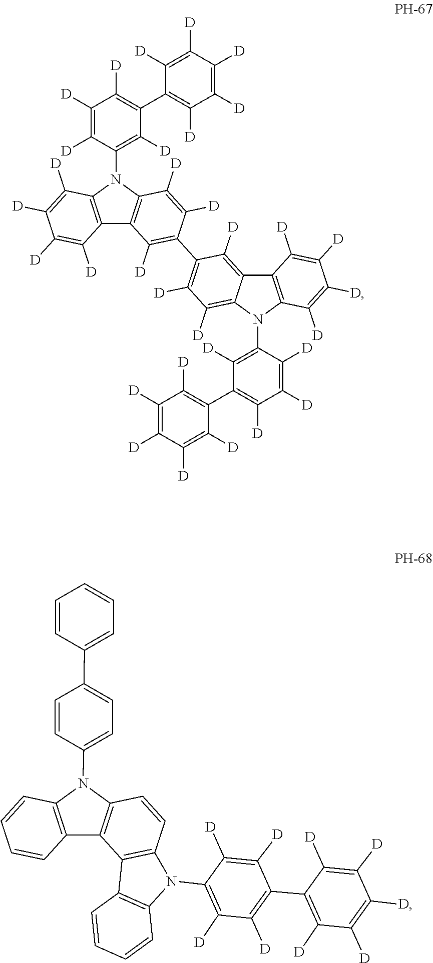

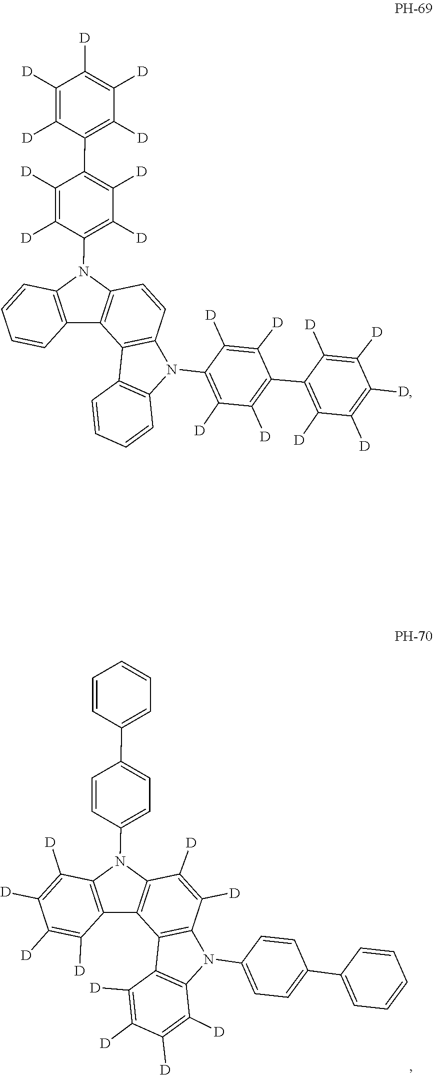

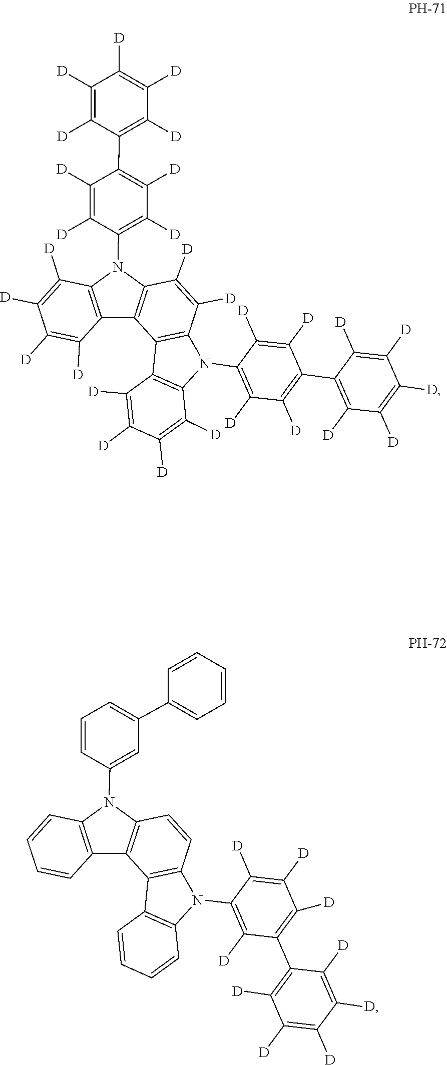

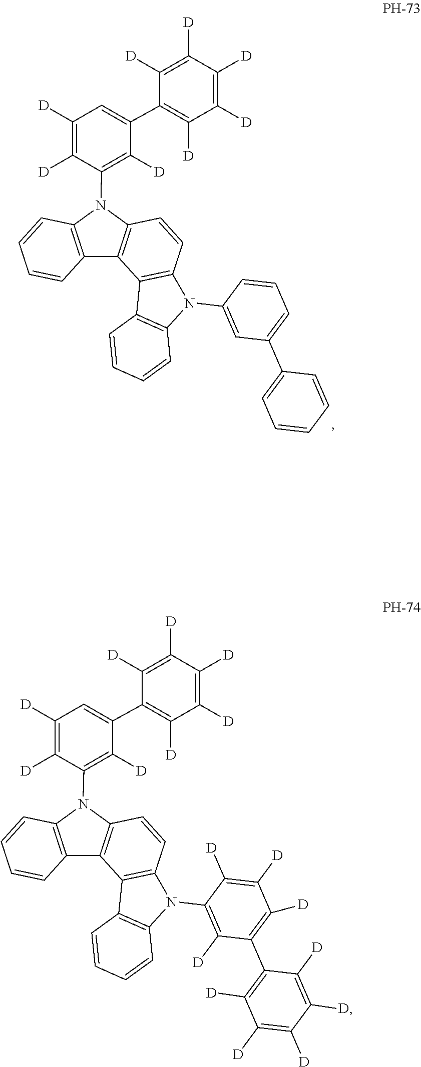

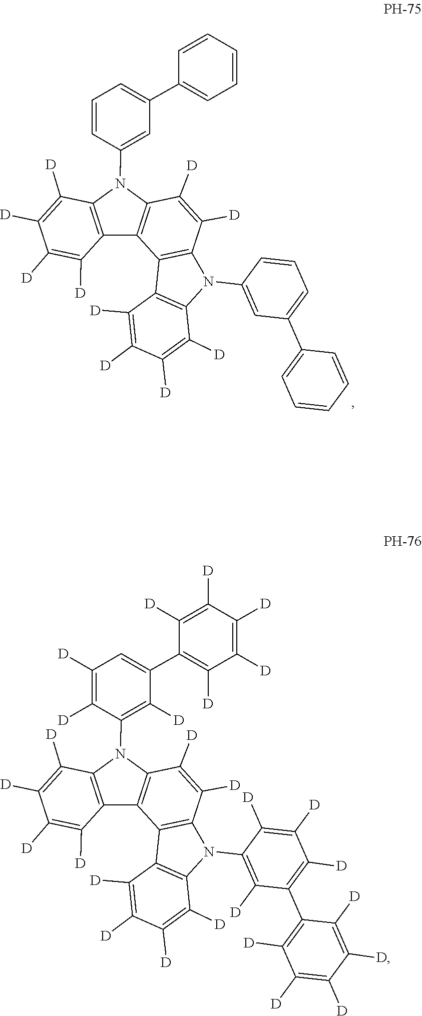

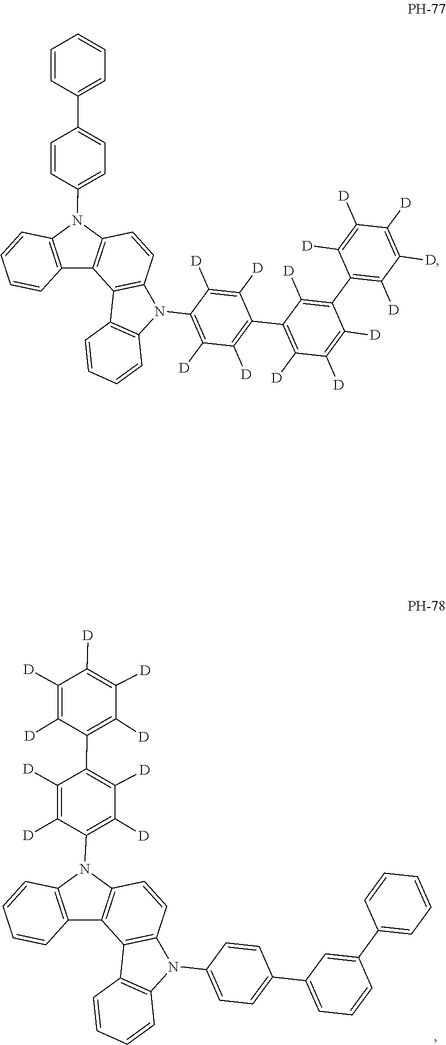

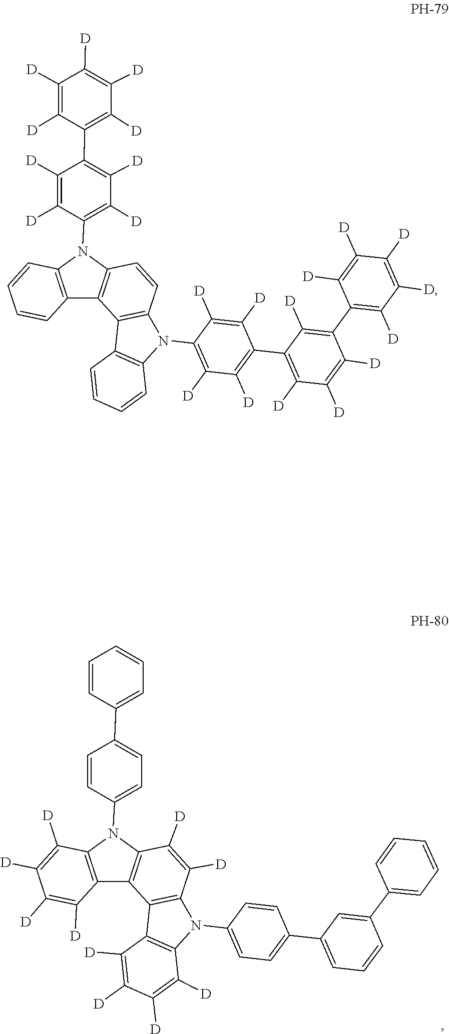

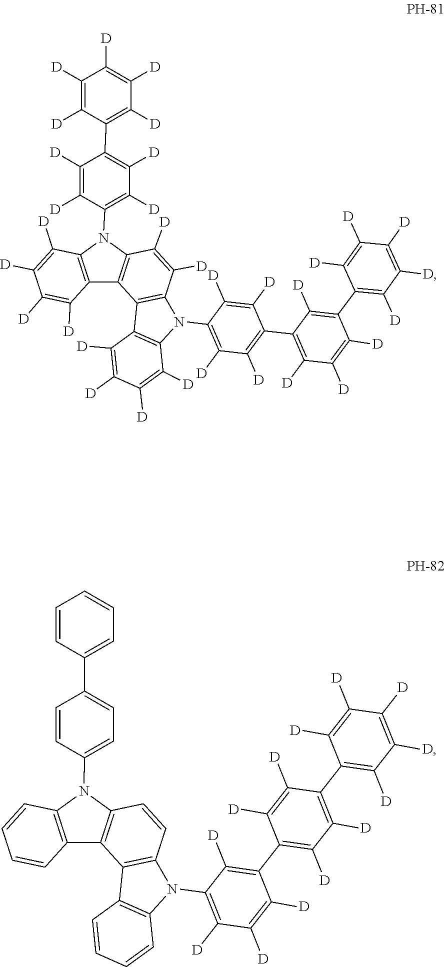

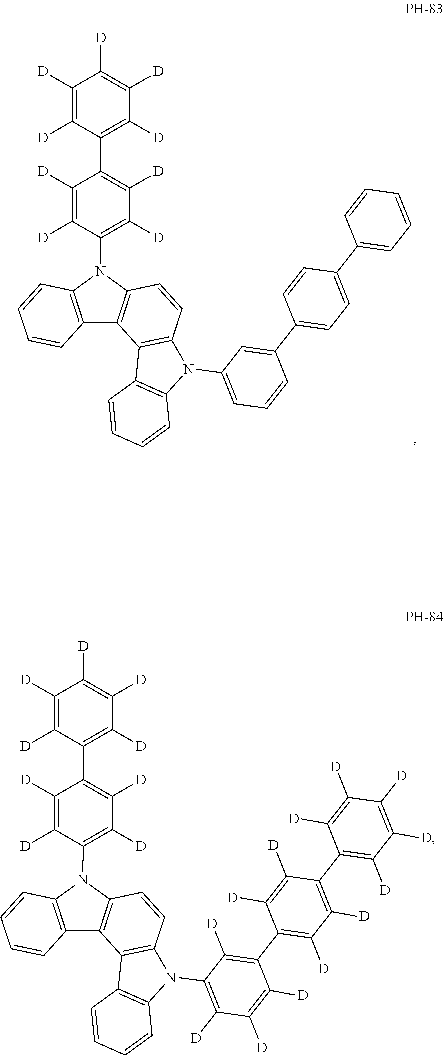

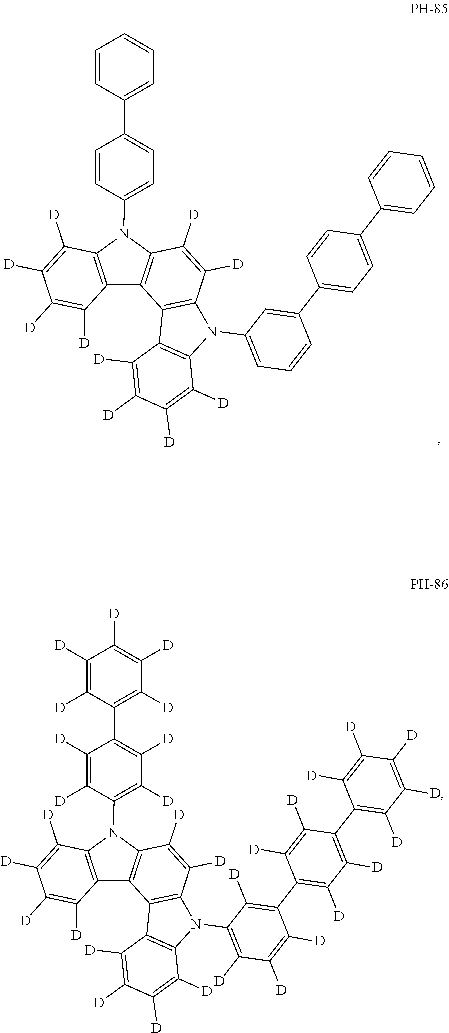

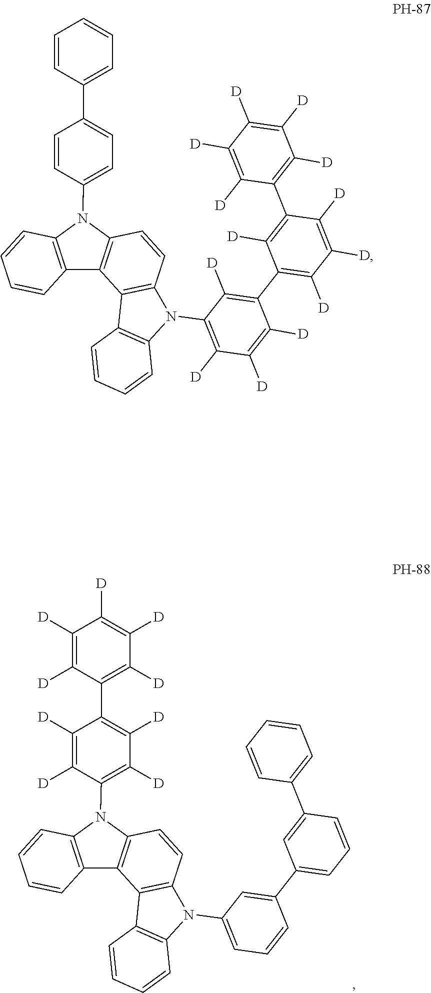

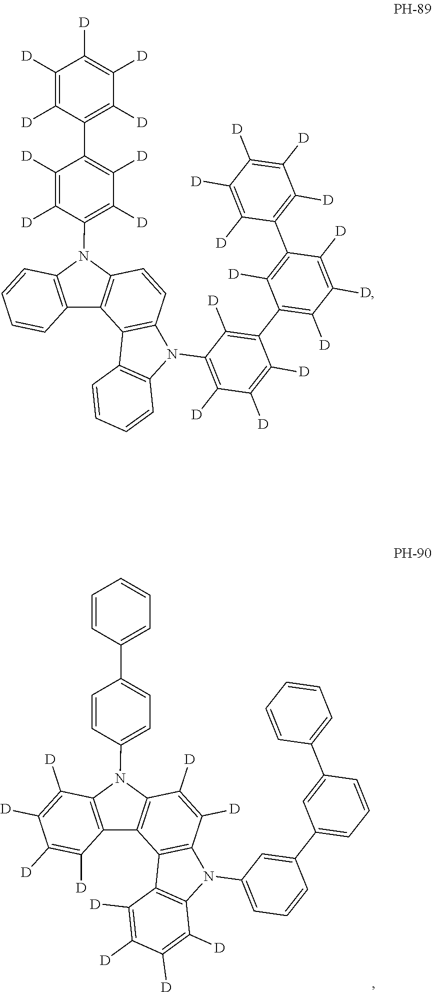

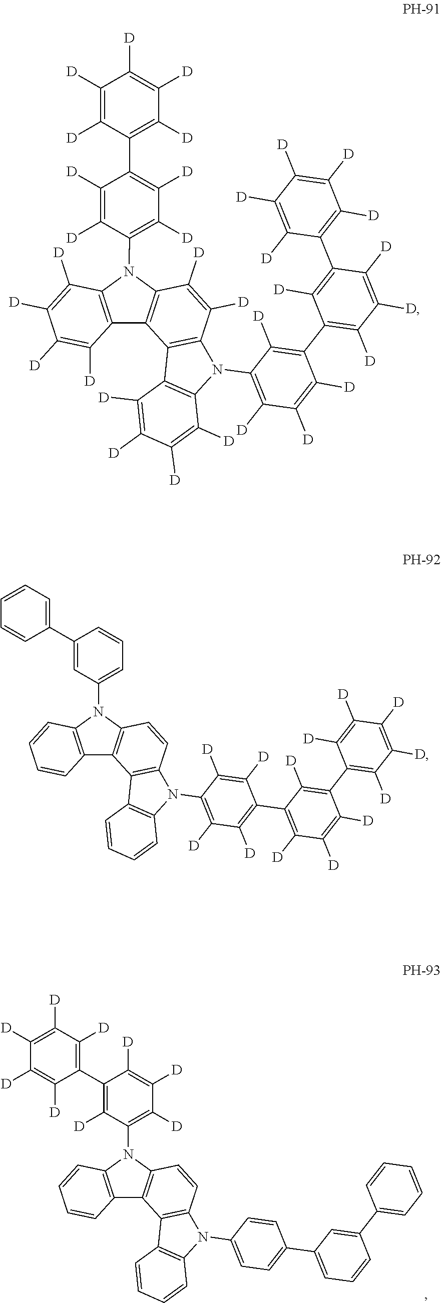

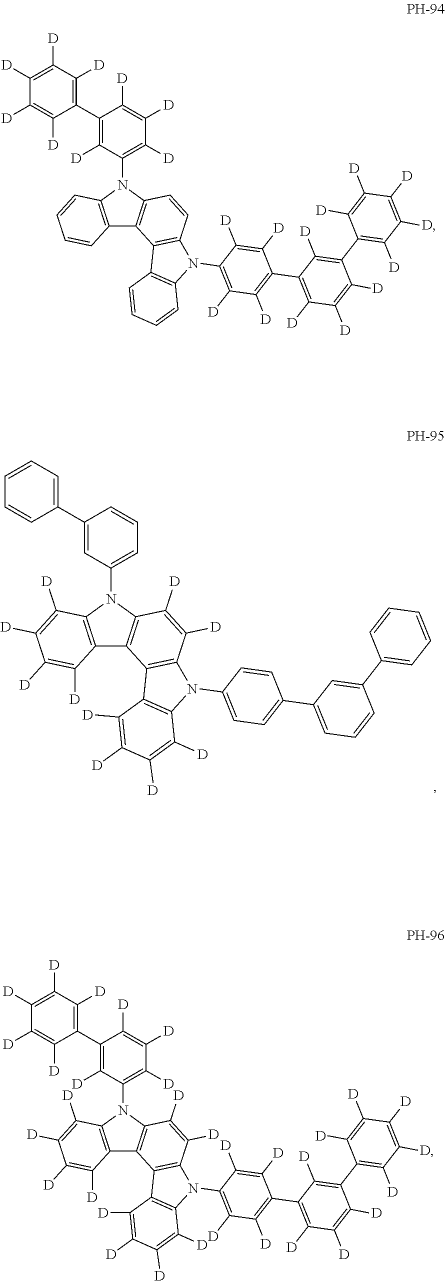

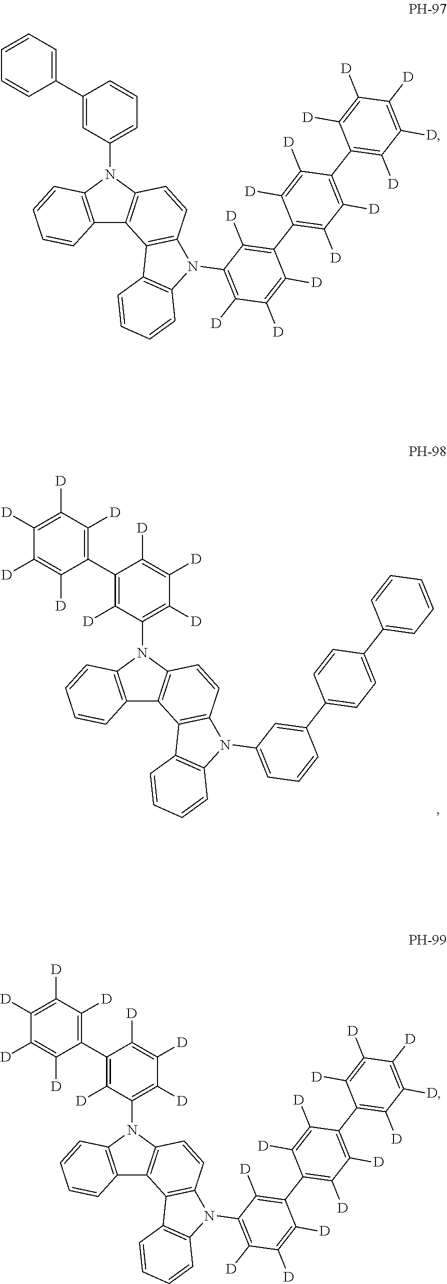

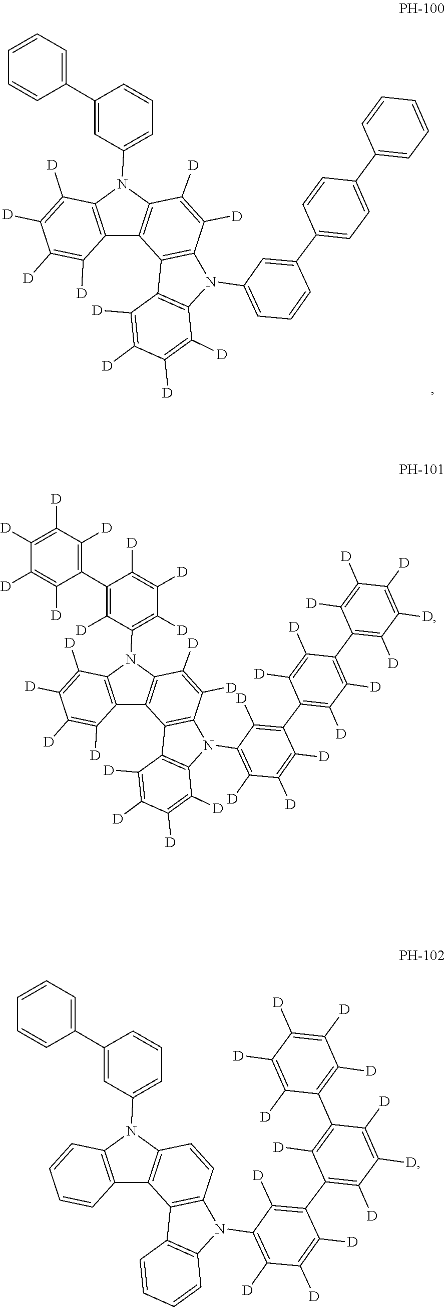

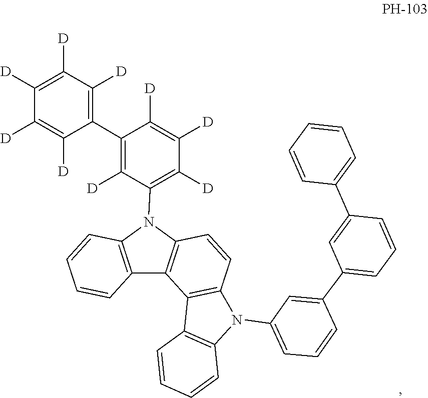 [SMILES: [2H]c1c([2H])c([2H])c(-c2c([2H])c([2H])c([2H])c(-n3c4ccccc4c4c5c6ccccc6n(-c6cccc(-c7cccc(-c8ccccc8)c7)c6)c5ccc43)c2[2H])c([2H])c1[2H]]

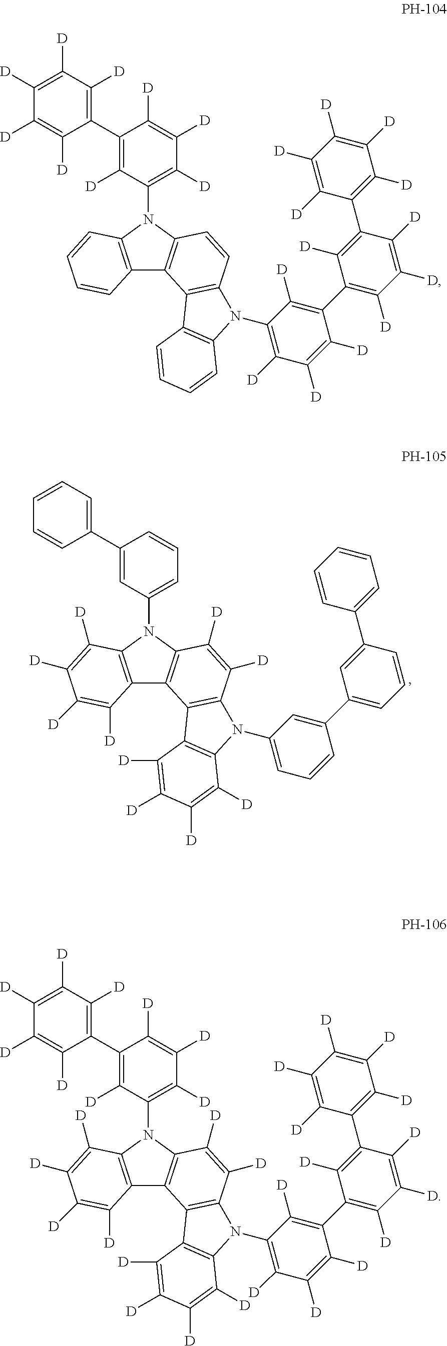

According to an embodiment of the present disclosure, the organic electroluminescent device emits green light.

According to an embodiment of the present disclosure, the organic electroluminescent device emits white light.

According to an embodiment of the present disclosure, further disclosed is a compound composition comprising a compound represented by Formula 1, wherein the compound is shown in any one of the preceding embodiments.

Combination with Other Materials

The materials described in the present disclosure for a particular layer in an organic light emitting device can be used in combination with various other materials present in the device. The combinations of these materials are described in more detail in U.S. Pat. App. No. 20160359122 at paragraphs 0132-0161, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

The materials described herein as useful for a particular layer in an organic light emitting device may be used in combination with a variety of other materials present in the device. For example, compounds disclosed herein may be used in combination with a wide variety of light-emitting dopants, hosts, transporting layers, blocking layers, injection layers, electrodes, and other layers that may be present. The combination of these materials is described in detail in paragraphs 0080-0101 of U.S. Pat. App. No. 20150349273, which is incorporated by reference herein in its entirety. The materials described or referred to the disclosure are non-limiting examples of materials that may be useful in combination with the compounds disclosed herein, and one of skill in the art can readily consult the literature to identify other materials that may be useful in combination.

In the embodiments of material synthesis, all reactions were performed under nitrogen protection unless otherwise stated. All reaction solvents were anhydrous and used as received from commercial sources. Synthetic products were structurally confirmed and tested for properties using one or more conventional equipment in the art (including, but not limited to, nuclear magnetic resonance instrument produced by BRUKER, liquid chromatograph produced by SHIMADZU, liquid chromatograph-mass spectrometry produced by SHIMADZU, gas chromatograph-mass spectrometry produced by SHIMADZU, differential Scanning calorimeters produced by SHIMADZU, fluorescence spectrophotometer produced by SHANGHAI LENGGUANG TECH., electrochemical workstation produced by WUHAN CORRTEST, and sublimation apparatus produced by ANHUI BEQ, etc.) by methods well known to the persons skilled in the art. In the embodiments of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well known to the persons skilled in the art. As the persons skilled in the art are aware of the above-mentioned equipment use, test methods and other related contents, the inherent data of the sample can be obtained with certainty and without influence, so the above related contents are not further described in this patent.

### Material Synthesis Example

The method for preparing a compound of the present disclosure is not limited herein. Typically, the following compounds are used as examples without limitation, and synthesis routes and preparation methods thereof are described below.

### Synthesis Example 1: Synthesis of Compound A-2

### Step 1: Synthesis of Intermediate C

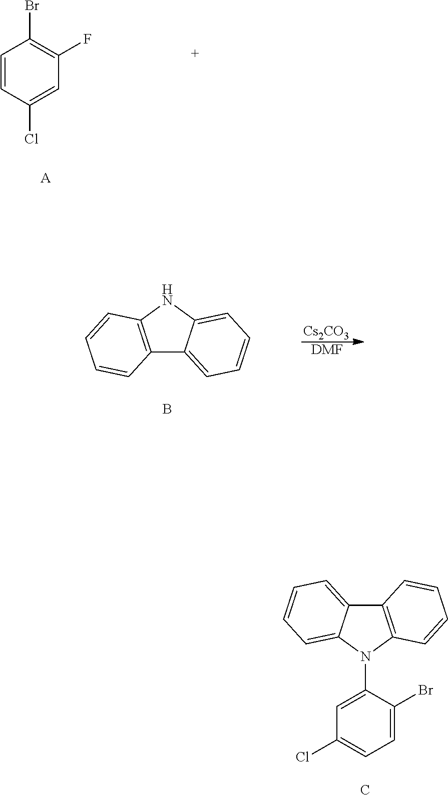

In a three-necked round-bottom flask, Intermediate A (22.2 g, 106.0 mmol), Intermediate B (17.7 g, 106.0 mmol), cesium carbonate (Cs₂CO₃) (69.1 g, 212.0 mmol) and 200 mL of N,N-dimethylformamide (DMF) were added in sequence. Under N₂ protection, the system was heated to reflux overnight. After the reaction was completed as confirmed by TLC, heating was stopped and the system was cooled to room temperature. The reaction solution was poured into a large amount of water and extracted with ethyl acetate. The organic phases were collected and concentrated under reduced pressure to obtain a crude product. The crude product was pulped with absolute ethanol to obtain Intermediate C (26.7 g, 74.9 mmol) as a white solid with a yield of 70.7%.

### Step 2: Synthesis of Intermediate E

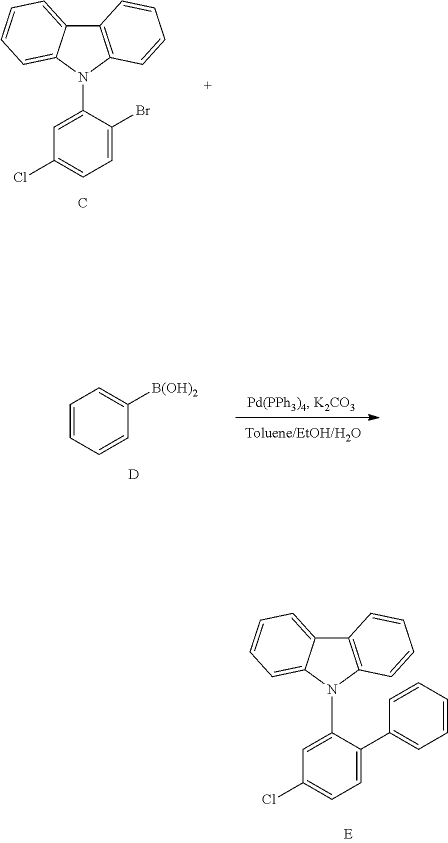

In a three-necked round-bottom flask, Intermediate C (18.9 g, 53.0 mmol), Intermediate D (9.5 g, 77.9 mmol), Pd(PPh₃)₄ (2.4 g, 2.1 mmol), K₂CO₃ (14.6 g, 106.0 mmol), 160 mL of toluene, 40 mL of EtOH and 40 mL of H₂O were added in sequence. Under N₂ protection, the system was heated to reflux overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through silica gel column chromatography (PE:DCM=10:1 to 8:1) to obtain Intermediate E (16.8 g, 47.5 mmol) as a white solid with a yield of 89.6%.

### Step 3: Synthesis of Intermediate F

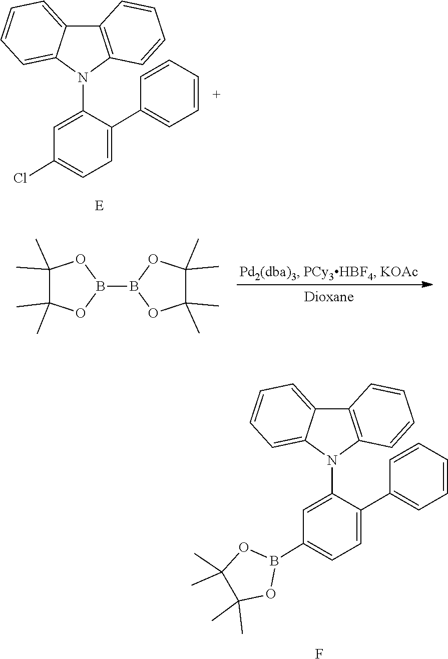

In a three-necked round-bottom flask, Intermediate E (16.8 g, 47.5 mmol), bis(pinacolato)diboron (18.1 g, 71.3 mmol), Pd₂(dba)₃ (0.87 g, 0.95 mmol), tricyclohexylphosphonium tetrafluoroborate (PCy₃·HBF₄) (0.87 g, 0.95 mmol), KOAc (9.3 g, 95.0 mmol) and 150 mL of 1,4-dioxane were added in sequence. Under N₂ protection, the system was heated to reflux overnight. After the reaction was completed as confirmed by TLC, heating was stopped and the system was cooled to room temperature. The reaction system was filtered through Celite, and the filtrate was concentrated under reduced pressure to obtain a crude product. The crude product was purified through silica gel column chromatography (PE:DCM=5:1 to 2:1) to obtain Intermediate F (18.0 g, 40.4 mmol) as a white solid with a yield of 85.0%.

### Step 4: Synthesis of Intermediate H

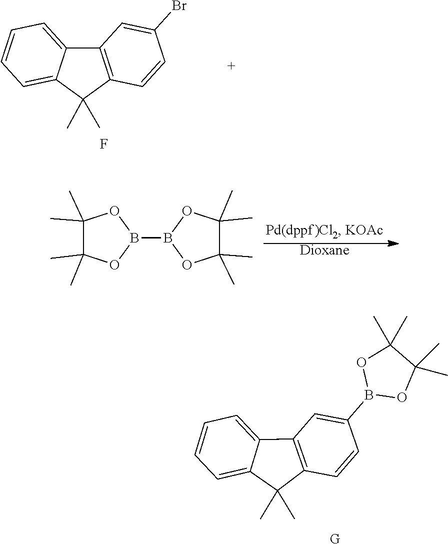

In a three-necked round-bottom flask, Intermediate G (27.3 g, 100.0 mmol), bis(pinacolato)diboron (33.0 g, 130 mmol), Pd(dppf)Cl₂ (1.46 g, 2.0 mmol) and KOAc (19.6 g, 200.0 mmol) were added to 1,4-dioxane (500 mL). Under N₂ protection, the system was heated to reflux overnight. After the reaction was completed as confirmed by TLC, heating was stopped and the system was cooled to room temperature. The reaction system was filtered through Celite, the filtrate was concentrated under reduced pressure, and the crude product was subjected to silica gel column chromatography (PE:DCM=10:1 to 1:1) to obtain Intermediate H (27.0 g, 84.3 mmol) as a white solid with a yield of 84.3%.

### Step 5: Synthesis of Intermediate J

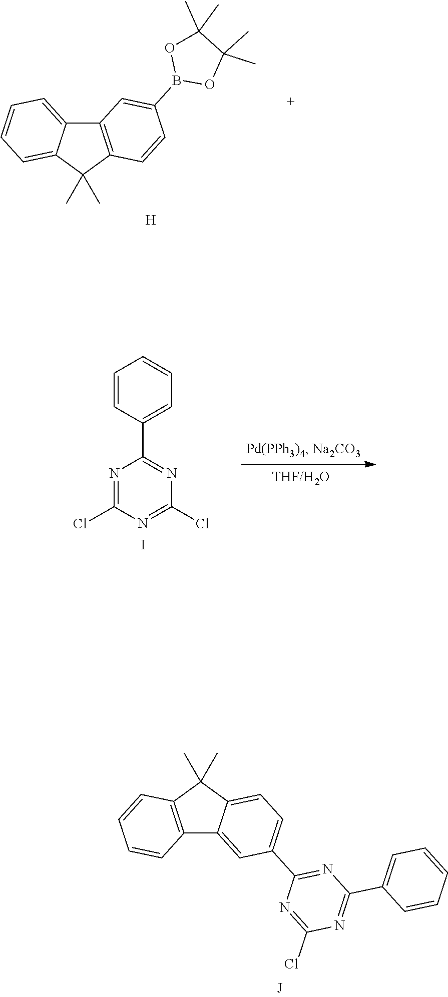

In a three-necked round-bottom flask, Intermediate H (6.4 g, 20.0 mmol), Intermediate I (6.78 g, 30.0 mmol), Pd(PPh₃)₄ (0.69 g, 0.60 mmol) and Na₂CO₃ (6.24 g, 40.0 mmol) were added to THE (96 mL) and H₂O (24 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=5:1 to 2:1) to obtain Intermediate J (3.9 g, 10.1 mmol) as a white solid with a yield of 50.5%.

### Step 6: Synthesis of Compound A-2

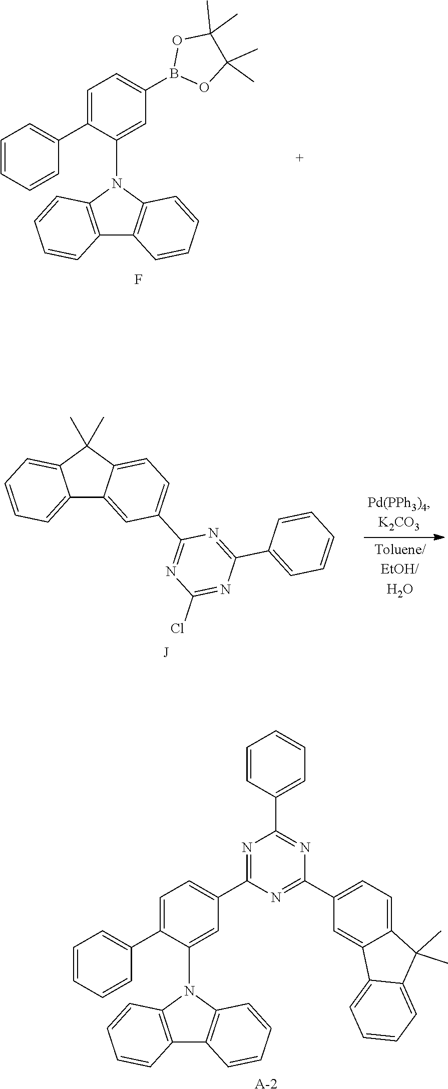

In a three-necked round-bottom flask, Intermediate F (3.56 g, 8.0 mmol), Intermediate J (3.07 g, 8.0 mmol), Pd(PPh₃)₄ (0.28 g, 0.24 mmol) and K₂CO₃ (2.21 g, 16.0 mmol) were added to toluene (40 mL), EtOH (10 mL) and H₂O (10 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=5:1 to 3:1) to obtain a white solid (4.2 g, 6.3 mmol) with a yield of 78.7%. The product was confirmed as the target product A-2 with a molecular weight of 666.3.

### Synthesis Example 2: Synthesis of Compound A-3

### Step 1: Synthesis of Intermediate L

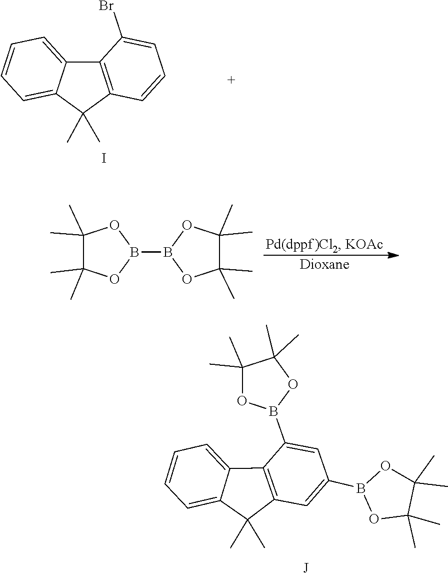

In a three-necked round-bottom flask, Intermediate K (27.3 g, 100.0 mmol), bis(pinacolato)diboron (50.8 g, 200.0 mmol), Pd(dppf)Cl₂ (1.46 g, 2.0 mmol), KOAc (19.6 g, 200.0 mmol) and 200 mL of 1,4-dioxane were added in sequence. Under N₂ protection, the system was heated to reflux overnight. After the reaction was completed as confirmed by TLC, heating was stopped and the system was cooled to room temperature. The reaction system was filtered through Celite, the filtrate was concentrated under reduced pressure, and the crude product was subjected to silica gel column chromatography (PE:DCM=5:1 to 2:1) to obtain Intermediate L (28.0 g, 87.4 mmol) as a white solid with a yield of 87.4%.

### Step 2: Synthesis of Intermediate M

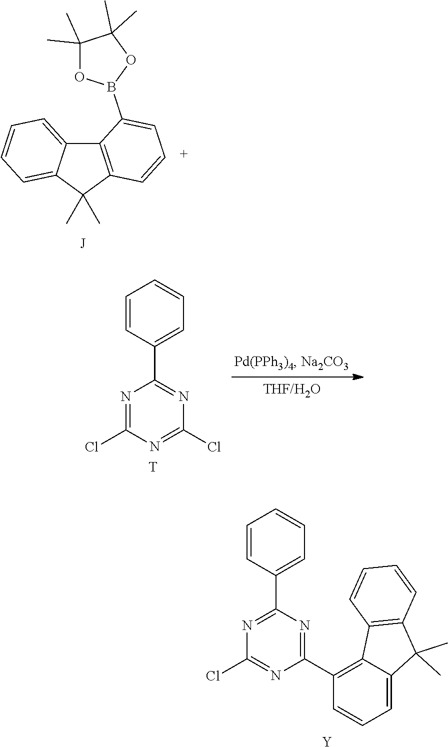

In a three-necked round-bottom flask, Intermediate L (10.0 g, 31.2 mmol), Intermediate I (8.5 g, 37.5 mmol), Pd(PPh₃)₄ (1.1 g, 0.98 mmol), Na₂CO₃ (6.6 g, 62.4 mmol), 240 mL of THF and 60 mL of H₂O were added in sequence. Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=8:1 to 4:1) to obtain Intermediate M (6.9 g, 18.0 mmol) as a white solid with a yield of 57.7%.

### Step 3: Synthesis of Compound A-3

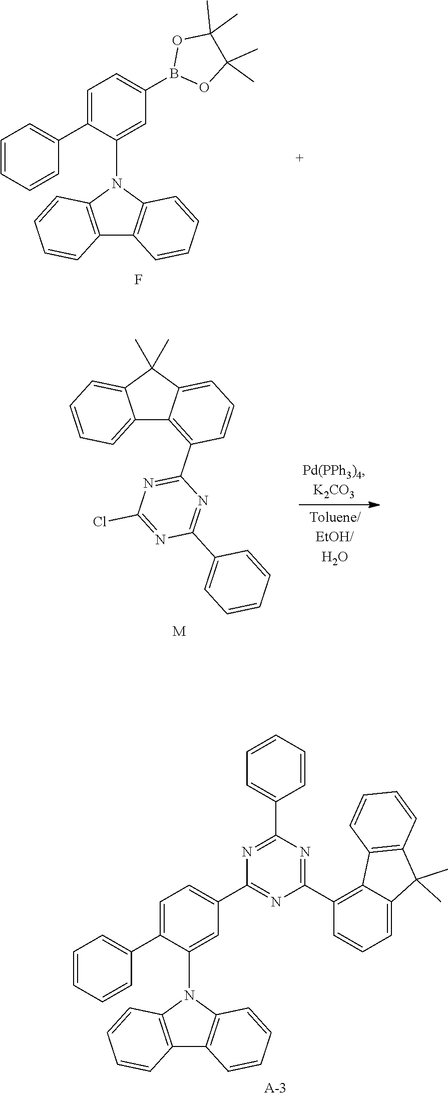

In a three-necked round-bottom flask, Intermediate F (3.50 g, 7.87 mmol), Intermediate M (3.02 g, 7.87 mmol), Pd(PPh₃)₄ (0.28 g, 0.24 mmol) and K₂CO₃ (2.18 g, 15.74 mmol) were added to toluene (40 mL), EtOH (10 mL) and H₂O (10 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=5:1 to 3:1) to obtain a white solid (4.2 g, 6.3 mmol) with a yield of 80.0%. The product was confirmed as the target product A-3 with a molecular weight of 666.3.

### Synthesis Example 3: Synthesis of Compound A-11

### Step 1: Synthesis of Intermediate O

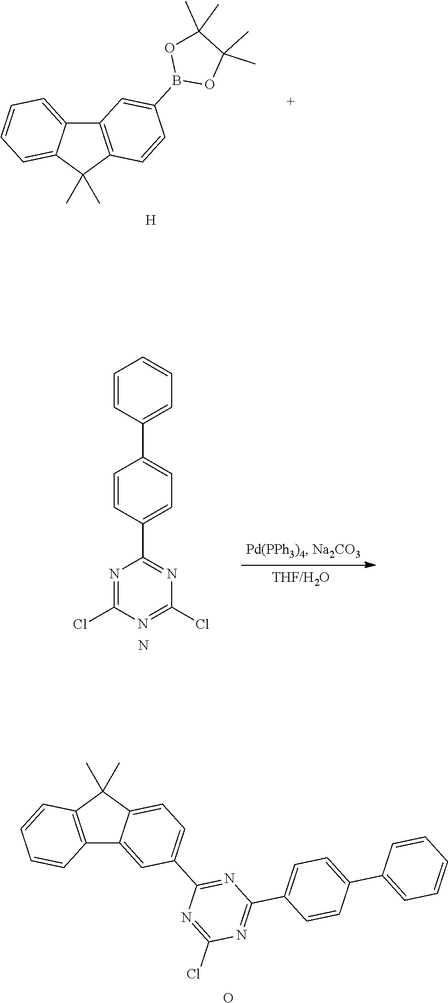

In a three-necked round-bottom flask, Intermediate H (9.6 g, 30.0 mmol), Intermediate N (11.8 g, 39.0 mmol), Pd(PPh₃)₄ (0.69 g, 0.60 mmol), Na₂CO₃ (6.36 g, 60.0 mmol), 240 mL of THE and 60 mL of H₂O were added in sequence. Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=8:1 to 3:1) to obtain Intermediate O (8.3 g, 18.0 mmol) as a white solid with a yield of 60.0%.

### Step 2: Synthesis of Intermediate Q

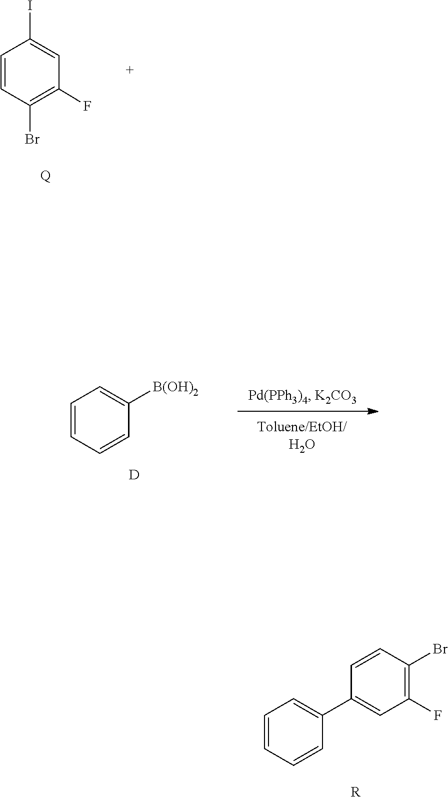

In a three-necked round-bottom flask, Intermediate P (30.0 g, 99.7 mmol), Intermediate D (13.4 g, 109.7 mmol), Pd(PPh₃)₄ (1.2 g, 1.0 mmol), K₂CO₃ (27.5 g, 199.4 mmol), 320 mL of toluene, 80 mL of EtOH and 80 mL of H₂O were added in sequence. Under N₂ protection, the system was heated to reflux overnight. After the reaction was completed as confirmed by TLC, heating was stopped and the system was cooled to room temperature. The reaction solution was separated into layers, the aqueous phase was extracted with DCM, and the organic phases were combined, dried over anhydrous Na₂SO₄, filtered, and concentrated under reduced pressure to obtain a crude product. The crude product was purified through column chromatography (PE) to obtain Intermediate Q (22.6 g, 90.0 mmol) as a white solid with a yield of 90.3%.

### Step 3: Synthesis of Intermediate R

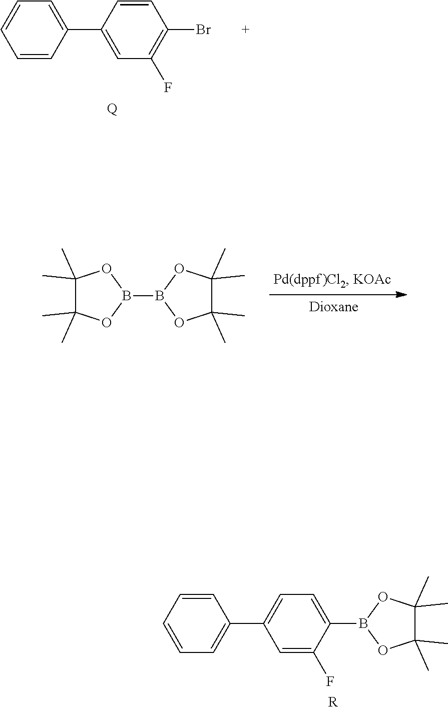

In a three-necked round-bottom flask, Intermediate Q (14.5 g, 57.7 mmol), bis(pinacolato)diboron (22.0 g, 86.6 mmol), Pd(dppf)Cl₂ (0.84 g, 1.15 mmol), KOAc (11.3 g, 115.4 mmol) and 300 mL of 1,4-dioxane were added in sequence. Under N₂ protection, the system was heated to reflux overnight. After the reaction was completed as confirmed by TLC, heating was stopped and the system was cooled to room temperature. The reaction system was filtered through Celite, the filtrate was concentrated under reduced pressure to obtain a crude product, and the crude product was purified through silica gel column chromatography (PE:DCM=10:1 to 2:1) to obtain Intermediate R (15.0 g, 50.3 mmol) as a white solid with a yield of 87.2%.

### Step 4: Synthesis of Intermediate S

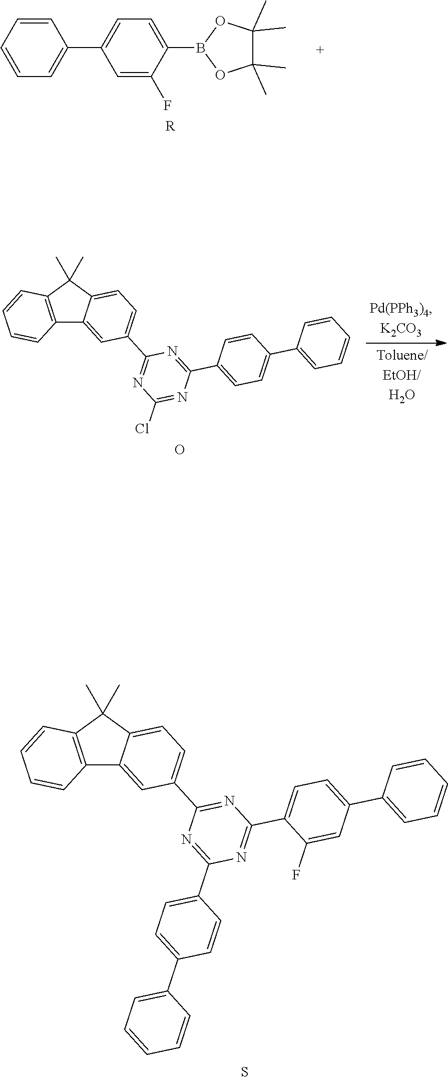

In a three-necked round-bottom flask, Intermediate R (5.4 g, 18.0 mmol), Intermediate O (8.3 g, 18.0 mmol), Pd(PPh₃)₄ (0.42 g, 0.36 mmol) and K₂CO₃ (5.0 g, 36.0 mmol) were added to toluene (60 mL), EtOH (15 mL) and H₂O (15 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=6:1 to 3:1) to obtain Intermediate S (10.4 g, 17.46 mmol) as a white solid with a yield of 97.0%.

### Step 5: Synthesis of Compound A-11

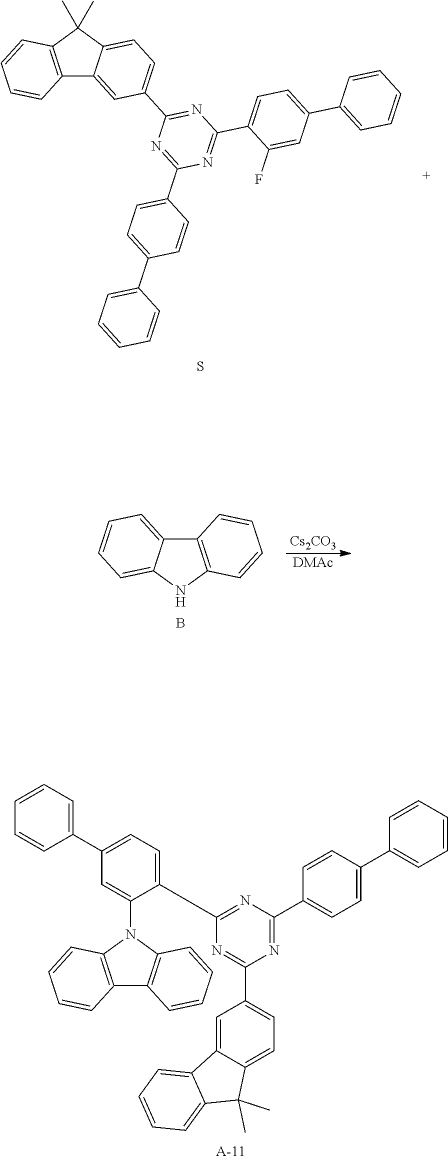

In a three-necked round-bottom flask, Intermediate S (5.5 g, 9.2 mmol), Intermediate B (1.7 g, 10.1 mmol), Cs₂CO₃ (6.0 g, 18.4 mmol) and 45 mL of N,N-dimethylacetamide (DMAc) were added in sequence. Under N₂ protection, the system was heated to reflux overnight. After the reaction was completed as confirmed by TLC, heating was stopped and the system was cooled to room temperature. The reaction solution was poured into a large amount of water to precipitate a solid and suction filtered under reduced pressure. The obtained solid was purified through silica gel column chromatography (PE:DCM=10:1 to 3:1) to obtain a light yellow solid (2.6 g, 2.5 mmol) with a yield of 38.0%. The product was confirmed as the target product A-11 with a molecular weight of 742.3.

### Synthesis Example 4: Synthesis of Compound A-12

### Step 1: Synthesis of Intermediate T

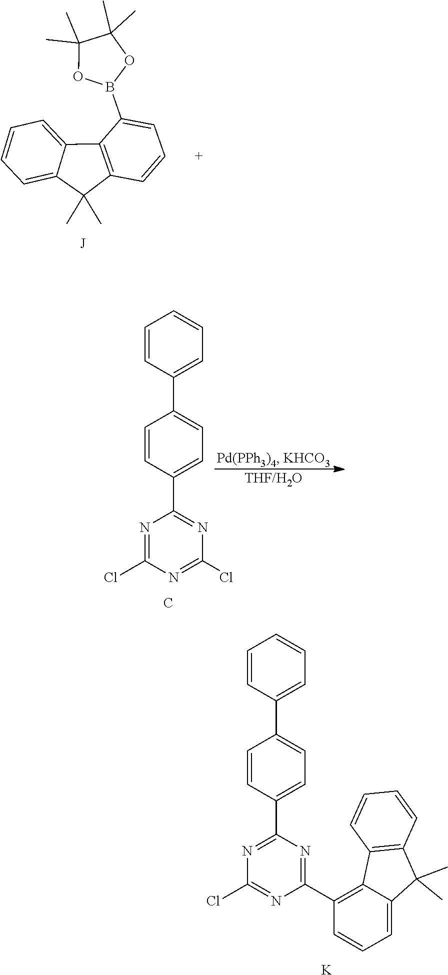

In a three-necked round-bottom flask, Intermediate L (9.6 g, 30.0 mmol), Intermediate N (13.6 g, 45.0 mmol), Pd(PPh₃)₄ (1.04 g, 0.90 mmol), Na₂CO₃ (6.36 g, 60.0 mmol), 200 mL of THE and 50 mL of H₂O were added in sequence. Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=8:1 to 3:1) to obtain Intermediate T (8.8 g, 19.13 mmol) as a white solid with a yield of 63.8%.

### Step 2: Synthesis of Intermediate U

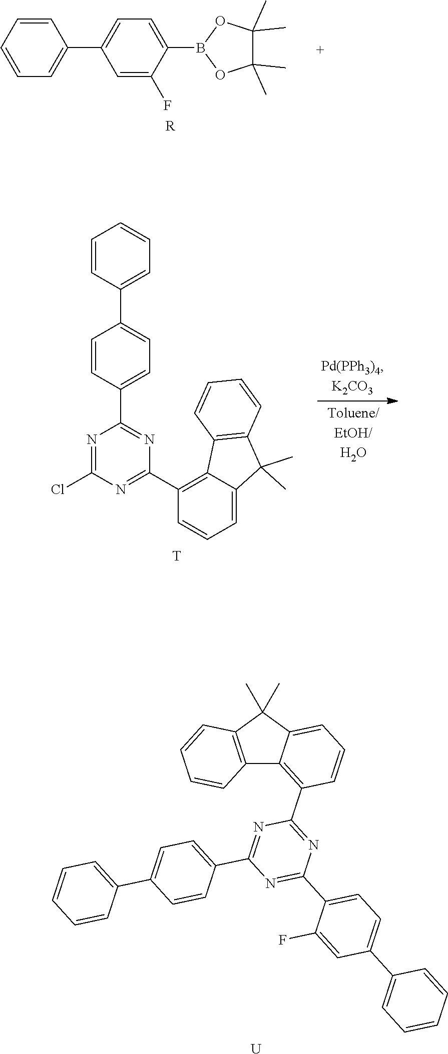

In a three-necked round-bottom flask, Intermediate R (3.63 g, 12.17 mmol), Intermediate T (5.6 g, 12.17 mmol), Pd(PPh₃)₄ (0.42 g, 0.36 mmol) and K₂CO₃ (3.36 g, 24.34 mmol) were added to toluene (48 mL), EtOH (12 mL) and H₂O (12 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=6:1 to 2:1) to obtain Intermediate U (6.6 g, 11.08 mmol) as a white solid with a yield of 91.0%.

### Step 3: Synthesis of Compound A-12

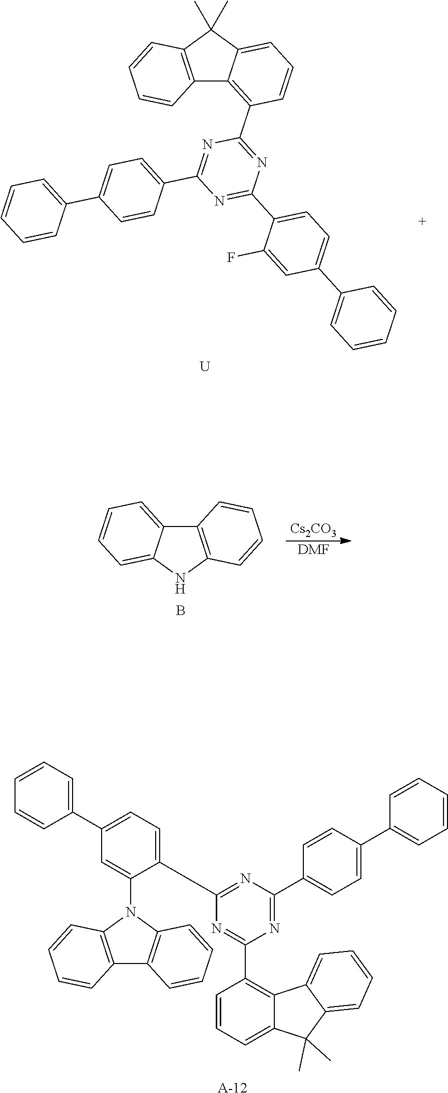

In a three-necked round-bottom flask, Intermediate U (4.2 g, 7.05 mmol), Intermediate B (1.3 g, 7.76 mmol), Cs₂CO₃ (4.6 g, 14.1 mmol) and 50 mL of DMF were added in sequence. Under N₂ protection, the system was heated to reflux overnight. After the reaction was completed as confirmed by TLC, heating was stopped and the system was cooled to room temperature. The reaction solution was poured into a large amount of water to precipitate a solid and suction filtered under reduced pressure. The obtained solid was purified through silica gel column chromatography (PE:DCM=6:1 to 2:1) to obtain a yellow solid (4.5 g, 6.06 mmol) with a yield of 85.9%. The product was confirmed as the target product A-12 with a molecular weight of 742.3.

### Synthesis Example 5: Synthesis of Compound A-301

### Step 1: Synthesis of Intermediate V

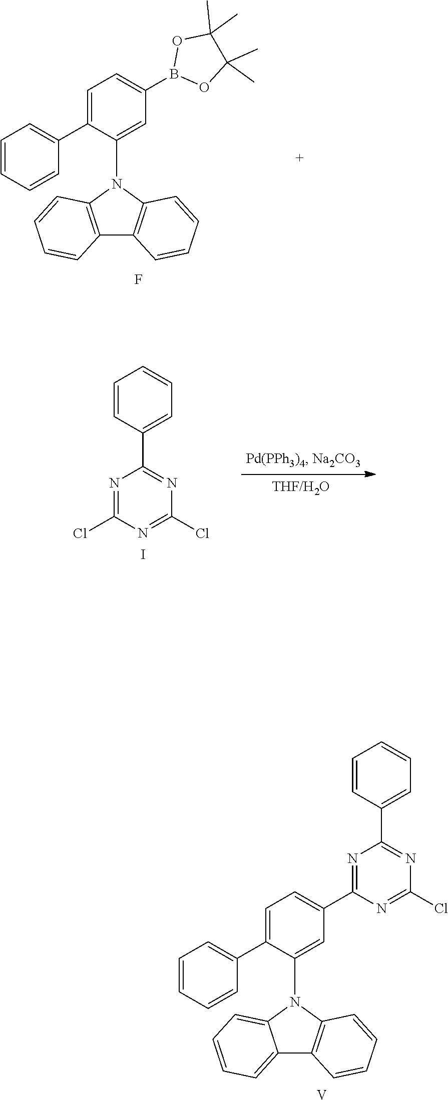

In a three-necked round-bottom flask, Intermediate F (15.6 g, 35.0 mmol), Intermediate I (10.3 g, 45.5 mmol), Pd(PPh₃)₄ (0.81 g, 0.70 mmol), Na₂CO₃ (7.4 g, 70.0 mmol), 280 mL of THE and 70 mL of H₂O were added in sequence. Under N₂ protection, the system was heated to reflux. After 7 h, the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=8:1 to 3:1) to obtain Intermediate V (10.5 g, 20.6 mmol) as a light yellow solid with a yield of 58.9%.

### Step 2: Synthesis of Intermediate X

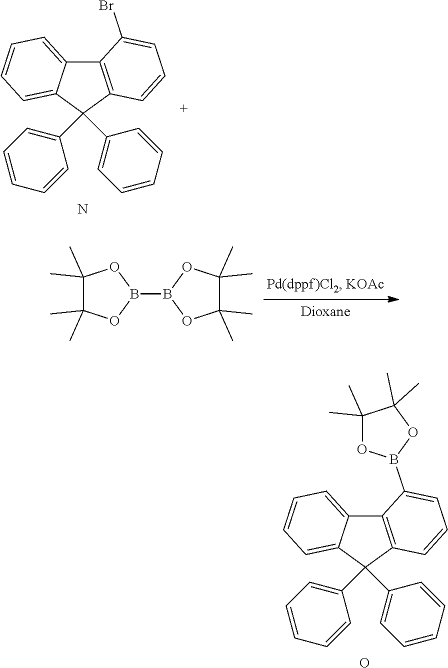

In a three-necked round-bottom flask, Intermediate W (9.9 g, 25.0 mmol), bis(pinacolato)diboron (9.5 g, 37.5 mmol), Pd(dppf)Cl₂ (0.55 g, 0.75 mmol) and KOAc (4.9 g, 50.0 mmol) were added to 1,4-dioxane (80 mL). Under N₂ protection, the system was heated to reflux overnight. After the reaction was completed as confirmed by TLC, heating was stopped and the system was cooled to room temperature. The reaction system was filtered through Celite, the filtrate was concentrated under reduced pressure, and the crude product was subjected to silica gel column chromatography (PE:DCM=5:1 to 4:1) to obtain Intermediate X (8.1 g, 18.2 mmol) as a white solid with a yield of 72.9%.

### Step 3: Synthesis of Compound A-301

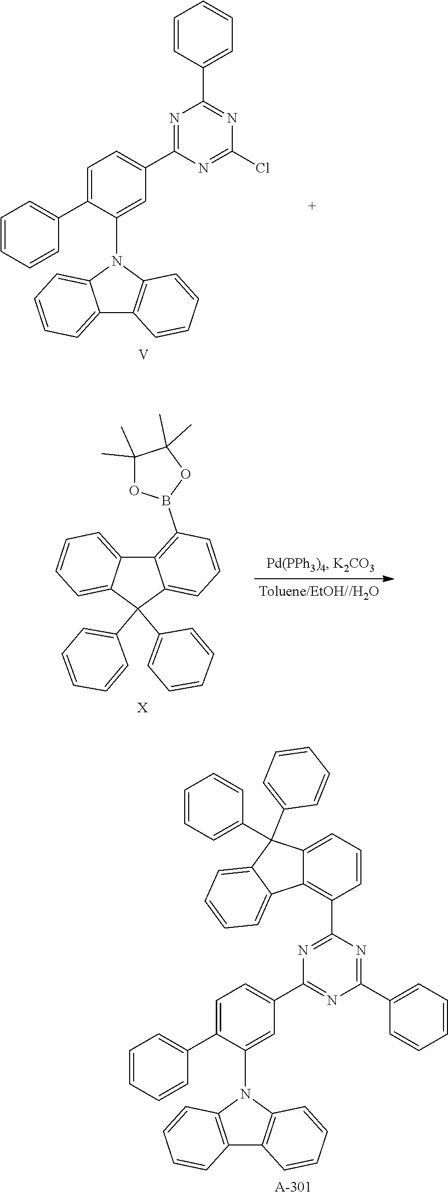

In a three-necked round-bottom flask, Intermediate V (3.0 g, 5.9 mmol), Intermediate X (2.6 g, 5.9 mmol), Pd(PPh₃)₄ (0.14 g, 0.12 mmol) and K₂CO₃ (1.6 g, 11.8 mmol) were added to toluene (40 mL), EtOH (10 mL) and H₂O (10 mL). Under N₂ protection, the system was heated to reflux overnight. After the reaction was completed as confirmed by TLC, heating was stopped, the solution was cooled to room temperature and then separated into layers, the aqueous phase was extracted with DCM, and organic phases were combined, dried over anhydrous Na₂SO₄, filtered and concentrated under reduced pressure. The crude product was subjected to silica gel column chromatography (PE:DCM=5:1 to 3:1) to obtain a white solid (3.0 g, 3.8 mmol) with a yield of 64.3%. The product was confirmed as the target product A-301 with a molecular weight of 790.3.

Those skilled in the art will appreciate that the above preparation methods are merely exemplary. Those skilled in the art can obtain other compound structures of the present disclosure through the modifications of the preparation methods.

The method for preparing an electroluminescent device is not limited. The preparation methods in the following examples are merely examples and are not to be construed as limitations.

Those skilled in the art can make reasonable improvements on the preparation methods in the following examples based on the related art. Exemplarily, the proportions of various materials in an emissive layer are not particularly limited. Those skilled in the art can reasonably select the proportions within a certain range based on the related art. For example, taking the total weight of the materials in the emissive layer as reference, a host material may account for 80% to 99% and a light-emitting material may account for 1% to 20%; or the host material may account for 90% to 99% and the light-emitting material may account for 1% to 10%; or the host material may account for 95% to 99% and the light-emitting material may account for 1% to 5%. Further, the host material may include one material or two materials, where a ratio of two host materials may be 100:0 to 1:99; or the ratio may be 80:20 to 20:80; or the ratio may be 60:40 to 40:60. In the examples of the device, the characteristics of the device were also tested using conventional equipment in the art (including, but not limited to, evaporator produced by ANGSTROM ENGINEERING, optical testing system produced by SUZHOU FSTAR, life testing system produced by SUZHOU FSTAR, and ellipsometer produced by BEIJING ELLITOP, etc.) by methods well-known to the persons skilled in the art.

### Device Example

### Device Example 1

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL, with a thickness of 100 Å). Compound HT was used as a hole transporting layer (HTL, with a thickness of 350 Å). Compound PH-23 was used as an electron blocking layer (EBL, with a thickness of 50 Å). Then, Compound GD23 was doped in Compound PH-23 and Compound A-2 of the present disclosure and co-deposited for use as an emissive layer (EML, with a thickness of 400 Å and a weight ratio of 8:64:28). Compound HB was used as a hole blocking layer (HBL, with a thickness of 50 Å). On the HBL, Compound ET and 8-hydroxyquinolinolato-lithium (Liq) were co-deposited for use as an electron transporting layer (ETL, with a thickness of 350 Å and a weight ratio of 40:60). Finally, 8-hydroxyquinolinolato-lithium (Liq) was deposited for use as an electron injection layer with a thickness of 1 nm and Al was deposited for use as a cathode with a thickness of 120 nm. The device was transferred back to the glovebox and encapsulated with a glass lid to complete the device.

### Device Example 2

The preparation method in Device Example 2 was the same as that in Device Example 1, except that Compound A-2 of the present disclosure was replaced with Compound A-3 of the present disclosure in the emissive layer (EML).

### Device Example 3

The preparation method in Device Example 3 was the same as that in Device Example 1, except that Compound A-2 of the present disclosure was replaced with Compound A-11 of the present disclosure in the emissive layer (EML).

### Device Comparative Example 1

The preparation method in Device Comparative Example 1 was the same as that in Device Example 1, except that Compound A-2 of the present disclosure was replaced with Compound C-1 in the emissive layer (EML).

### Device Comparative Example 2

The preparation method in Device Comparative Example 2 was the same as that in Device Example 1, except that Compound A-2 of the present disclosure was replaced with Compound C-2 in the emissive layer (EML).

### Device Comparative Example 3

The preparation method in Device Comparative Example 3 was the same as that in Device Example 1, except that Compound A-2 of the present disclosure was replaced with Compound C-3 in the emissive layer (EML).

Detailed structures and thicknesses of layers of the devices are shown in the following table. A layer using more than one material is obtained by doping different compounds at their weight ratio as recorded.

TABLE 1
Part of device structures in Device Examples 1 to 3 and Comparative Examples 1 to 3

| Device ID | HIL | HTL | EBL | EML | HBL | ETI |
|---|---|---|---|---|---|---|
| Example 1 | Compound | Compound | Compound | Compound | Compound | Compound |
| | HI | HT | PH-23 | PH-23:Compound | HB | ET:Liq |
| | (100 Å) | (350 Å) | (50 Å) | A-2:Compound GD23 | (50 Å) | (40:60) |
| | | | | (64:28:8) (400 Å) | | (350 Å) |
| Example 2 | Compound | Compound | Compound | Compound | Compound | Compound |
| | HI | HT | PH-23 | PH-23:Compound | HB | ET:Liq |
| | (100 Å) | (350 Å) | (50 Å) | A-3:Compound GD23 | (50 Å) | (40:60) |
| | | | | (64:28:8) (400 Å) | | (350 Å) |
| Example 3 | Compound | Compound | Compound | Compound | Compound | Compound |
| | HI | HT | PH-23 | PH-23: Compound | HB | ET:Liq |
| | (100 Å) | (350 Å) | (50 Å) | A-11:Compound GD23 | (50 Å) | (40:60) |
| | | | | (64:28:8) (400 Å) | | (350 Å) |
| Comparative | Compound | Compound | Compound | Compound | Compound | Compound |
| Example 1 | HI | HT | PH-23 | PH-23:Compound | HB | ET:Liq |
| | (100 Å) | (350 Å) | (50 Å) | C-1:Compound GD23 | (50 Å) | (40:60) |
| | | | | (64:28:8) (400 Å) | | (350 Å) |
| Comparative | Compound | Compound | Compound | Compound | Compound | Compound |
| Example 2 | HI | HT | PH-23 | PH-23:Compound | HB | ET:Liq |
| | (100 Å) | (350 Å) | (50 Å) | C-2:Compound GD23 | (50 Å) | (40:60) |
| | | | | (64:28:8) (400 Å) | | (350 Å) |
| Comparative | Compound | Compound | Compound | Compound | Compound | Compound |
| Example 3 | HI | HT | PH-23 | PH-23:Compound | HB | ET:Liq |
| | (100 Å) | (350 Å) | (50 Å) | C-3:Compound GD23 | (50 Å) | (40:60) |
| | | | | (64:28:8) (400 Å) | | (350 Å) |

The materials used in the devices have the following structures:

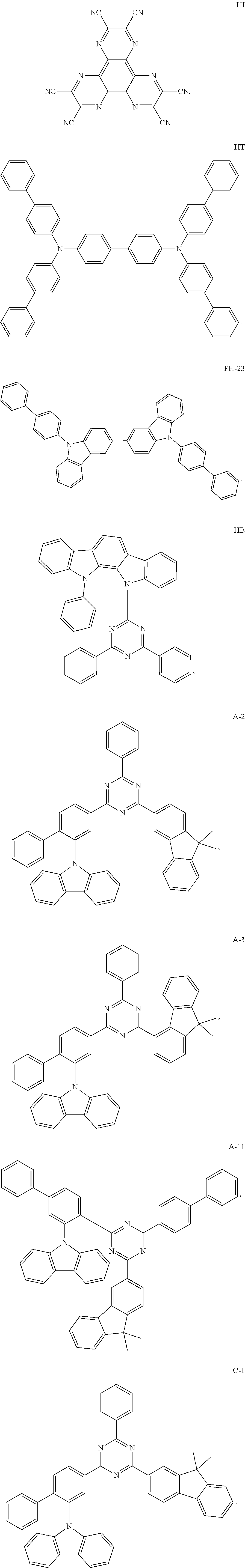

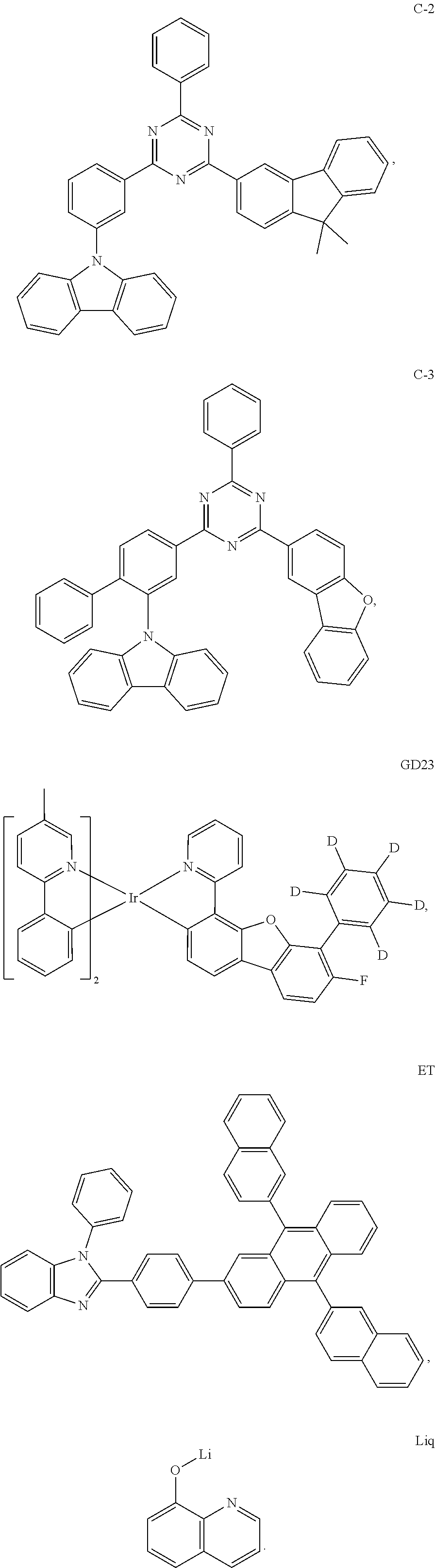

Table 2 shows the CIE data, driving voltage, external quantum efficiency (EQE), and current efficiency (CE) measured at a constant current of 15 mA/cma and the device lifetime (LT95) measured at a constant current of 80 mA/cma.

TABLE 2
Device data in Examples 1 to 3 and Comparative Examples 1 to 3

| | | Driving | | CE | LT95 |
|---|---|---|---|---|---|
| Device ID | CIE (x, y) | voltage (V) | EQE (%) | (cd/A) | (h) |
| Example 1 | (0.351, 0.623) | 4.08 | 21.8 | 83.4 | 74 |
| Example 2 | (0.353, 0.621) | 3.96 | 21.4 | 82.0 | 63 |
| Example 3 | (0.353, 0.621) | 4.11 | 22.0 | 83.9 | 72 |
| Comparative | (0.354, 0.621) | 3.83 | 21.5 | 82.0 | 28 |
| Example 1 | | | | | |
| Comparative | (0.348, 0.626) | 4.03 | 21.4 | 82.1 | 42 |
| Example 2 | | | | | |
| Comparative | (0.350, 0.624) | 4.20 | 22.0 | 84.0 | 5.4 |
| Example 3 | | | | | |

### Discussion

As can be seen from Table 2, the voltage of Comparative Example 1 is at a low voltage level, and although the voltages of Examples 1 and 2 are slightly higher than that of Comparative Example 1, the voltages of Examples 1 and 2 are still at a low voltage level. The external quantum efficiency (EQE) and current efficiency (CE) of Comparative Example 1 have reached very high levels, while Examples 1 and 2 can maintain the very high efficiency levels of Comparative Example 1 or achieve a further improvement compared with Comparative Example 1. More importantly, compared with Comparative Example 1, the device lifetime of Example 1 is significantly improved by 1.64 times, and the device lifetime of Example 2 is significantly improved by 1.25 times. While, the devices of Examples 1 and 2 differ from that of Comparative Example 1 only in the connection position of triazine on fluorenyl in the host compound. Since it is unexpected that the particular structure of the compound of Formula 1 of the present disclosure can achieve a significant improvement in device lifetime, the superiority of the compound having the structure of Formula 1 of the present disclosure is proved.

Similarly, the voltage of Comparative Example 2 is at a low voltage level, while the voltage of Example 1 is also at a low voltage level, which is substantially equivalent to the voltage of Comparative Example 2. The external quantum efficiency (EQE) and current efficiency (CE) of Comparative Example 2 have reached very high levels, while Example 1, compared with Comparative Example 2, can achieve a further improvement than the very high efficiency levels of Comparative Example 2. More importantly, compared with Comparative Example 2, the device lifetime of Example 1 is significantly improved by 76.2%. While, the device of Example 1 differs from that of Comparative Example 2 only in the presence or absence of a phenyl substituent on the bridging group, i.e., the phenylene group, connecting between the carbazole and triazine in the host compound. Since it is unexpected that the particular structure of the compound of Formula 1 of the present disclosure can achieve the significant improvement in device lifetime, the superiority of the compound having the structure of Formula 1 of the present disclosure is proved.

Similarly, the voltage of Example 1 is at a low voltage level and slightly reduced relative to the voltage of Comparative Example 3. The external quantum efficiency (EQE) and current efficiency (CE) of Comparative Example 3 have reached very high levels, while Example 1 can maintain a relatively high efficiency levels of Comparative Example 3 compared with Comparative Example 3. More importantly, compared with Comparative Example 3, the device lifetime of Example 1 is significantly improved by 12.7 times. While, the device of Example 1 differs from that of Comparative Example 3 only in that dibenzofuranyl is replaced with dimethylfluorenyl in the host compound. Since it is unexpected that the particular structure of the compound of Formula 1 of the present disclosure can achieve the very significant improvement in device lifetime, the superiority of the compound having the structure of Formula 1 of the present disclosure is proved.

In addition, the compound used in Example 3 has a different carbazole-joined position and a different Ar structure compared with those of Example 1, and a low voltage, high efficiency and a long lifetime equivalent to those of Example 1 are also obtained, further proving the superiority of the compound having the structure of Formula 1 of the present disclosure.

### Device Example 4

The preparation method in Device Example 4 was the same as that in Device Example 1, except that Compound A-2 of the present disclosure was replaced with Compound A-301 of the present disclosure in the emissive layer (EML).

### Device Comparative Example 4

The preparation method in Device Comparative Example 4 was the same as that in Device Example 1, except that Compound A-2 of the present disclosure was replaced with Compound C-4 in the emissive layer (EML).

Detailed structures and thicknesses of layers of the devices are shown in the following table. A layer using more than one material is obtained by doping different compounds at their weight ratio as recorded.

TABLE 3
Part of device structures in Device Example 4 and Comparative Example 4

| Device ID | HIL | HTL | EBL | EML | HBL | ETL |
|---|---|---|---|---|---|---|
| Example 4 | Compound | Compound | Compound | Compound | Compound | Compound |
| | HI | HT | PH-23 | PH-23:Compound | HB | ET:Liq |
| | (100 Å) | (350 Å) | (50 Å) | A-301:Compound | (50 Å) | (40:60) |
| | | | | GD23 (64:28:8) | | (350 Å) |
| | | | | (400 Å) | | |
| Comparative | Compound | Compound | Compound | Compound | Compound | Compound |
| Example 4 | HI | HT | PH-23 | PH-23:Compound | HB | ET:Liq |
| | (100 Å) | (350 Å) | (50 Å) | C-4:Compound GD23 | (50 Å) | (40:60) |
| | | | | (64:28:8) (400 Å) | | (350 Å) |

The new materials used in the devices have the following structures:

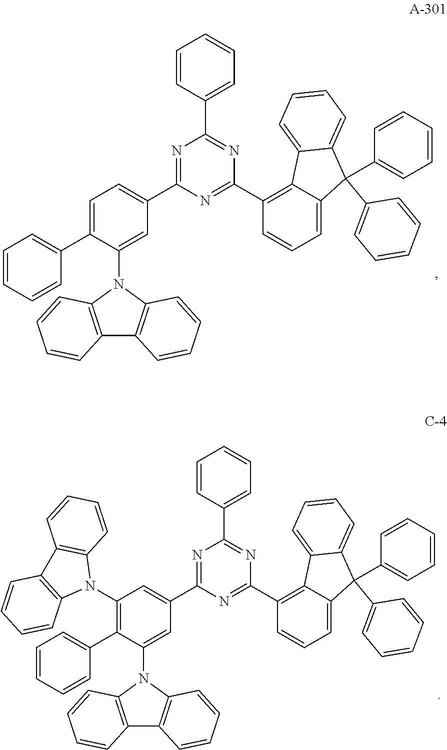 [SMILES: c1ccc(-c2nc(-c3ccc(-c4ccccc4)c(-n4c5ccccc5c5ccccc54)c3)nc(-c3cccc4c3-c3ccccc3C4(c3ccccc3)c3ccccc3)n2)cc1 ; c1ccc(-c2nc(-c3cc(-n4c5ccccc5c5ccccc54)c(-c4ccccc4)c(-n4c5ccccc5c5ccccc54)c3)nc(-c3cccc4c3-c3ccccc3C4(c3ccccc3)c3ccccc3)n2)cc1]

Table 4 shows the CIE data, driving voltage, external quantum efficiency (EQE) and current efficiency (CE) measured at a constant current of 15 mA/cm².

TABLE 4
Device data in Example 4 and Comparative Example 4

| | | Driving | | CE |
|---|---|---|---|---|
| Device ID | CIE (x, y) | voltage (V) | EQE (%) | (cd/A) |
| Example 4 | (0.352, 0.622) | 4.14 | 21.7 | 83.0 |
| Comparative | (0.355, 0.620) | 4.42 | 18.4 | 69.7 |
| Example 4 | | | | |

The device of Example 4 differs from that of Comparative Example 4 only in the presence or absence of an additional heteroaryl substituent on the bridging group, i.e., the phenylene group, connecting between the carbazole and triazine in the host compound. It can be seen from Table 4 that the voltage of Example 4 is at a low voltage level and the driving voltage is reduced by 0.28 V, the EQE is improved by 17.9% and the CE is improved by 19.1% relative to those of Comparative Example 4, further improving the overall performance of the device and proving the superiority of the compound having the structure of Formula 1 of the present disclosure.

To conclude, the compound of the present disclosure improves an electron-hole transporting balance ability of the material when used as the host material of the emissive layer and can significantly improve the overall performance of the device compared with the case where the compound is used as host material of the emissive layer instead of the compound of the present disclosure, for example, high device efficiency (EQE and CE), a low driving voltage and/or a significantly improved lifetime of the device. The compound of the present disclosure is of great help to the industry.

### Device Example 5

Firstly, a glass substrate having an indium tin oxide (ITO) anode with a thickness of 80 nm was cleaned and then treated with oxygen plasma and UV ozone. After the treatment, the substrate was dried in a glovebox to remove moisture. Then, the substrate was mounted on a substrate holder and placed in a vacuum chamber. Organic layers specified below were sequentially deposited through vacuum thermal evaporation on the ITO anode at a rate of 0.2 to 2 Angstroms per second and a vacuum degree of about 10⁻⁸ torr. Compound HI was used as a hole injection layer (HIL, with a thickness of 100 Å). Compound HT was used as a hole transporting layer (HTL, with a thickness of 350 Å). Compound PH-23 was used as an electron blocking layer (EBL, with a thickness of 50 Å). Then, Compound GD23 was doped in Compound PH-23 and Compound NH-1 and co-deposited for use as an emissive layer (EML, with a thickness of 400 Å and a weight ratio of 8:46:46). Compound HB was used as a hole blocking layer (HBL, with a thickness of 50 Å). On the HBL, Compound A-2 of the present disclosure and 8-hydroxyquinolinolato-lithium (Liq) were co-deposited for use as an electron transporting layer (ETL, with a thickness of 350 Å and a weight ratio of 40:60). Finally, 8-hydroxyquinolinolato-lithium (Liq) was deposited for use as an electron injection layer with a thickness of 1 nm and Al was deposited for use as a cathode with a thickness of 120 nm. The device was transferred back to the glovebox and encapsulated with a glass lid to complete the device.

### Device Example 6

The preparation method in Device Example 6 was the same as that in Device Example 5, except that Compound A-2 of the present disclosure was replaced with Compound A-3 of the present disclosure in the electron transporting layer (ETL).

### Device Comparative Example 5

The preparation method in Device Comparative Example 5 was the same as that in Device Example 5, except that Compound A-2 of the present disclosure was replaced with Compound ET in the electron transporting layer (ETL).

Detailed structures and thicknesses of layers of the devices are shown in the following table. A layer using more than one material is obtained by doping different compounds at their weight ratio as recorded.

TABLE 5
Part of device structures in Device Examples 5 and 6 and Comparative Example 5

| Device ID | HIL | HTL | EBL | EML | HBL | ETL |
|---|---|---|---|---|---|---|
| Example 5 | Compound | Compound | Compound | Compound | Compound | Compound |
| | HI | HT | PH-23 | PH-23:Compound | HB | A-2:Liq |
| | (100 Å) | (350 Å) | (50 Å) | NH-1:Compound GD23 | (50 Å) | (40:60) |
| | | | | (46:46:8) (400 Å) | | (350 Å) |
| Example 6 | Compound | Compound | Compound | Compound | Compound | Compound |
| | HI | HT | PH-23 | PH-23:Compound | HB | A-3:Liq |
| | (100 Å) | (350 Å) | (50 Å) | NH-1:Compound GD23 | (50 Å) | (40:60) |
| | | | | (46:46:8) (400 Å) | | (350 Å) |
| Comparative | Compound | Compound | Compound | Compound | Compound | Compound |
| Example 5 | HI | HT | PH-23 | PH-23:Compound | HB | ET:Liq |
| | (100 Å) | (350 Å) | (50 Å) | NH-1:Compound GD23 | (50 Å) | (40:60) |
| | | | | (46:46:8) (400 Å) | | (350 Å) |

The new material used in the devices has the following structure:

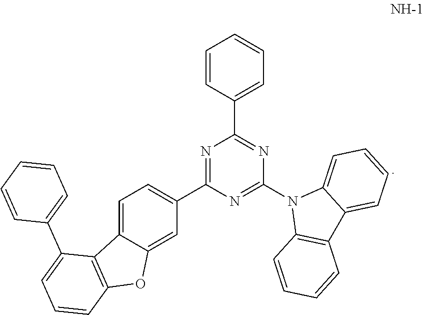 [SMILES: c1ccc(-c2nc(-c3ccc4c(c3)oc3cccc(-c5ccccc5)c34)nc(-n3c4ccccc4c4ccccc43)n2)cc1]

Table 6 shows the CIE data and external quantum efficiency (EQE) measured at a constant current of 15 mA/cm² and the device lifetime (LT95) measured at a constant current of 80 mA/cm².

TABLE 6
Device data in Examples 5 and 6 and Comparative Example 5

| Device ID | CIE (x, y) | EQE (%) | LT95 (h) |
|---|---|---|---|
| Example 5 | (0.356, 0.619) | 21.25 | 85 |
| Example 6 | (0.355, 0.620) | 21.11 | 79 |
| Comparative | (0.354, 0.621) | 20.96 | 78 |
| Example 5 | | | |

### Discussion

Compounds A-2 and A-3 of the present disclosure and Comparative Compound ET are used as electron transporting materials in Examples 5 and 6 and Comparative Example 5, respectively. The EQE and device lifetimes of Examples 5 and 6 are improved compared with those of Comparative Example 5. It is to be noted that Compound ET is a commercially available electron transporting material at present. Compared with Compound ET, the compound of the present disclosure can obtain a further improved device level when applied to an electroluminescent device, which is very rare. It can be seen that the compound of the present disclosure is also a very excellent type of electron transporting material having commercially available potential.

To conclude, the compound of the present disclosure can improve the overall performance of the device when applied to the organic electroluminescent device, for example, the high device efficiency, the low driving voltage and/or the significantly improved lifetime of the device, which has a broad application prospect.

It should be understood that various embodiments described herein are merely embodiments and not intended to limit the scope of the present disclosure. Therefore, it is apparent to those skilled in the art that the present disclosure as claimed may include variations of specific embodiments and preferred embodiments described herein. Many of the materials and structures described herein may be replaced with other materials and structures without departing from the spirit of the present disclosure. It should be understood that various theories as to why the present disclosure works are not intended to be limitative.

1. A compound having a structure of Formula 1: 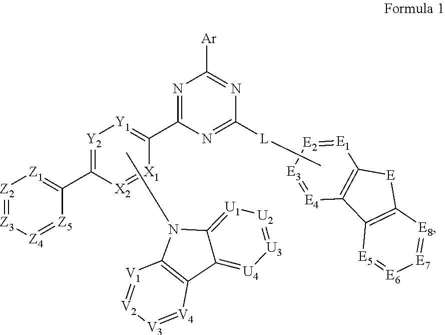
wherein in Formula 1,
one of X₁ and X₂ is selected from C, and the other is selected from CRₓ or N;
Y₁ and Y₂ are, at each occurrence identically or differently, selected from CR_{y} or N;
Z₁ to Z₅ are, at each occurrence identically or differently, selected from CR_{z} or N;
U₁ to U₄ are, at each occurrence identically or differently, selected from CRᵤ or N;
V₁ to V₄ are, at each occurrence identically or differently, selected from CRᵥ or N;
E is, at each occurrence identically or differently, selected from CRR or SiRR;
one of E₁, E₃ and E₄ is selected from C and joined to L in Formula 1, and the other two are, at each occurrence identically or differently, selected from CRₑ or N;
E₂ and E₅ to E₈ are, at each occurrence identically or differently, selected from CRₑ or N;
Ar is, at each occurrence identically or differently, selected from substituted or unsubstituted aryl having 6 to 30 carbon atoms, substituted or unsubstituted heteroaryl having 3 to 30 carbon atoms or a combination thereof;
L is selected from a single bond, substituted or unsubstituted arylene having 6 to 30 carbon atoms, substituted or unsubstituted heteroarylene having 3 to 30 carbon atoms or a combination thereof;
Rₓ, R_{z}, Rᵤ Rᵥ, Rₑ and R are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents R_{y} can be optionally joined to form a ring; adjacent substituents R_{z} can be optionally joined to form a ring; adjacent substituents Rᵤ can be optionally joined to form a ring; adjacent substituents Rᵥ can be optionally joined to form a ring; adjacent substituents Rₑ can be optionally joined to form a ring; adjacent substituents R can be optionally joined to form a ring.

2. The compound according to claim 1, wherein E is selected from CRR;
preferably, R is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group and combinations thereof; and
more preferably, R is, at each occurrence identically or differently, selected from the group consisting of: hydrogen, deuterium, fluorine, substituted or unsubstituted methyl, substituted or unsubstituted ethyl, substituted or unsubstituted n-propyl, substituted or unsubstituted isopropyl, substituted or unsubstituted cyclopropyl, substituted or unsubstituted n-butyl, substituted or unsubstituted isobutyl, substituted or unsubstituted t-butyl, substituted or unsubstituted cyclopentyl, substituted or unsubstituted neopentyl, substituted or unsubstituted cyclohexyl, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

3. The compound according to claim 1, wherein one of X₁ and X₂ is selected from C, and the other is selected from CRₓ; and/or Y₁ and Y₂ are, at each occurrence identically or differently, selected from CR_{y}; and/or Zᵢ to Z₅ are, at each occurrence identically or differently, selected from CR_{z}.

4. The compound according to claim 1, wherein U₁ to U₄ are, at each occurrence identically or differently, selected from CRᵤ; and/or V₁ to V₄ are, at each occurrence identically or differently, selected from CRᵥ; and/or one of E₁, E₃ and E₄ is selected from C and joined to L in Formula 1, the other two are selected from CRₑ, and E₂ and E₅ to E₈ are, at each occurrence identically or differently, selected from CRₑ.

5. The compound according to claim 1, wherein Ar is, at each occurrence identically or differently, selected from substituted or unsubstituted aryl having 6 to 20 carbon atoms, substituted or unsubstituted heteroaryl having 3 to 20 carbon atoms or a combination thereof; and
preferably, Ar is, at each occurrence identically or differently, selected from the group consisting of: substituted or unsubstituted phenyl, substituted or unsubstituted naphthyl, substituted or unsubstituted phenanthryl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

6. The compound according to claim 1, wherein L is, at each occurrence identically or differently, selected from a single bond, substituted or unsubstituted arylene having 6 to 20 carbon atoms, substituted or unsubstituted heteroarylene having 3 to 20 carbon atoms or a combination thereof;
preferably, L is, at each occurrence identically or differently, selected from a single bond, substituted or unsubstituted phenylene, substituted or unsubstituted biphenylylene, substituted or unsubstituted naphthylene, substituted or unsubstituted pyridylene or a combination thereof; and
more preferably, L is, at each occurrence identically or differently, selected from a single bond or substituted or unsubstituted phenylene.

7. The compound according to claim 1, wherein Rₓ, R_{z}, Rᵤ, Rᵥ and Rₑ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, substituted or unsubstituted heteroaryl having 3 to 20 carbon atoms, a cyano group and combinations thereof; and
preferably, Rₓ, R_{z}, Rᵤ, Rᵥ and Rₑ are, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl, substituted or unsubstituted carbazolyl, substituted or unsubstituted dibenzofuranyl, substituted or unsubstituted dibenzothienyl and combinations thereof.

8. The compound according to claim 1, wherein R_{y} is, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted alkenyl having 2 to 20 carbon atoms, substituted or unsubstituted aryl having 6 to 20 carbon atoms, a cyano group and combinations thereof; and
preferably, R_{y} is, at each occurrence identically or differently, selected from the group consisting of: hydrogen, deuterium, fluorine, substituted or unsubstituted phenyl, substituted or unsubstituted naphthyl, substituted or unsubstituted biphenyl, substituted or unsubstituted terphenyl, substituted or unsubstituted triphenylene, substituted or unsubstituted fluorenyl and combinations thereof.

9. The compound according to claim 1, wherein the compound is selected from the group consisting of the following structures: 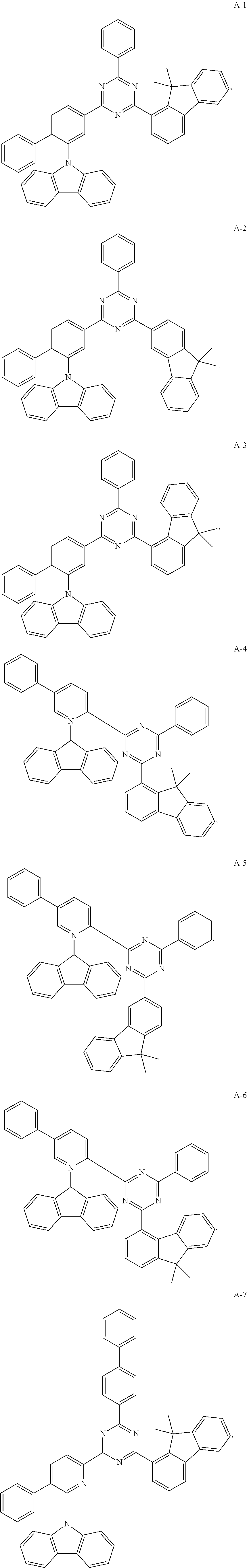 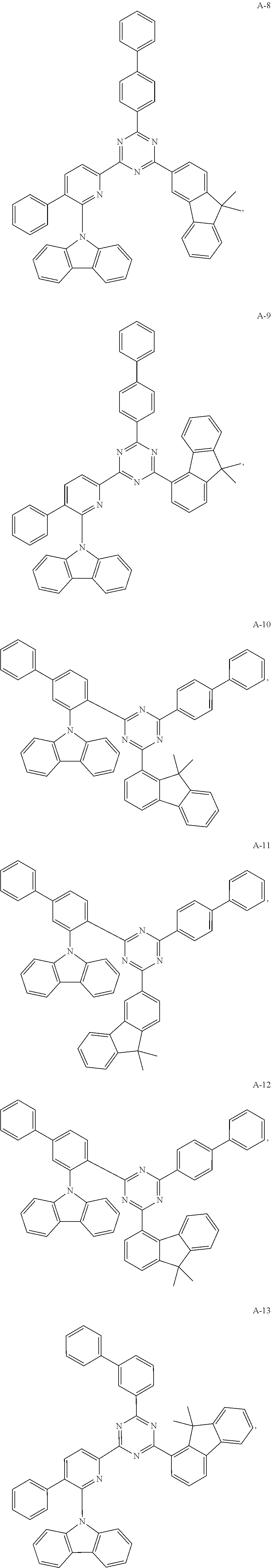 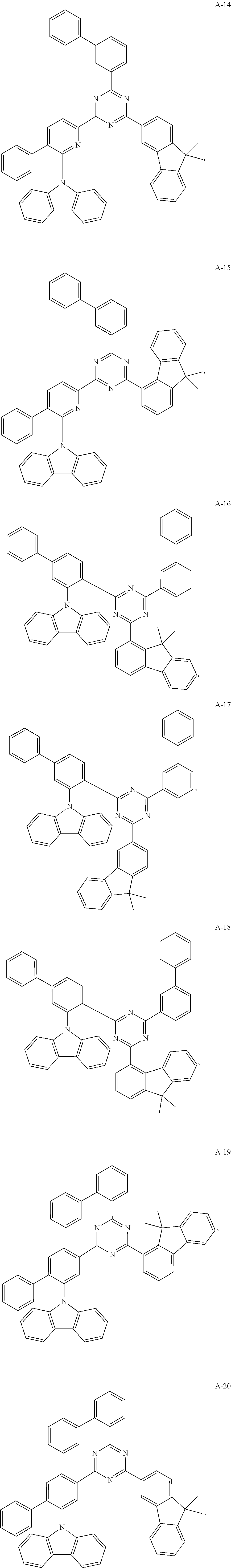 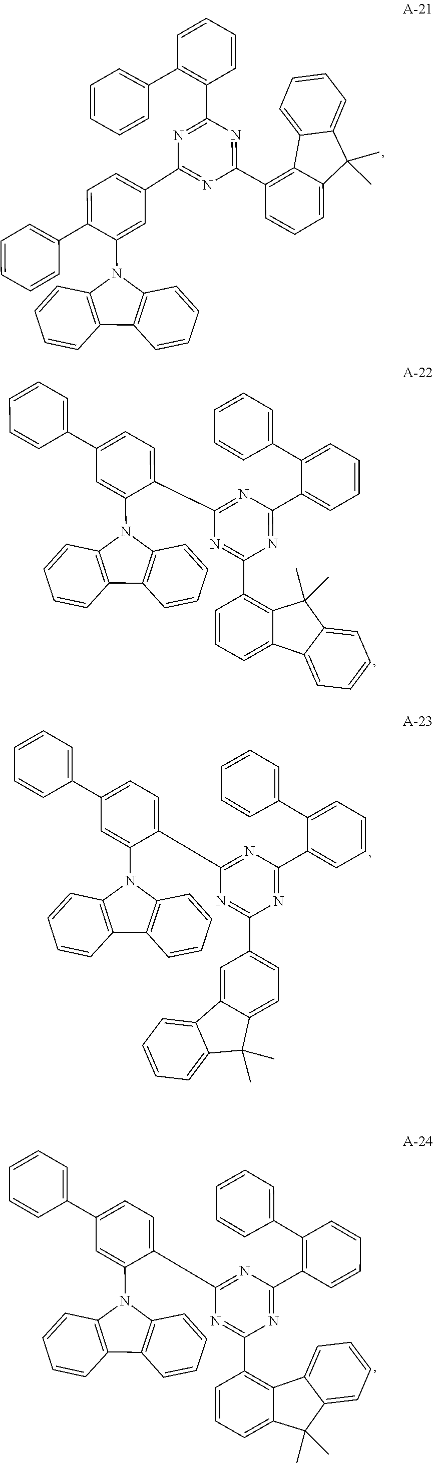 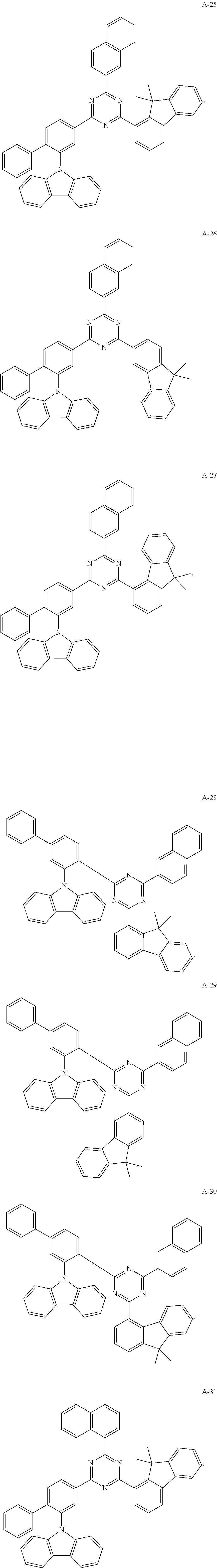 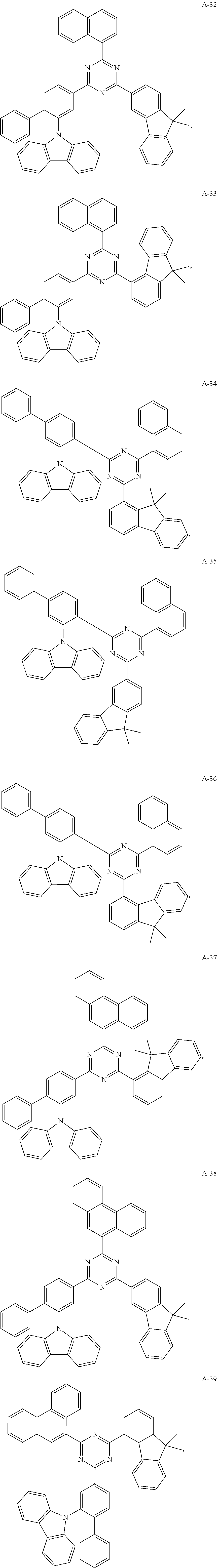 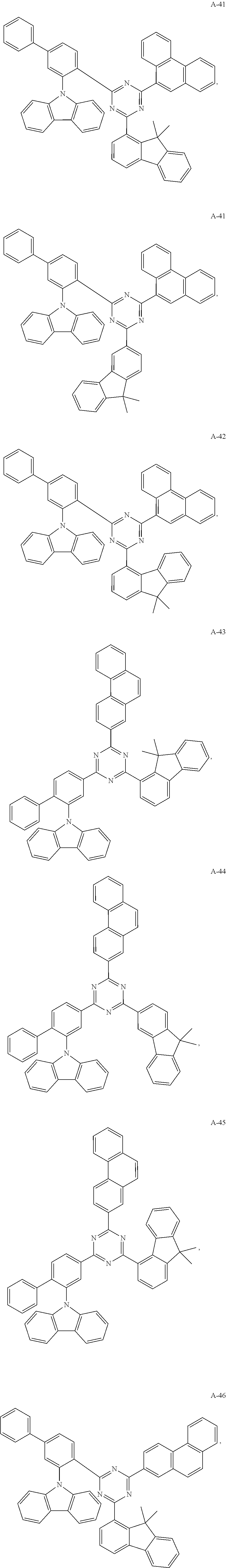 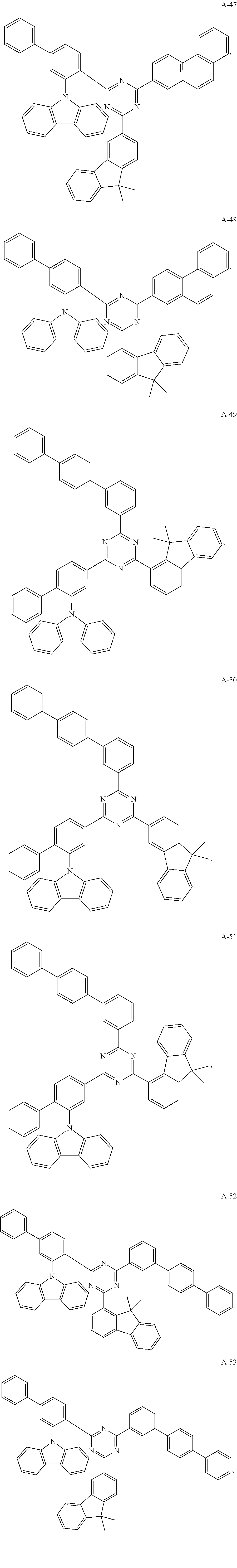 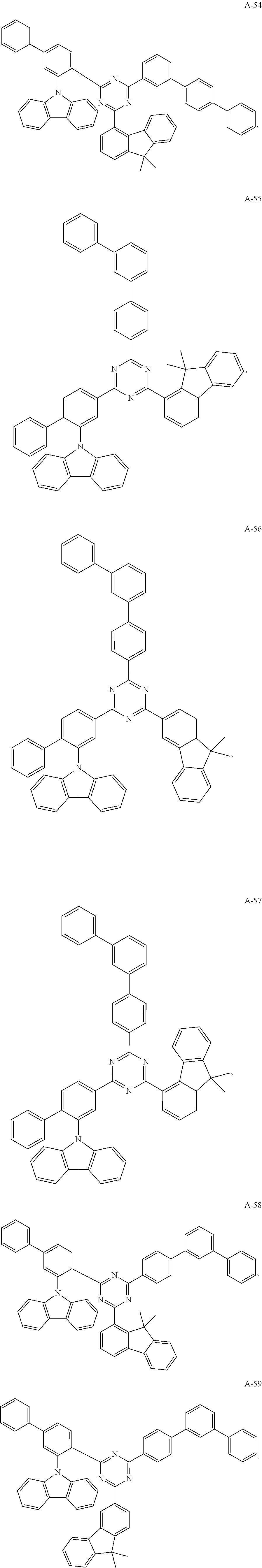 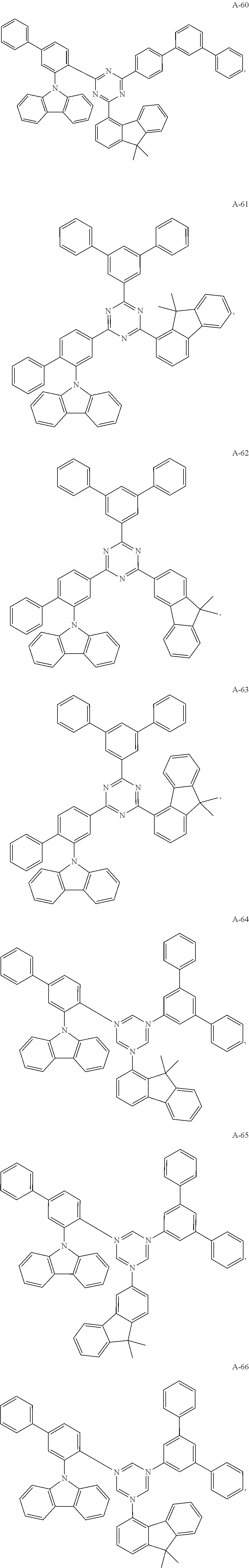 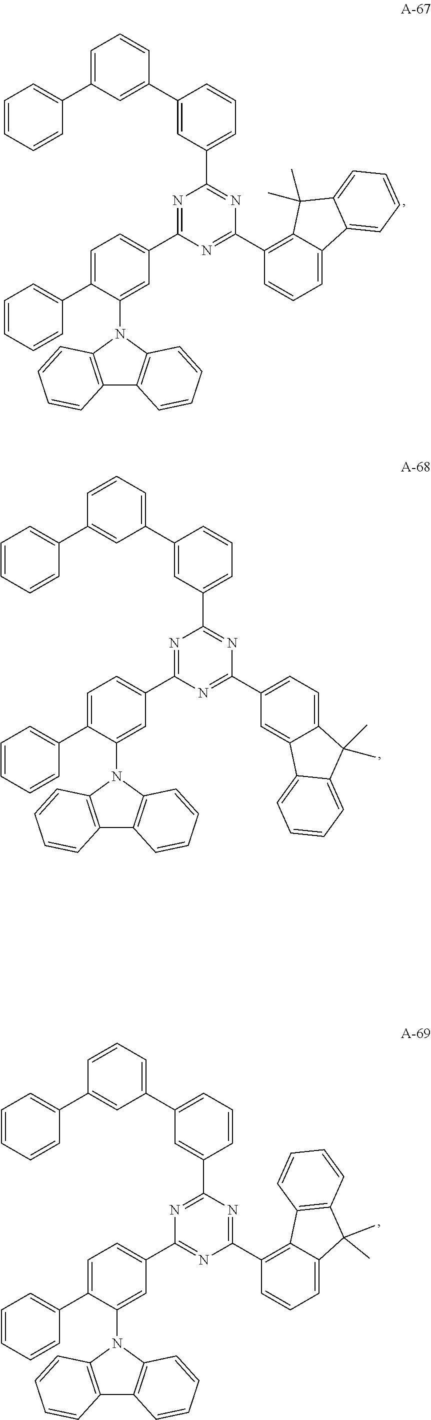 [SMILES: CC1(C)c2ccccc2-c2cccc(-c3nc(-c4cccc(-c5cccc(-c6ccccc6)c5)c4)nc(-c4ccc(-c5ccccc5)c(-n5c6ccccc6c6ccccc65)c4)n3)c21 ; CC1(C)c2ccccc2-c2cc(-c3nc(-c4cccc(-c5cccc(-c6ccccc6)c5)c4)nc(-c4ccc(-c5ccccc5)c(-n5c6ccccc6c6ccccc65)c4)n3)ccc21 ; CC1(C)c2ccccc2-c2c(-c3nc(-c4cccc(-c5cccc(-c6ccccc6)c5)c4)nc(-c4ccc(-c5ccccc5)c(-n5c6ccccc6c6ccccc65)c4)n3)cccc21] 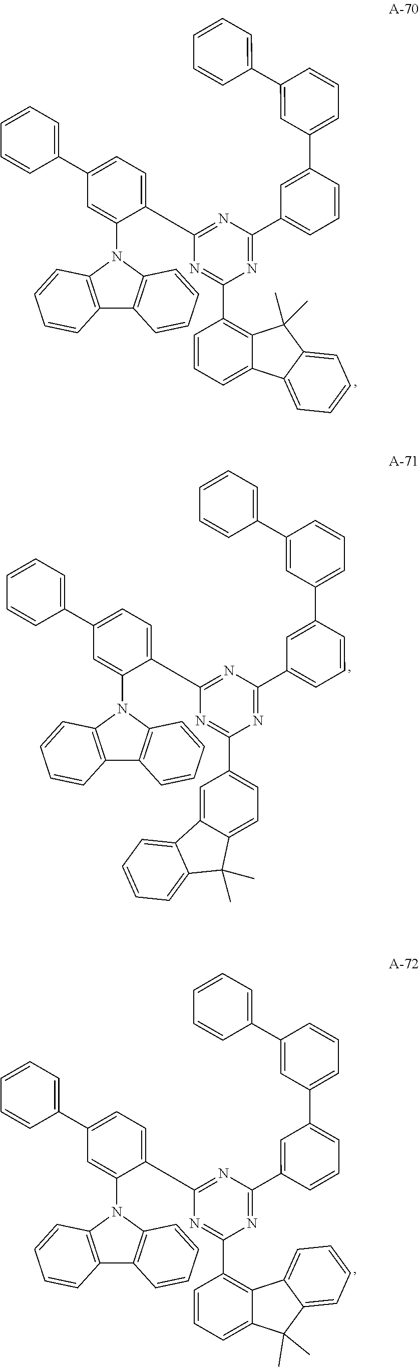 [SMILES: CC1(C)c2ccccc2-c2cccc(-c3nc(-c4cccc(-c5cccc(-c6ccccc6)c5)c4)nc(-c4ccc(-c5ccccc5)cc4-n4c5ccccc5c5ccccc54)n3)c21 ; CC1(C)c2ccccc2-c2cc(-c3nc(-c4cccc(-c5cccc(-c6ccccc6)c5)c4)nc(-c4ccc(-c5ccccc5)cc4-n4c5ccccc5c5ccccc54)n3)ccc21 ; CC1(C)c2ccccc2-c2c(-c3nc(-c4cccc(-c5cccc(-c6ccccc6)c5)c4)nc(-c4ccc(-c5ccccc5)cc4-n4c5ccccc5c5ccccc54)n3)cccc21] 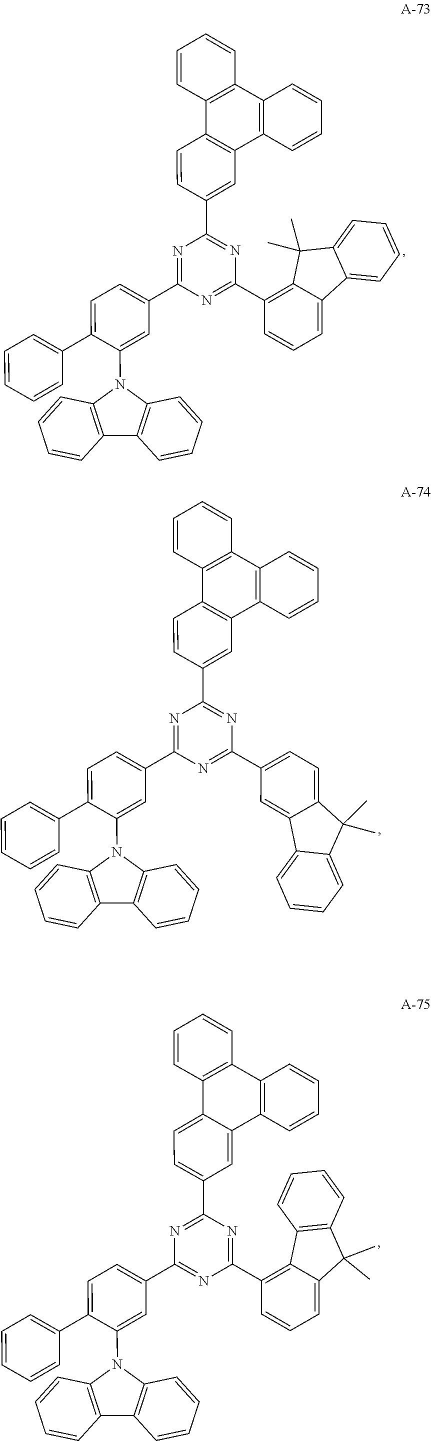 [SMILES: CC1(C)c2ccccc2-c2cccc(-c3nc(-c4ccc(-c5ccccc5)c(-n5c6ccccc6c6ccccc65)c4)nc(-c4ccc5c6ccccc6c6ccccc6c5c4)n3)c21 ; CC1(C)c2ccccc2-c2cc(-c3nc(-c4ccc(-c5ccccc5)c(-n5c6ccccc6c6ccccc65)c4)nc(-c4ccc5c6ccccc6c6ccccc6c5c4)n3)ccc21 ; CC1(C)c2ccccc2-c2c(-c3nc(-c4ccc(-c5ccccc5)c(-n5c6ccccc6c6ccccc65)c4)nc(-c4ccc5c6ccccc6c6ccccc6c5c4)n3)cccc21] 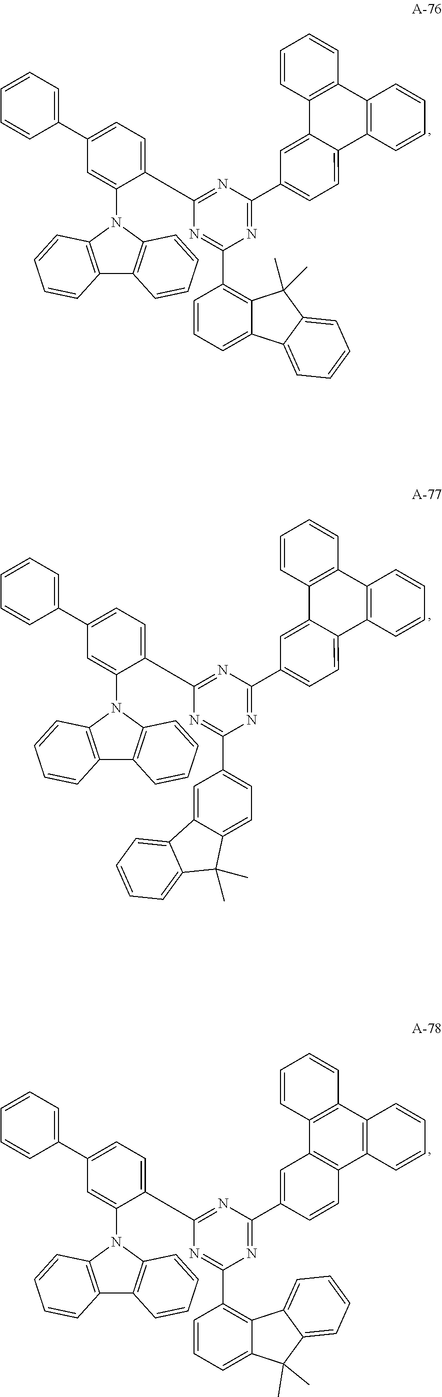 [SMILES: CC1(C)c2ccccc2-c2cccc(-c3nc(-c4ccc5c6ccccc6c6ccccc6c5c4)nc(-c4ccc(-c5ccccc5)cc4-n4c5ccccc5c5ccccc54)n3)c21 ; CC1(C)c2ccccc2-c2cc(-c3nc(-c4ccc5c6ccccc6c6ccccc6c5c4)nc(-c4ccc(-c5ccccc5)cc4-n4c5ccccc5c5ccccc54)n3)ccc21 ; CC1(C)c2ccccc2-c2c(-c3nc(-c4ccc5c6ccccc6c6ccccc6c5c4)nc(-c4ccc(-c5ccccc5)cc4-n4c5ccccc5c5ccccc54)n3)cccc21] 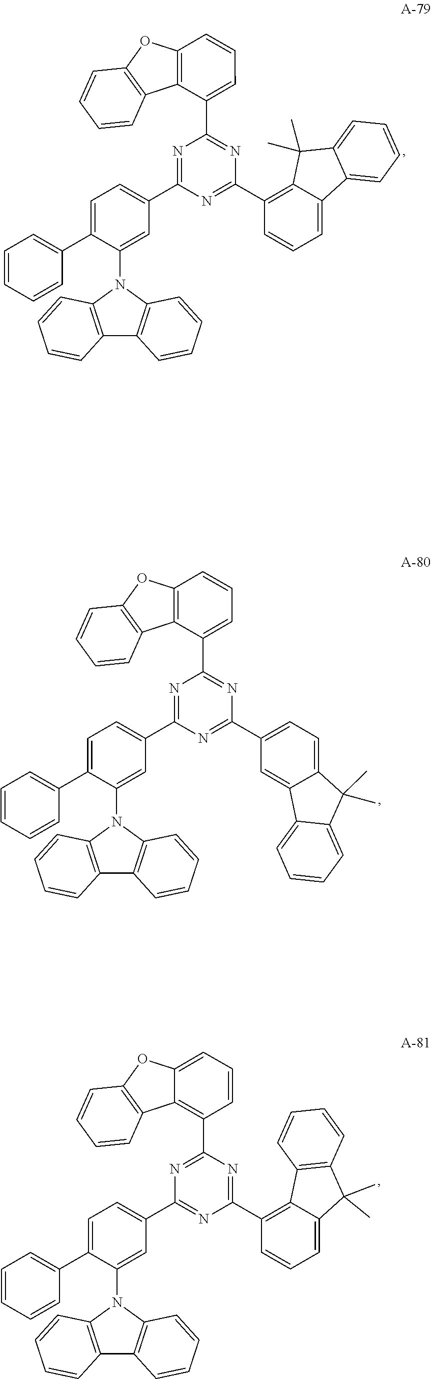 [SMILES: CC1(C)c2ccccc2-c2cccc(-c3nc(-c4ccc(-c5ccccc5)c(-n5c6ccccc6c6ccccc65)c4)nc(-c4cccc5oc6ccccc6c45)n3)c21 ; CC1(C)c2ccccc2-c2cc(-c3nc(-c4ccc(-c5ccccc5)c(-n5c6ccccc6c6ccccc65)c4)nc(-c4cccc5oc6ccccc6c45)n3)ccc21 ; CC1(C)c2ccccc2-c2c(-c3nc(-c4ccc(-c5ccccc5)c(-n5c6ccccc6c6ccccc65)c4)nc(-c4cccc5oc6ccccc6c45)n3)cccc21] 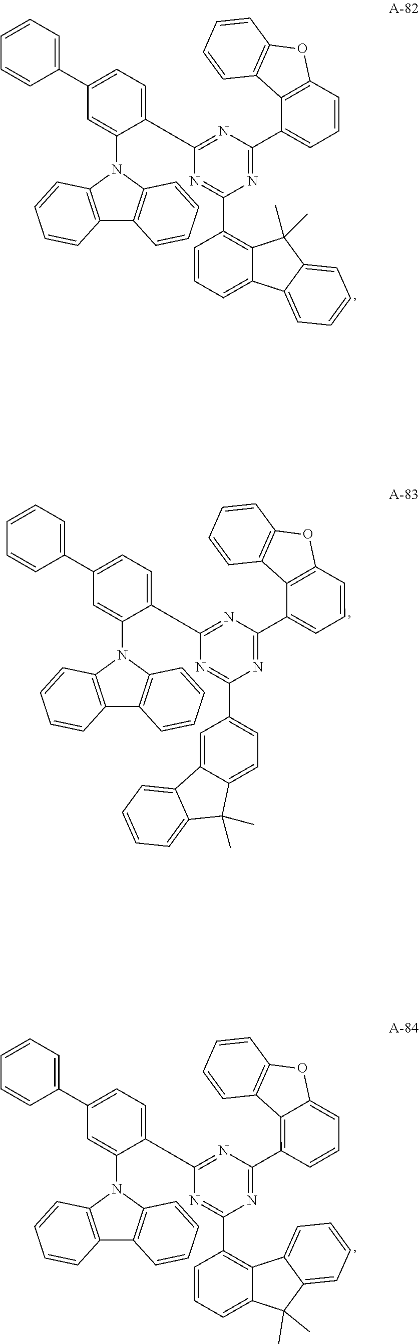 [SMILES: CC1(C)c2ccccc2-c2cccc(-c3nc(-c4ccc(-c5ccccc5)cc4-n4c5ccccc5c5ccccc54)nc(-c4cccc5oc6ccccc6c45)n3)c21 ; CC1(C)c2ccccc2-c2cc(-c3nc(-c4ccc(-c5ccccc5)cc4-n4c5ccccc5c5ccccc54)nc(-c4cccc5oc6ccccc6c45)n3)ccc21 ; CC1(C)c2ccccc2-c2c(-c3nc(-c4ccc(-c5ccccc5)cc4-n4c5ccccc5c5ccccc54)nc(-c4cccc5oc6ccccc6c45)n3)cccc21] 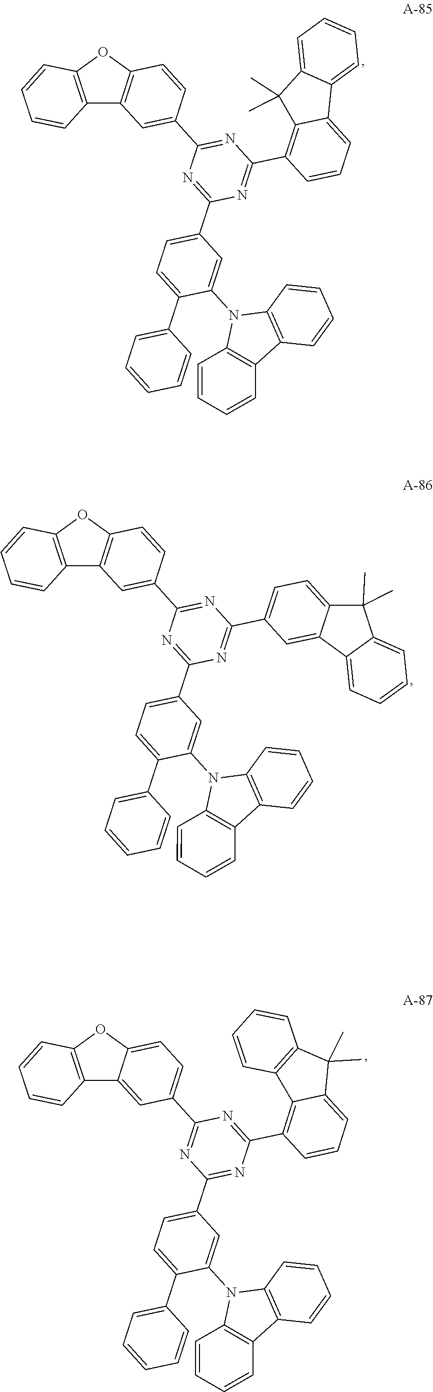 [SMILES: CC1(C)c2ccccc2-c2cccc(-c3nc(-c4ccc(-c5ccccc5)c(-n5c6ccccc6c6ccccc65)c4)nc(-c4ccc5oc6ccccc6c5c4)n3)c21 ; CC1(C)c2ccccc2-c2cc(-c3nc(-c4ccc(-c5ccccc5)c(-n5c6ccccc6c6ccccc65)c4)nc(-c4ccc5oc6ccccc6c5c4)n3)ccc21 ; CC1(C)c2ccccc2-c2c(-c3nc(-c4ccc(-c5ccccc5)c(-n5c6ccccc6c6ccccc65)c4)nc(-c4ccc5oc6ccccc6c5c4)n3)cccc21] 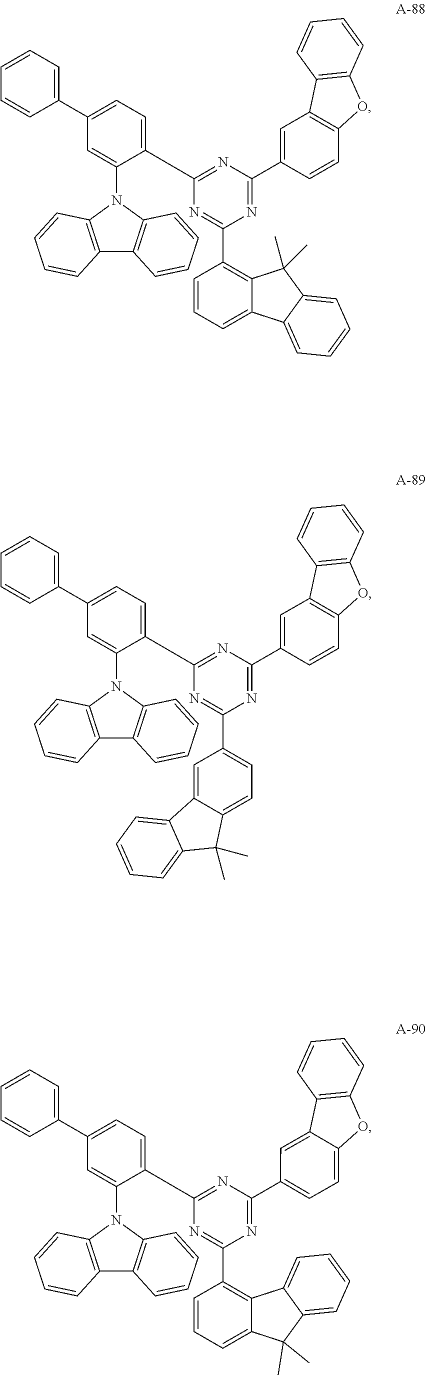 [SMILES: CC1(C)c2ccccc2-c2cccc(-c3nc(-c4ccc5oc6ccccc6c5c4)nc(-c4ccc(-c5ccccc5)cc4-n4c5ccccc5c5ccccc54)n3)c21 ; CC1(C)c2ccccc2-c2cc(-c3nc(-c4ccc5oc6ccccc6c5c4)nc(-c4ccc(-c5ccccc5)cc4-n4c5ccccc5c5ccccc54)n3)ccc21 ; CC1(C)c2ccccc2-c2c(-c3nc(-c4ccc5oc6ccccc6c5c4)nc(-c4ccc(-c5ccccc5)cc4-n4c5ccccc5c5ccccc54)n3)cccc21] 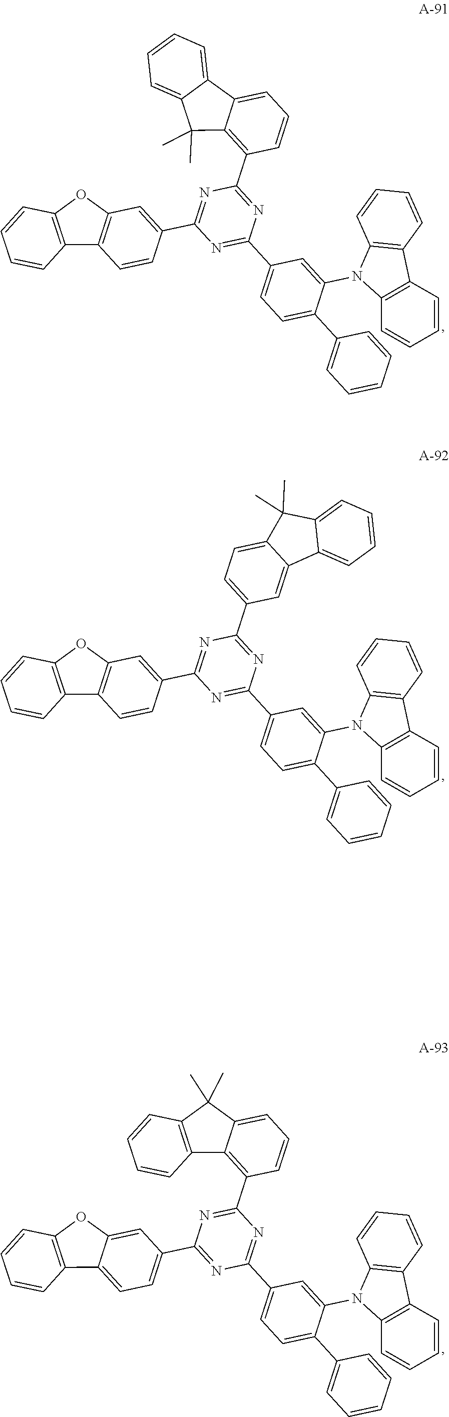 [SMILES: CC1(C)c2ccccc2-c2cccc(-c3nc(-c4ccc(-c5ccccc5)c(-n5c6ccccc6c6ccccc65)c4)nc(-c4ccc5c(c4)oc4ccccc45)n3)c21 ; CC1(C)c2ccccc2-c2cc(-c3nc(-c4ccc(-c5ccccc5)c(-n5c6ccccc6c6ccccc65)c4)nc(-c4ccc5c(c4)oc4ccccc45)n3)ccc21 ; CC1(C)c2ccccc2-c2c(-c3nc(-c4ccc(-c5ccccc5)c(-n5c6ccccc6c6ccccc65)c4)nc(-c4ccc5c(c4)oc4ccccc45)n3)cccc21] 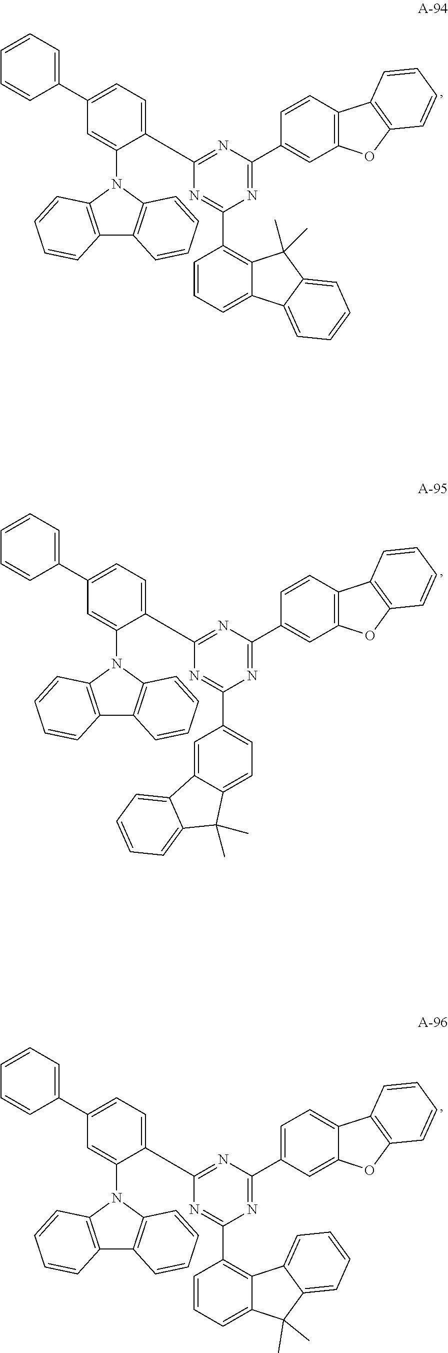 [SMILES: CC1(C)c2ccccc2-c2cccc(-c3nc(-c4ccc5c(c4)oc4ccccc45)nc(-c4ccc(-c5ccccc5)cc4-n4c5ccccc5c5ccccc54)n3)c21 ; CC1(C)c2ccccc2-c2cc(-c3nc(-c4ccc5c(c4)oc4ccccc45)nc(-c4ccc(-c5ccccc5)cc4-n4c5ccccc5c5ccccc54)n3)ccc21 ; CC1(C)c2ccccc2-c2c(-c3nc(-c4ccc5c(c4)oc4ccccc45)nc(-c4ccc(-c5ccccc5)cc4-n4c5ccccc5c5ccccc54)n3)cccc21] 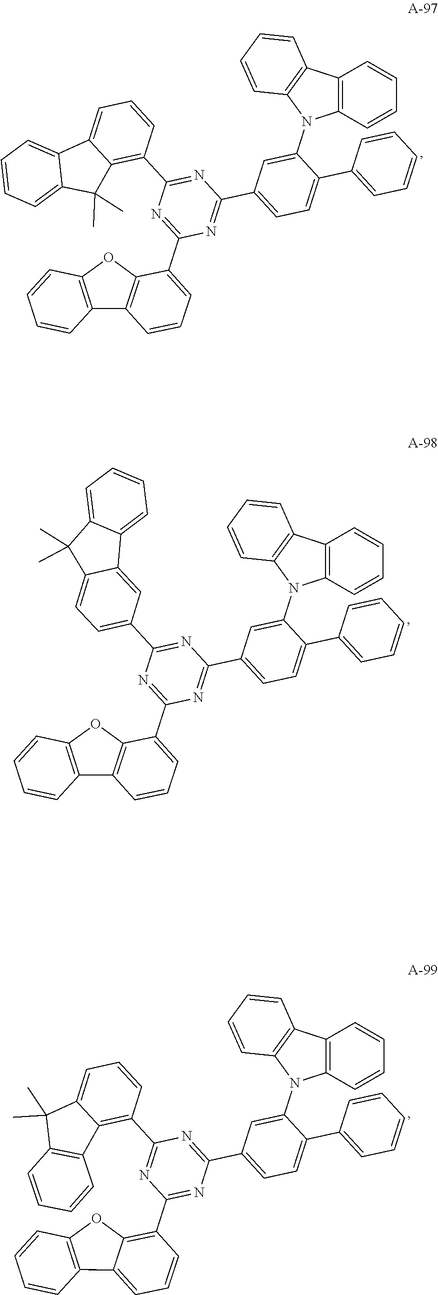 [SMILES: CC1(C)c2ccccc2-c2cccc(-c3nc(-c4ccc(-c5ccccc5)c(-n5c6ccccc6c6ccccc65)c4)nc(-c4cccc5c4oc4ccccc45)n3)c21 ; CC1(C)c2ccccc2-c2cc(-c3nc(-c4ccc(-c5ccccc5)c(-n5c6ccccc6c6ccccc65)c4)nc(-c4cccc5c4oc4ccccc45)n3)ccc21 ; CC1(C)c2ccccc2-c2c(-c3nc(-c4ccc(-c5ccccc5)c(-n5c6ccccc6c6ccccc65)c4)nc(-c4cccc5c4oc4ccccc45)n3)cccc21] 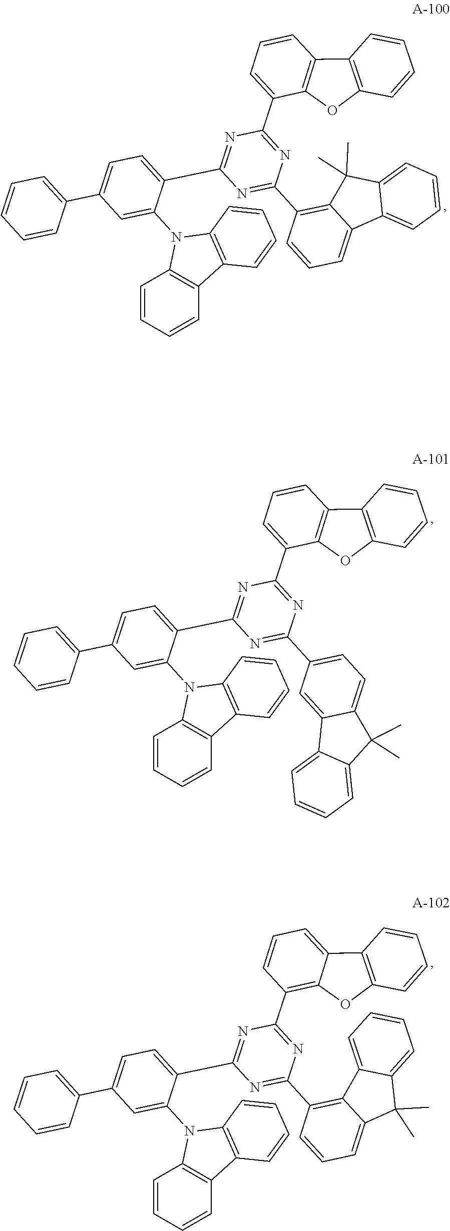 [SMILES: CC1(C)c2ccccc2-c2cccc(-c3nc(-c4ccc(-c5ccccc5)cc4-n4c5ccccc5c5ccccc54)nc(-c4cccc5c4oc4ccccc45)n3)c21 ; CC1(C)c2ccccc2-c2cc(-c3nc(-c4ccc(-c5ccccc5)cc4-n4c5ccccc5c5ccccc54)nc(-c4cccc5c4oc4ccccc45)n3)ccc21 ; CC1(C)c2ccccc2-c2c(-c3nc(-c4ccc(-c5ccccc5)cc4-n4c5ccccc5c5ccccc54)nc(-c4cccc5c4oc4ccccc45)n3)cccc21] 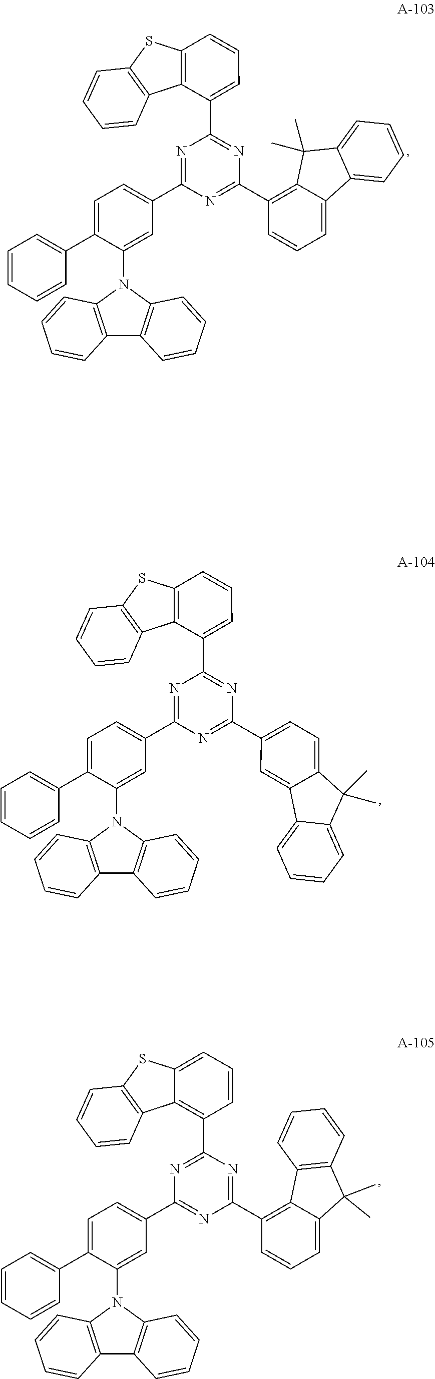 [SMILES: CC1(C)c2ccccc2-c2cccc(-c3nc(-c4ccc(-c5ccccc5)c(-n5c6ccccc6c6ccccc65)c4)nc(-c4cccc5sc6ccccc6c45)n3)c21 ; CC1(C)c2ccccc2-c2cc(-c3nc(-c4ccc(-c5ccccc5)c(-n5c6ccccc6c6ccccc65)c4)nc(-c4cccc5sc6ccccc6c45)n3)ccc21 ; CC1(C)c2ccccc2-c2c(-c3nc(-c4ccc(-c5ccccc5)c(-n5c6ccccc6c6ccccc65)c4)nc(-c4cccc5sc6ccccc6c45)n3)cccc21] 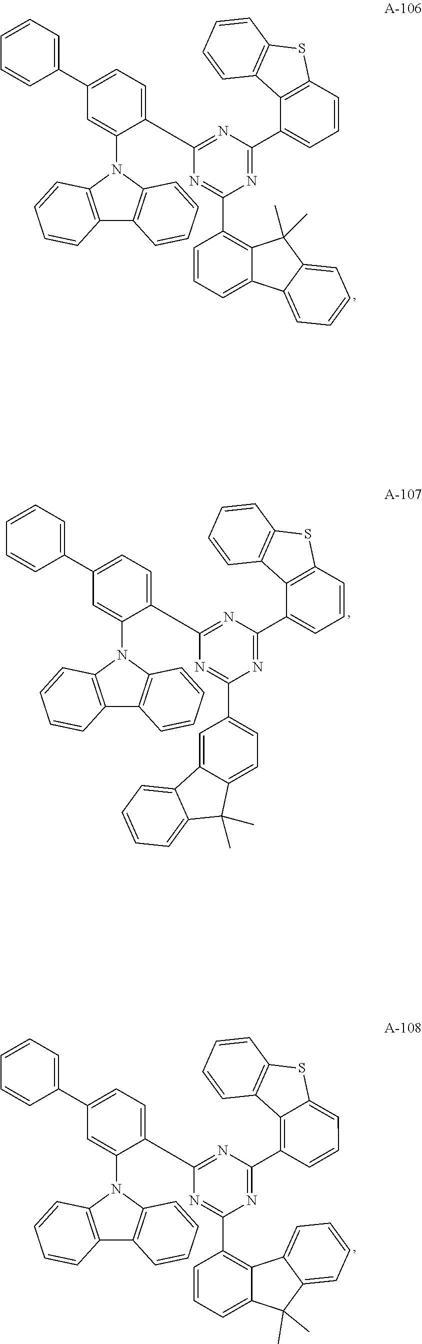 [SMILES: CC1(C)c2ccccc2-c2cccc(-c3nc(-c4ccc(-c5ccccc5)cc4-n4c5ccccc5c5ccccc54)nc(-c4cccc5sc6ccccc6c45)n3)c21 ; CC1(C)c2ccccc2-c2cc(-c3nc(-c4ccc(-c5ccccc5)cc4-n4c5ccccc5c5ccccc54)nc(-c4cccc5sc6ccccc6c45)n3)ccc21 ; CC1(C)c2ccccc2-c2c(-c3nc(-c4ccc(-c5ccccc5)cc4-n4c5ccccc5c5ccccc54)nc(-c4cccc5sc6ccccc6c45)n3)cccc21] 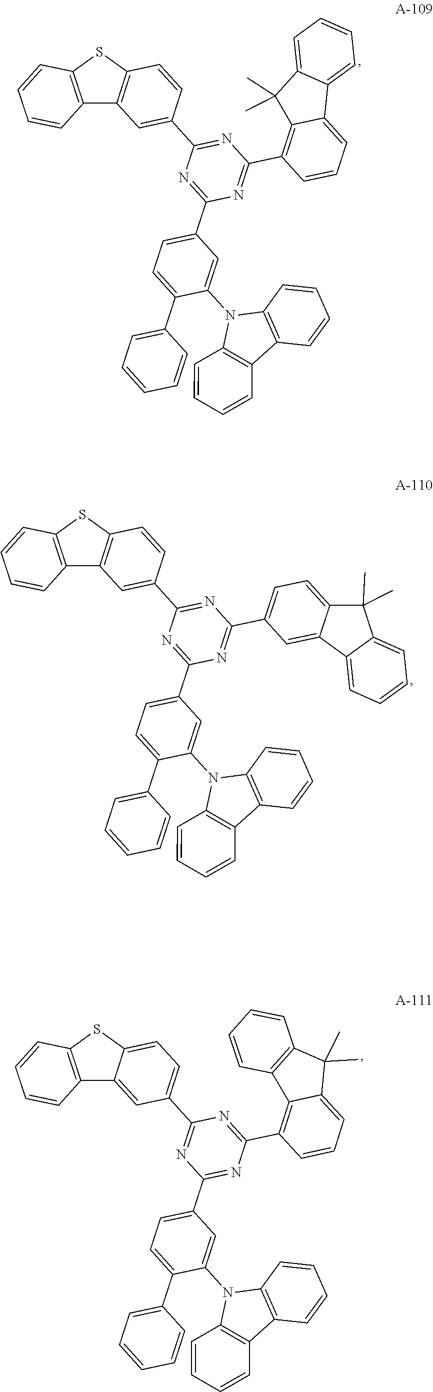 [SMILES: CC1(C)c2ccccc2-c2cccc(-c3nc(-c4ccc(-c5ccccc5)c(-n5c6ccccc6c6ccccc65)c4)nc(-c4ccc5sc6ccccc6c5c4)n3)c21 ; CC1(C)c2ccccc2-c2cc(-c3nc(-c4ccc(-c5ccccc5)c(-n5c6ccccc6c6ccccc65)c4)nc(-c4ccc5sc6ccccc6c5c4)n3)ccc21 ; CC1(C)c2ccccc2-c2c(-c3nc(-c4ccc(-c5ccccc5)c(-n5c6ccccc6c6ccccc65)c4)nc(-c4ccc5sc6ccccc6c5c4)n3)cccc21] 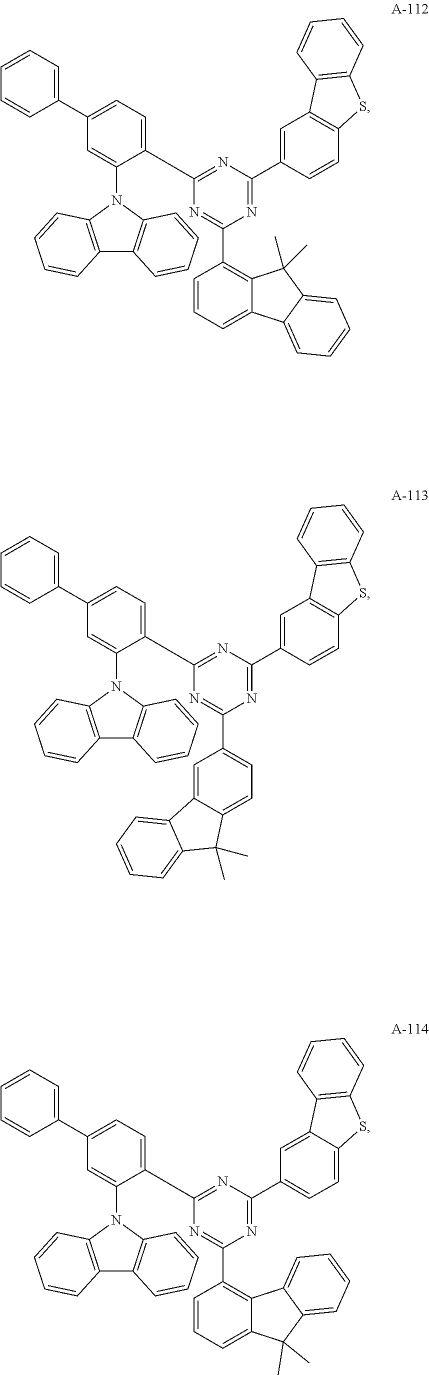 [SMILES: CC1(C)c2ccccc2-c2cccc(-c3nc(-c4ccc5sc6ccccc6c5c4)nc(-c4ccc(-c5ccccc5)cc4-n4c5ccccc5c5ccccc54)n3)c21 ; CC1(C)c2ccccc2-c2cc(-c3nc(-c4ccc5sc6ccccc6c5c4)nc(-c4ccc(-c5ccccc5)cc4-n4c5ccccc5c5ccccc54)n3)ccc21 ; CC1(C)c2ccccc2-c2c(-c3nc(-c4ccc5sc6ccccc6c5c4)nc(-c4ccc(-c5ccccc5)cc4-n4c5ccccc5c5ccccc54)n3)cccc21] 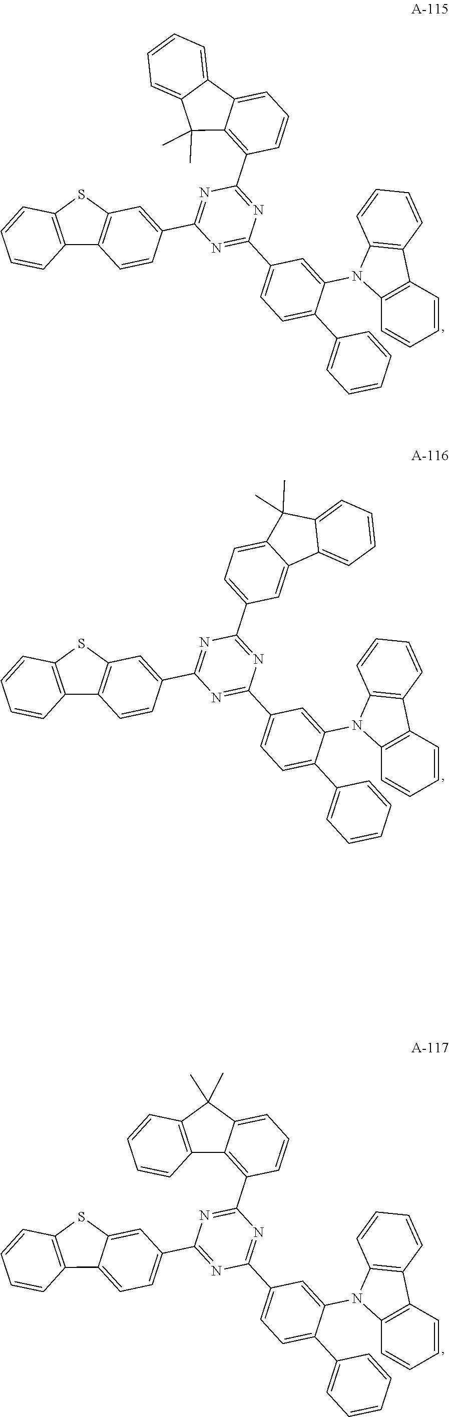 [SMILES: CC1(C)c2ccccc2-c2cccc(-c3nc(-c4ccc(-c5ccccc5)c(-n5c6ccccc6c6ccccc65)c4)nc(-c4ccc5c(c4)sc4ccccc45)n3)c21 ; CC1(C)c2ccccc2-c2cc(-c3nc(-c4ccc(-c5ccccc5)c(-n5c6ccccc6c6ccccc65)c4)nc(-c4ccc5c(c4)sc4ccccc45)n3)ccc21 ; CC1(C)c2ccccc2-c2c(-c3nc(-c4ccc(-c5ccccc5)c(-n5c6ccccc6c6ccccc65)c4)nc(-c4ccc5c(c4)sc4ccccc45)n3)cccc21] 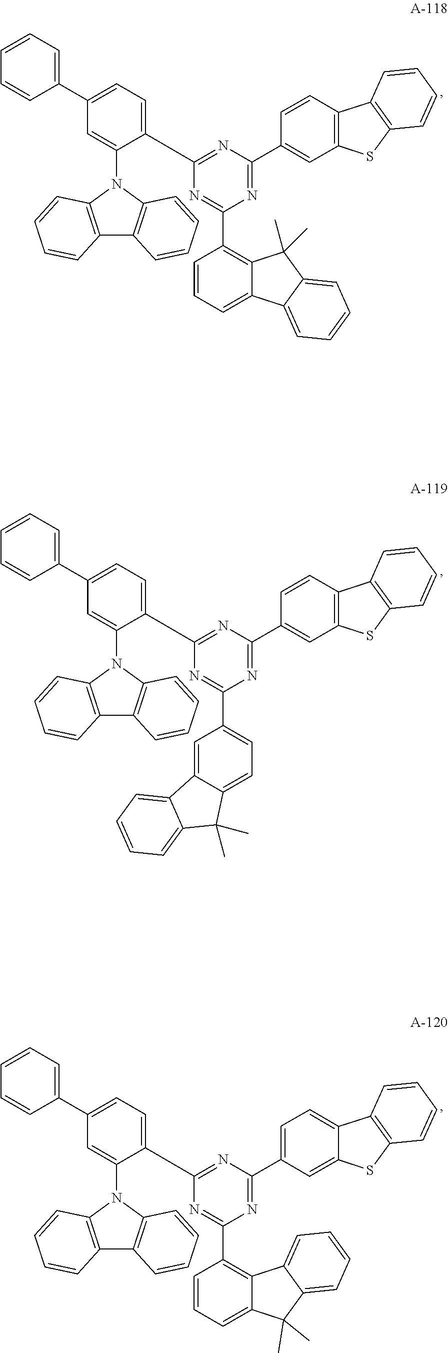 [SMILES: CC1(C)c2ccccc2-c2cccc(-c3nc(-c4ccc5c(c4)sc4ccccc45)nc(-c4ccc(-c5ccccc5)cc4-n4c5ccccc5c5ccccc54)n3)c21 ; CC1(C)c2ccccc2-c2cc(-c3nc(-c4ccc5c(c4)sc4ccccc45)nc(-c4ccc(-c5ccccc5)cc4-n4c5ccccc5c5ccccc54)n3)ccc21 ; CC1(C)c2ccccc2-c2c(-c3nc(-c4ccc5c(c4)sc4ccccc45)nc(-c4ccc(-c5ccccc5)cc4-n4c5ccccc5c5ccccc54)n3)cccc21] 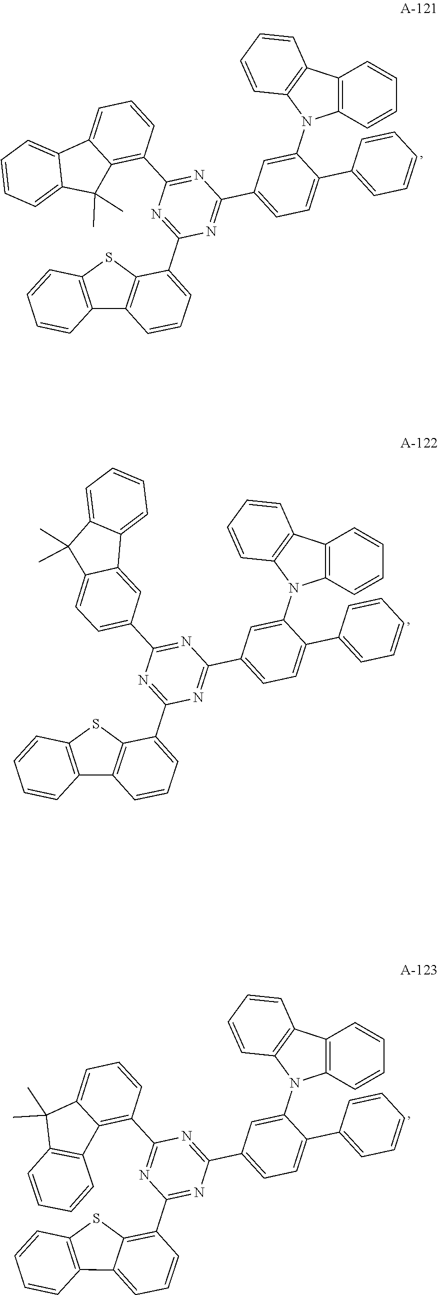 [SMILES: CC1(C)c2ccccc2-c2cccc(-c3nc(-c4ccc(-c5ccccc5)c(-n5c6ccccc6c6ccccc65)c4)nc(-c4cccc5c4sc4ccccc45)n3)c21 ; CC1(C)c2ccccc2-c2cc(-c3nc(-c4ccc(-c5ccccc5)c(-n5c6ccccc6c6ccccc65)c4)nc(-c4cccc5c4sc4ccccc45)n3)ccc21 ; CC1(C)c2ccccc2-c2c(-c3nc(-c4ccc(-c5ccccc5)c(-n5c6ccccc6c6ccccc65)c4)nc(-c4cccc5c4sc4ccccc45)n3)cccc21] 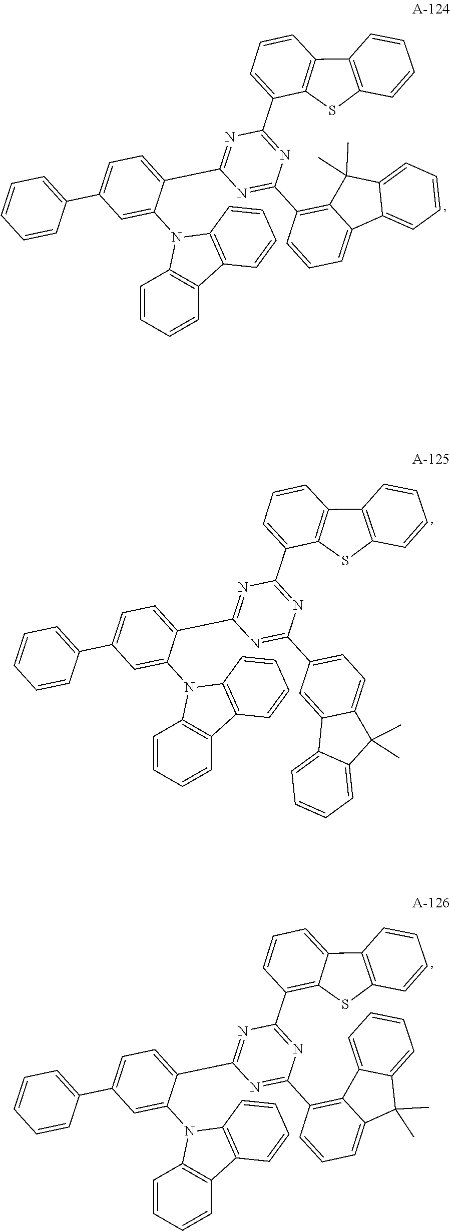 [SMILES: CC1(C)c2ccccc2-c2cccc(-c3nc(-c4ccc(-c5ccccc5)cc4-n4c5ccccc5c5ccccc54)nc(-c4cccc5c4sc4ccccc45)n3)c21 ; CC1(C)c2ccccc2-c2cc(-c3nc(-c4ccc(-c5ccccc5)cc4-n4c5ccccc5c5ccccc54)nc(-c4cccc5c4sc4ccccc45)n3)ccc21 ; CC1(C)c2ccccc2-c2c(-c3nc(-c4ccc(-c5ccccc5)cc4-n4c5ccccc5c5ccccc54)nc(-c4cccc5c4sc4ccccc45)n3)cccc21] 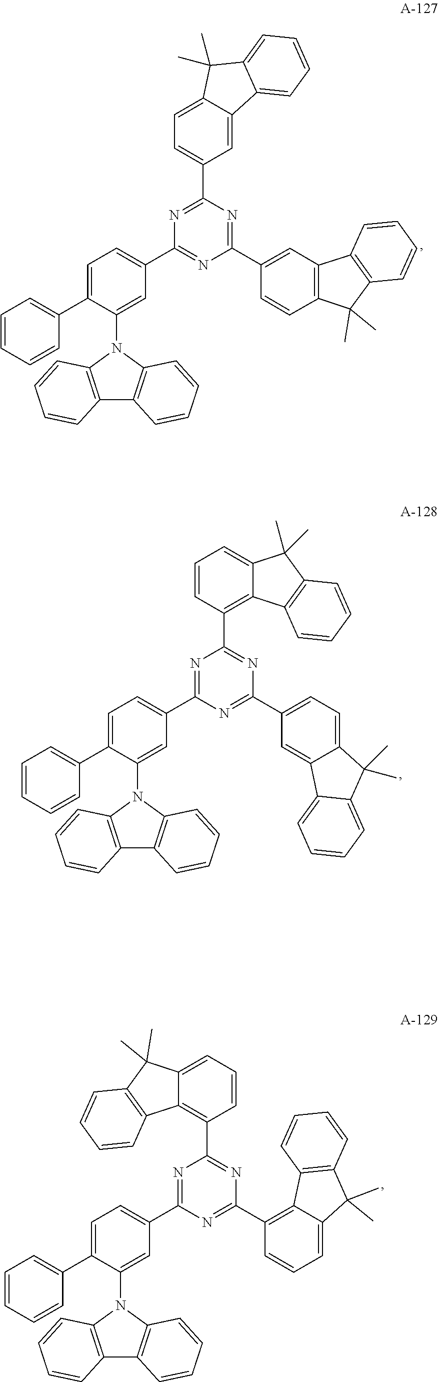 [SMILES: CC1(C)c2ccccc2-c2cc(-c3nc(-c4ccc5c(c4)-c4ccccc4C5(C)C)nc(-c4ccc(-c5ccccc5)c(-n5c6ccccc6c6ccccc65)c4)n3)ccc21 ; CC1(C)c2ccccc2-c2cc(-c3nc(-c4ccc(-c5ccccc5)c(-n5c6ccccc6c6ccccc65)c4)nc(-c4cccc5c4-c4ccccc4C5(C)C)n3)ccc21 ; CC1(C)c2ccccc2-c2c(-c3nc(-c4ccc(-c5ccccc5)c(-n5c6ccccc6c6ccccc65)c4)nc(-c4cccc5c4-c4ccccc4C5(C)C)n3)cccc21] 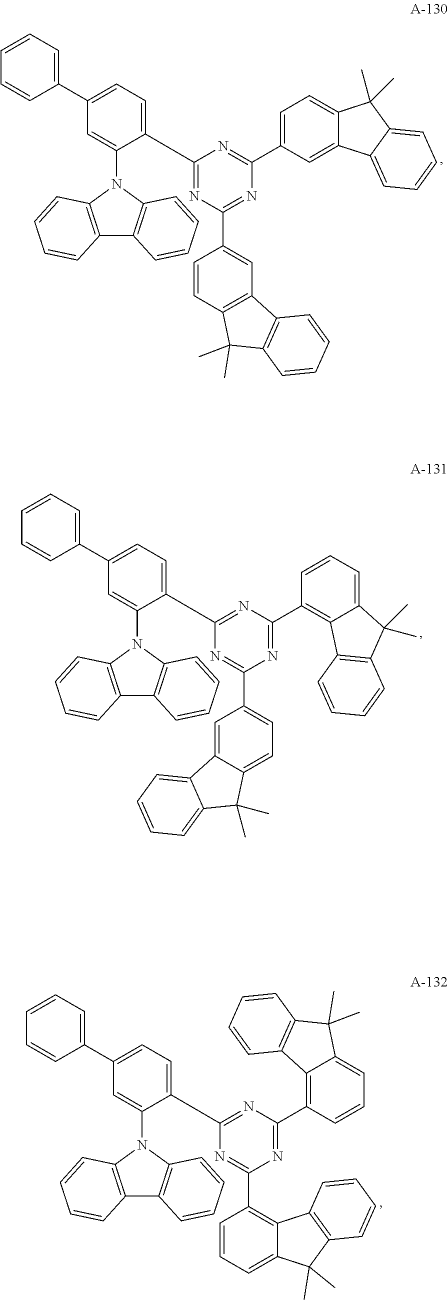 [SMILES: CC1(C)c2ccccc2-c2cc(-c3nc(-c4ccc5c(c4)-c4ccccc4C5(C)C)nc(-c4ccc(-c5ccccc5)cc4-n4c5ccccc5c5ccccc54)n3)ccc21 ; CC1(C)c2ccccc2-c2cc(-c3nc(-c4ccc(-c5ccccc5)cc4-n4c5ccccc5c5ccccc54)nc(-c4cccc5c4-c4ccccc4C5(C)C)n3)ccc21 ; CC1(C)c2ccccc2-c2c(-c3nc(-c4ccc(-c5ccccc5)cc4-n4c5ccccc5c5ccccc54)nc(-c4cccc5c4-c4ccccc4C5(C)C)n3)cccc21] 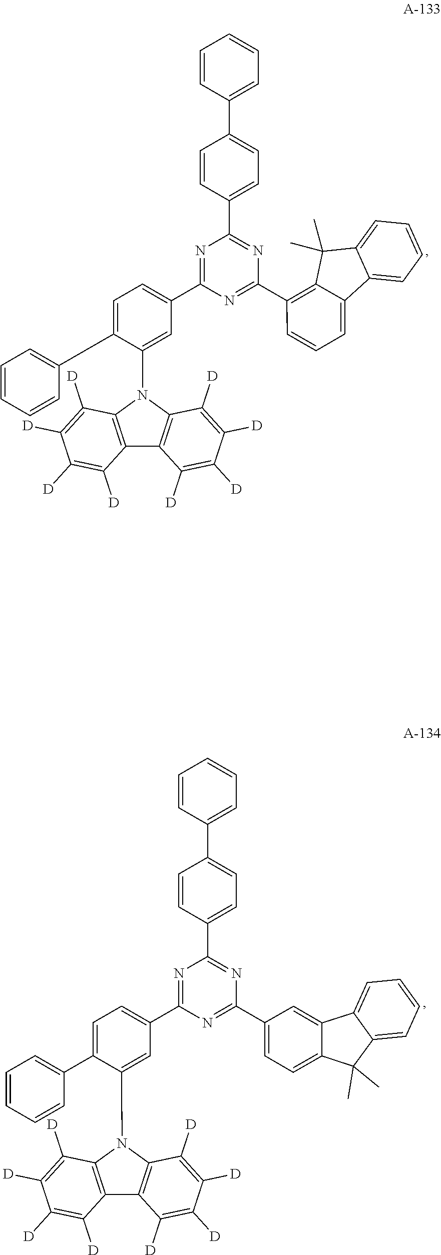 [SMILES: [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3cccc4c3C(C)(C)c3ccccc3-4)n2)ccc1-c1ccccc1 ; [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3ccc4c(c3)-c3ccccc3C4(C)C)n2)ccc1-c1ccccc1] 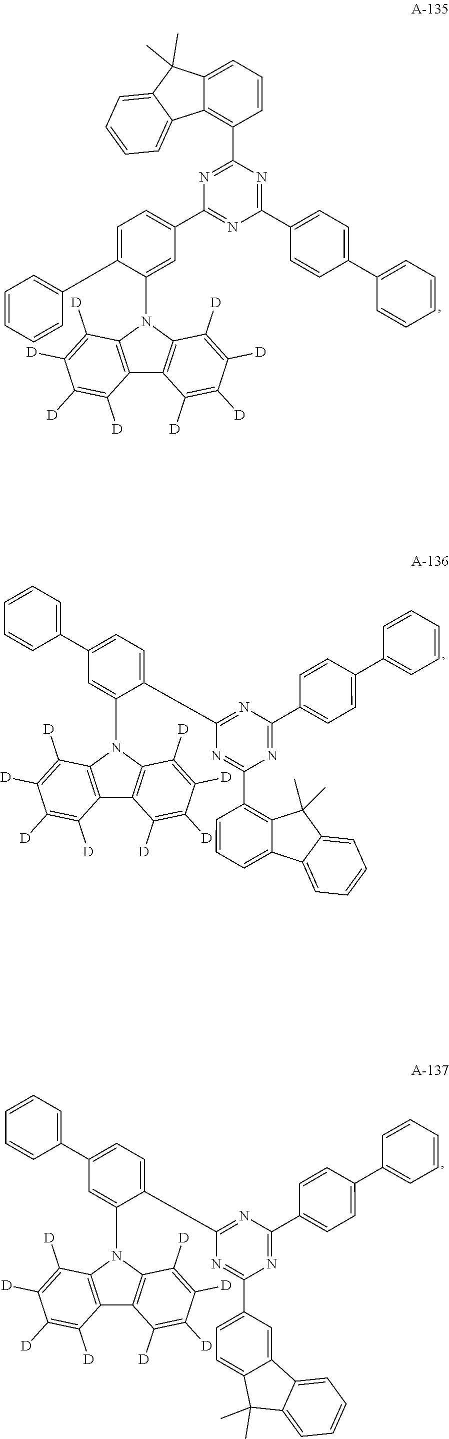 [SMILES: [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3cccc4c3-c3ccccc3C4(C)C)n2)ccc1-c1ccccc1 ; [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2cccc3c2C(C)(C)c2ccccc2-3)n1 ; [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2ccc3c(c2)-c2ccccc2C3(C)C)n1] 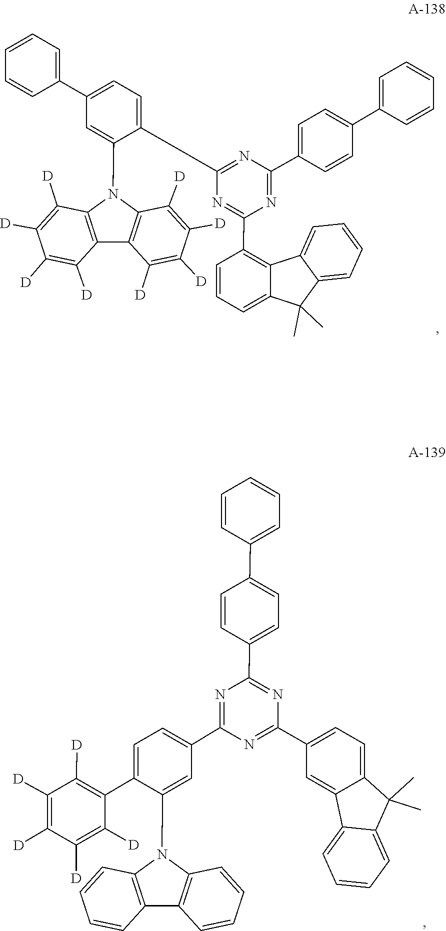 [SMILES: [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2cccc3c2-c2ccccc2C3(C)C)n1 ; [2H]c1c([2H])c([2H])c(-c2ccc(-c3nc(-c4ccc(-c5ccccc5)cc4)nc(-c4ccc5c(c4)-c4ccccc4C5(C)C)n3)cc2-n2c3ccccc3c3ccccc32)c([2H])c1[2H]] 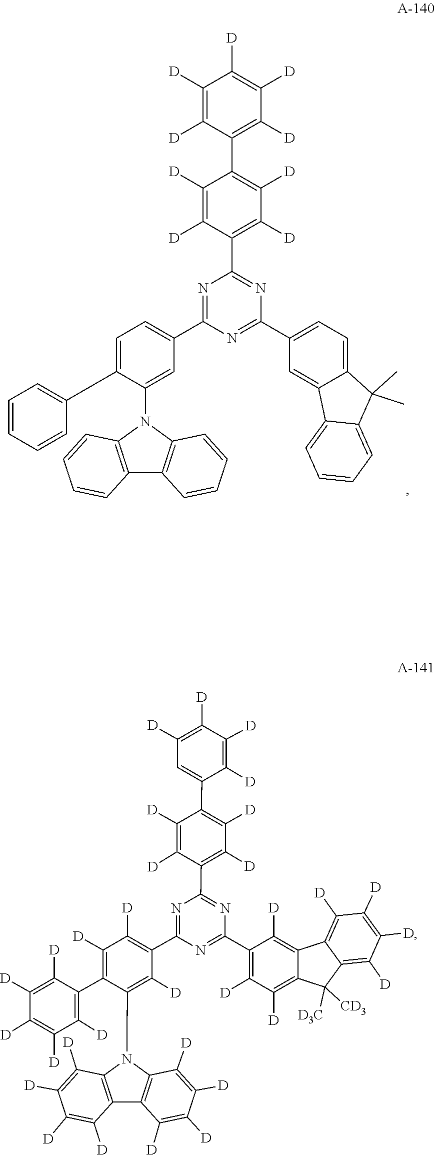 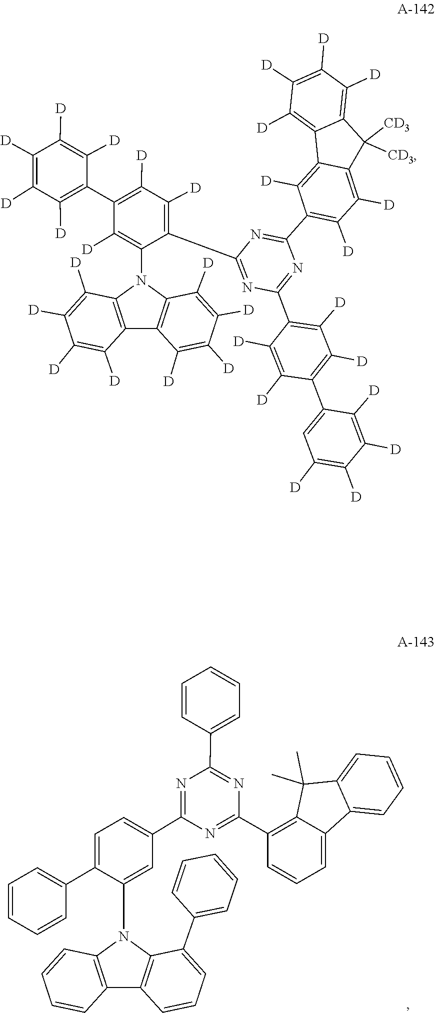 [SMILES: [2H]c1cc(-c2c([2H])c([2H])c(-c3nc(-c4c([2H])c([2H])c5c(c4[2H])-c4c([2H])c([2H])c([2H])c([2H])c4C5(C([2H])([2H])[2H])C([2H])([2H])[2H])nc(-c4c([2H])c([2H])c(-c5c([2H])c([2H])c([2H])c([2H])c5[2H])c([2H])c4-n4c5c([2H])c([2H])c([2H])c([2H])c5c5c([2H])c([2H])c([2H])c([2H])c54)n3)c([2H])c2[2H])c([2H])c([2H])c1[2H] ; CC1(C)c2ccccc2-c2cccc(-c3nc(-c4ccccc4)nc(-c4ccc(-c5ccccc5)c(-n5c6ccccc6c6cccc(-c7ccccc7)c65)c4)n3)c21] 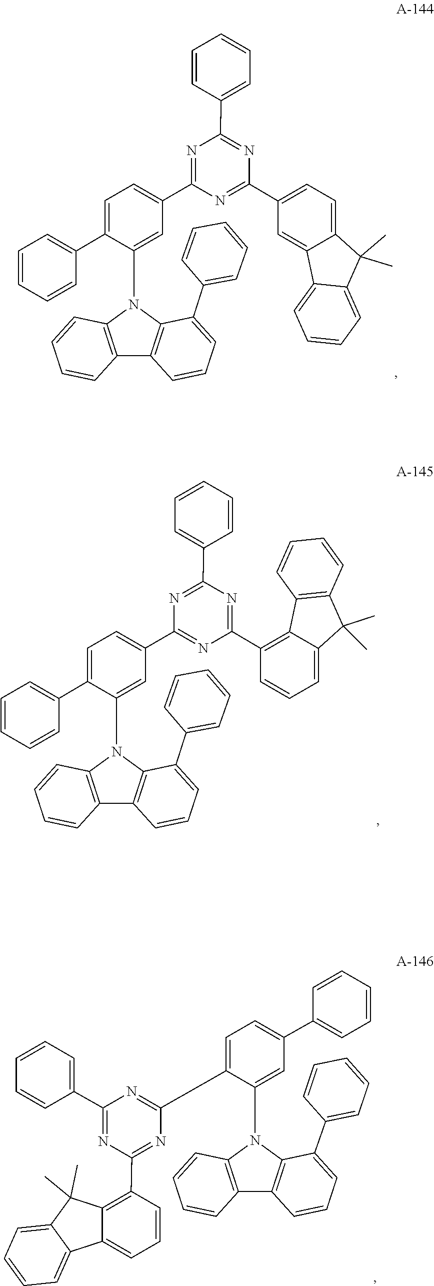 [SMILES: CC1(C)c2ccccc2-c2cc(-c3nc(-c4ccccc4)nc(-c4ccc(-c5ccccc5)c(-n5c6ccccc6c6cccc(-c7ccccc7)c65)c4)n3)ccc21 ; CC1(C)c2ccccc2-c2c(-c3nc(-c4ccccc4)nc(-c4ccc(-c5ccccc5)c(-n5c6ccccc6c6cccc(-c7ccccc7)c65)c4)n3)cccc21 ; CC1(C)c2ccccc2-c2cccc(-c3nc(-c4ccccc4)nc(-c4ccc(-c5ccccc5)cc4-n4c5ccccc5c5cccc(-c6ccccc6)c54)n3)c21] 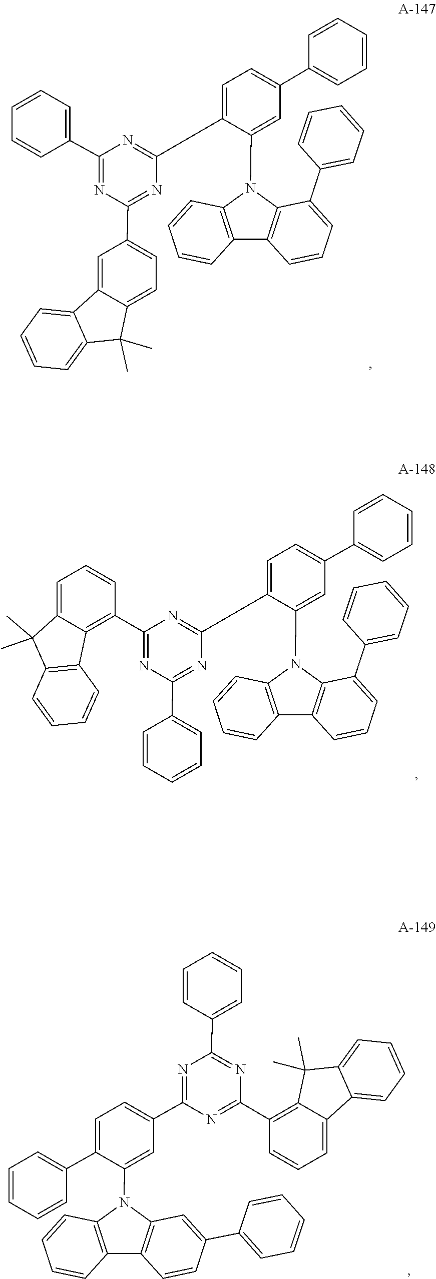 [SMILES: CC1(C)c2ccccc2-c2cc(-c3nc(-c4ccccc4)nc(-c4ccc(-c5ccccc5)cc4-n4c5ccccc5c5cccc(-c6ccccc6)c54)n3)ccc21 ; CC1(C)c2ccccc2-c2c(-c3nc(-c4ccccc4)nc(-c4ccc(-c5ccccc5)cc4-n4c5ccccc5c5cccc(-c6ccccc6)c54)n3)cccc21 ; CC1(C)c2ccccc2-c2cccc(-c3nc(-c4ccccc4)nc(-c4ccc(-c5ccccc5)c(-n5c6ccccc6c6ccc(-c7ccccc7)cc65)c4)n3)c21] 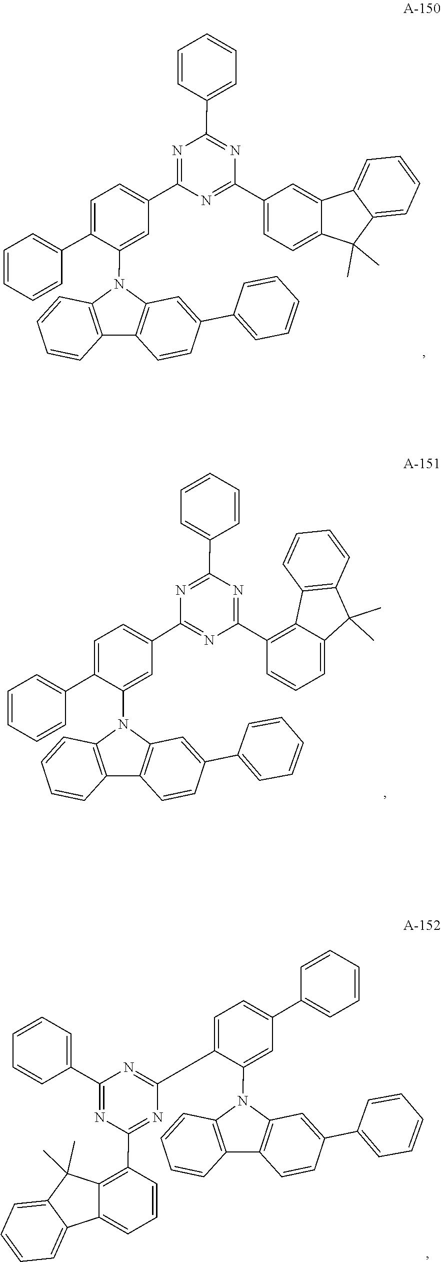 [SMILES: CC1(C)c2ccccc2-c2cc(-c3nc(-c4ccccc4)nc(-c4ccc(-c5ccccc5)c(-n5c6ccccc6c6ccc(-c7ccccc7)cc65)c4)n3)ccc21 ; CC1(C)c2ccccc2-c2c(-c3nc(-c4ccccc4)nc(-c4ccc(-c5ccccc5)c(-n5c6ccccc6c6ccc(-c7ccccc7)cc65)c4)n3)cccc21 ; CC1(C)c2ccccc2-c2cccc(-c3nc(-c4ccccc4)nc(-c4ccc(-c5ccccc5)cc4-n4c5ccccc5c5ccc(-c6ccccc6)cc54)n3)c21] 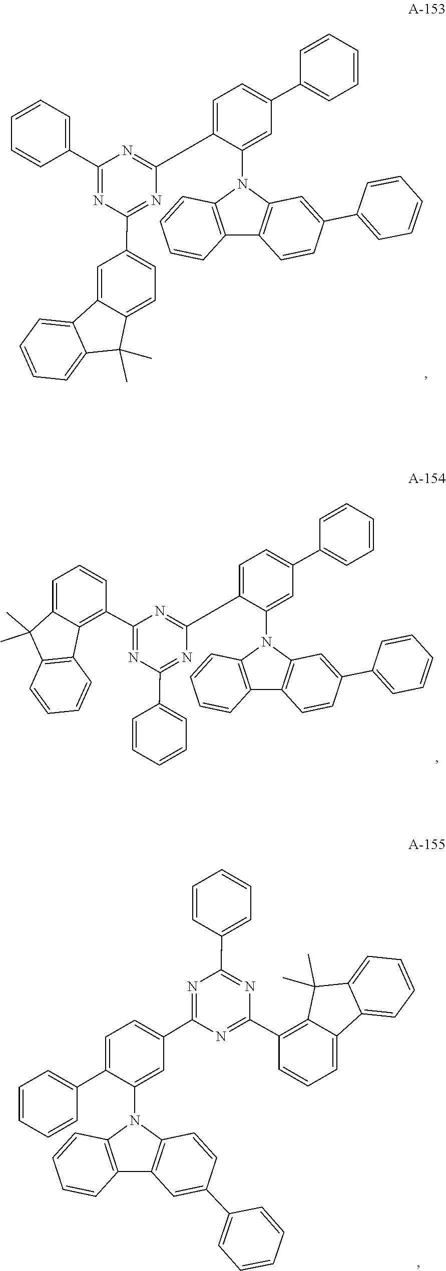 [SMILES: CC1(C)c2ccccc2-c2cc(-c3nc(-c4ccccc4)nc(-c4ccc(-c5ccccc5)cc4-n4c5ccccc5c5ccc(-c6ccccc6)cc54)n3)ccc21 ; CC1(C)c2ccccc2-c2c(-c3nc(-c4ccccc4)nc(-c4ccc(-c5ccccc5)cc4-n4c5ccccc5c5ccc(-c6ccccc6)cc54)n3)cccc21 ; CC1(C)c2ccccc2-c2cccc(-c3nc(-c4ccccc4)nc(-c4ccc(-c5ccccc5)c(-n5c6ccccc6c6cc(-c7ccccc7)ccc65)c4)n3)c21] 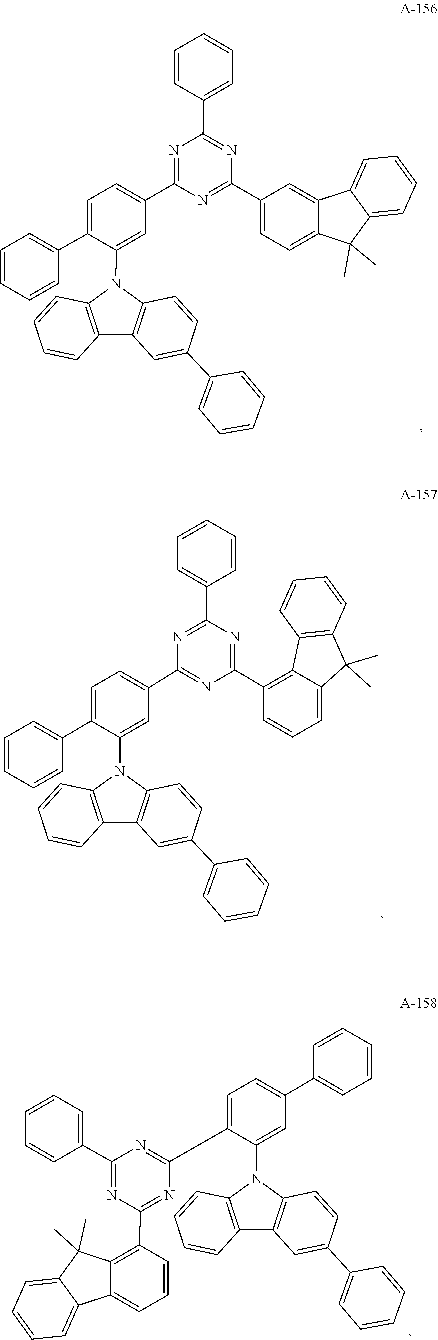 [SMILES: CC1(C)c2ccccc2-c2cc(-c3nc(-c4ccccc4)nc(-c4ccc(-c5ccccc5)c(-n5c6ccccc6c6cc(-c7ccccc7)ccc65)c4)n3)ccc21 ; CC1(C)c2ccccc2-c2c(-c3nc(-c4ccccc4)nc(-c4ccc(-c5ccccc5)c(-n5c6ccccc6c6cc(-c7ccccc7)ccc65)c4)n3)cccc21 ; CC1(C)c2ccccc2-c2cccc(-c3nc(-c4ccccc4)nc(-c4ccc(-c5ccccc5)cc4-n4c5ccccc5c5cc(-c6ccccc6)ccc54)n3)c21] 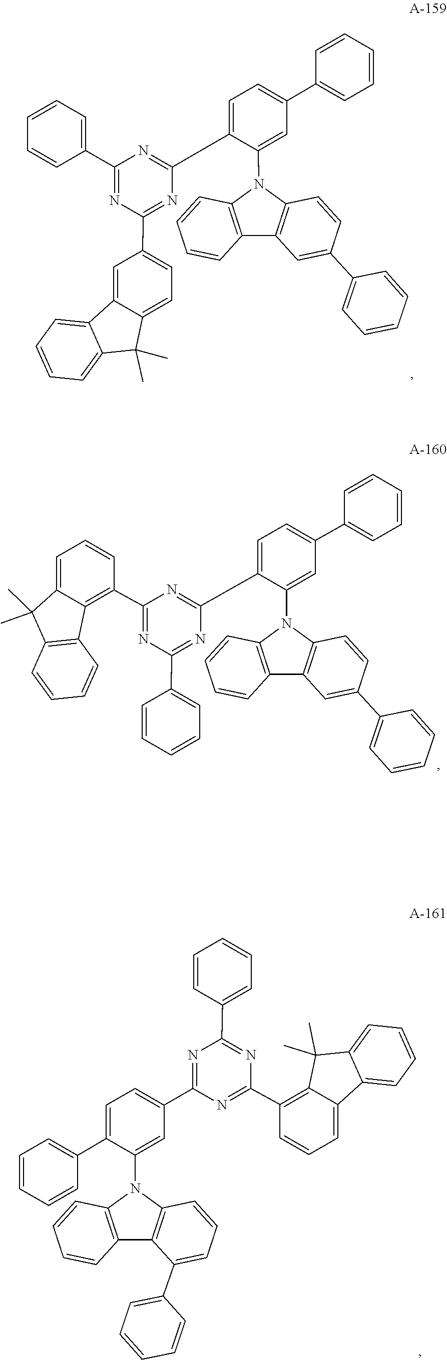 [SMILES: CC1(C)c2ccccc2-c2cc(-c3nc(-c4ccccc4)nc(-c4ccc(-c5ccccc5)cc4-n4c5ccccc5c5cc(-c6ccccc6)ccc54)n3)ccc21 ; CC1(C)c2ccccc2-c2c(-c3nc(-c4ccccc4)nc(-c4ccc(-c5ccccc5)cc4-n4c5ccccc5c5cc(-c6ccccc6)ccc54)n3)cccc21 ; CC1(C)c2ccccc2-c2cccc(-c3nc(-c4ccccc4)nc(-c4ccc(-c5ccccc5)c(-n5c6ccccc6c6c(-c7ccccc7)cccc65)c4)n3)c21] 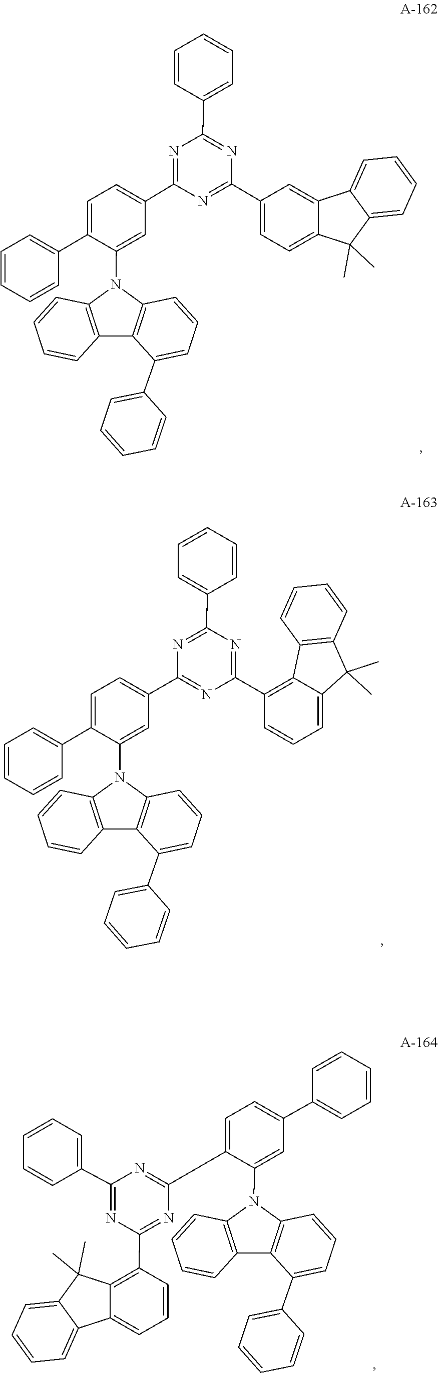 [SMILES: CC1(C)c2ccccc2-c2cc(-c3nc(-c4ccccc4)nc(-c4ccc(-c5ccccc5)c(-n5c6ccccc6c6c(-c7ccccc7)cccc65)c4)n3)ccc21 ; CC1(C)c2ccccc2-c2c(-c3nc(-c4ccccc4)nc(-c4ccc(-c5ccccc5)c(-n5c6ccccc6c6c(-c7ccccc7)cccc65)c4)n3)cccc21 ; CC1(C)c2ccccc2-c2cccc(-c3nc(-c4ccccc4)nc(-c4ccc(-c5ccccc5)cc4-n4c5ccccc5c5c(-c6ccccc6)cccc54)n3)c21] 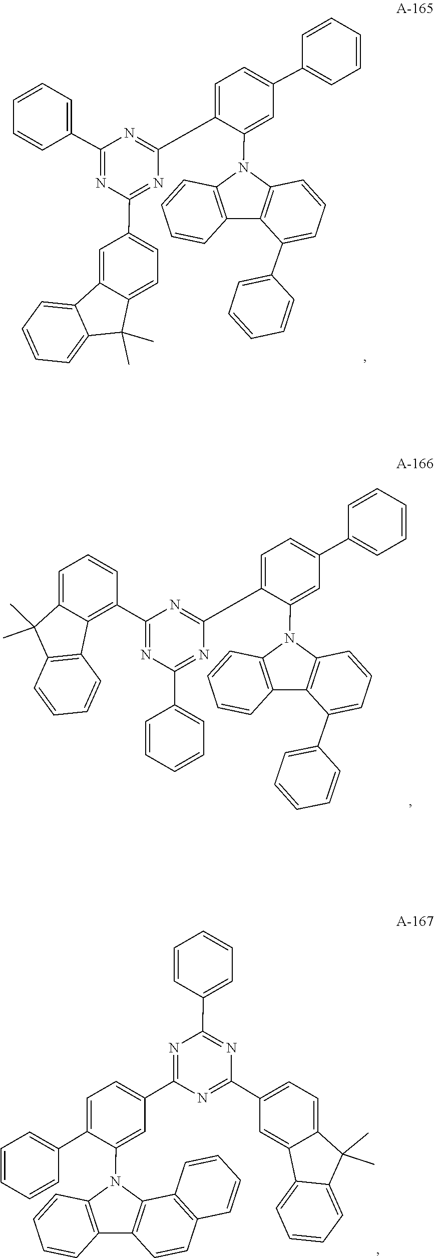 [SMILES: CC1(C)c2ccccc2-c2cc(-c3nc(-c4ccccc4)nc(-c4ccc(-c5ccccc5)cc4-n4c5ccccc5c5c(-c6ccccc6)cccc54)n3)ccc21 ; CC1(C)c2ccccc2-c2c(-c3nc(-c4ccccc4)nc(-c4ccc(-c5ccccc5)cc4-n4c5ccccc5c5c(-c6ccccc6)cccc54)n3)cccc21 ; CC1(C)c2ccccc2-c2cc(-c3nc(-c4ccccc4)nc(-c4ccc(-c5ccccc5)c(-n5c6ccccc6c6ccc7ccccc7c65)c4)n3)ccc21] 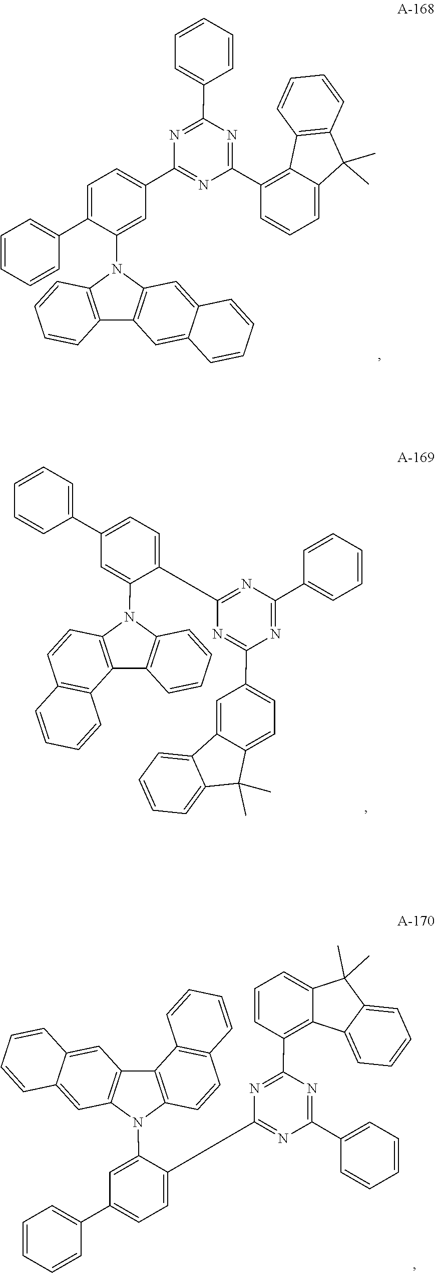 [SMILES: CC1(C)c2ccccc2-c2c(-c3nc(-c4ccccc4)nc(-c4ccc(-c5ccccc5)c(-n5c6ccccc6c6cc7ccccc7cc65)c4)n3)cccc21 ; CC1(C)c2ccccc2-c2cc(-c3nc(-c4ccccc4)nc(-c4ccc(-c5ccccc5)cc4-n4c5ccccc5c5c6ccccc6ccc54)n3)ccc21 ; CC1(C)c2ccccc2-c2c(-c3nc(-c4ccccc4)nc(-c4ccc(-c5ccccc5)cc4-n4c5cc6ccccc6cc5c5c6ccccc6ccc54)n3)cccc21] 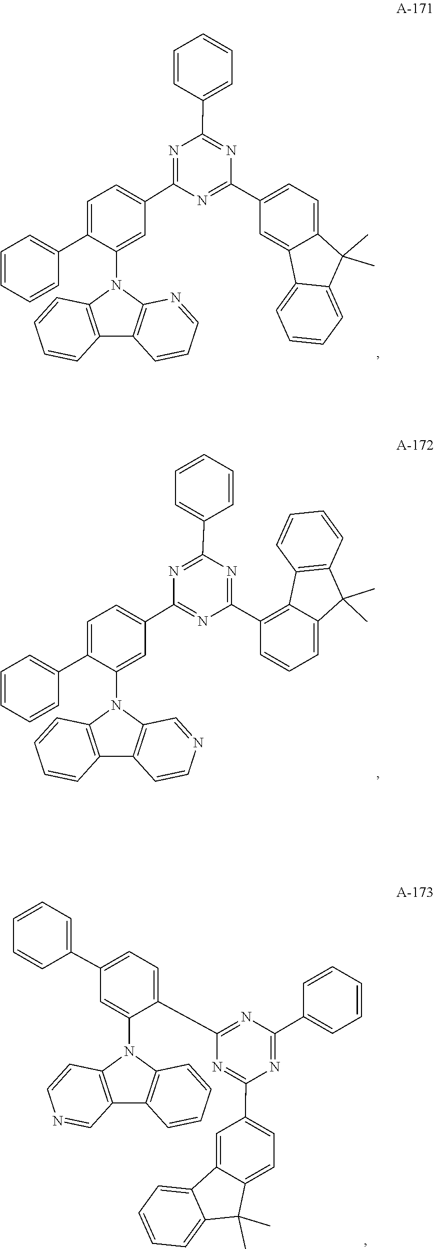 [SMILES: CC1(C)c2ccccc2-c2cc(-c3nc(-c4ccccc4)nc(-c4ccc(-c5ccccc5)c(-n5c6ccccc6c6cccnc65)c4)n3)ccc21 ; CC1(C)c2ccccc2-c2c(-c3nc(-c4ccccc4)nc(-c4ccc(-c5ccccc5)c(-n5c6ccccc6c6ccncc65)c4)n3)cccc21 ; CC1(C)c2ccccc2-c2cc(-c3nc(-c4ccccc4)nc(-c4ccc(-c5ccccc5)cc4-n4c5ccccc5c5cnccc54)n3)ccc21] 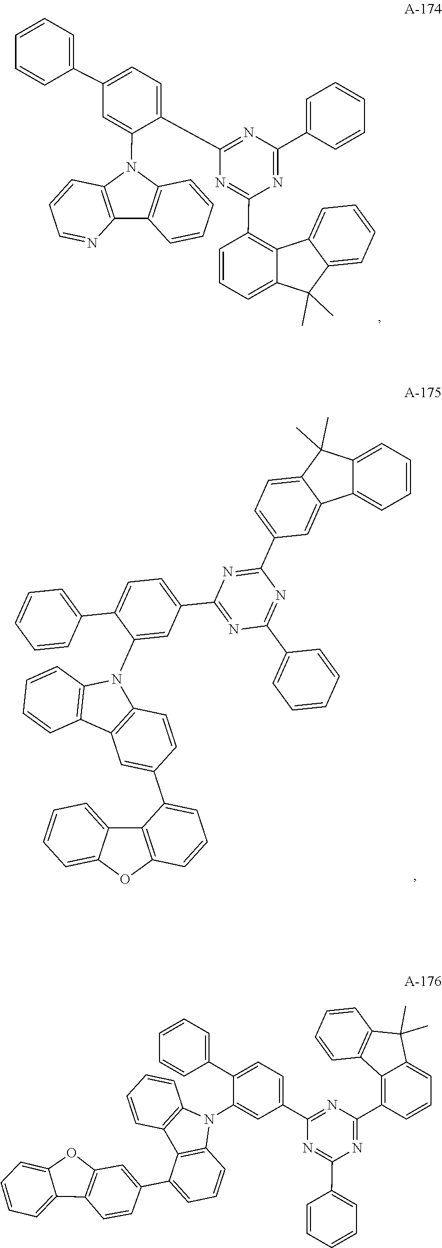 [SMILES: CC1(C)c2ccccc2-c2c(-c3nc(-c4ccccc4)nc(-c4ccc(-c5ccccc5)cc4-n4c5ccccc5c5ncccc54)n3)cccc21 ; CC1(C)c2ccccc2-c2cc(-c3nc(-c4ccccc4)nc(-c4ccc(-c5ccccc5)c(-n5c6ccccc6c6cc(-c7cccc8oc9ccccc9c78)ccc65)c4)n3)ccc21 ; CC1(C)c2ccccc2-c2c(-c3nc(-c4ccccc4)nc(-c4ccc(-c5ccccc5)c(-n5c6ccccc6c6c(-c7ccc8c(c7)oc7ccccc78)cccc65)c4)n3)cccc21] 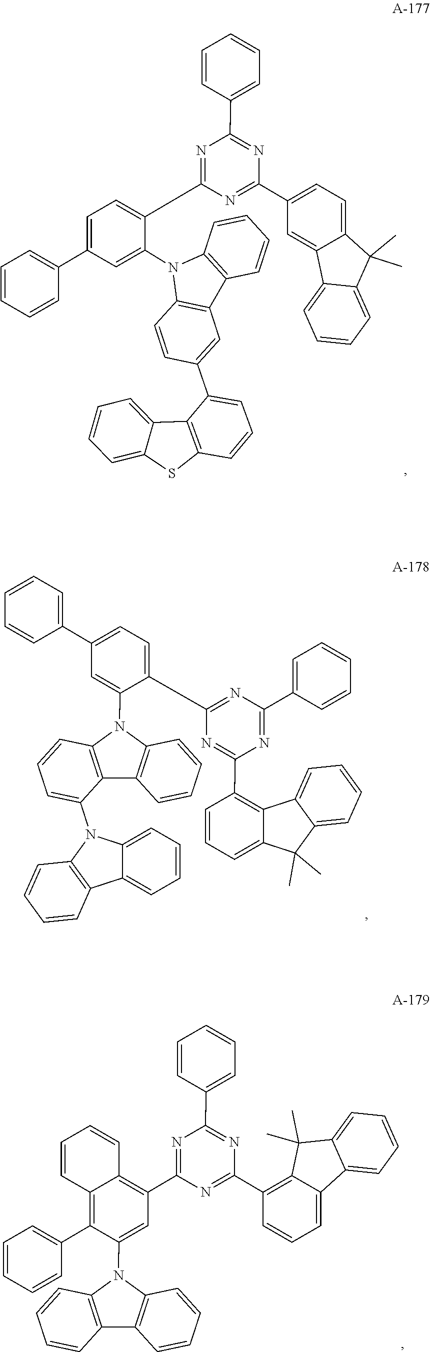 [SMILES: CC1(C)c2ccccc2-c2cc(-c3nc(-c4ccccc4)nc(-c4ccc(-c5ccccc5)cc4-n4c5ccccc5c5cc(-c6cccc7sc8ccccc8c67)ccc54)n3)ccc21 ; CC1(C)c2ccccc2-c2c(-c3nc(-c4ccccc4)nc(-c4ccc(-c5ccccc5)cc4-n4c5ccccc5c5c(-n6c7ccccc7c7ccccc76)cccc54)n3)cccc21 ; CC1(C)c2ccccc2-c2cccc(-c3nc(-c4ccccc4)nc(-c4cc(-n5c6ccccc6c6ccccc65)c(-c5ccccc5)c5ccccc45)n3)c21] 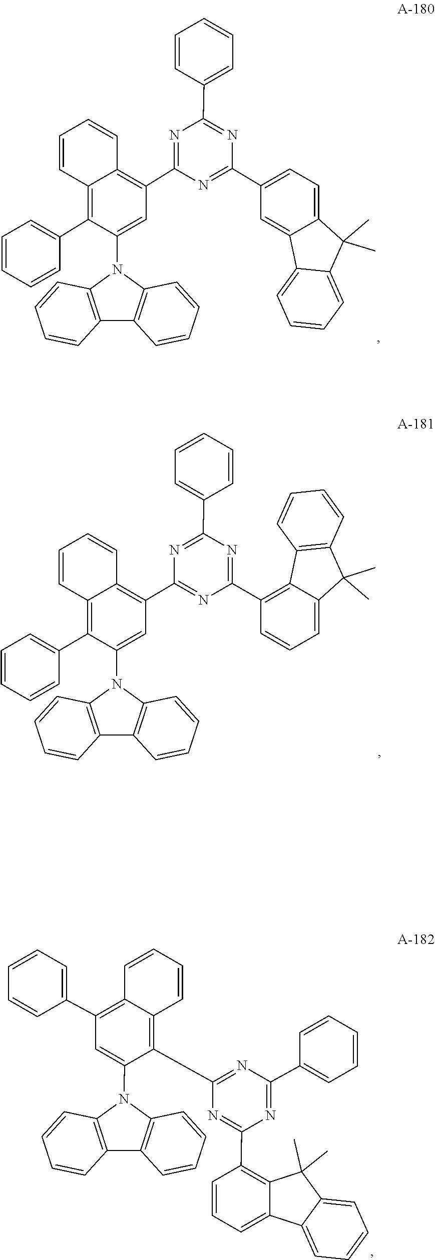 [SMILES: CC1(C)c2ccccc2-c2cc(-c3nc(-c4ccccc4)nc(-c4cc(-n5c6ccccc6c6ccccc65)c(-c5ccccc5)c5ccccc45)n3)ccc21 ; CC1(C)c2ccccc2-c2c(-c3nc(-c4ccccc4)nc(-c4cc(-n5c6ccccc6c6ccccc65)c(-c5ccccc5)c5ccccc45)n3)cccc21 ; CC1(C)c2ccccc2-c2cccc(-c3nc(-c4ccccc4)nc(-c4c(-n5c6ccccc6c6ccccc65)cc(-c5ccccc5)c5ccccc45)n3)c21] 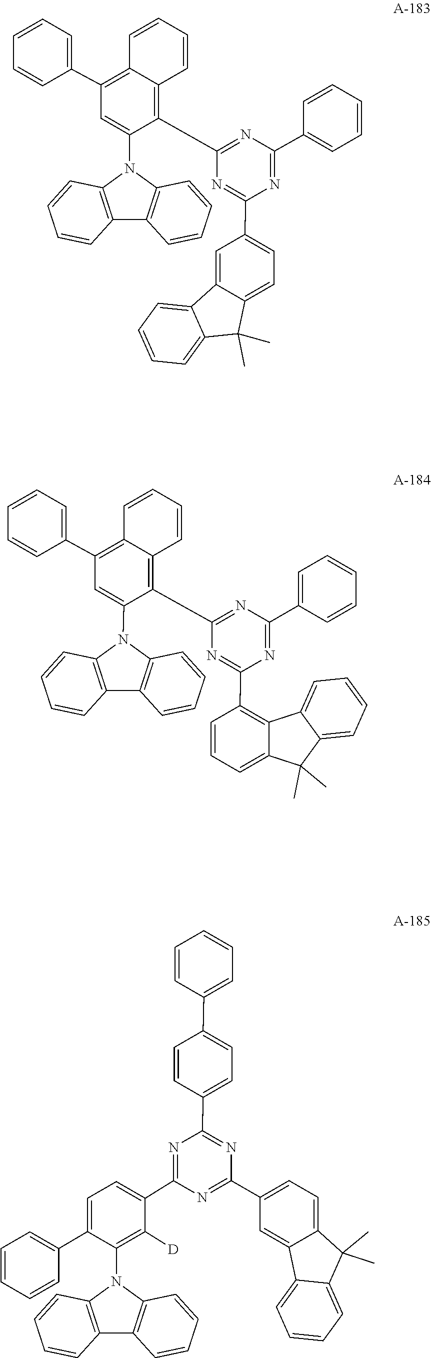 [SMILES: CC1(C)c2ccccc2-c2cc(-c3nc(-c4ccccc4)nc(-c4c(-n5c6ccccc6c6ccccc65)cc(-c5ccccc5)c5ccccc45)n3)ccc21 ; CC1(C)c2ccccc2-c2c(-c3nc(-c4ccccc4)nc(-c4c(-n5c6ccccc6c6ccccc65)cc(-c5ccccc5)c5ccccc45)n3)cccc21 ; [2H]c1c(-c2nc(-c3ccc(-c4ccccc4)cc3)nc(-c3ccc4c(c3)-c3ccccc3C4(C)C)n2)ccc(-c2ccccc2)c1-n1c2ccccc2c2ccccc21] 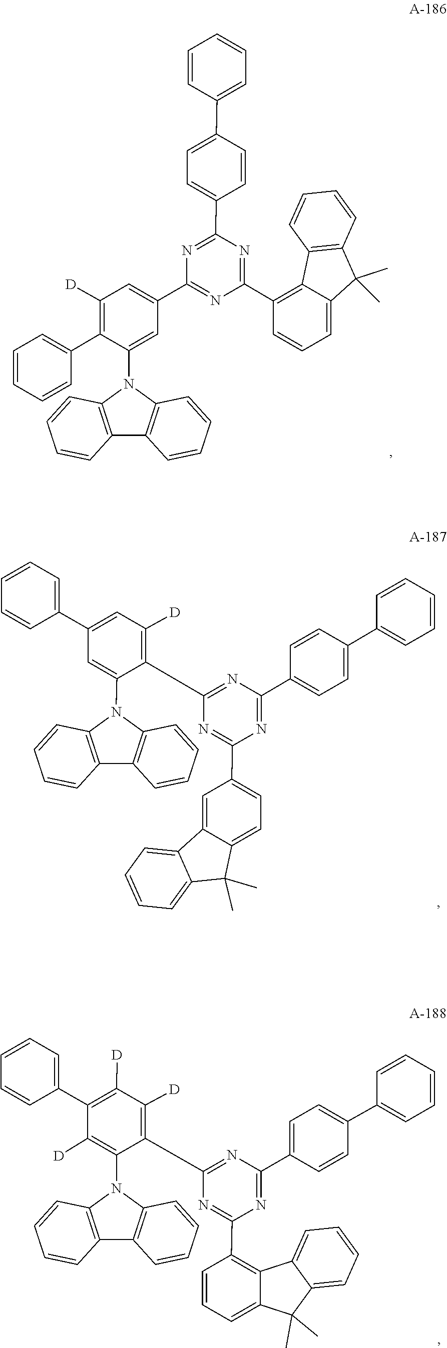 [SMILES: [2H]c1cc(-c2nc(-c3ccc(-c4ccccc4)cc3)nc(-c3cccc4c3-c3ccccc3C4(C)C)n2)cc(-n2c3ccccc3c3ccccc32)c1-c1ccccc1 ; [2H]c1cc(-c2ccccc2)cc(-n2c3ccccc3c3ccccc32)c1-c1nc(-c2ccc(-c3ccccc3)cc2)nc(-c2ccc3c(c2)-c2ccccc2C3(C)C)n1 ; [2H]c1c([2H])c(-c2nc(-c3ccc(-c4ccccc4)cc3)nc(-c3cccc4c3-c3ccccc3C4(C)C)n2)c(-n2c3ccccc3c3ccccc32)c([2H])c1-c1ccccc1] 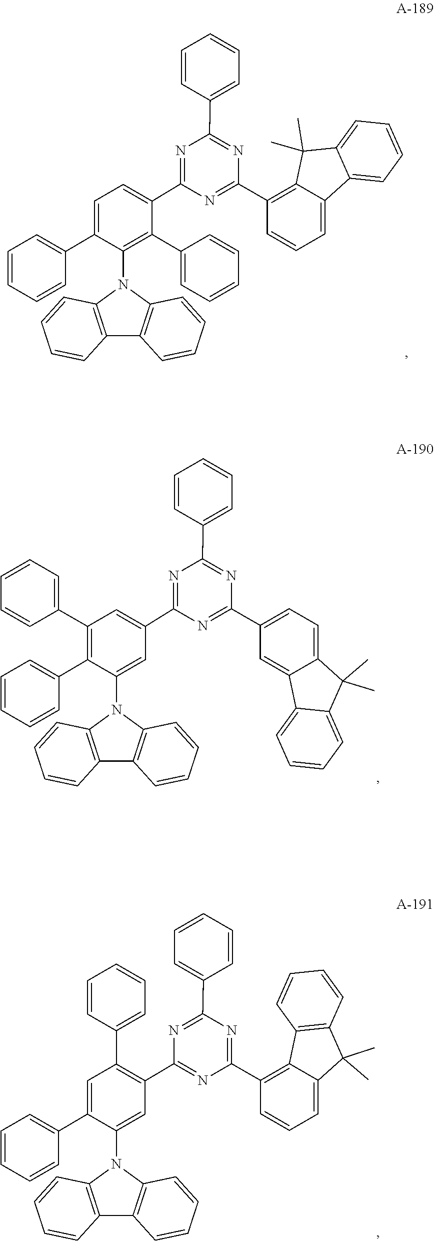 [SMILES: CC1(C)c2ccccc2-c2cccc(-c3nc(-c4ccccc4)nc(-c4ccc(-c5ccccc5)c(-n5c6ccccc6c6ccccc65)c4-c4ccccc4)n3)c21 ; CC1(C)c2ccccc2-c2cc(-c3nc(-c4ccccc4)nc(-c4cc(-c5ccccc5)c(-c5ccccc5)c(-n5c6ccccc6c6ccccc65)c4)n3)ccc21 ; CC1(C)c2ccccc2-c2c(-c3nc(-c4ccccc4)nc(-c4cc(-n5c6ccccc6c6ccccc65)c(-c5ccccc5)cc4-c4ccccc4)n3)cccc21] 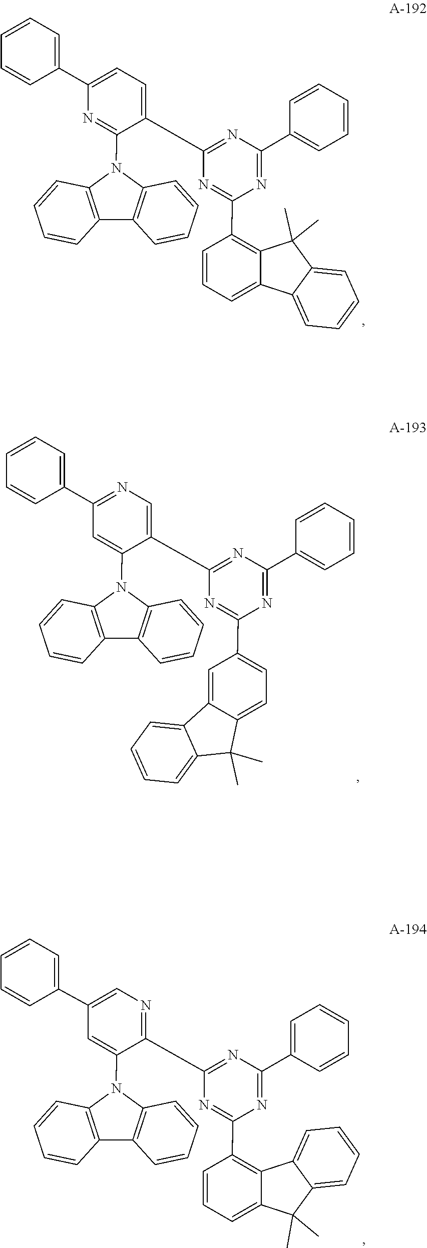 [SMILES: CC1(C)c2ccccc2-c2cccc(-c3nc(-c4ccccc4)nc(-c4ccc(-c5ccccc5)nc4-n4c5ccccc5c5ccccc54)n3)c21 ; CC1(C)c2ccccc2-c2cc(-c3nc(-c4ccccc4)nc(-c4cnc(-c5ccccc5)cc4-n4c5ccccc5c5ccccc54)n3)ccc21 ; CC1(C)c2ccccc2-c2c(-c3nc(-c4ccccc4)nc(-c4ncc(-c5ccccc5)cc4-n4c5ccccc5c5ccccc54)n3)cccc21] 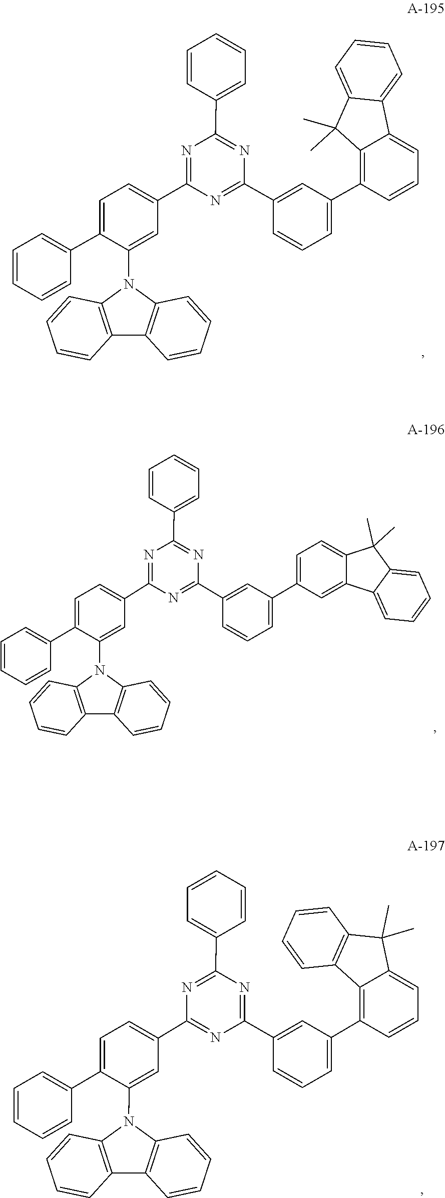 [SMILES: CC1(C)c2ccccc2-c2cccc(-c3cccc(-c4nc(-c5ccccc5)nc(-c5ccc(-c6ccccc6)c(-n6c7ccccc7c7ccccc76)c5)n4)c3)c21 ; CC1(C)c2ccccc2-c2cc(-c3cccc(-c4nc(-c5ccccc5)nc(-c5ccc(-c6ccccc6)c(-n6c7ccccc7c7ccccc76)c5)n4)c3)ccc21 ; CC1(C)c2ccccc2-c2c(-c3cccc(-c4nc(-c5ccccc5)nc(-c5ccc(-c6ccccc6)c(-n6c7ccccc7c7ccccc76)c5)n4)c3)cccc21] 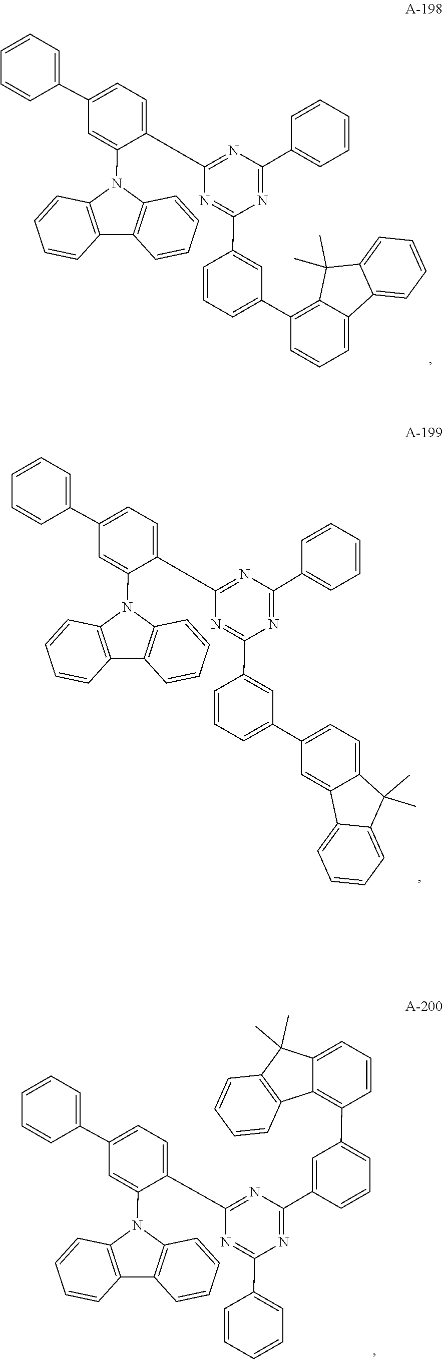 [SMILES: CC1(C)c2ccccc2-c2cccc(-c3cccc(-c4nc(-c5ccccc5)nc(-c5ccc(-c6ccccc6)cc5-n5c6ccccc6c6ccccc65)n4)c3)c21 ; CC1(C)c2ccccc2-c2cc(-c3cccc(-c4nc(-c5ccccc5)nc(-c5ccc(-c6ccccc6)cc5-n5c6ccccc6c6ccccc65)n4)c3)ccc21 ; CC1(C)c2ccccc2-c2c(-c3cccc(-c4nc(-c5ccccc5)nc(-c5ccc(-c6ccccc6)cc5-n5c6ccccc6c6ccccc65)n4)c3)cccc21] 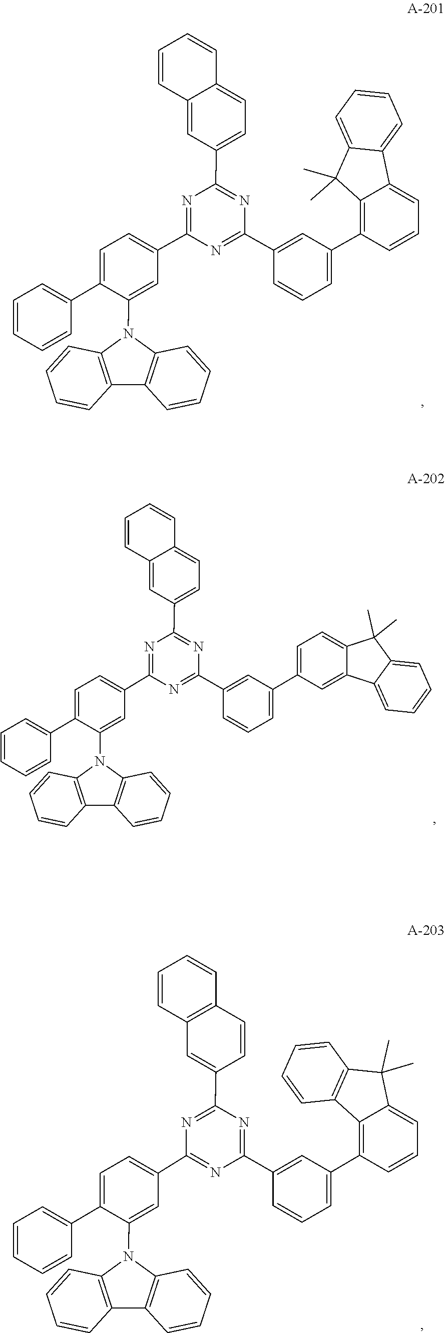 [SMILES: CC1(C)c2ccccc2-c2cccc(-c3cccc(-c4nc(-c5ccc(-c6ccccc6)c(-n6c7ccccc7c7ccccc76)c5)nc(-c5ccc6ccccc6c5)n4)c3)c21 ; CC1(C)c2ccccc2-c2cc(-c3cccc(-c4nc(-c5ccc(-c6ccccc6)c(-n6c7ccccc7c7ccccc76)c5)nc(-c5ccc6ccccc6c5)n4)c3)ccc21 ; CC1(C)c2ccccc2-c2c(-c3cccc(-c4nc(-c5ccc(-c6ccccc6)c(-n6c7ccccc7c7ccccc76)c5)nc(-c5ccc6ccccc6c5)n4)c3)cccc21] 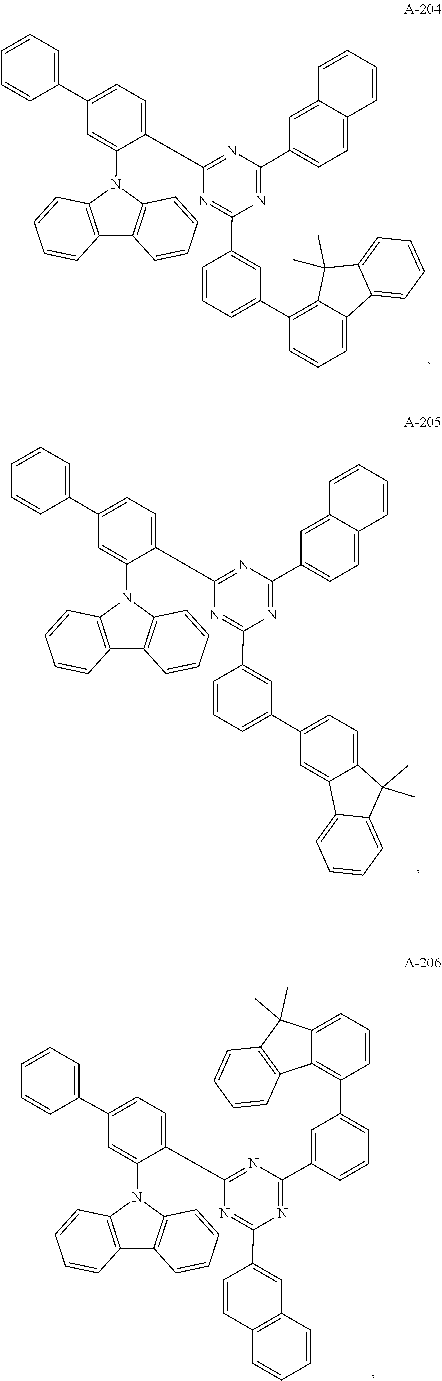 [SMILES: CC1(C)c2ccccc2-c2cccc(-c3cccc(-c4nc(-c5ccc6ccccc6c5)nc(-c5ccc(-c6ccccc6)cc5-n5c6ccccc6c6ccccc65)n4)c3)c21 ; CC1(C)c2ccccc2-c2cc(-c3cccc(-c4nc(-c5ccc6ccccc6c5)nc(-c5ccc(-c6ccccc6)cc5-n5c6ccccc6c6ccccc65)n4)c3)ccc21 ; CC1(C)c2ccccc2-c2c(-c3cccc(-c4nc(-c5ccc6ccccc6c5)nc(-c5ccc(-c6ccccc6)cc5-n5c6ccccc6c6ccccc65)n4)c3)cccc21] 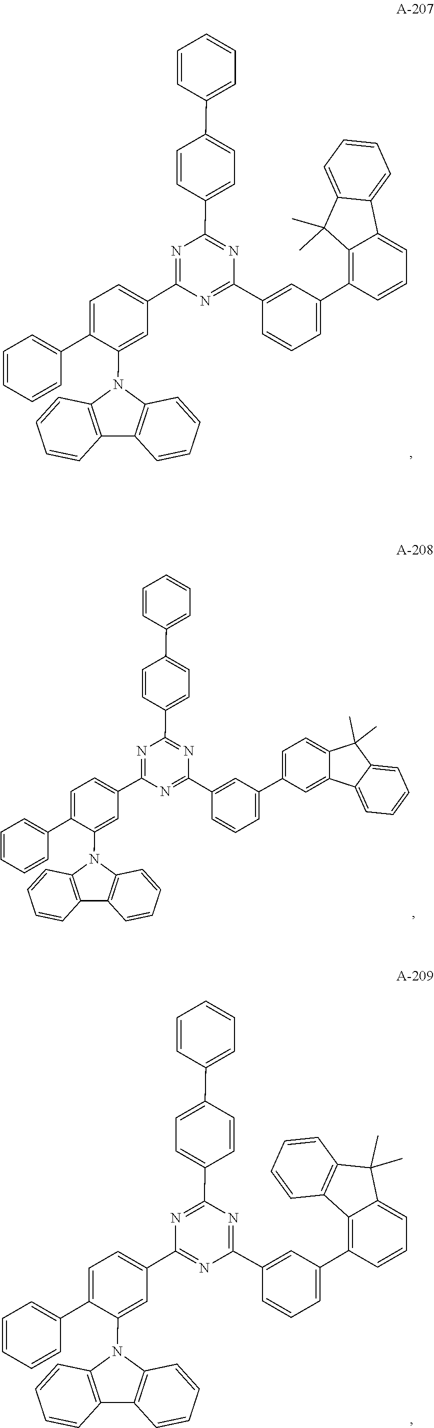 [SMILES: CC1(C)c2ccccc2-c2cccc(-c3cccc(-c4nc(-c5ccc(-c6ccccc6)cc5)nc(-c5ccc(-c6ccccc6)c(-n6c7ccccc7c7ccccc76)c5)n4)c3)c21 ; CC1(C)c2ccccc2-c2cc(-c3cccc(-c4nc(-c5ccc(-c6ccccc6)cc5)nc(-c5ccc(-c6ccccc6)c(-n6c7ccccc7c7ccccc76)c5)n4)c3)ccc21 ; CC1(C)c2ccccc2-c2c(-c3cccc(-c4nc(-c5ccc(-c6ccccc6)cc5)nc(-c5ccc(-c6ccccc6)c(-n6c7ccccc7c7ccccc76)c5)n4)c3)cccc21] 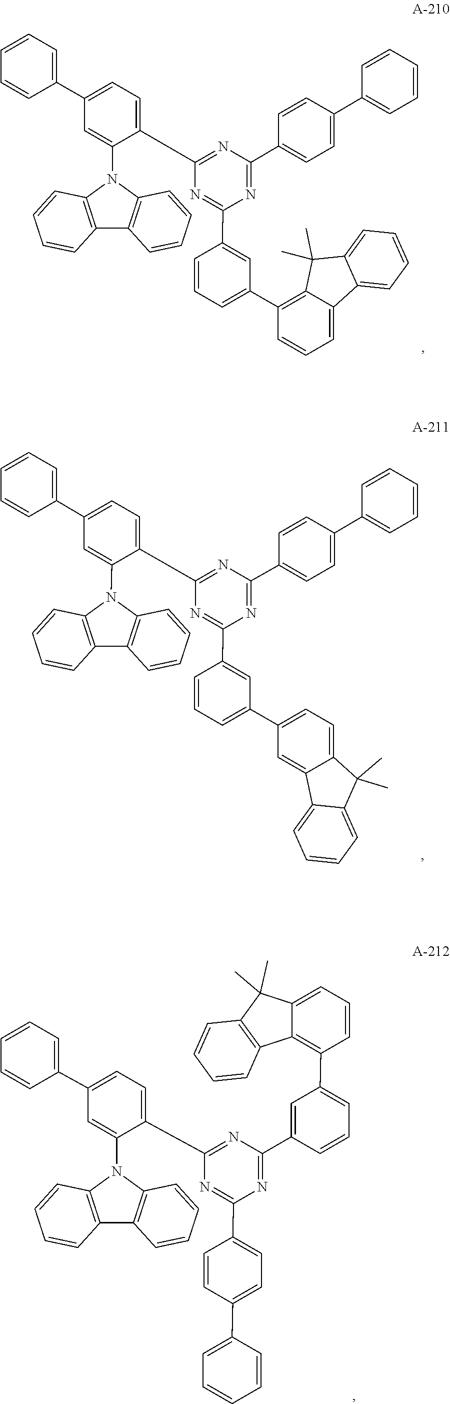 [SMILES: CC1(C)c2ccccc2-c2cccc(-c3cccc(-c4nc(-c5ccc(-c6ccccc6)cc5)nc(-c5ccc(-c6ccccc6)cc5-n5c6ccccc6c6ccccc65)n4)c3)c21 ; CC1(C)c2ccccc2-c2cc(-c3cccc(-c4nc(-c5ccc(-c6ccccc6)cc5)nc(-c5ccc(-c6ccccc6)cc5-n5c6ccccc6c6ccccc65)n4)c3)ccc21 ; CC1(C)c2ccccc2-c2c(-c3cccc(-c4nc(-c5ccc(-c6ccccc6)cc5)nc(-c5ccc(-c6ccccc6)cc5-n5c6ccccc6c6ccccc65)n4)c3)cccc21] 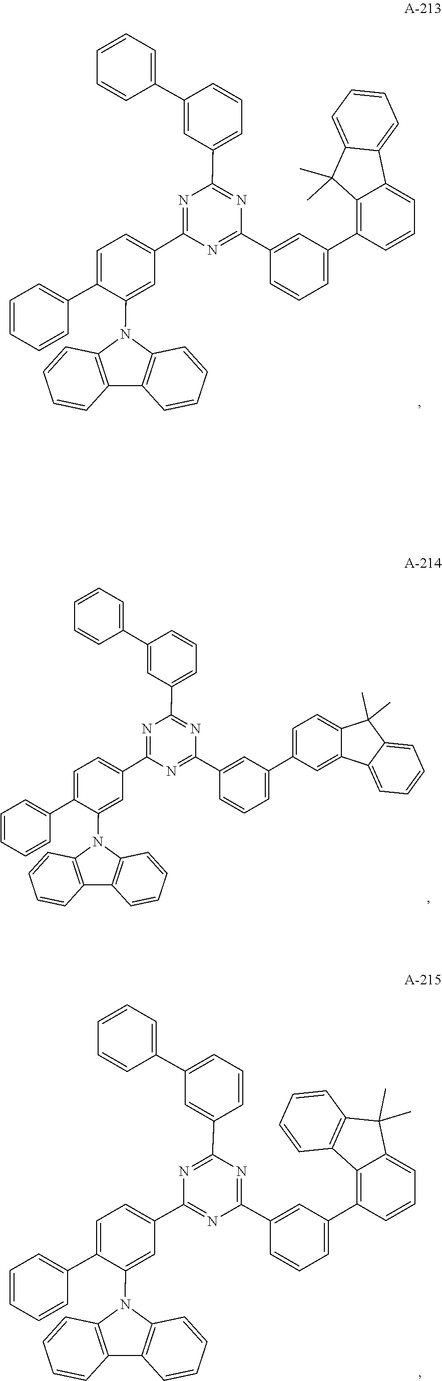 [SMILES: CC1(C)c2ccccc2-c2cccc(-c3cccc(-c4nc(-c5cccc(-c6ccccc6)c5)nc(-c5ccc(-c6ccccc6)c(-n6c7ccccc7c7ccccc76)c5)n4)c3)c21 ; CC1(C)c2ccccc2-c2cc(-c3cccc(-c4nc(-c5cccc(-c6ccccc6)c5)nc(-c5ccc(-c6ccccc6)c(-n6c7ccccc7c7ccccc76)c5)n4)c3)ccc21 ; CC1(C)c2ccccc2-c2c(-c3cccc(-c4nc(-c5cccc(-c6ccccc6)c5)nc(-c5ccc(-c6ccccc6)c(-n6c7ccccc7c7ccccc76)c5)n4)c3)cccc21] 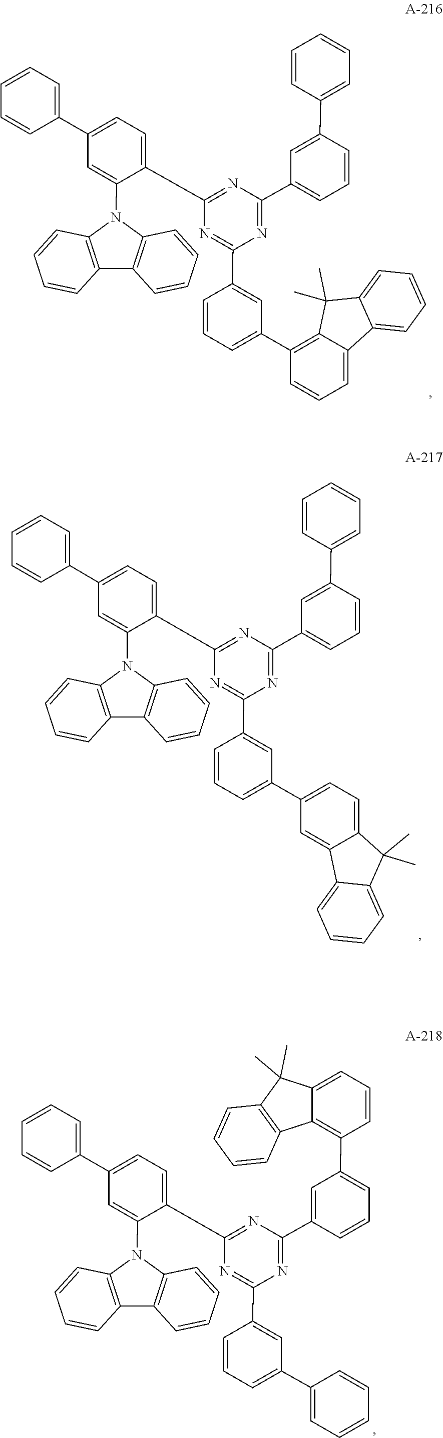 [SMILES: CC1(C)c2ccccc2-c2cccc(-c3cccc(-c4nc(-c5cccc(-c6ccccc6)c5)nc(-c5ccc(-c6ccccc6)cc5-n5c6ccccc6c6ccccc65)n4)c3)c21 ; CC1(C)c2ccccc2-c2cc(-c3cccc(-c4nc(-c5cccc(-c6ccccc6)c5)nc(-c5ccc(-c6ccccc6)cc5-n5c6ccccc6c6ccccc65)n4)c3)ccc21 ; CC1(C)c2ccccc2-c2c(-c3cccc(-c4nc(-c5cccc(-c6ccccc6)c5)nc(-c5ccc(-c6ccccc6)cc5-n5c6ccccc6c6ccccc65)n4)c3)cccc21] 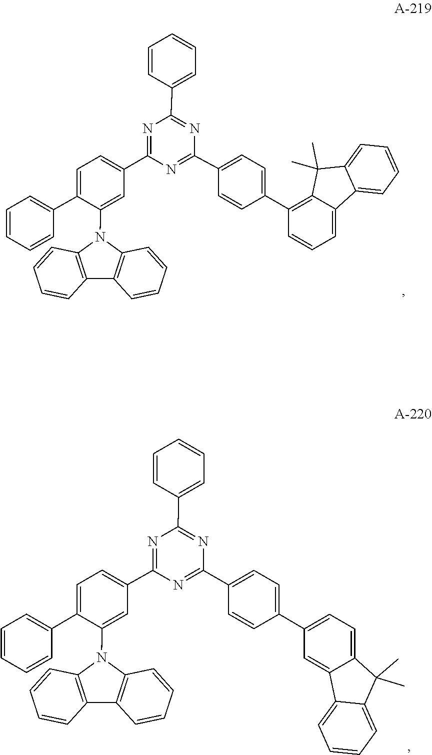 [SMILES: CC1(C)c2ccccc2-c2cccc(-c3ccc(-c4nc(-c5ccccc5)nc(-c5ccc(-c6ccccc6)c(-n6c7ccccc7c7ccccc76)c5)n4)cc3)c21 ; CC1(C)c2ccccc2-c2cc(-c3ccc(-c4nc(-c5ccccc5)nc(-c5ccc(-c6ccccc6)c(-n6c7ccccc7c7ccccc76)c5)n4)cc3)ccc21] 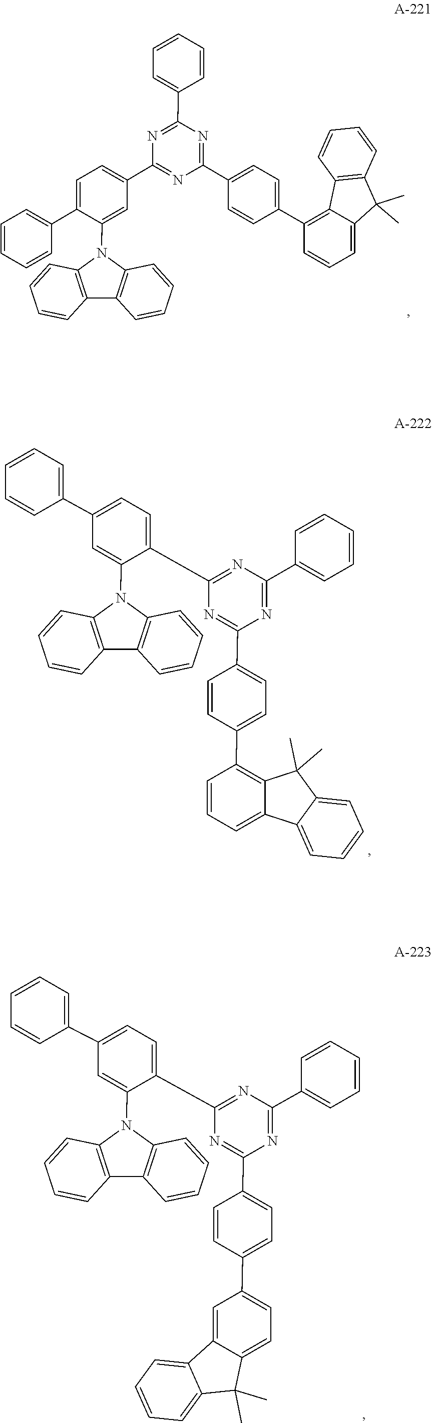 [SMILES: CC1(C)c2ccccc2-c2c(-c3ccc(-c4nc(-c5ccccc5)nc(-c5ccc(-c6ccccc6)c(-n6c7ccccc7c7ccccc76)c5)n4)cc3)cccc21 ; CC1(C)c2ccccc2-c2cccc(-c3ccc(-c4nc(-c5ccccc5)nc(-c5ccc(-c6ccccc6)cc5-n5c6ccccc6c6ccccc65)n4)cc3)c21 ; CC1(C)c2ccccc2-c2cc(-c3ccc(-c4nc(-c5ccccc5)nc(-c5ccc(-c6ccccc6)cc5-n5c6ccccc6c6ccccc65)n4)cc3)ccc21] 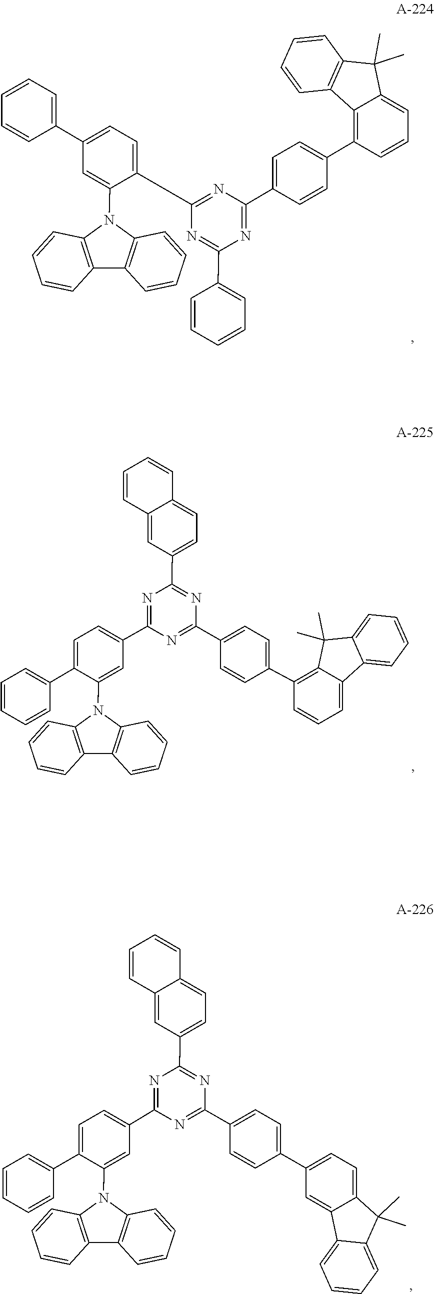 [SMILES: CC1(C)c2ccccc2-c2c(-c3ccc(-c4nc(-c5ccccc5)nc(-c5ccc(-c6ccccc6)cc5-n5c6ccccc6c6ccccc65)n4)cc3)cccc21 ; CC1(C)c2ccccc2-c2cccc(-c3ccc(-c4nc(-c5ccc(-c6ccccc6)c(-n6c7ccccc7c7ccccc76)c5)nc(-c5ccc6ccccc6c5)n4)cc3)c21 ; CC1(C)c2ccccc2-c2cc(-c3ccc(-c4nc(-c5ccc(-c6ccccc6)c(-n6c7ccccc7c7ccccc76)c5)nc(-c5ccc6ccccc6c5)n4)cc3)ccc21] 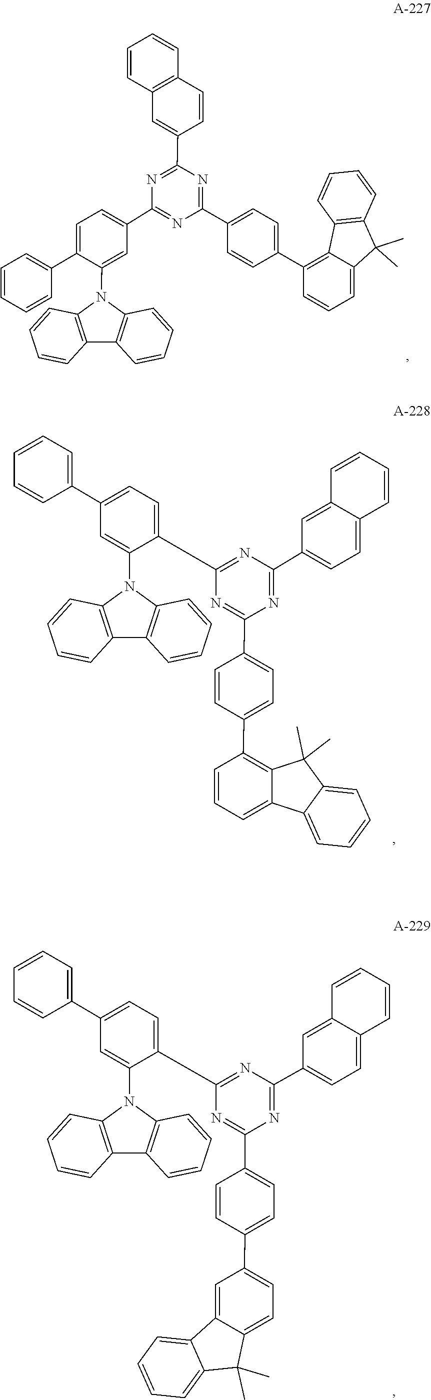 [SMILES: CC1(C)c2ccccc2-c2c(-c3ccc(-c4nc(-c5ccc(-c6ccccc6)c(-n6c7ccccc7c7ccccc76)c5)nc(-c5ccc6ccccc6c5)n4)cc3)cccc21 ; CC1(C)c2ccccc2-c2cccc(-c3ccc(-c4nc(-c5ccc6ccccc6c5)nc(-c5ccc(-c6ccccc6)cc5-n5c6ccccc6c6ccccc65)n4)cc3)c21 ; CC1(C)c2ccccc2-c2cc(-c3ccc(-c4nc(-c5ccc6ccccc6c5)nc(-c5ccc(-c6ccccc6)cc5-n5c6ccccc6c6ccccc65)n4)cc3)ccc21] 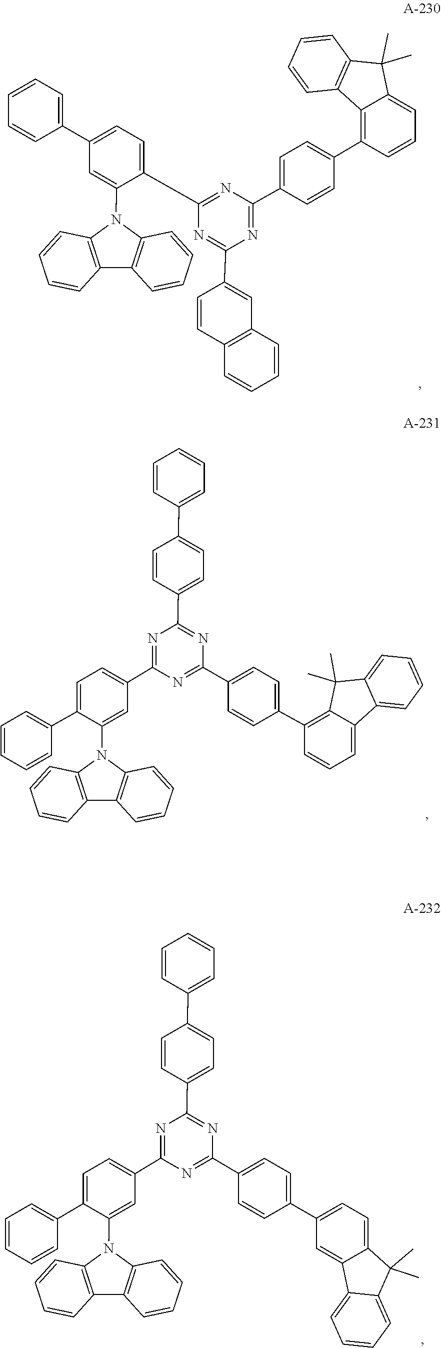 [SMILES: CC1(C)c2ccccc2-c2c(-c3ccc(-c4nc(-c5ccc6ccccc6c5)nc(-c5ccc(-c6ccccc6)cc5-n5c6ccccc6c6ccccc65)n4)cc3)cccc21 ; CC1(C)c2ccccc2-c2cccc(-c3ccc(-c4nc(-c5ccc(-c6ccccc6)cc5)nc(-c5ccc(-c6ccccc6)c(-n6c7ccccc7c7ccccc76)c5)n4)cc3)c21 ; CC1(C)c2ccccc2-c2cc(-c3ccc(-c4nc(-c5ccc(-c6ccccc6)cc5)nc(-c5ccc(-c6ccccc6)c(-n6c7ccccc7c7ccccc76)c5)n4)cc3)ccc21] 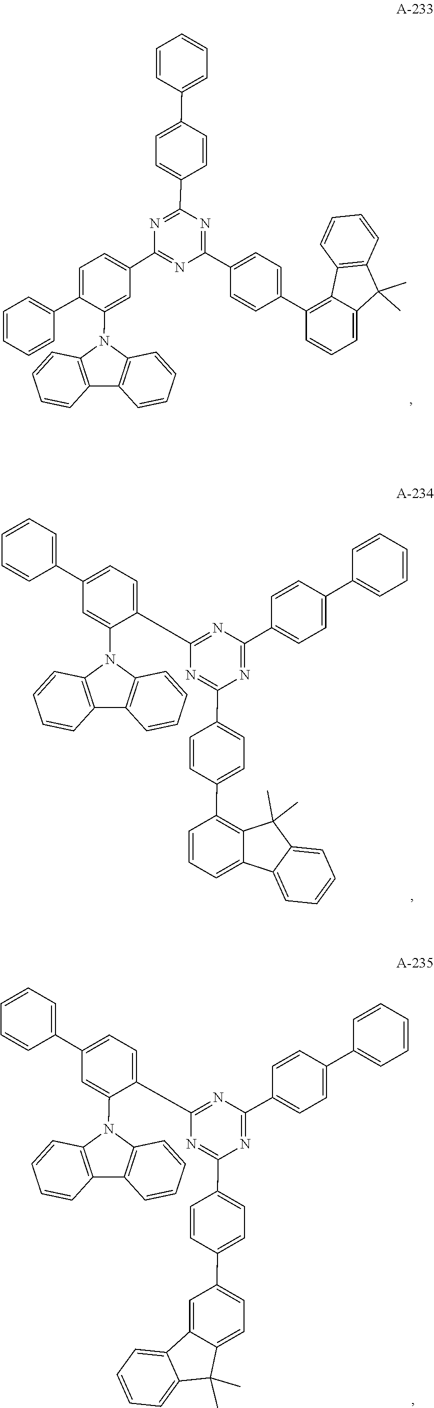 [SMILES: CC1(C)c2ccccc2-c2c(-c3ccc(-c4nc(-c5ccc(-c6ccccc6)cc5)nc(-c5ccc(-c6ccccc6)c(-n6c7ccccc7c7ccccc76)c5)n4)cc3)cccc21 ; CC1(C)c2ccccc2-c2cccc(-c3ccc(-c4nc(-c5ccc(-c6ccccc6)cc5)nc(-c5ccc(-c6ccccc6)cc5-n5c6ccccc6c6ccccc65)n4)cc3)c21 ; CC1(C)c2ccccc2-c2cc(-c3ccc(-c4nc(-c5ccc(-c6ccccc6)cc5)nc(-c5ccc(-c6ccccc6)cc5-n5c6ccccc6c6ccccc65)n4)cc3)ccc21] 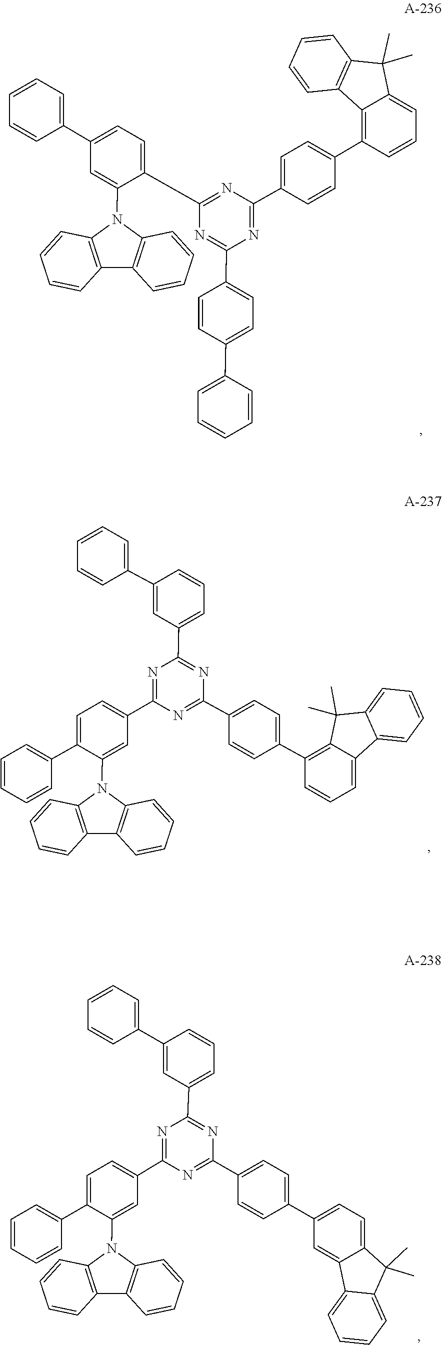 [SMILES: CC1(C)c2ccccc2-c2c(-c3ccc(-c4nc(-c5ccc(-c6ccccc6)cc5)nc(-c5ccc(-c6ccccc6)cc5-n5c6ccccc6c6ccccc65)n4)cc3)cccc21 ; CC1(C)c2ccccc2-c2cccc(-c3ccc(-c4nc(-c5cccc(-c6ccccc6)c5)nc(-c5ccc(-c6ccccc6)c(-n6c7ccccc7c7ccccc76)c5)n4)cc3)c21 ; CC1(C)c2ccccc2-c2cc(-c3ccc(-c4nc(-c5cccc(-c6ccccc6)c5)nc(-c5ccc(-c6ccccc6)c(-n6c7ccccc7c7ccccc76)c5)n4)cc3)ccc21] 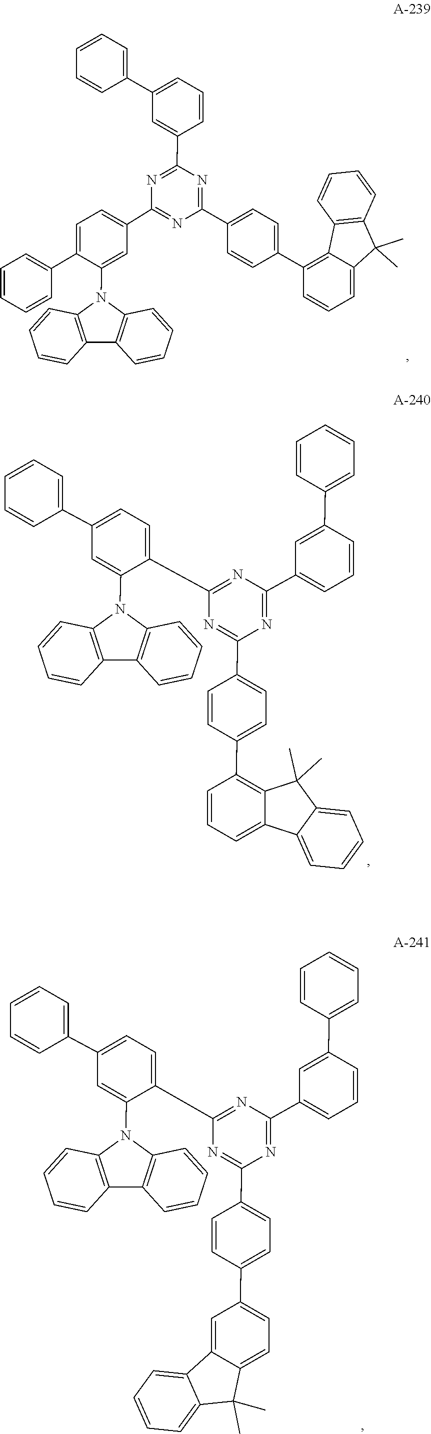 [SMILES: CC1(C)c2ccccc2-c2c(-c3ccc(-c4nc(-c5cccc(-c6ccccc6)c5)nc(-c5ccc(-c6ccccc6)c(-n6c7ccccc7c7ccccc76)c5)n4)cc3)cccc21 ; CC1(C)c2ccccc2-c2cccc(-c3ccc(-c4nc(-c5cccc(-c6ccccc6)c5)nc(-c5ccc(-c6ccccc6)cc5-n5c6ccccc6c6ccccc65)n4)cc3)c21 ; CC1(C)c2ccccc2-c2cc(-c3ccc(-c4nc(-c5cccc(-c6ccccc6)c5)nc(-c5ccc(-c6ccccc6)cc5-n5c6ccccc6c6ccccc65)n4)cc3)ccc21] 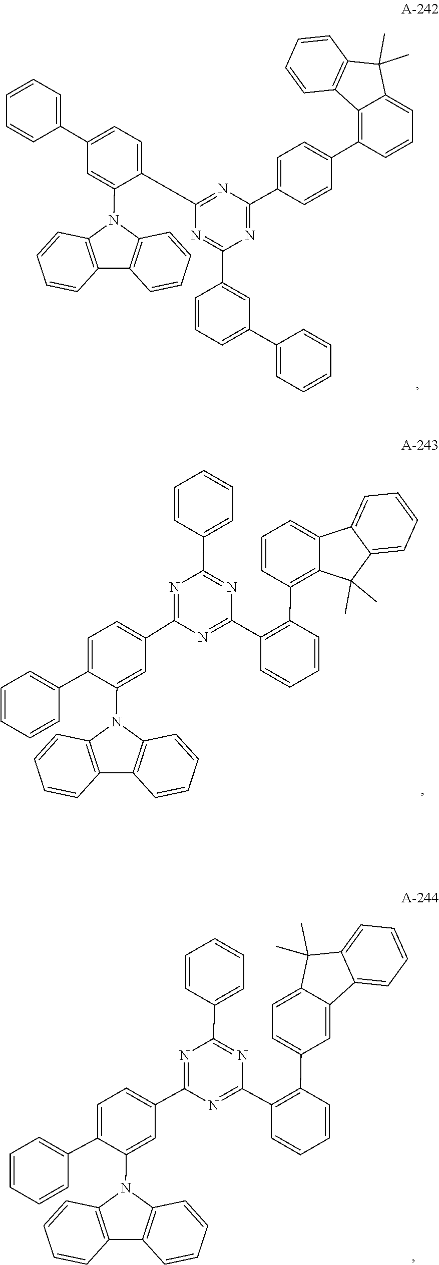 [SMILES: CC1(C)c2ccccc2-c2c(-c3ccc(-c4nc(-c5cccc(-c6ccccc6)c5)nc(-c5ccc(-c6ccccc6)cc5-n5c6ccccc6c6ccccc65)n4)cc3)cccc21 ; CC1(C)c2ccccc2-c2cccc(-c3ccccc3-c3nc(-c4ccccc4)nc(-c4ccc(-c5ccccc5)c(-n5c6ccccc6c6ccccc65)c4)n3)c21 ; CC1(C)c2ccccc2-c2cc(-c3ccccc3-c3nc(-c4ccccc4)nc(-c4ccc(-c5ccccc5)c(-n5c6ccccc6c6ccccc65)c4)n3)ccc21] 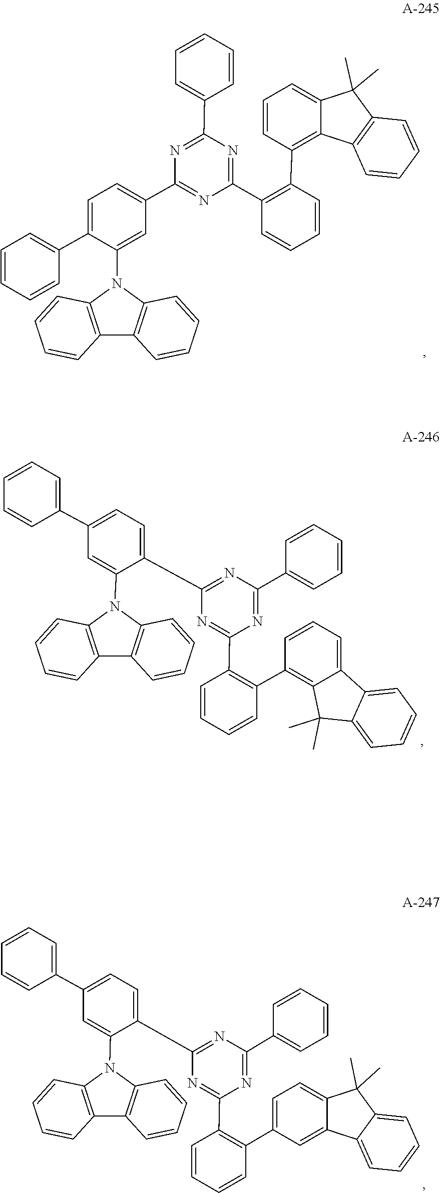 [SMILES: CC1(C)c2ccccc2-c2c(-c3ccccc3-c3nc(-c4ccccc4)nc(-c4ccc(-c5ccccc5)c(-n5c6ccccc6c6ccccc65)c4)n3)cccc21 ; CC1(C)c2ccccc2-c2cccc(-c3ccccc3-c3nc(-c4ccccc4)nc(-c4ccc(-c5ccccc5)cc4-n4c5ccccc5c5ccccc54)n3)c21 ; CC1(C)c2ccccc2-c2cc(-c3ccccc3-c3nc(-c4ccccc4)nc(-c4ccc(-c5ccccc5)cc4-n4c5ccccc5c5ccccc54)n3)ccc21] 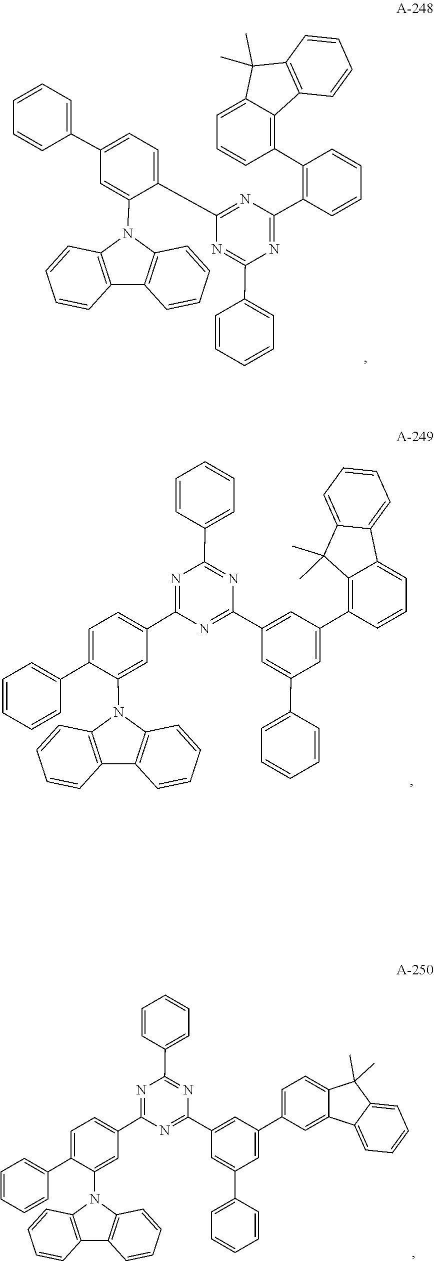 [SMILES: CC1(C)c2ccccc2-c2c(-c3ccccc3-c3nc(-c4ccccc4)nc(-c4ccc(-c5ccccc5)cc4-n4c5ccccc5c5ccccc54)n3)cccc21 ; CC1(C)c2ccccc2-c2cccc(-c3cc(-c4ccccc4)cc(-c4nc(-c5ccccc5)nc(-c5ccc(-c6ccccc6)c(-n6c7ccccc7c7ccccc76)c5)n4)c3)c21 ; CC1(C)c2ccccc2-c2cc(-c3cc(-c4ccccc4)cc(-c4nc(-c5ccccc5)nc(-c5ccc(-c6ccccc6)c(-n6c7ccccc7c7ccccc76)c5)n4)c3)ccc21] 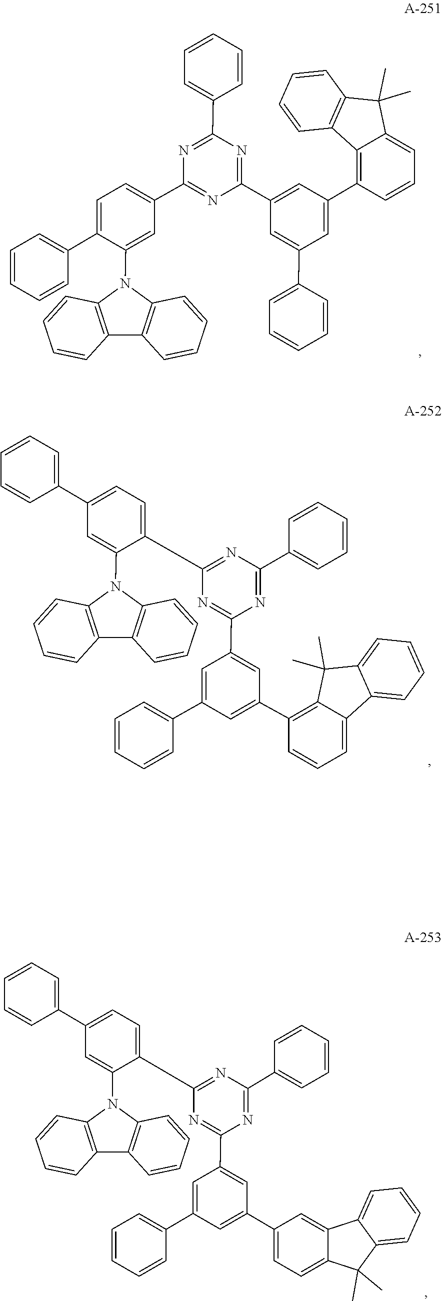 [SMILES: CC1(C)c2ccccc2-c2c(-c3cc(-c4ccccc4)cc(-c4nc(-c5ccccc5)nc(-c5ccc(-c6ccccc6)c(-n6c7ccccc7c7ccccc76)c5)n4)c3)cccc21 ; CC1(C)c2ccccc2-c2cccc(-c3cc(-c4ccccc4)cc(-c4nc(-c5ccccc5)nc(-c5ccc(-c6ccccc6)cc5-n5c6ccccc6c6ccccc65)n4)c3)c21 ; CC1(C)c2ccccc2-c2cc(-c3cc(-c4ccccc4)cc(-c4nc(-c5ccccc5)nc(-c5ccc(-c6ccccc6)cc5-n5c6ccccc6c6ccccc65)n4)c3)ccc21] 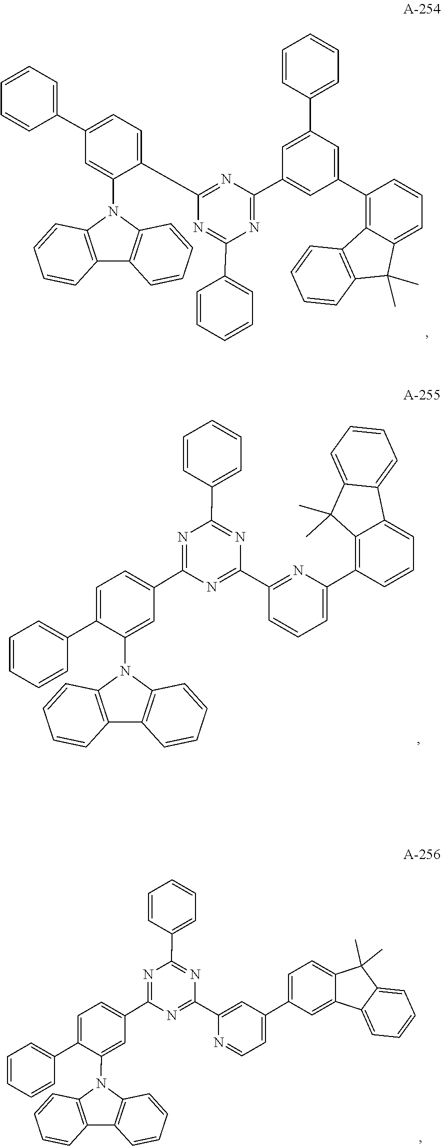 [SMILES: CC1(C)c2ccccc2-c2c(-c3cc(-c4ccccc4)cc(-c4nc(-c5ccccc5)nc(-c5ccc(-c6ccccc6)cc5-n5c6ccccc6c6ccccc65)n4)c3)cccc21 ; CC1(C)c2ccccc2-c2cccc(-c3cccc(-c4nc(-c5ccccc5)nc(-c5ccc(-c6ccccc6)c(-n6c7ccccc7c7ccccc76)c5)n4)n3)c21 ; CC1(C)c2ccccc2-c2cc(-c3ccnc(-c4nc(-c5ccccc5)nc(-c5ccc(-c6ccccc6)c(-n6c7ccccc7c7ccccc76)c5)n4)c3)ccc21] 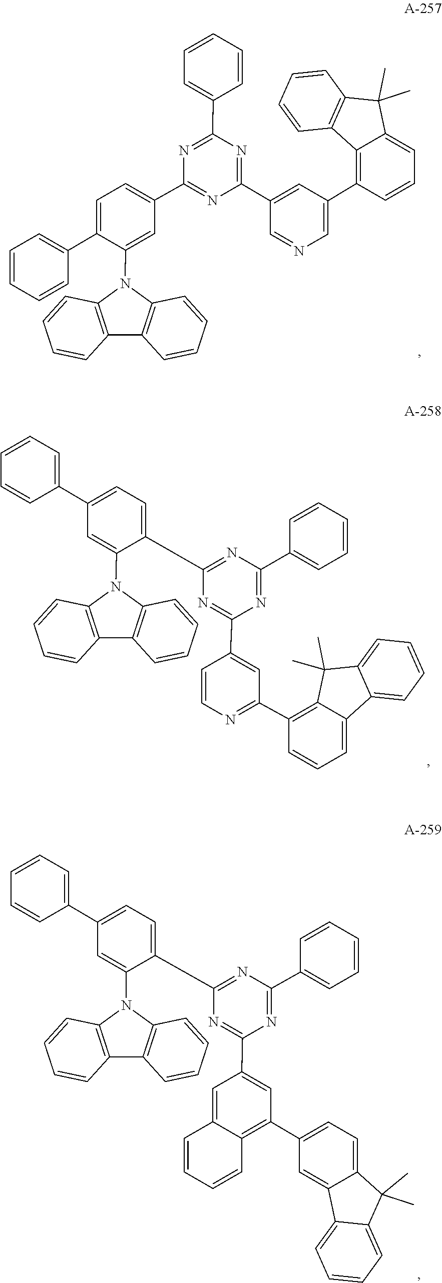 [SMILES: CC1(C)c2ccccc2-c2c(-c3cncc(-c4nc(-c5ccccc5)nc(-c5ccc(-c6ccccc6)c(-n6c7ccccc7c7ccccc76)c5)n4)c3)cccc21 ; CC1(C)c2ccccc2-c2cccc(-c3cc(-c4nc(-c5ccccc5)nc(-c5ccc(-c6ccccc6)cc5-n5c6ccccc6c6ccccc65)n4)ccn3)c21 ; CC1(C)c2ccccc2-c2cc(-c3cc(-c4nc(-c5ccccc5)nc(-c5ccc(-c6ccccc6)cc5-n5c6ccccc6c6ccccc65)n4)cc4ccccc34)ccc21] 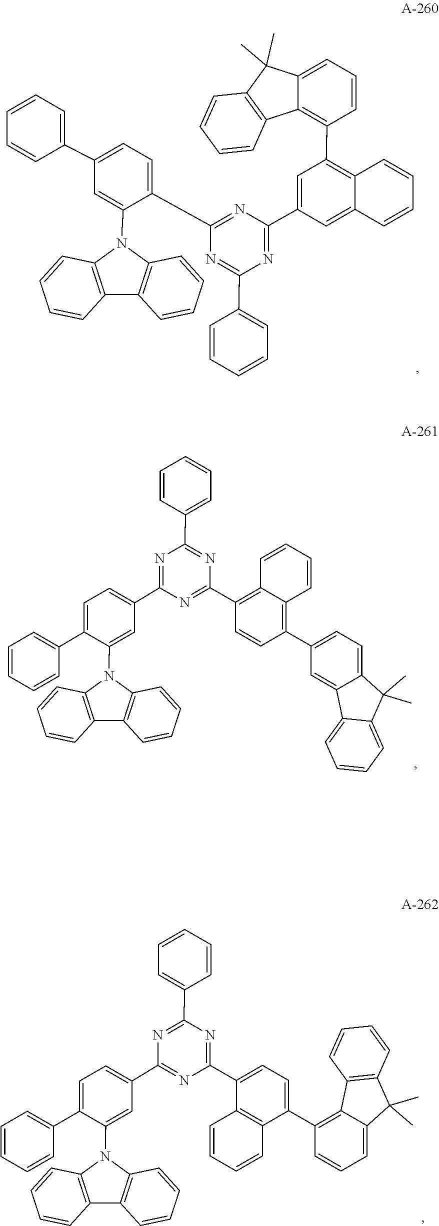 [SMILES: CC1(C)c2ccccc2-c2c(-c3cc(-c4nc(-c5ccccc5)nc(-c5ccc(-c6ccccc6)cc5-n5c6ccccc6c6ccccc65)n4)cc4ccccc34)cccc21 ; CC1(C)c2ccccc2-c2cc(-c3ccc(-c4nc(-c5ccccc5)nc(-c5ccc(-c6ccccc6)c(-n6c7ccccc7c7ccccc76)c5)n4)c4ccccc34)ccc21 ; CC1(C)c2ccccc2-c2c(-c3ccc(-c4nc(-c5ccccc5)nc(-c5ccc(-c6ccccc6)c(-n6c7ccccc7c7ccccc76)c5)n4)c4ccccc34)cccc21] 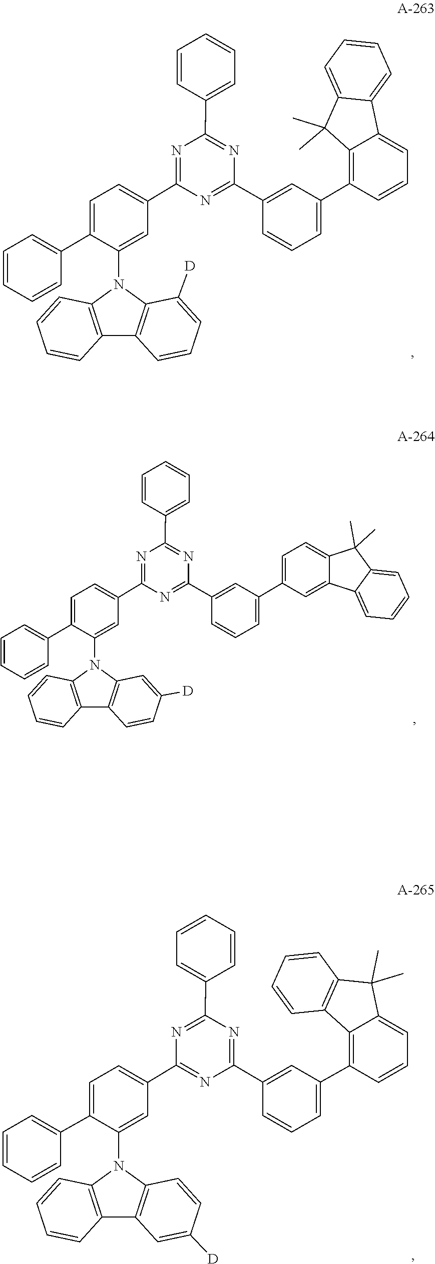 [SMILES: [2H]c1cccc2c3ccccc3n(-c3cc(-c4nc(-c5ccccc5)nc(-c5cccc(-c6cccc7c6C(C)(C)c6ccccc6-7)c5)n4)ccc3-c3ccccc3)c12 ; [2H]c1ccc2c3ccccc3n(-c3cc(-c4nc(-c5ccccc5)nc(-c5cccc(-c6ccc7c(c6)-c6ccccc6C7(C)C)c5)n4)ccc3-c3ccccc3)c2c1 ; [2H]c1ccc2c(c1)c1ccccc1n2-c1cc(-c2nc(-c3ccccc3)nc(-c3cccc(-c4cccc5c4-c4ccccc4C5(C)C)c3)n2)ccc1-c1ccccc1] 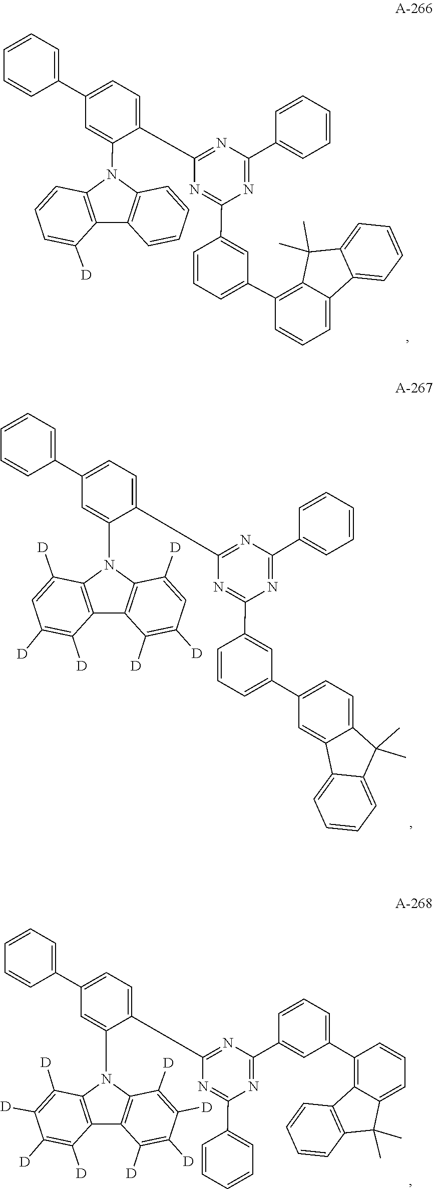 [SMILES: [2H]c1cccc2c1c1ccccc1n2-c1cc(-c2ccccc2)ccc1-c1nc(-c2ccccc2)nc(-c2cccc(-c3cccc4c3C(C)(C)c3ccccc3-4)c2)n1 ; [2H]c1cc([2H])c2c(c1[2H])c1c([2H])c([2H])cc([2H])c1n2-c1cc(-c2ccccc2)ccc1-c1nc(-c2ccccc2)nc(-c2cccc(-c3ccc4c(c3)-c3ccccc3C4(C)C)c2)n1 ; [2H]c1c([2H])c([2H])c2c(c1[2H])c1c([2H])c([2H])c([2H])c([2H])c1n2-c1cc(-c2ccccc2)ccc1-c1nc(-c2ccccc2)nc(-c2cccc(-c3cccc4c3-c3ccccc3C4(C)C)c2)n1] 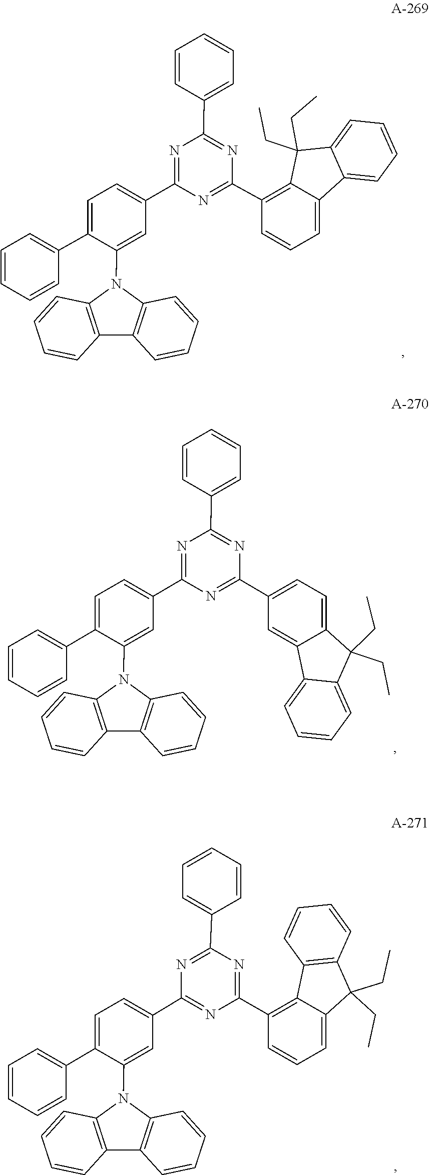 [SMILES: CCC1(CC)c2ccccc2-c2cccc(-c3nc(-c4ccccc4)nc(-c4ccc(-c5ccccc5)c(-n5c6ccccc6c6ccccc65)c4)n3)c21 ; CCC1(CC)c2ccccc2-c2cc(-c3nc(-c4ccccc4)nc(-c4ccc(-c5ccccc5)c(-n5c6ccccc6c6ccccc65)c4)n3)ccc21 ; CCC1(CC)c2ccccc2-c2c(-c3nc(-c4ccccc4)nc(-c4ccc(-c5ccccc5)c(-n5c6ccccc6c6ccccc65)c4)n3)cccc21] 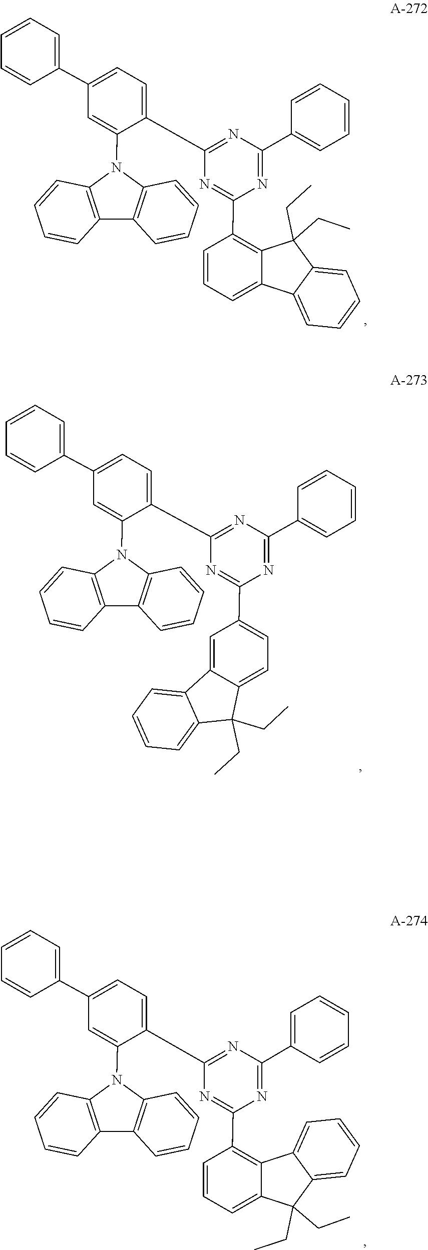 [SMILES: CCC1(CC)c2ccccc2-c2cccc(-c3nc(-c4ccccc4)nc(-c4ccc(-c5ccccc5)cc4-n4c5ccccc5c5ccccc54)n3)c21 ; CCC1(CC)c2ccccc2-c2cc(-c3nc(-c4ccccc4)nc(-c4ccc(-c5ccccc5)cc4-n4c5ccccc5c5ccccc54)n3)ccc21 ; CCC1(CC)c2ccccc2-c2c(-c3nc(-c4ccccc4)nc(-c4ccc(-c5ccccc5)cc4-n4c5ccccc5c5ccccc54)n3)cccc21] 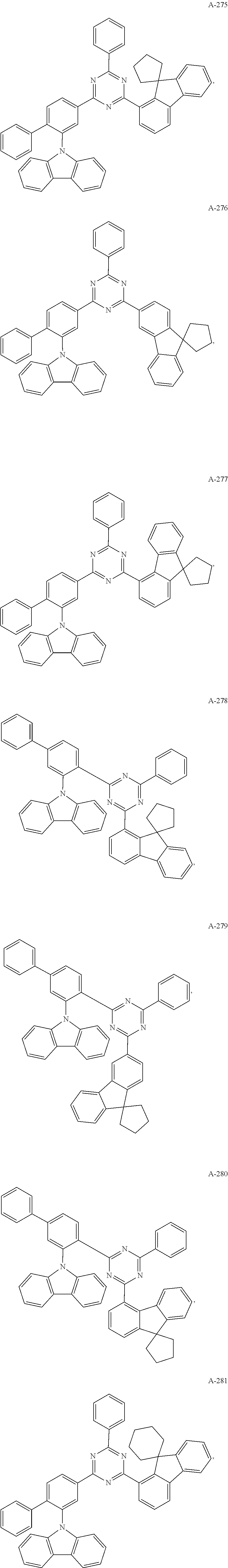 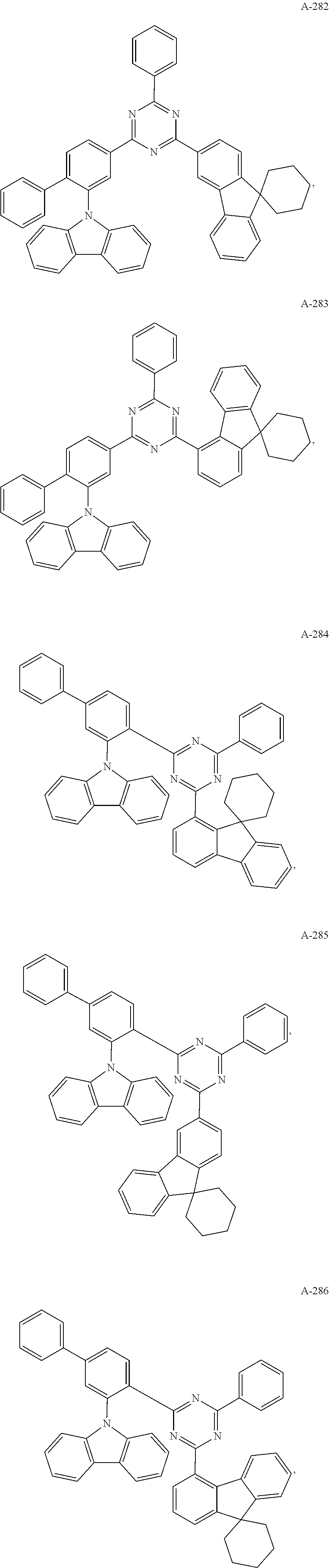 [SMILES: c1ccc(-c2nc(-c3ccc4c(c3)-c3ccccc3C43CCCCC3)nc(-c3ccc(-c4ccccc4)c(-n4c5ccccc5c5ccccc54)c3)n2)cc1 ; c1ccc(-c2nc(-c3ccc(-c4ccccc4)c(-n4c5ccccc5c5ccccc54)c3)nc(-c3cccc4c3-c3ccccc3C43CCCCC3)n2)cc1 ; c1ccc(-c2ccc(-c3nc(-c4ccccc4)nc(-c4cccc5c4C4(CCCCC4)c4ccccc4-5)n3)c(-n3c4ccccc4c4ccccc43)c2)cc1 ; c1ccc(-c2ccc(-c3nc(-c4ccccc4)nc(-c4ccc5c(c4)-c4ccccc4C54CCCCC4)n3)c(-n3c4ccccc4c4ccccc43)c2)cc1 ; c1ccc(-c2ccc(-c3nc(-c4ccccc4)nc(-c4cccc5c4-c4ccccc4C54CCCCC4)n3)c(-n3c4ccccc4c4ccccc43)c2)cc1] 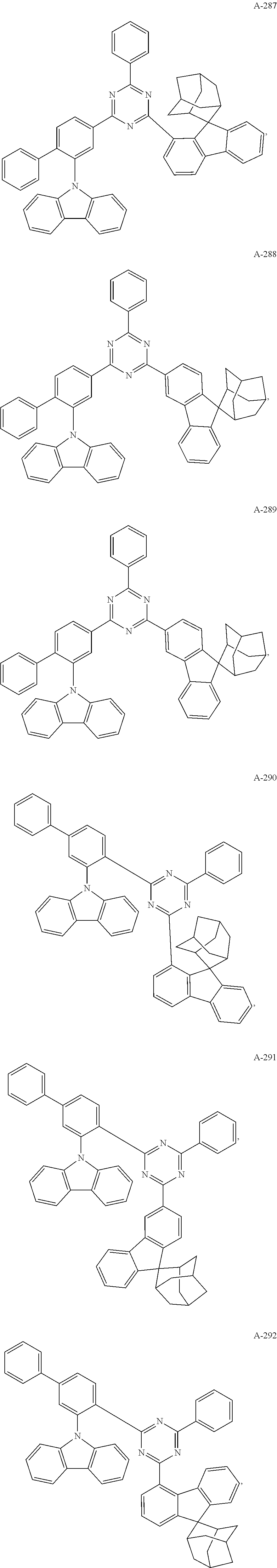 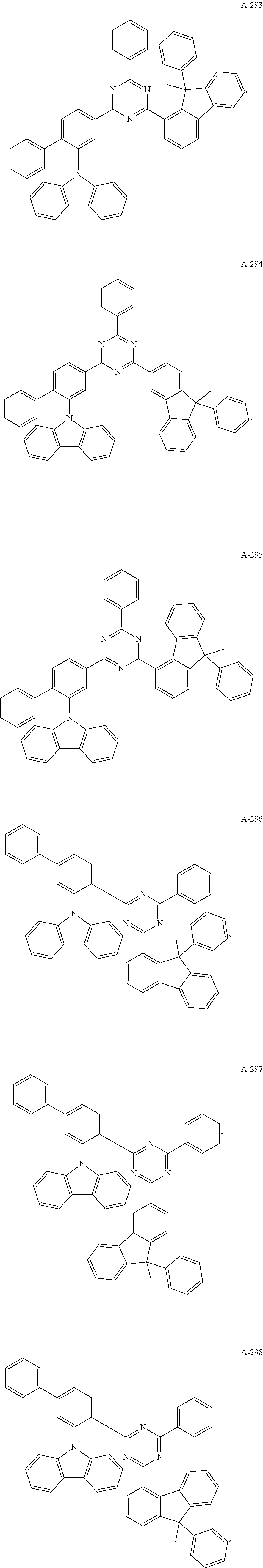 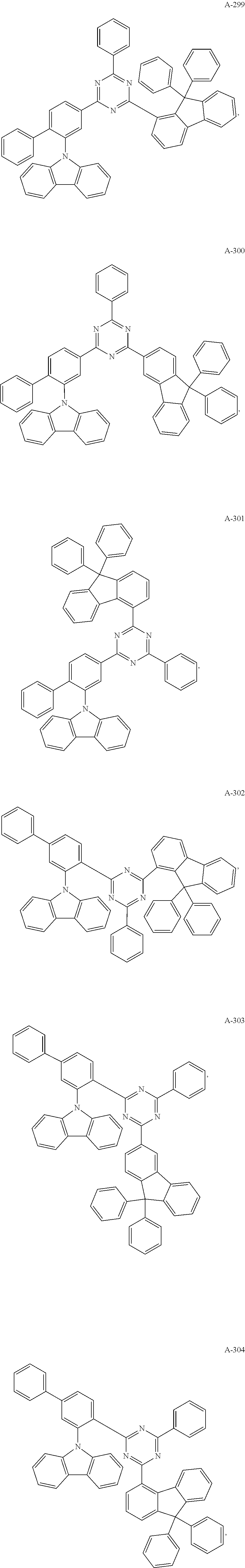 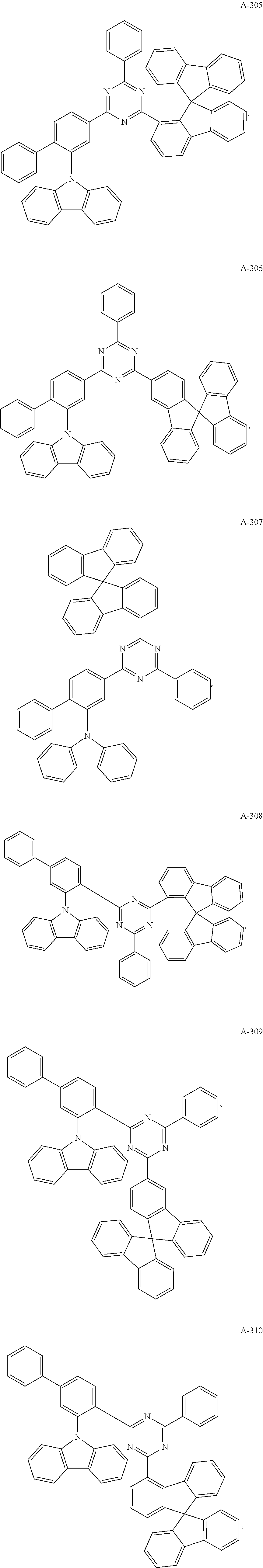 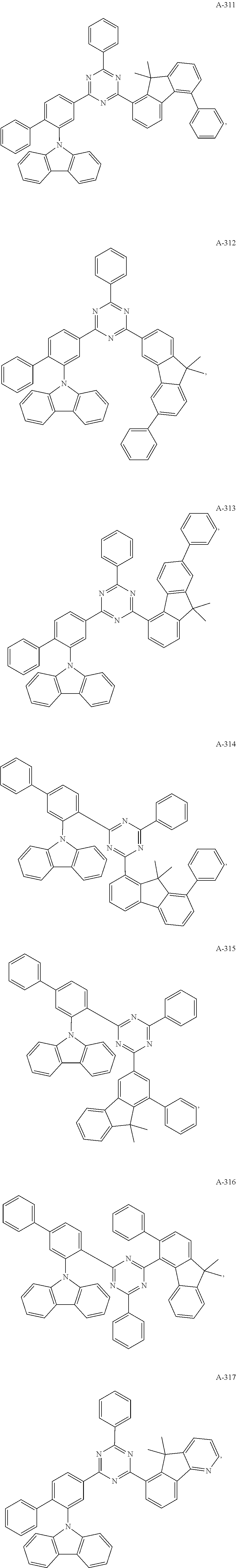 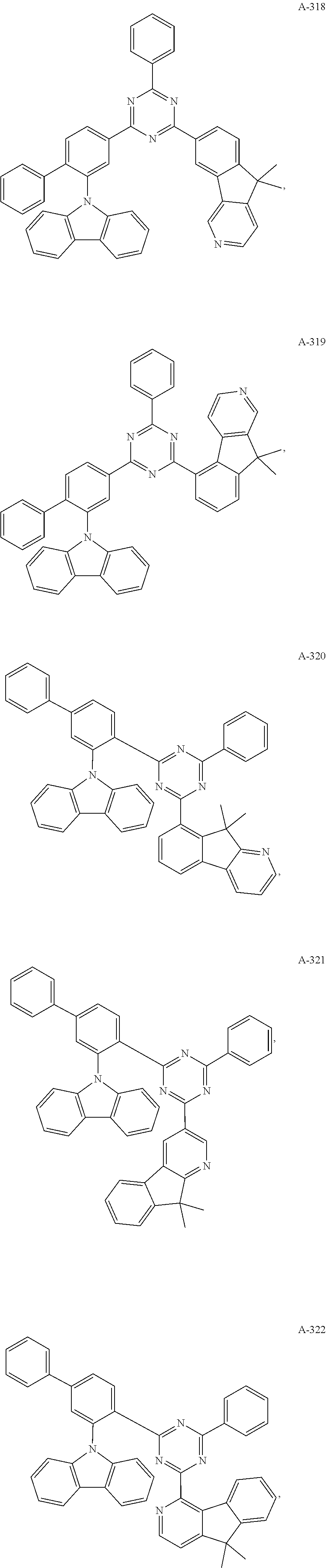 [SMILES: CC1(C)c2ccncc2-c2cc(-c3nc(-c4ccccc4)nc(-c4ccc(-c5ccccc5)c(-n5c6ccccc6c6ccccc65)c4)n3)ccc21 ; CC1(C)c2cnccc2-c2c(-c3nc(-c4ccccc4)nc(-c4ccc(-c5ccccc5)c(-n5c6ccccc6c6ccccc65)c4)n3)cccc21 ; CC1(C)c2ncccc2-c2cccc(-c3nc(-c4ccccc4)nc(-c4ccc(-c5ccccc5)cc4-n4c5ccccc5c5ccccc54)n3)c21 ; CC1(C)c2ccccc2-c2cc(-c3nc(-c4ccccc4)nc(-c4ccc(-c5ccccc5)cc4-n4c5ccccc5c5ccccc54)n3)cnc21 ; CC1(C)c2ccccc2-c2c1ccnc2-c1nc(-c2ccccc2)nc(-c2ccc(-c3ccccc3)cc2-n2c3ccccc3c3ccccc32)n1] 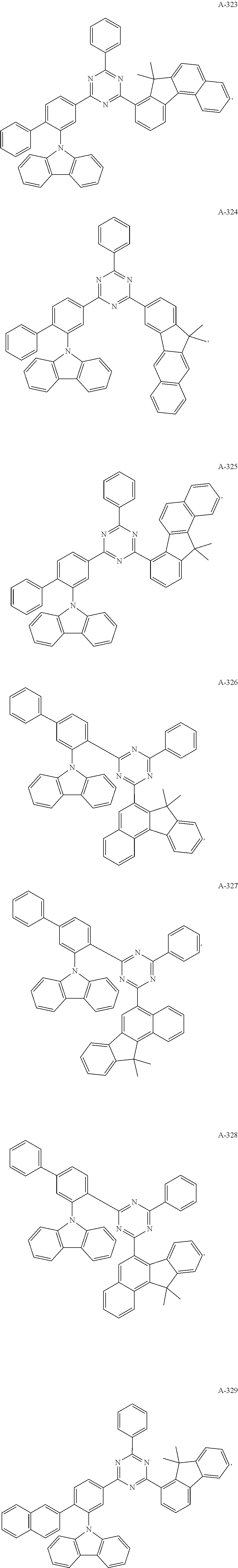 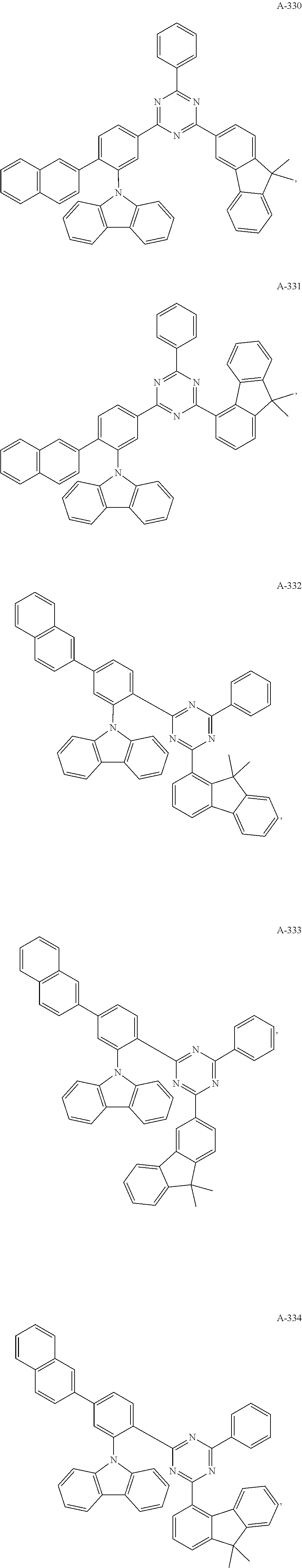 [SMILES: CC1(C)c2ccccc2-c2cc(-c3nc(-c4ccccc4)nc(-c4ccc(-c5ccc6ccccc6c5)c(-n5c6ccccc6c6ccccc65)c4)n3)ccc21 ; CC1(C)c2ccccc2-c2c(-c3nc(-c4ccccc4)nc(-c4ccc(-c5ccc6ccccc6c5)c(-n5c6ccccc6c6ccccc65)c4)n3)cccc21 ; CC1(C)c2ccccc2-c2cccc(-c3nc(-c4ccccc4)nc(-c4ccc(-c5ccc6ccccc6c5)cc4-n4c5ccccc5c5ccccc54)n3)c21 ; CC1(C)c2ccccc2-c2cc(-c3nc(-c4ccccc4)nc(-c4ccc(-c5ccc6ccccc6c5)cc4-n4c5ccccc5c5ccccc54)n3)ccc21 ; CC1(C)c2ccccc2-c2c(-c3nc(-c4ccccc4)nc(-c4ccc(-c5ccc6ccccc6c5)cc4-n4c5ccccc5c5ccccc54)n3)cccc21] 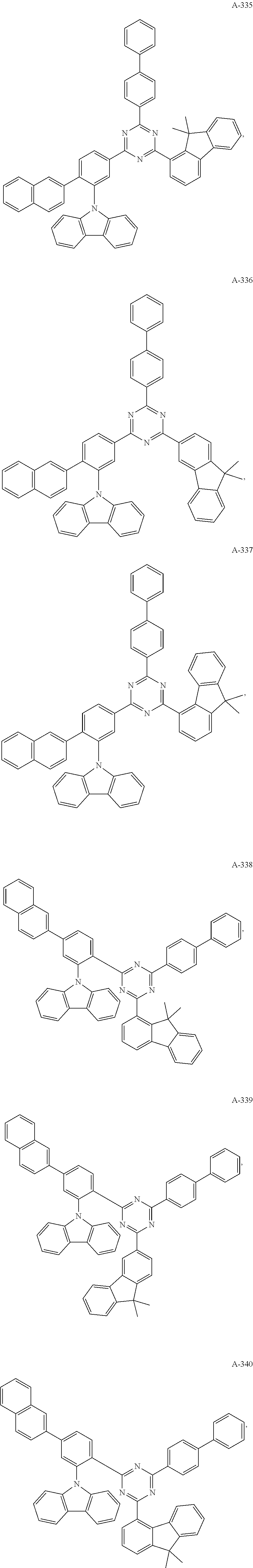 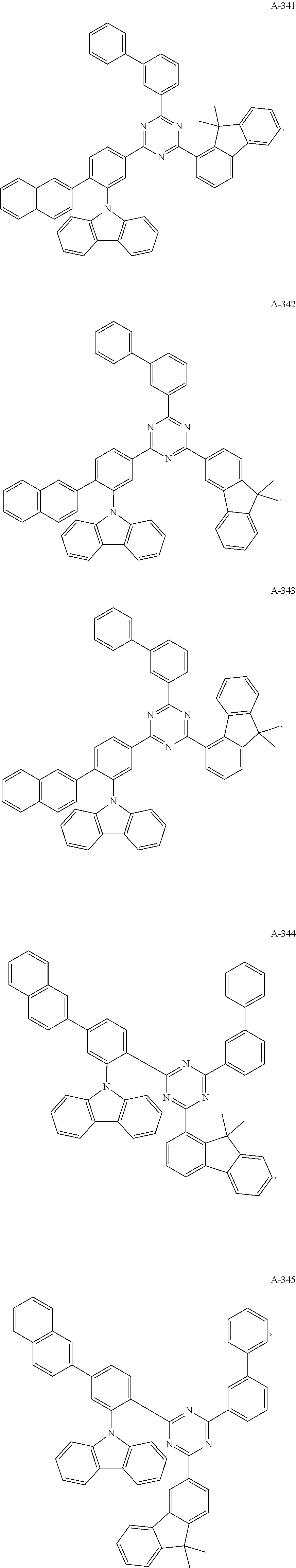 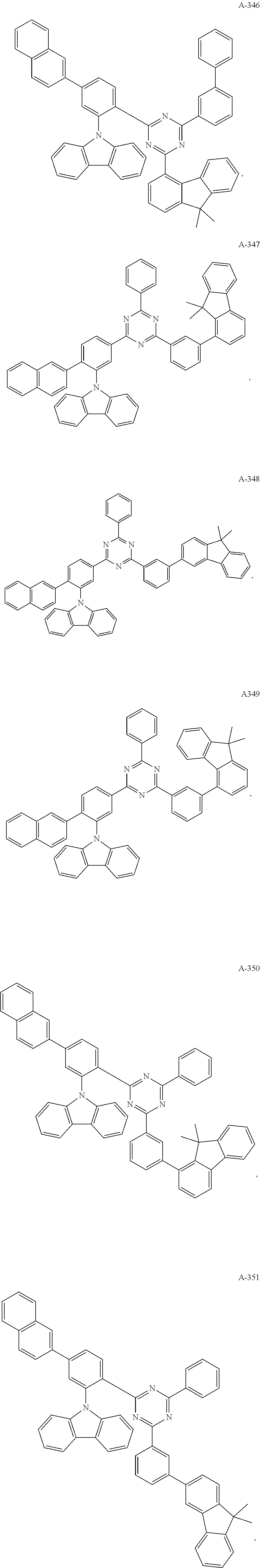 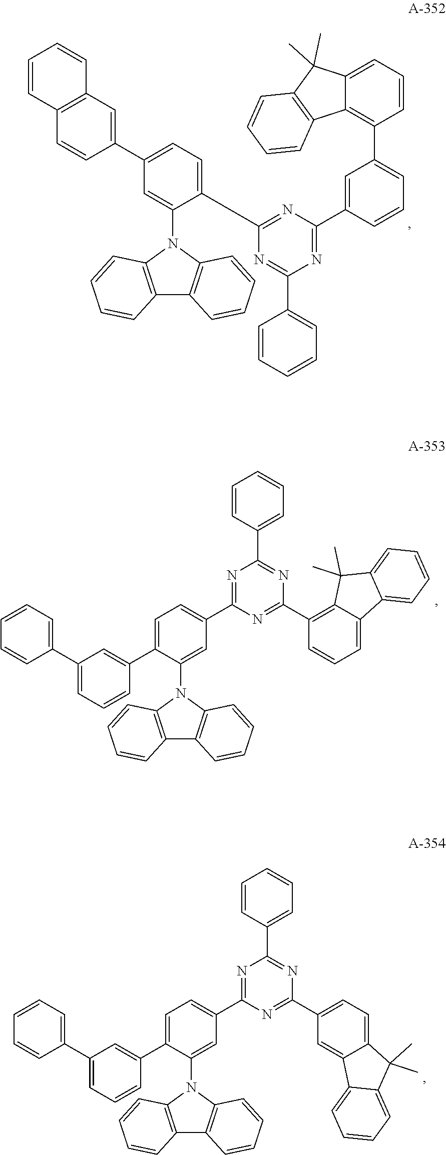 [SMILES: CC1(C)c2ccccc2-c2c(-c3cccc(-c4nc(-c5ccccc5)nc(-c5ccc(-c6ccc7ccccc7c6)cc5-n5c6ccccc6c6ccccc65)n4)c3)cccc21 ; CC1(C)c2ccccc2-c2cccc(-c3nc(-c4ccccc4)nc(-c4ccc(-c5cccc(-c6ccccc6)c5)c(-n5c6ccccc6c6ccccc65)c4)n3)c21 ; CC1(C)c2ccccc2-c2cc(-c3nc(-c4ccccc4)nc(-c4ccc(-c5cccc(-c6ccccc6)c5)c(-n5c6ccccc6c6ccccc65)c4)n3)ccc21] 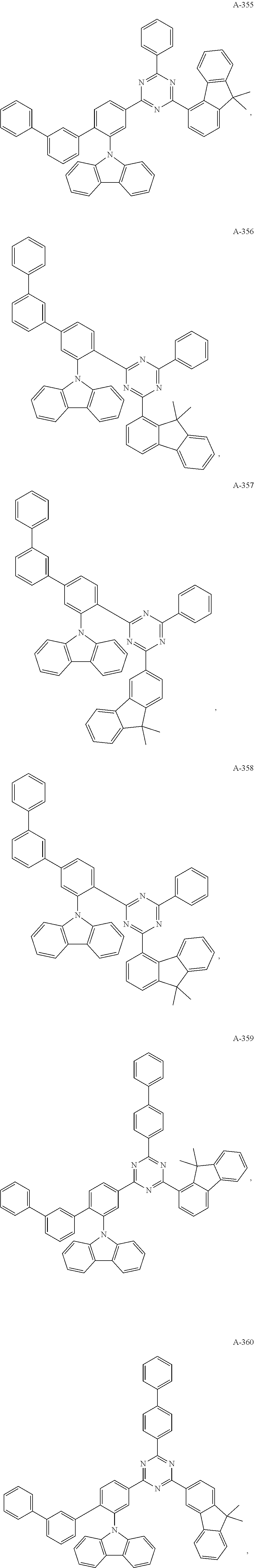 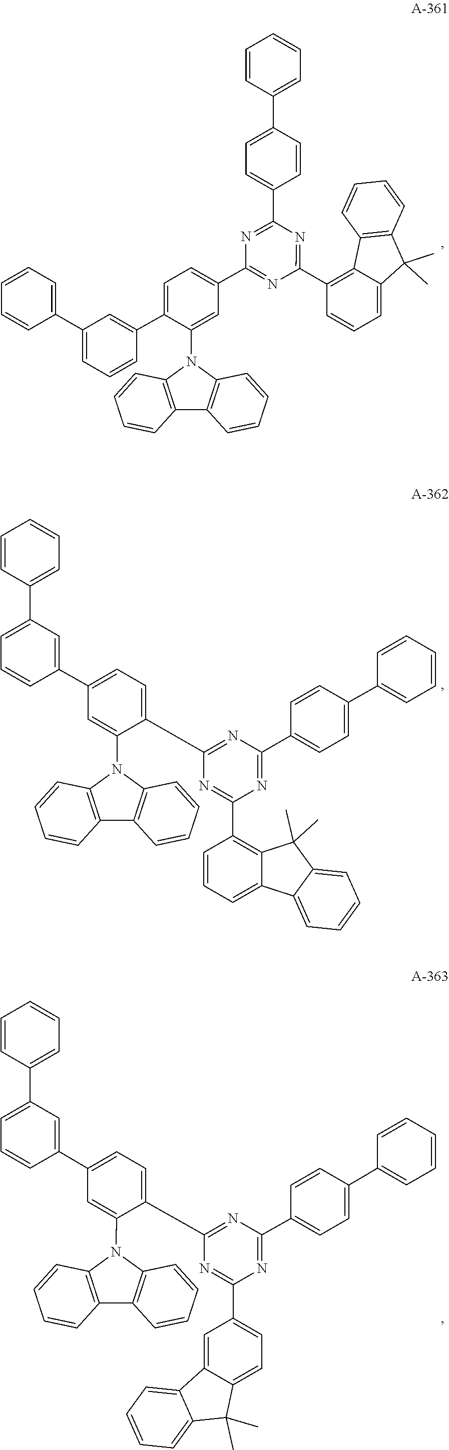 [SMILES: CC1(C)c2ccccc2-c2c(-c3nc(-c4ccc(-c5ccccc5)cc4)nc(-c4ccc(-c5cccc(-c6ccccc6)c5)c(-n5c6ccccc6c6ccccc65)c4)n3)cccc21 ; CC1(C)c2ccccc2-c2cccc(-c3nc(-c4ccc(-c5ccccc5)cc4)nc(-c4ccc(-c5cccc(-c6ccccc6)c5)cc4-n4c5ccccc5c5ccccc54)n3)c21 ; CC1(C)c2ccccc2-c2cc(-c3nc(-c4ccc(-c5ccccc5)cc4)nc(-c4ccc(-c5cccc(-c6ccccc6)c5)cc4-n4c5ccccc5c5ccccc54)n3)ccc21] 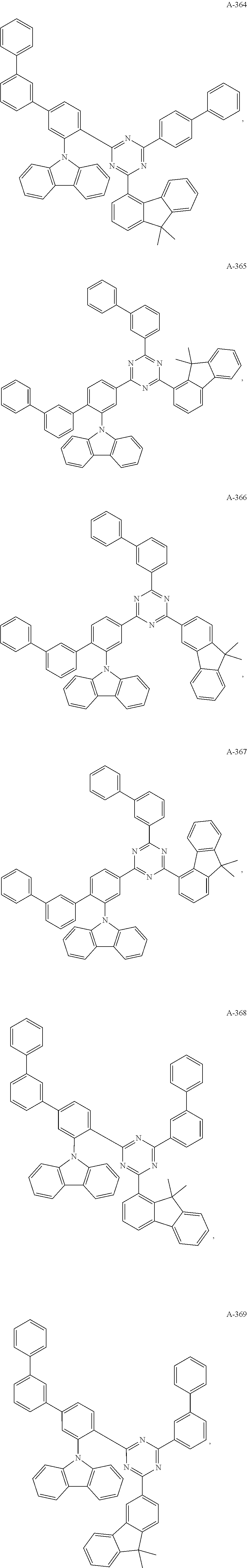 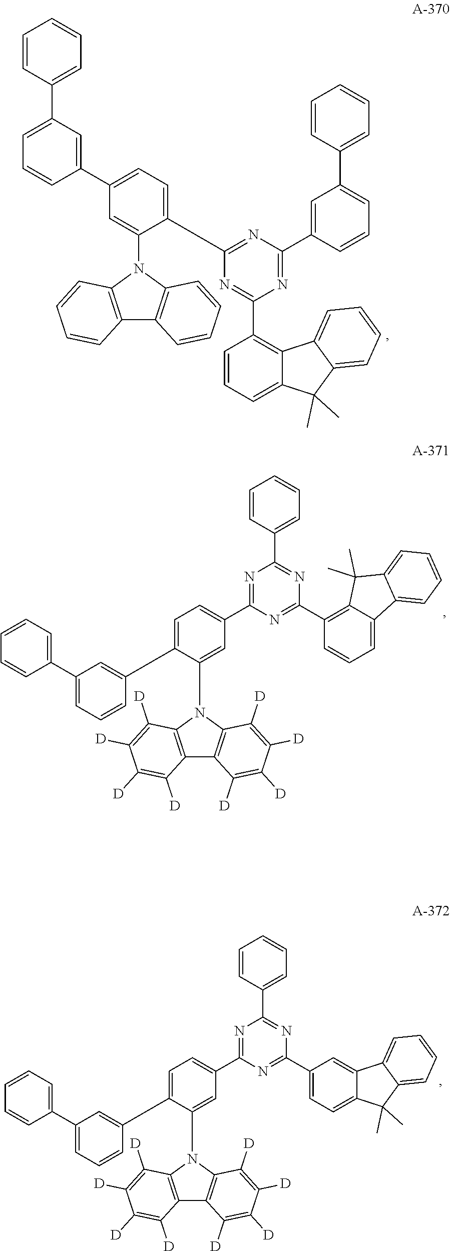 [SMILES: CC1(C)c2ccccc2-c2c(-c3nc(-c4cccc(-c5ccccc5)c4)nc(-c4ccc(-c5cccc(-c6ccccc6)c5)cc4-n4c5ccccc5c5ccccc54)n3)cccc21 ; [2H]c1c([2H])c([2H])c2c(c1[2H])c1c([2H])c([2H])c([2H])c([2H])c1n2-c1cc(-c2nc(-c3ccccc3)nc(-c3cccc4c3C(C)(C)c3ccccc3-4)n2)ccc1-c1cccc(-c2ccccc2)c1 ; [2H]c1c([2H])c([2H])c2c(c1[2H])c1c([2H])c([2H])c([2H])c([2H])c1n2-c1cc(-c2nc(-c3ccccc3)nc(-c3ccc4c(c3)-c3ccccc3C4(C)C)n2)ccc1-c1cccc(-c2ccccc2)c1] 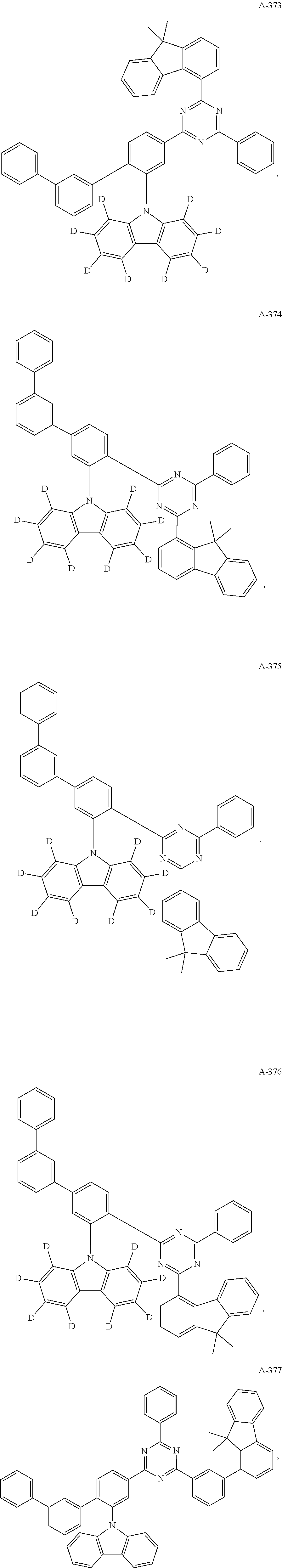 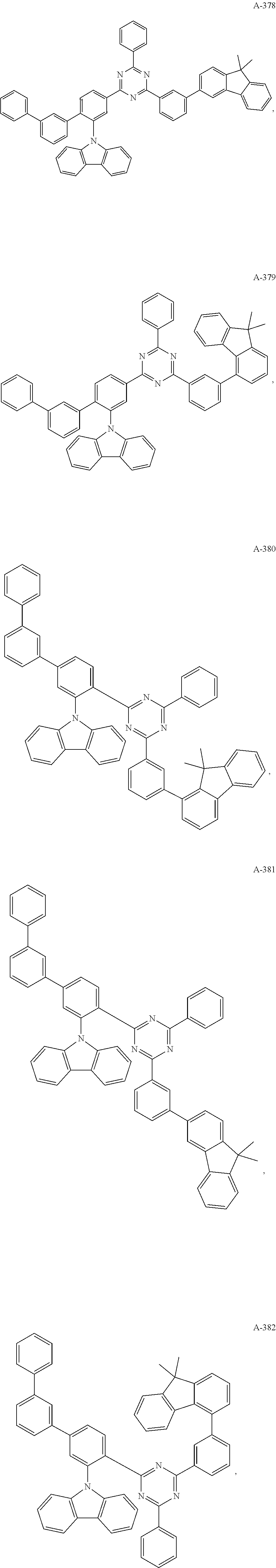 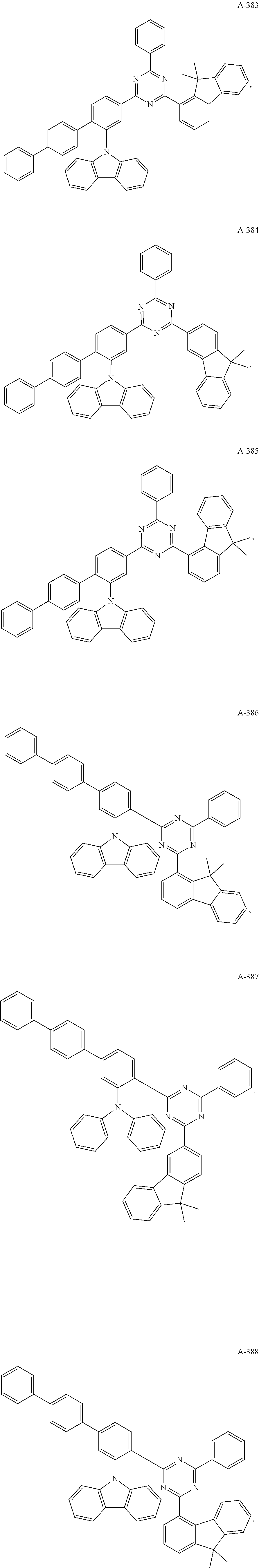 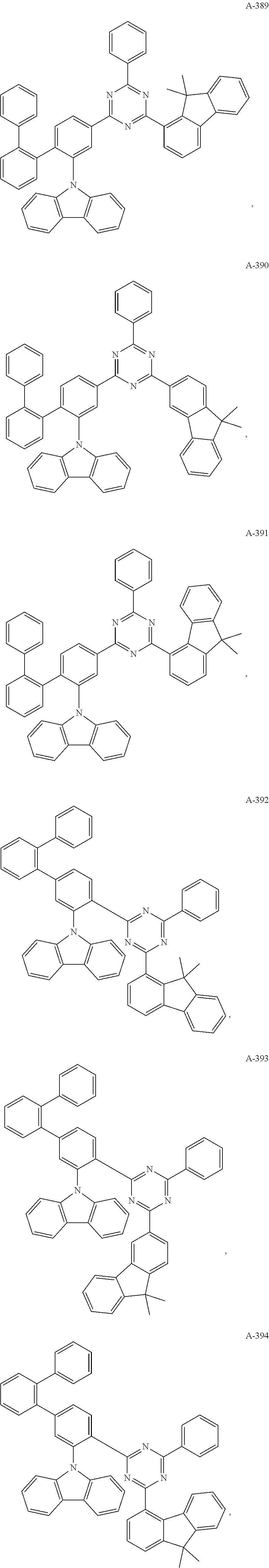 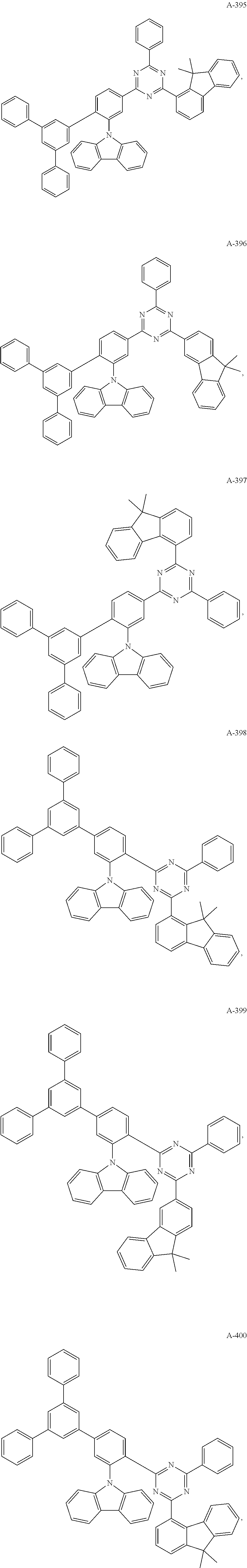 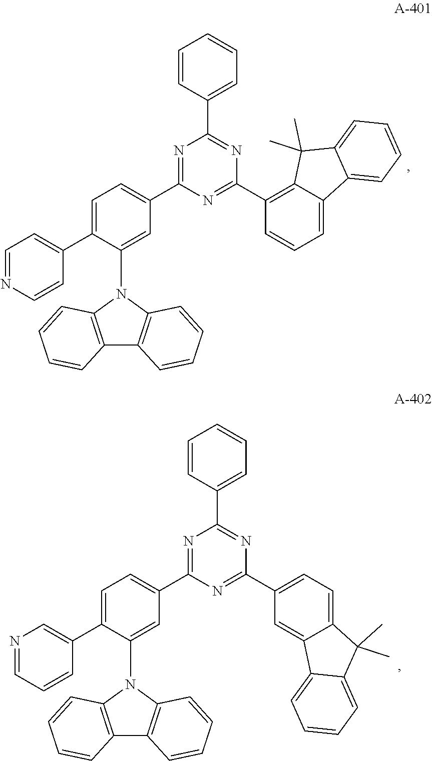 [SMILES: CC1(C)c2ccccc2-c2cccc(-c3nc(-c4ccccc4)nc(-c4ccc(-c5ccncc5)c(-n5c6ccccc6c6ccccc65)c4)n3)c21 ; CC1(C)c2ccccc2-c2cc(-c3nc(-c4ccccc4)nc(-c4ccc(-c5cccnc5)c(-n5c6ccccc6c6ccccc65)c4)n3)ccc21] 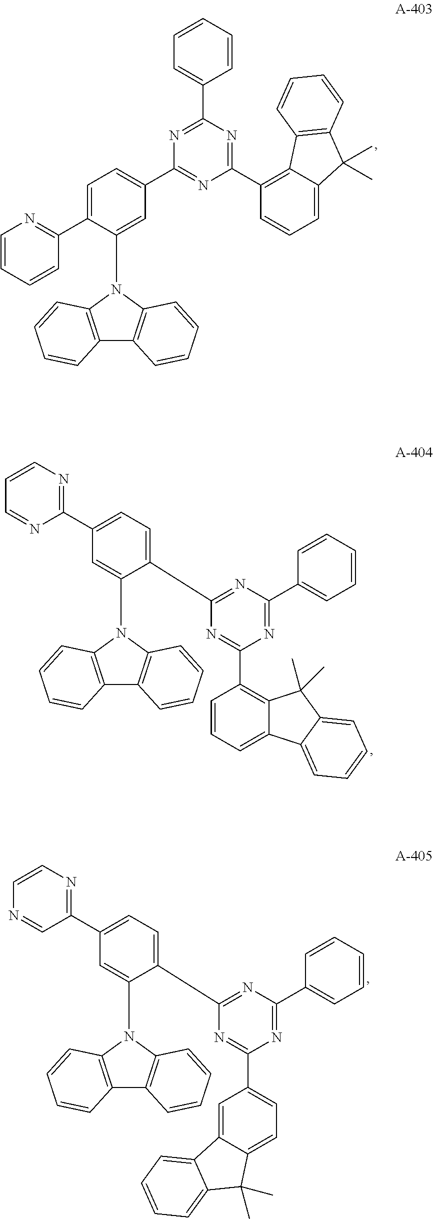 [SMILES: CC1(C)c2ccccc2-c2c(-c3nc(-c4ccccc4)nc(-c4ccc(-c5ccccn5)c(-n5c6ccccc6c6ccccc65)c4)n3)cccc21 ; CC1(C)c2ccccc2-c2cccc(-c3nc(-c4ccccc4)nc(-c4ccc(-c5ncccn5)cc4-n4c5ccccc5c5ccccc54)n3)c21 ; CC1(C)c2ccccc2-c2cc(-c3nc(-c4ccccc4)nc(-c4ccc(-c5cnccn5)cc4-n4c5ccccc5c5ccccc54)n3)ccc21] 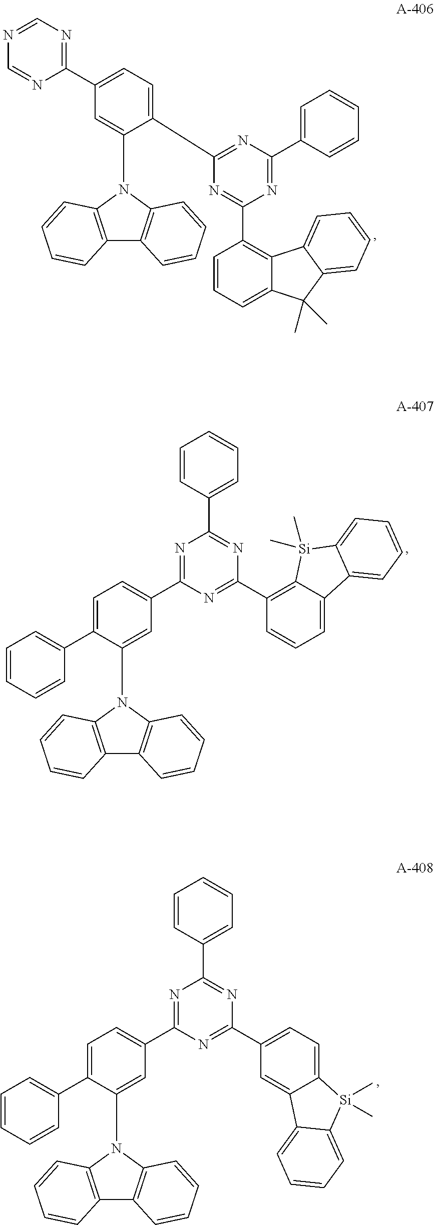 [SMILES: CC1(C)c2ccccc2-c2c(-c3nc(-c4ccccc4)nc(-c4ccc(-c5ncncn5)cc4-n4c5ccccc5c5ccccc54)n3)cccc21 ; C[Si]1(C)c2ccccc2-c2cccc(-c3nc(-c4ccccc4)nc(-c4ccc(-c5ccccc5)c(-n5c6ccccc6c6ccccc65)c4)n3)c21 ; C[Si]1(C)c2ccccc2-c2cc(-c3nc(-c4ccccc4)nc(-c4ccc(-c5ccccc5)c(-n5c6ccccc6c6ccccc65)c4)n3)ccc21] 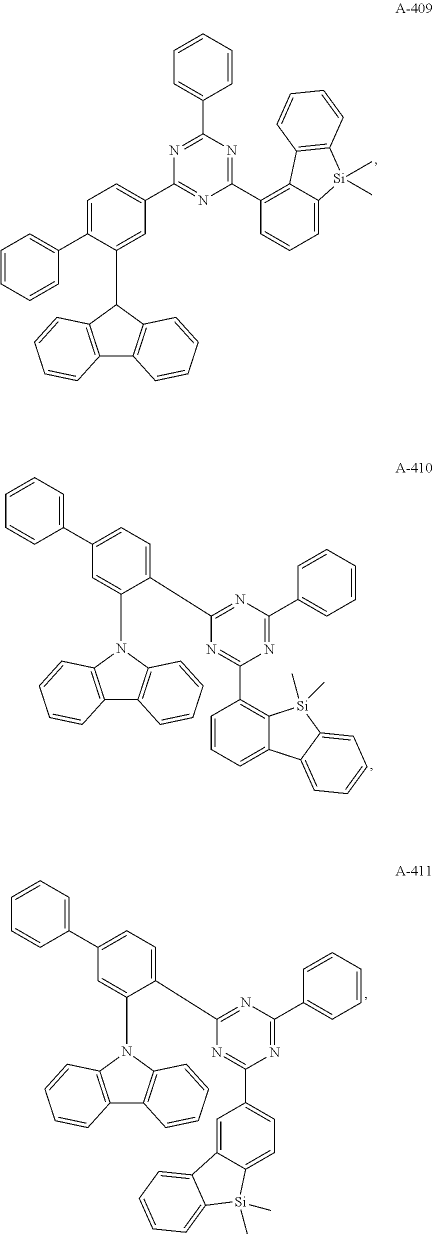 [SMILES: C[Si]1(C)c2ccccc2-c2c(-c3nc(-c4ccccc4)nc(-c4ccc(-c5ccccc5)c(C5c6ccccc6-c6ccccc65)c4)n3)cccc21 ; C[Si]1(C)c2ccccc2-c2cccc(-c3nc(-c4ccccc4)nc(-c4ccc(-c5ccccc5)cc4-n4c5ccccc5c5ccccc54)n3)c21 ; C[Si]1(C)c2ccccc2-c2cc(-c3nc(-c4ccccc4)nc(-c4ccc(-c5ccccc5)cc4-n4c5ccccc5c5ccccc54)n3)ccc21] 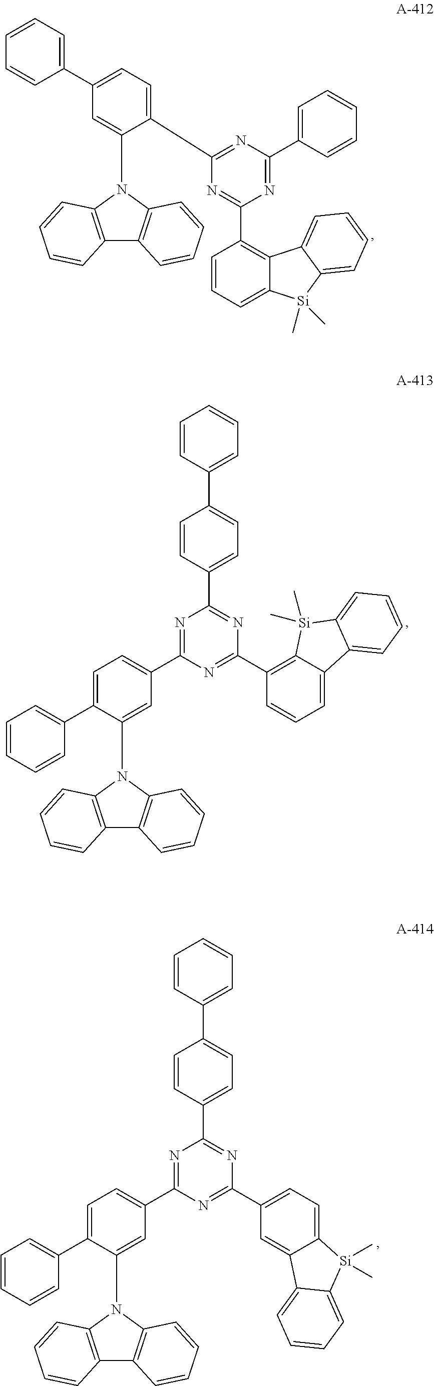 [SMILES: C[Si]1(C)c2ccccc2-c2c(-c3nc(-c4ccccc4)nc(-c4ccc(-c5ccccc5)cc4-n4c5ccccc5c5ccccc54)n3)cccc21 ; C[Si]1(C)c2ccccc2-c2cccc(-c3nc(-c4ccc(-c5ccccc5)cc4)nc(-c4ccc(-c5ccccc5)c(-n5c6ccccc6c6ccccc65)c4)n3)c21 ; C[Si]1(C)c2ccccc2-c2cc(-c3nc(-c4ccc(-c5ccccc5)cc4)nc(-c4ccc(-c5ccccc5)c(-n5c6ccccc6c6ccccc65)c4)n3)ccc21] 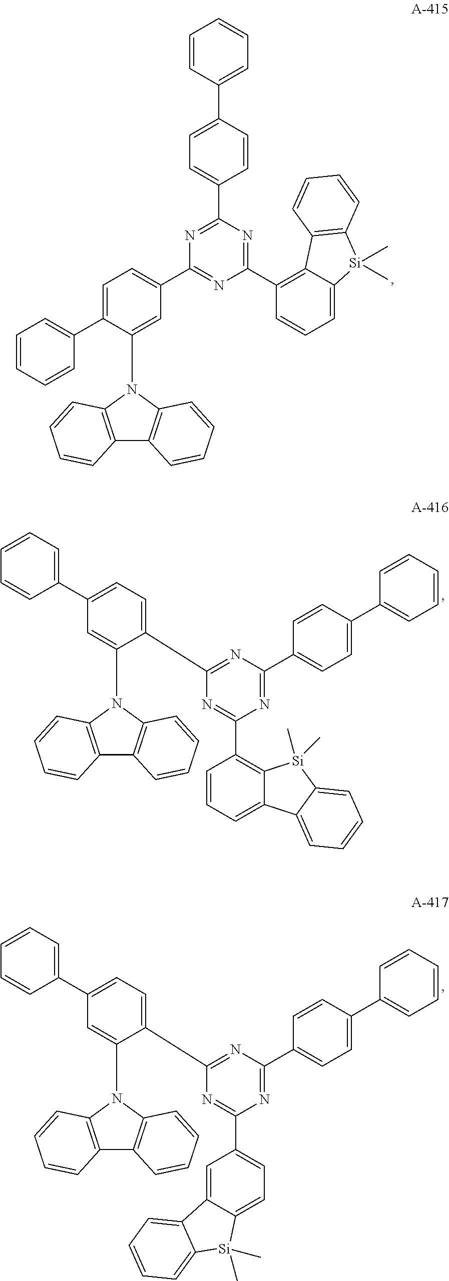 [SMILES: C[Si]1(C)c2ccccc2-c2c(-c3nc(-c4ccc(-c5ccccc5)cc4)nc(-c4ccc(-c5ccccc5)c(-n5c6ccccc6c6ccccc65)c4)n3)cccc21 ; C[Si]1(C)c2ccccc2-c2cccc(-c3nc(-c4ccc(-c5ccccc5)cc4)nc(-c4ccc(-c5ccccc5)cc4-n4c5ccccc5c5ccccc54)n3)c21 ; C[Si]1(C)c2ccccc2-c2cc(-c3nc(-c4ccc(-c5ccccc5)cc4)nc(-c4ccc(-c5ccccc5)cc4-n4c5ccccc5c5ccccc54)n3)ccc21] 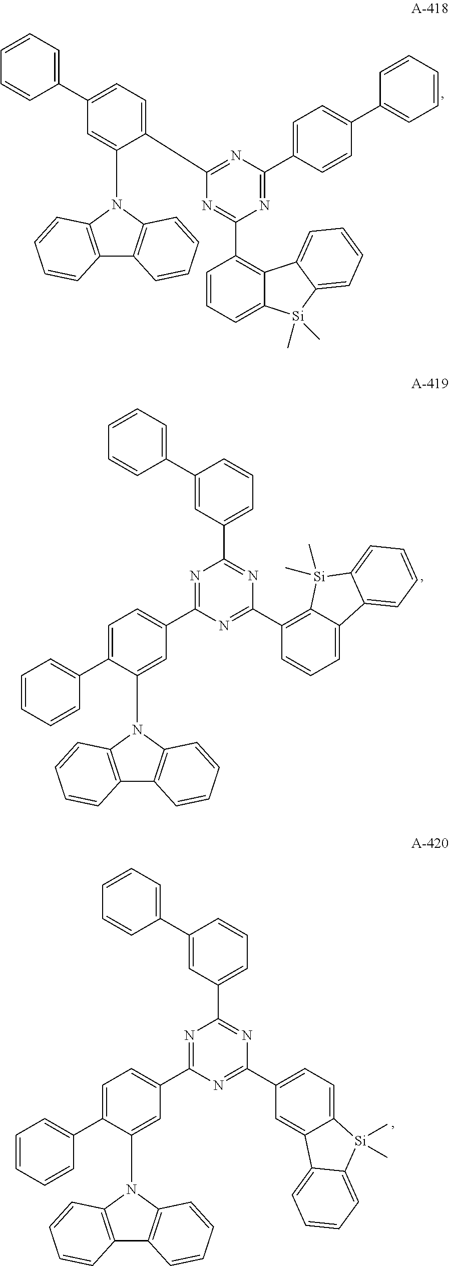 [SMILES: C[Si]1(C)c2ccccc2-c2c(-c3nc(-c4ccc(-c5ccccc5)cc4)nc(-c4ccc(-c5ccccc5)cc4-n4c5ccccc5c5ccccc54)n3)cccc21 ; C[Si]1(C)c2ccccc2-c2cccc(-c3nc(-c4cccc(-c5ccccc5)c4)nc(-c4ccc(-c5ccccc5)c(-n5c6ccccc6c6ccccc65)c4)n3)c21 ; C[Si]1(C)c2ccccc2-c2cc(-c3nc(-c4cccc(-c5ccccc5)c4)nc(-c4ccc(-c5ccccc5)c(-n5c6ccccc6c6ccccc65)c4)n3)ccc21] 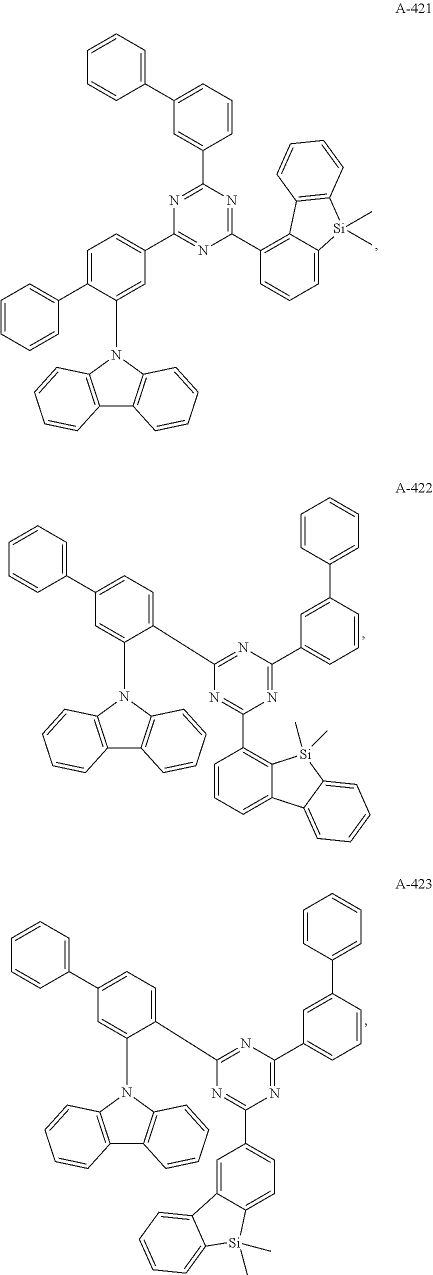 [SMILES: C[Si]1(C)c2ccccc2-c2c(-c3nc(-c4cccc(-c5ccccc5)c4)nc(-c4ccc(-c5ccccc5)c(-n5c6ccccc6c6ccccc65)c4)n3)cccc21 ; C[Si]1(C)c2ccccc2-c2cccc(-c3nc(-c4cccc(-c5ccccc5)c4)nc(-c4ccc(-c5ccccc5)cc4-n4c5ccccc5c5ccccc54)n3)c21 ; C[Si]1(C)c2ccccc2-c2cc(-c3nc(-c4cccc(-c5ccccc5)c4)nc(-c4ccc(-c5ccccc5)cc4-n4c5ccccc5c5ccccc54)n3)ccc21] 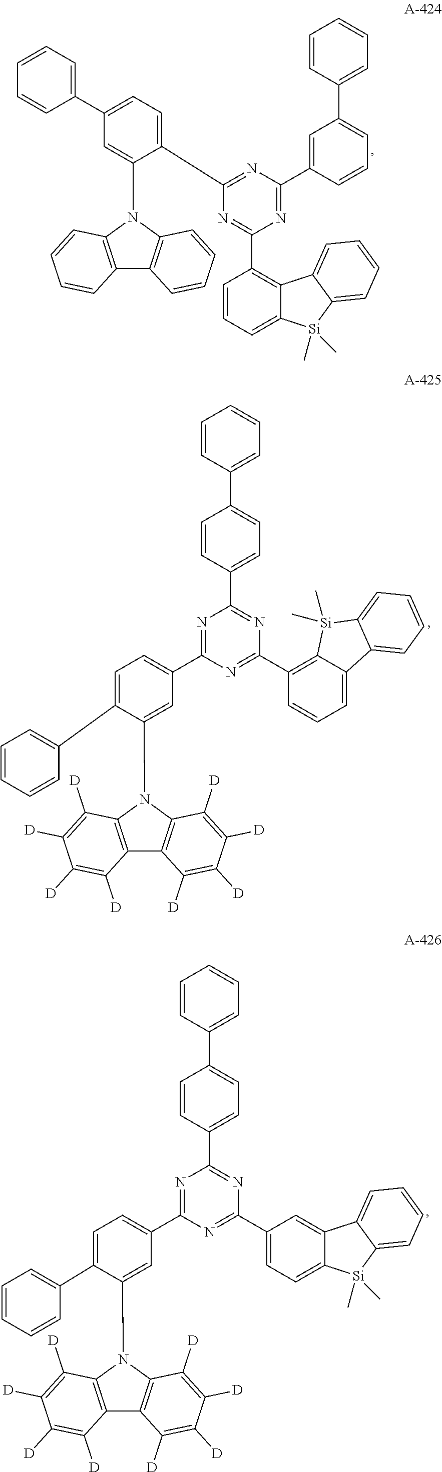 [SMILES: C[Si]1(C)c2ccccc2-c2c(-c3nc(-c4cccc(-c5ccccc5)c4)nc(-c4ccc(-c5ccccc5)cc4-n4c5ccccc5c5ccccc54)n3)cccc21 ; [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3cccc4c3[Si](C)(C)c3ccccc3-4)n2)ccc1-c1ccccc1 ; [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3ccc4c(c3)-c3ccccc3[Si]4(C)C)n2)ccc1-c1ccccc1] 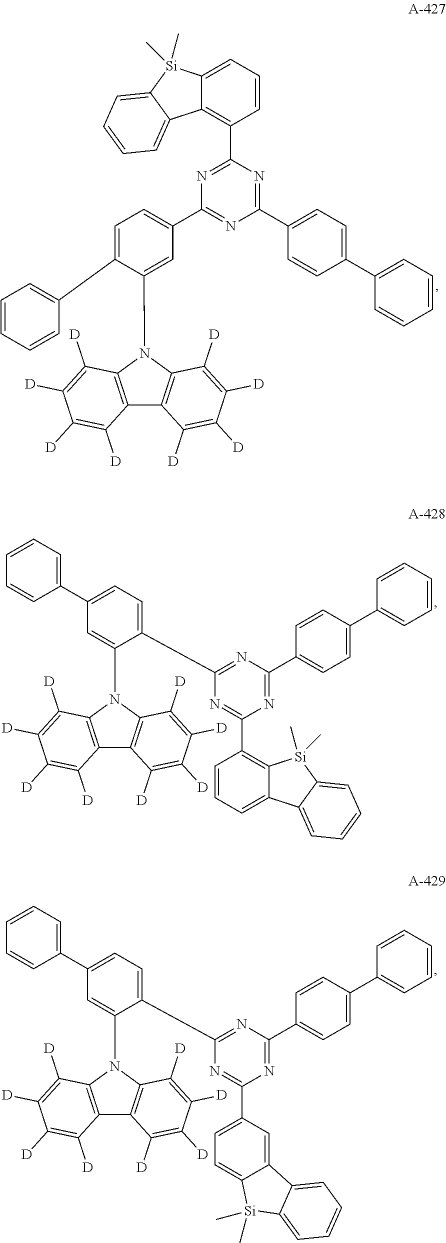 [SMILES: [2H]c1c([2H])c([2H])c2c(c1[2H])c1c([2H])c([2H])c([2H])c([2H])c1n2-c1cc(-c2nc(-c3ccc(-c4ccccc4)cc3)nc(-c3cccc4c3-c3ccccc3[Si]4(C)C)n2)ccc1-c1ccccc1 ; [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2cccc3c2[Si](C)(C)c2ccccc2-3)n1 ; [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2ccc3c(c2)-c2ccccc2[Si]3(C)C)n1] 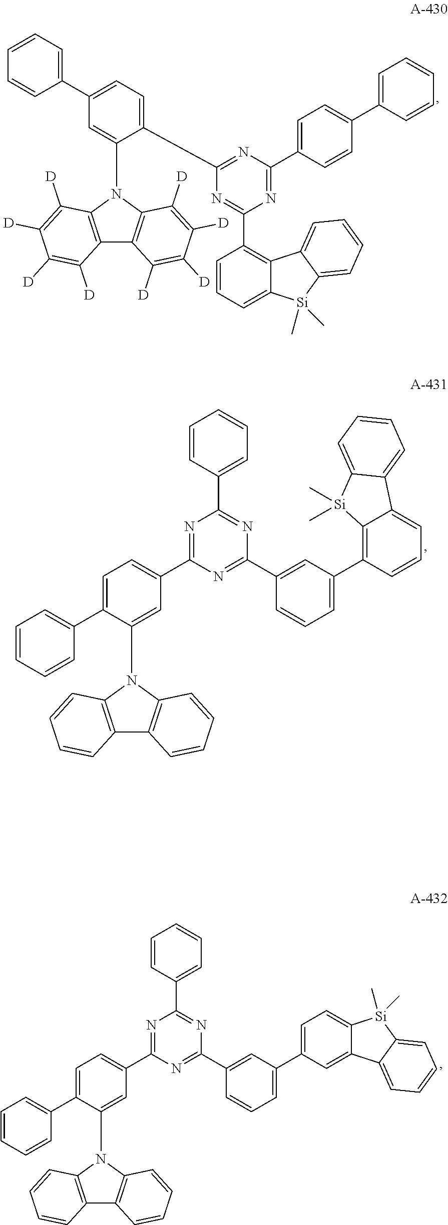 [SMILES: [2H]c1c([2H])c([2H])c2c(c1[2H])c1c([2H])c([2H])c([2H])c([2H])c1n2-c1cc(-c2ccccc2)ccc1-c1nc(-c2ccc(-c3ccccc3)cc2)nc(-c2cccc3c2-c2ccccc2[Si]3(C)C)n1 ; C[Si]1(C)c2ccccc2-c2cccc(-c3cccc(-c4nc(-c5ccccc5)nc(-c5ccc(-c6ccccc6)c(-n6c7ccccc7c7ccccc76)c5)n4)c3)c21 ; C[Si]1(C)c2ccccc2-c2cc(-c3cccc(-c4nc(-c5ccccc5)nc(-c5ccc(-c6ccccc6)c(-n6c7ccccc7c7ccccc76)c5)n4)c3)ccc21] 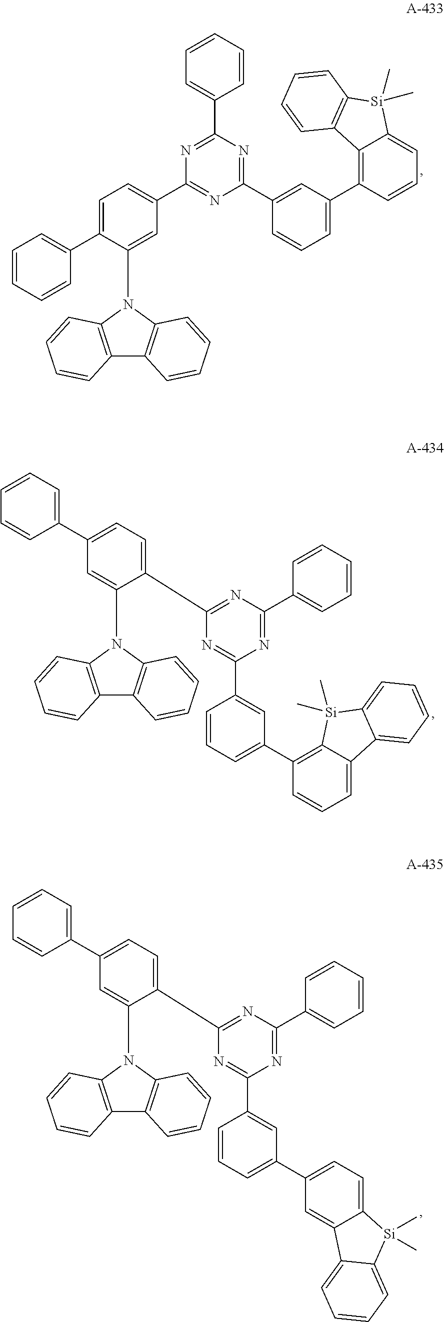 [SMILES: C[Si]1(C)c2ccccc2-c2c(-c3cccc(-c4nc(-c5ccccc5)nc(-c5ccc(-c6ccccc6)c(-n6c7ccccc7c7ccccc76)c5)n4)c3)cccc21 ; C[Si]1(C)c2ccccc2-c2cccc(-c3cccc(-c4nc(-c5ccccc5)nc(-c5ccc(-c6ccccc6)cc5-n5c6ccccc6c6ccccc65)n4)c3)c21 ; C[Si]1(C)c2ccccc2-c2cc(-c3cccc(-c4nc(-c5ccccc5)nc(-c5ccc(-c6ccccc6)cc5-n5c6ccccc6c6ccccc65)n4)c3)ccc21] 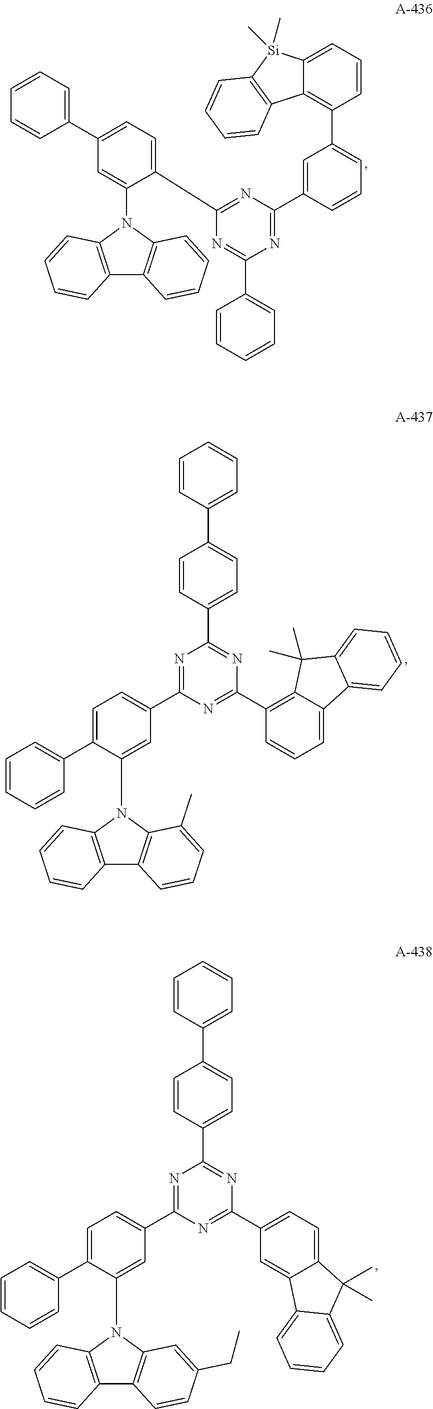 [SMILES: C[Si]1(C)c2ccccc2-c2c(-c3cccc(-c4nc(-c5ccccc5)nc(-c5ccc(-c6ccccc6)cc5-n5c6ccccc6c6ccccc65)n4)c3)cccc21 ; Cc1cccc2c3ccccc3n(-c3cc(-c4nc(-c5ccc(-c6ccccc6)cc5)nc(-c5cccc6c5C(C)(C)c5ccccc5-6)n4)ccc3-c3ccccc3)c12 ; CCc1ccc2c3ccccc3n(-c3cc(-c4nc(-c5ccc(-c6ccccc6)cc5)nc(-c5ccc6c(c5)-c5ccccc5C6(C)C)n4)ccc3-c3ccccc3)c2c1] 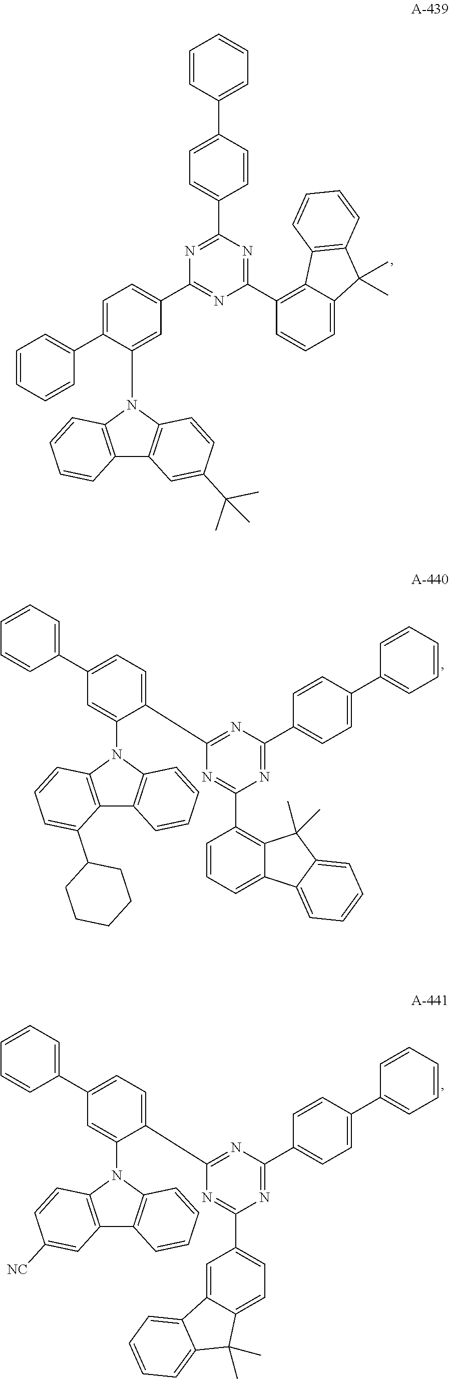 [SMILES: CC(C)(C)c1ccc2c(c1)c1ccccc1n2-c1cc(-c2nc(-c3ccc(-c4ccccc4)cc3)nc(-c3cccc4c3-c3ccccc3C4(C)C)n2)ccc1-c1ccccc1 ; CC1(C)c2ccccc2-c2cccc(-c3nc(-c4ccc(-c5ccccc5)cc4)nc(-c4ccc(-c5ccccc5)cc4-n4c5ccccc5c5c(C6CCCCC6)cccc54)n3)c21 ; CC1(C)c2ccccc2-c2cc(-c3nc(-c4ccc(-c5ccccc5)cc4)nc(-c4ccc(-c5ccccc5)cc4-n4c5ccccc5c5cc(C#N)ccc54)n3)ccc21] 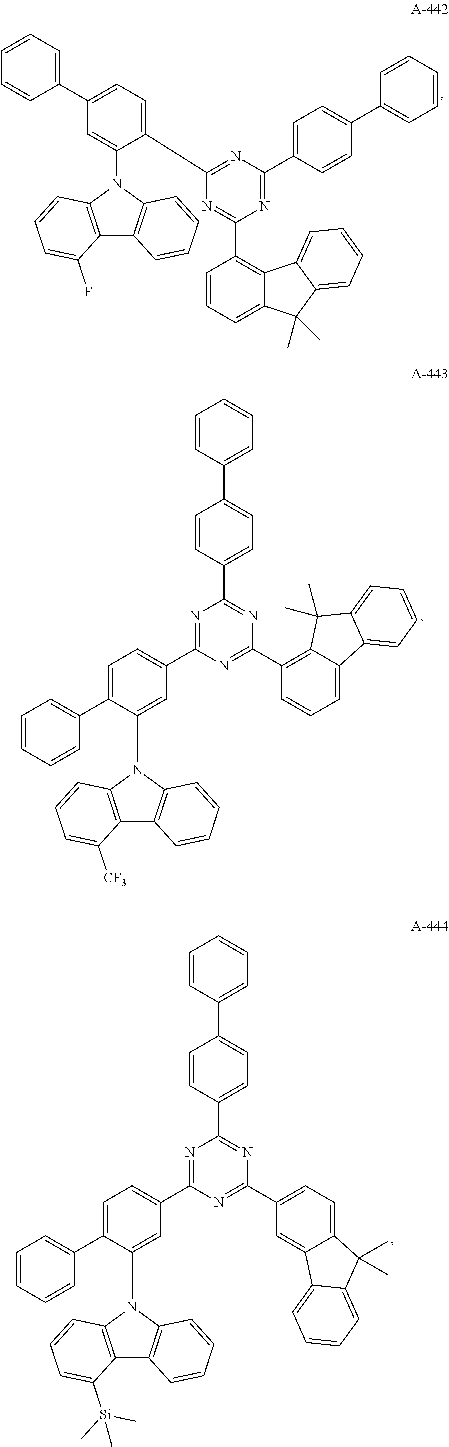 [SMILES: CC1(C)c2ccccc2-c2c(-c3nc(-c4ccc(-c5ccccc5)cc4)nc(-c4ccc(-c5ccccc5)cc4-n4c5ccccc5c5c(F)cccc54)n3)cccc21 ; CC1(C)c2ccccc2-c2cccc(-c3nc(-c4ccc(-c5ccccc5)cc4)nc(-c4ccc(-c5ccccc5)c(-n5c6ccccc6c6c(C(F)(F)F)cccc65)c4)n3)c21 ; CC1(C)c2ccccc2-c2cc(-c3nc(-c4ccc(-c5ccccc5)cc4)nc(-c4ccc(-c5ccccc5)c(-n5c6ccccc6c6c([Si](C)(C)C)cccc65)c4)n3)ccc21] 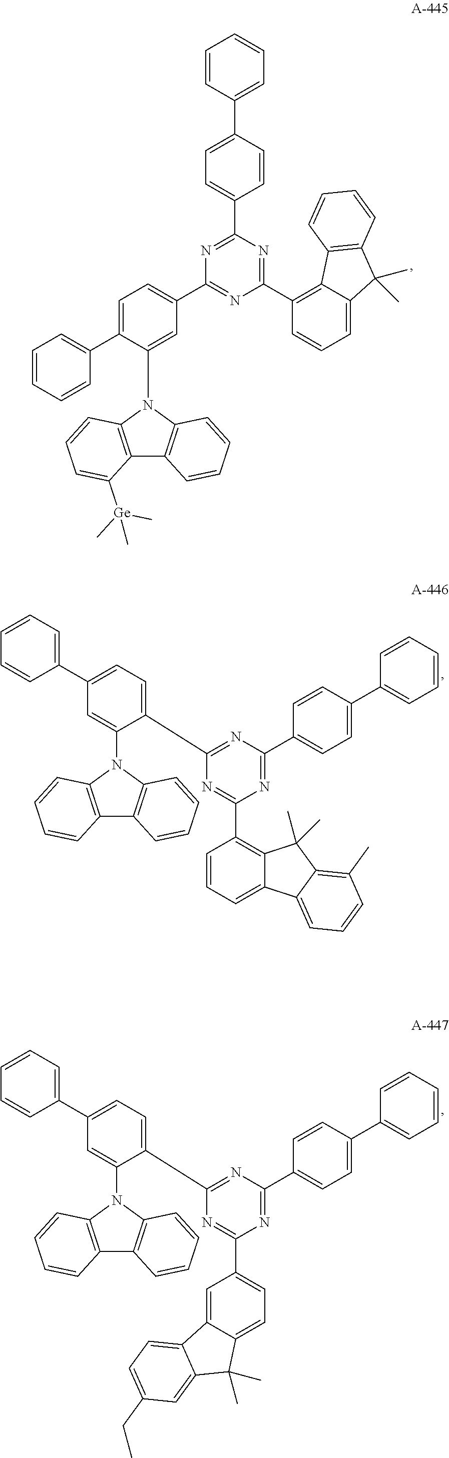 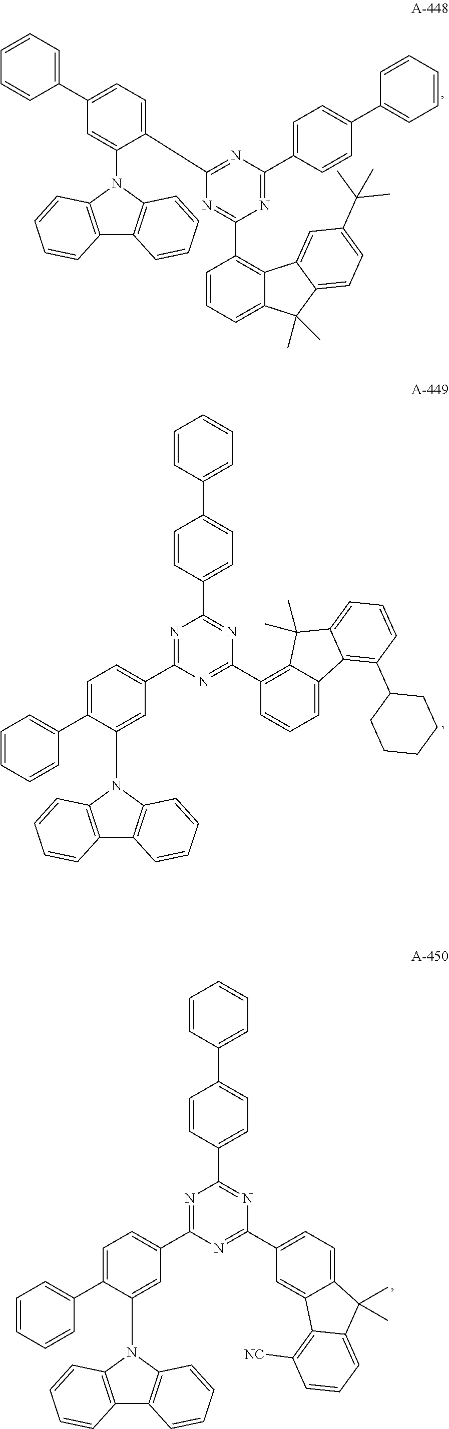 [SMILES: CC(C)(C)c1ccc2c(c1)-c1c(-c3nc(-c4ccc(-c5ccccc5)cc4)nc(-c4ccc(-c5ccccc5)cc4-n4c5ccccc5c5ccccc54)n3)cccc1C2(C)C ; CC1(C)c2cccc(C3CCCCC3)c2-c2cccc(-c3nc(-c4ccc(-c5ccccc5)cc4)nc(-c4ccc(-c5ccccc5)c(-n5c6ccccc6c6ccccc65)c4)n3)c21 ; CC1(C)c2ccc(-c3nc(-c4ccc(-c5ccccc5)cc4)nc(-c4ccc(-c5ccccc5)c(-n5c6ccccc6c6ccccc65)c4)n3)cc2-c2c(C#N)cccc21] 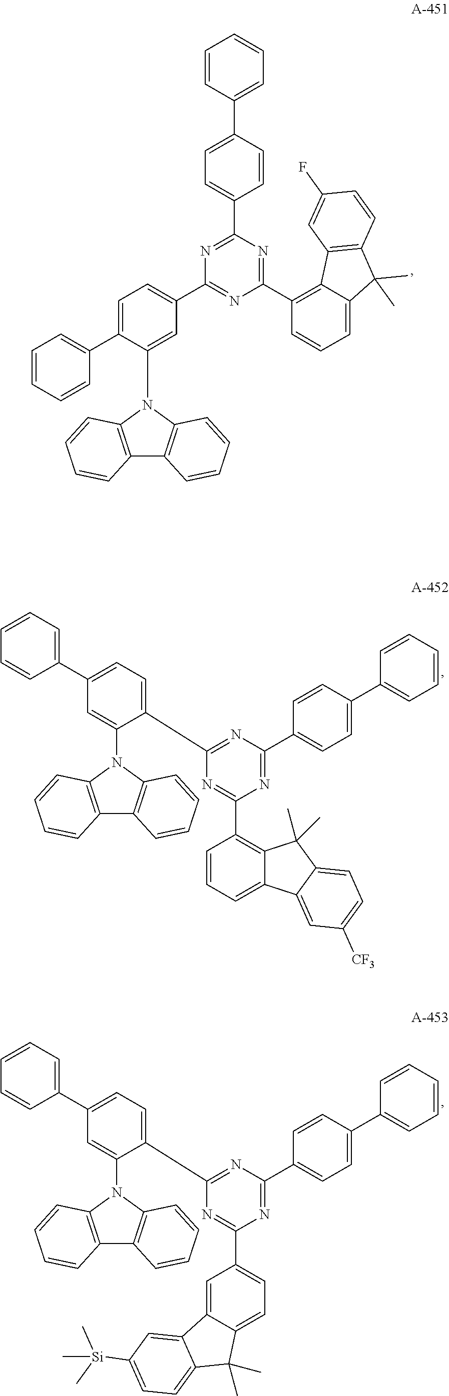 [SMILES: CC1(C)c2ccc(F)cc2-c2c(-c3nc(-c4ccc(-c5ccccc5)cc4)nc(-c4ccc(-c5ccccc5)c(-n5c6ccccc6c6ccccc65)c4)n3)cccc21 ; CC1(C)c2ccc(C(F)(F)F)cc2-c2cccc(-c3nc(-c4ccc(-c5ccccc5)cc4)nc(-c4ccc(-c5ccccc5)cc4-n4c5ccccc5c5ccccc54)n3)c21 ; CC1(C)c2ccc(-c3nc(-c4ccc(-c5ccccc5)cc4)nc(-c4ccc(-c5ccccc5)cc4-n4c5ccccc5c5ccccc54)n3)cc2-c2cc([Si](C)(C)C)ccc21] 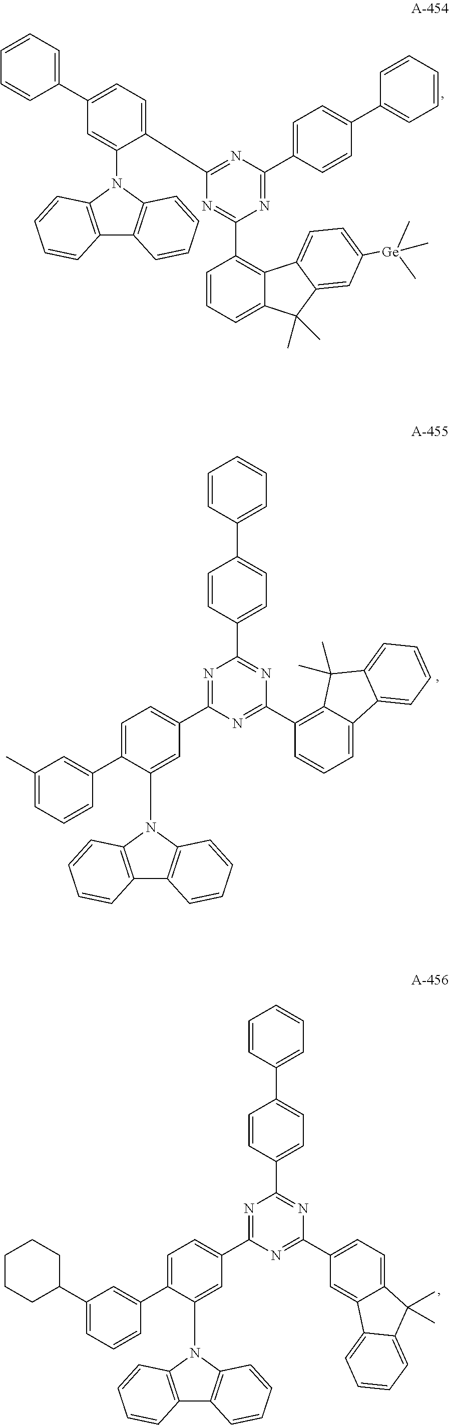 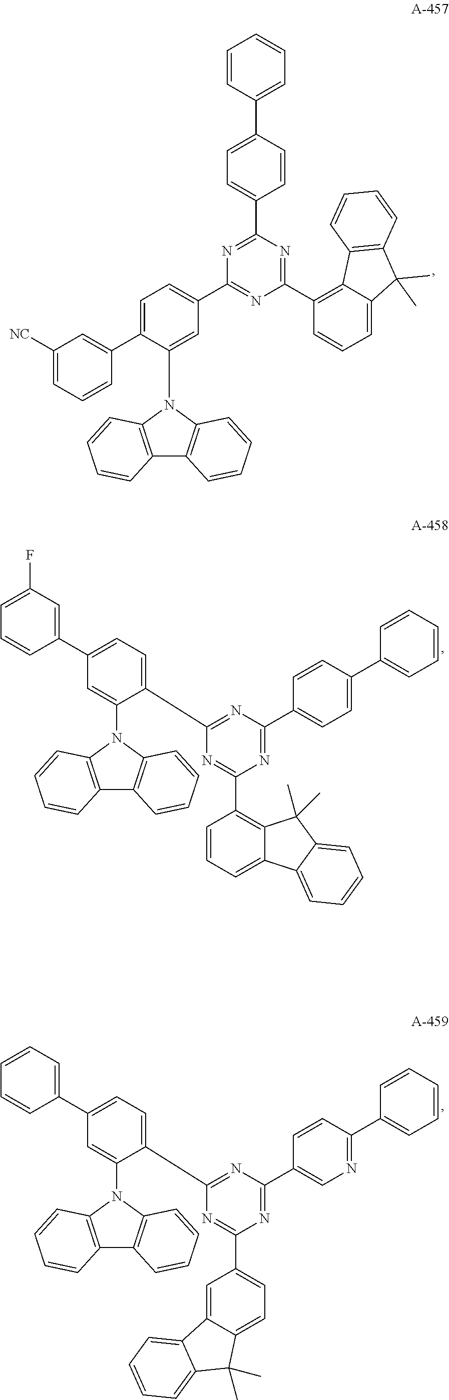 [SMILES: CC1(C)c2ccccc2-c2c(-c3nc(-c4ccc(-c5ccccc5)cc4)nc(-c4ccc(-c5cccc(C#N)c5)c(-n5c6ccccc6c6ccccc65)c4)n3)cccc21 ; CC1(C)c2ccccc2-c2cccc(-c3nc(-c4ccc(-c5ccccc5)cc4)nc(-c4ccc(-c5cccc(F)c5)cc4-n4c5ccccc5c5ccccc54)n3)c21 ; CC1(C)c2ccccc2-c2cc(-c3nc(-c4ccc(-c5ccccc5)nc4)nc(-c4ccc(-c5ccccc5)cc4-n4c5ccccc5c5ccccc54)n3)ccc21] 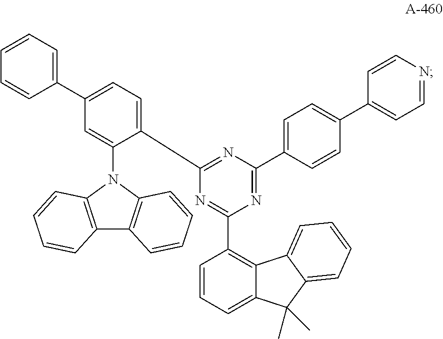 [SMILES: CC1(C)c2ccccc2-c2c(-c3nc(-c4ccc(-c5ccncc5)cc4)nc(-c4ccc(-c5ccccc5)cc4-n4c5ccccc5c5ccccc54)n3)cccc21]
wherein optionally, hydrogens in the above Compound A-1 to Compound A-460 can be partially or fully substituted with deuterium.

10. An organic electroluminescent device, comprising:
an anode,
a cathode, and
an organic layer disposed between the anode and the cathode, wherein the organic layer comprises the compound according to claim 1.

11. The organic electroluminescent device according to claim 10, wherein the organic layer is a light-emitting layer, and the compound is a host compound; or the organic layer is an electron transporting layer, and the compound is an electron transporting compound;
or the organic layer is a hole blocking layer, and the compound is a hole blocking compound.

12. The organic electroluminescent device according to claim 10, wherein the organic layer is a light-emitting layer, and the compound is a host compound; the light-emitting layer comprises a first metal complex, wherein the first metal complex has a general formula of M(Lₐ)ₘ(L_{b})ₙ(Lₑ)_{q};
the metal M is selected from a metal with a relative atomic mass greater than 40;
Lₐ, L_{b} and Lₑ are a first ligand, a second ligand and a third ligand coordinated to the metal M, respectively; the ligands Lₐ, L_{b} and Lₑ may be identical or different;
the ligands Lₐ, L_{b} and Lₑ can be optionally joined to form a multidentate ligand;
m is 1, 2 or 3, n is 0, 1 or 2, q is 0, 1 or 2, and m+n+q is equal to an oxidation state of the metal M; when m is greater than or equal to 2, a plurality of Lₐ may be identical or different;
when n is 2, two L_{b} may be identical or different; when q is 2, two Lₑ may be identical or different;
the ligand Lₐ has a structure represented by Formula 2: 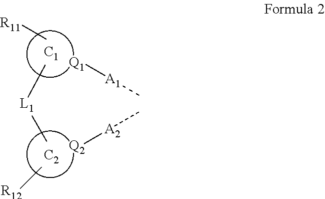
wherein the ring C₁ and the ring C₂ are, at each occurrence identically or differently, selected from an aromatic ring having 5 to 30 ring atoms, a heteroaromatic ring having 5 to 30 ring atoms or a combination thereof;
Q₁ and Q₂ are, at each occurrence identically or differently, selected from C or N;
A₁ and A₂ are, at each occurrence identically or differently, selected from a single bond, O or S;
L₁ is, at each occurrence identically or differently, selected from the group consisting of: a single bond, BR′, CR′R′, NR′, O, SiR′R′, PR′, S, GeR′R′, Se, substituted or unsubstituted vinylene, ethynylene, substituted or unsubstituted arylene having 6 to 30 carbon atoms, substituted or unsubstituted heteroarylene having 5 to 30 carbon atoms and combinations thereof; when two R′ are present at the same time, the two R′ are identical or different;
R₁₁ and R₁₂ represent, at each occurrence identically or differently, mono-substitution, multiple substitutions or non-substitution;
R′, R₁₁ and R₁₂ are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
adjacent substituents R′, R₁₁ and R₁₂ can be optionally joined to form a ring;
the ligands L_{b} and Lₑ are, at each occurrence identically or differently, selected from a monoanionic bidentate ligand;
preferably, the ligands L_{b} and Lₑ are, at each occurrence identically or differently, selected from any one or two of the following structures: 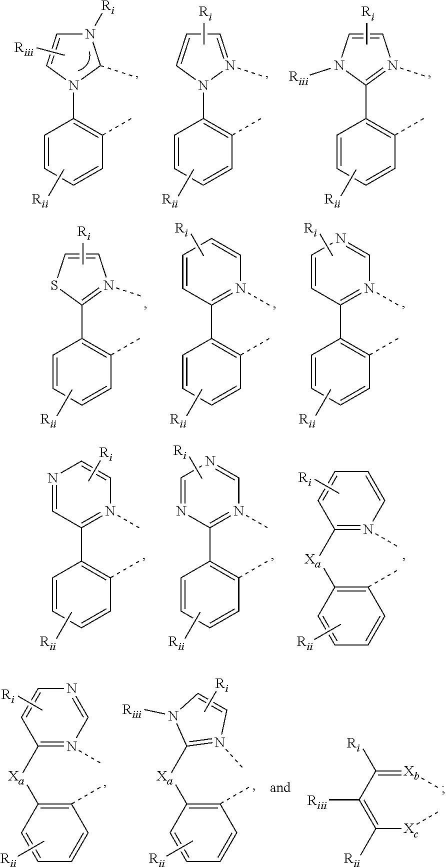
wherein
Rₐ, R_{b} and Rₑ represent, at each occurrence identically or differently, mono-substitution, multiple substitutions or non-substitution;
X_{b} is, at each occurrence identically or differently, selected from the group consisting of: O, S, Se, NR_{N1} and CR_{C1}R_{C2};
Xₑ and X_{d} are, at each occurrence identically or differently, selected from the group consisting of: O, S, Se and NR_{N2};
Rₐ, R_{b}, Rₑ, R_{N1}, R_{N2}, R_{C1} and R_{C2} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof; and
adjacent substituents Rₐ, R_{b}, Rₑ, R_{N1}, R_{N2}, R_{C1} and R_{C2} can be optionally joined to form a ring.

13. The organic electroluminescent device according to claim 12, wherein the first metal complex is selected from the group consisting of the following structures: 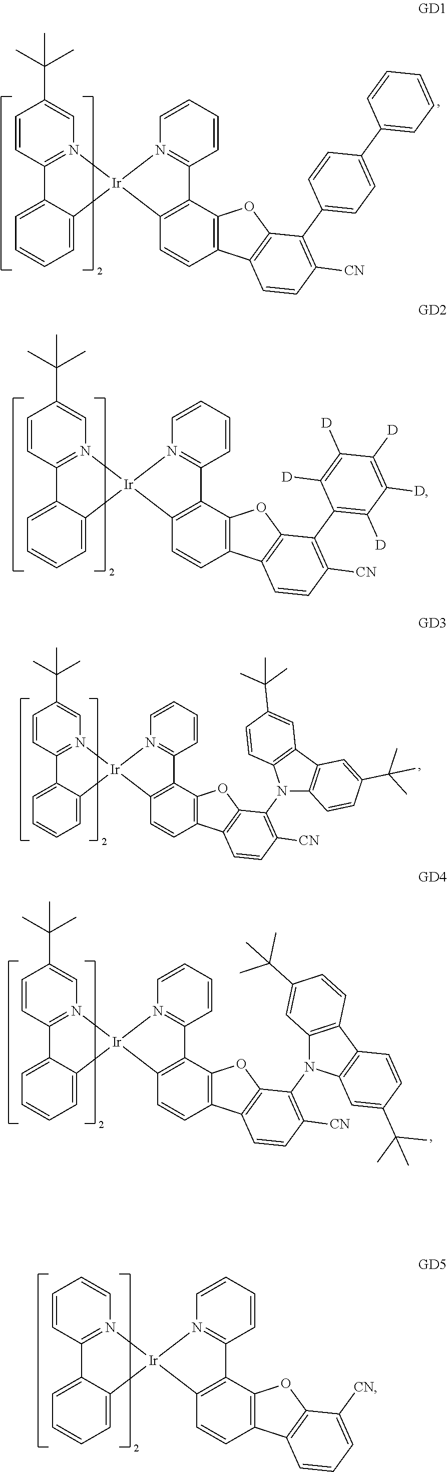 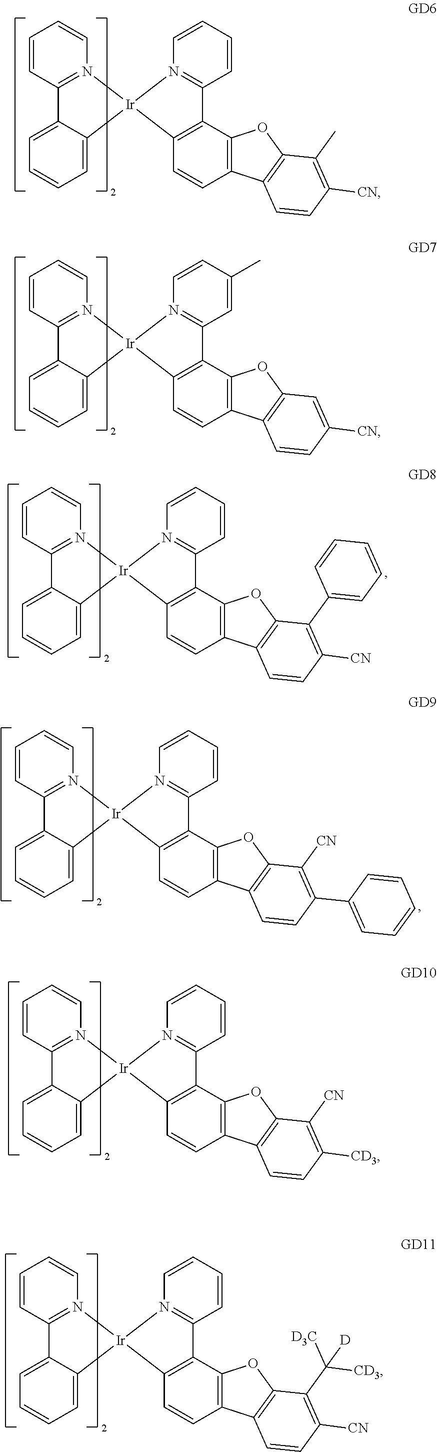 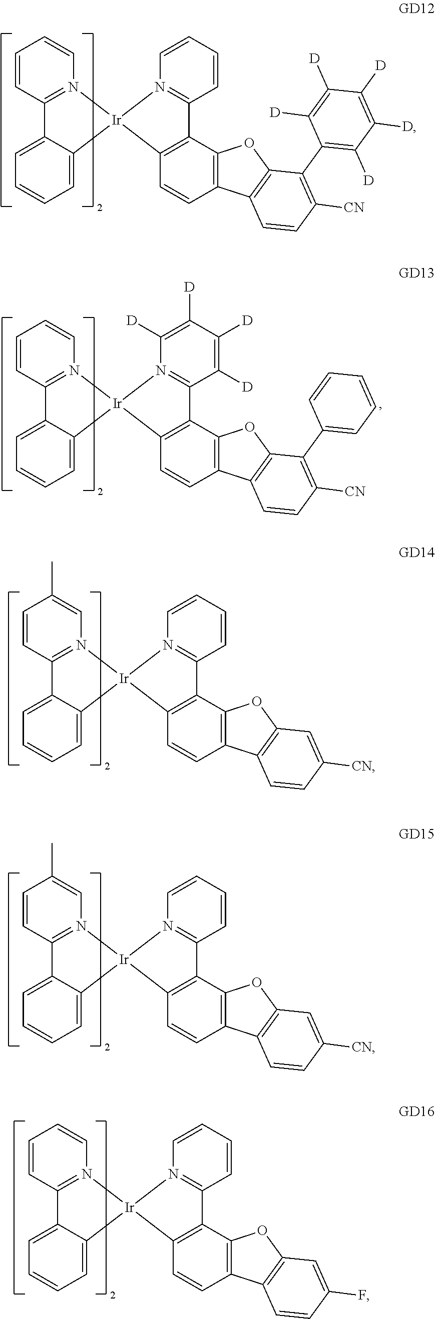 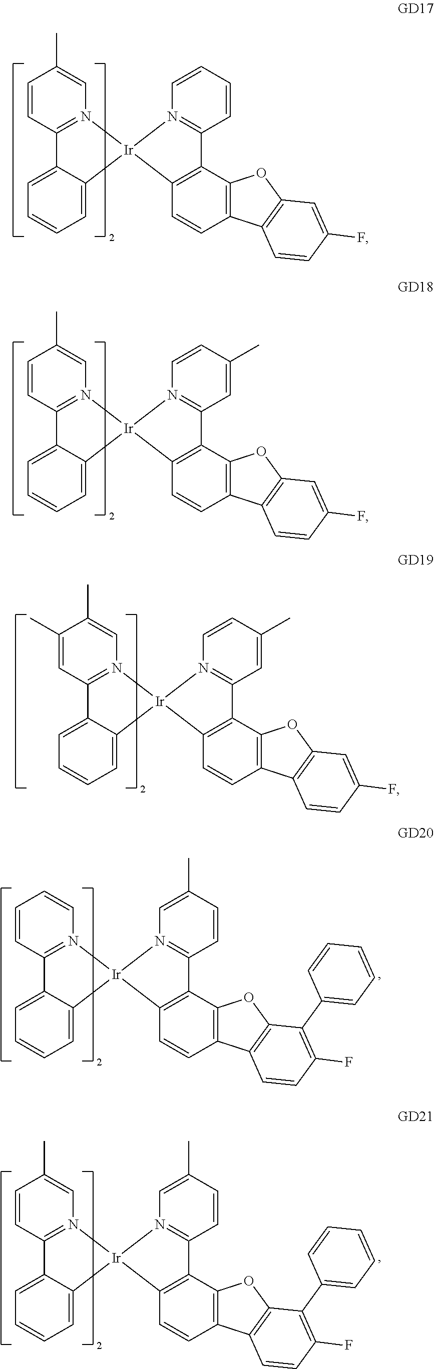 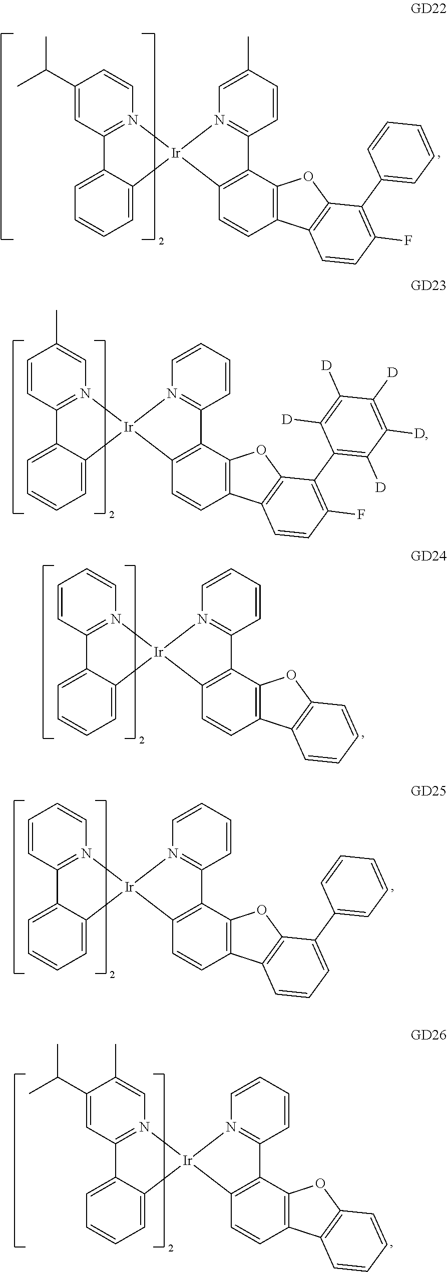 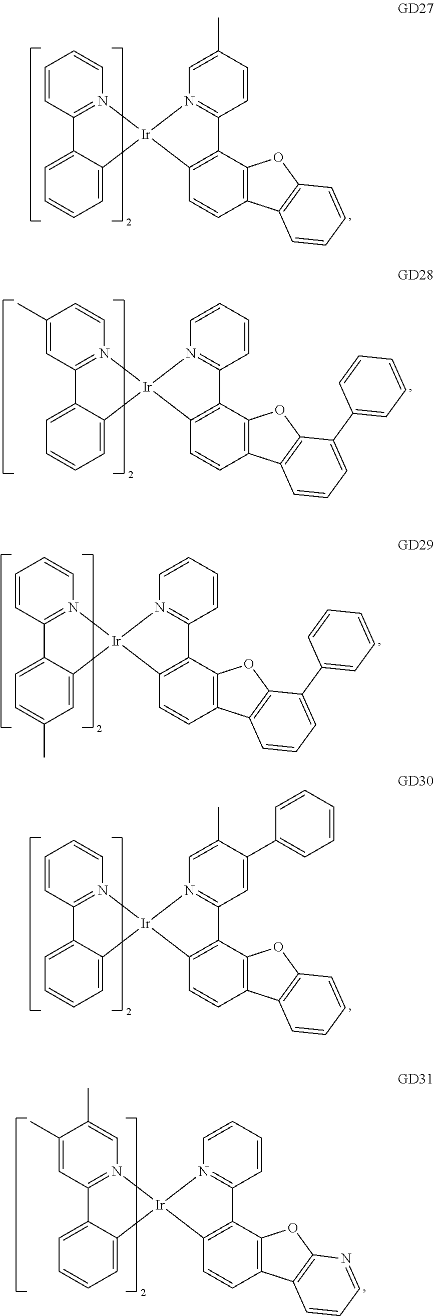 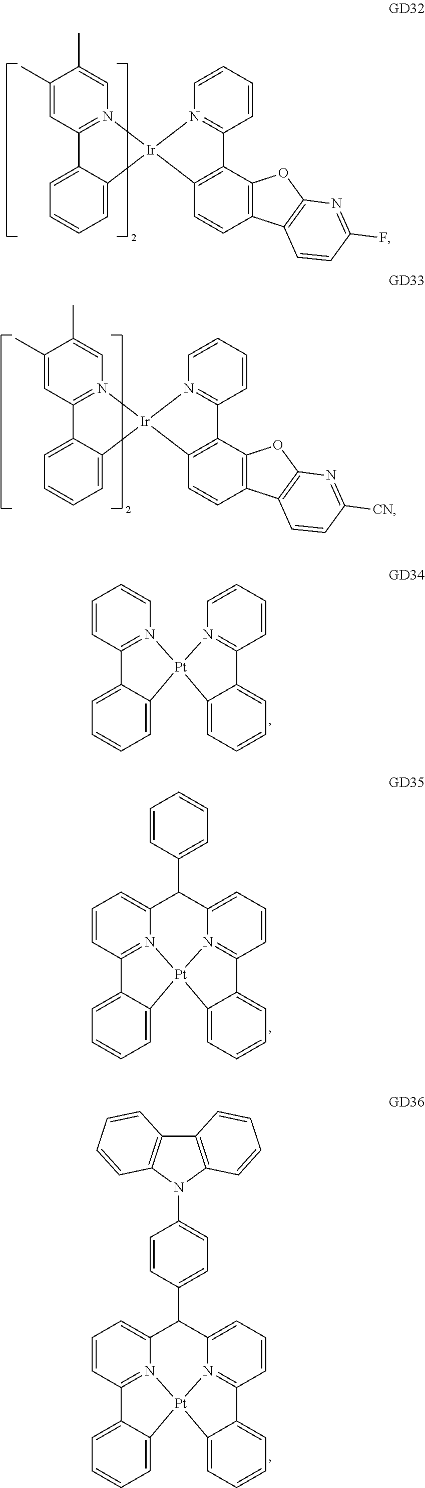 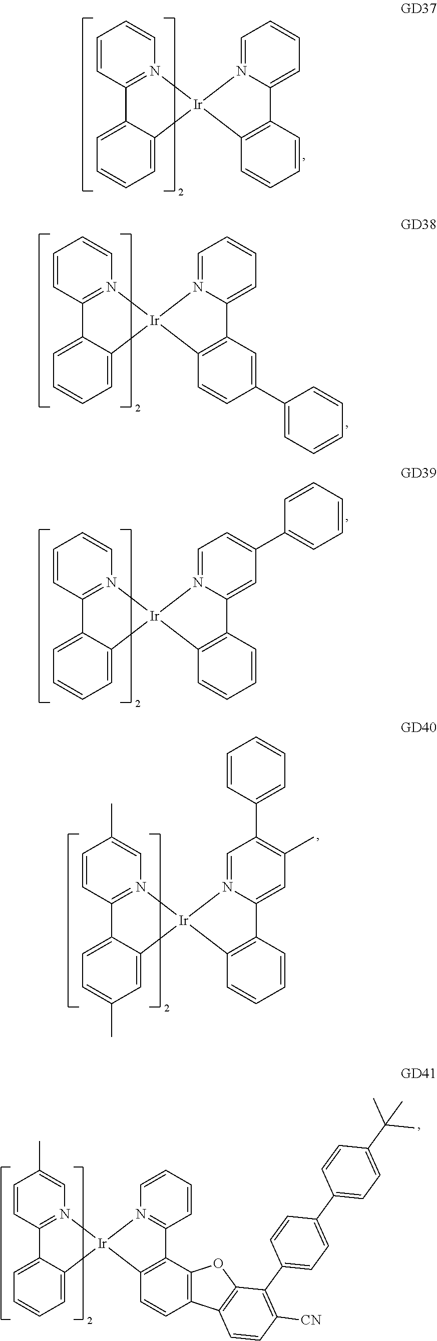 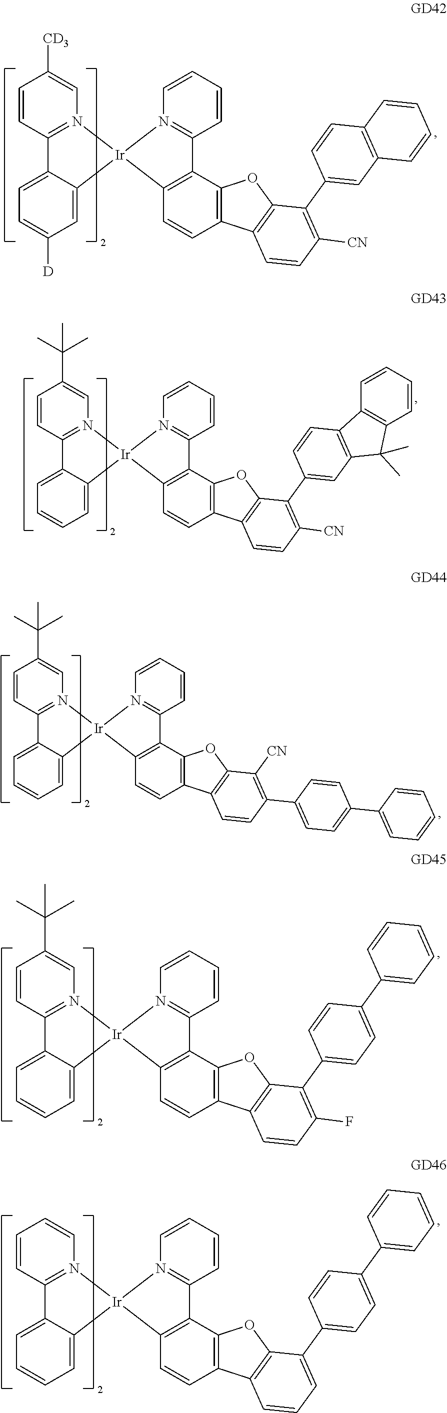 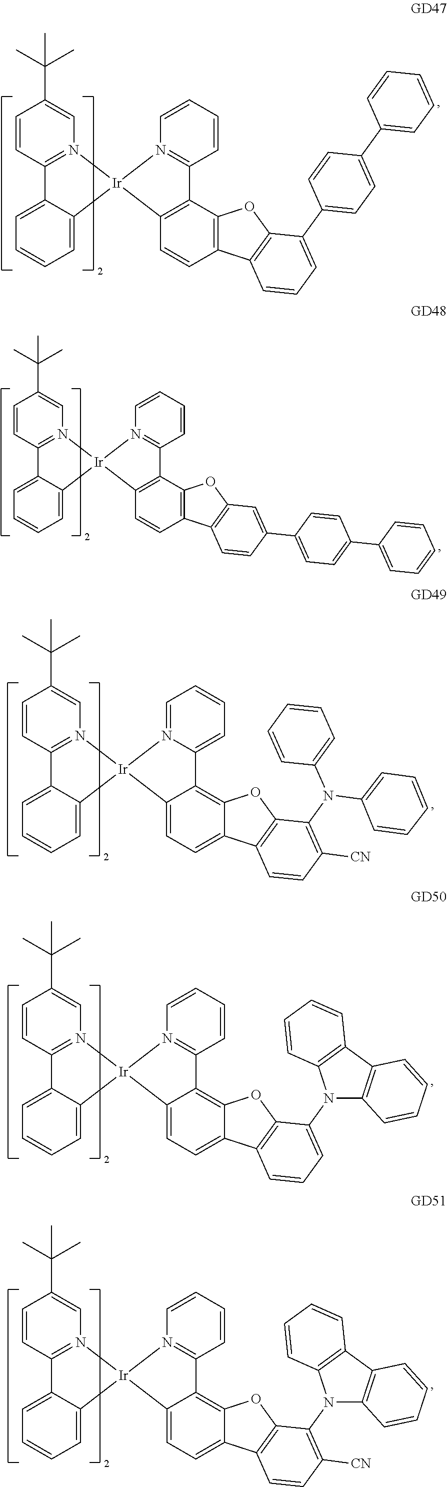 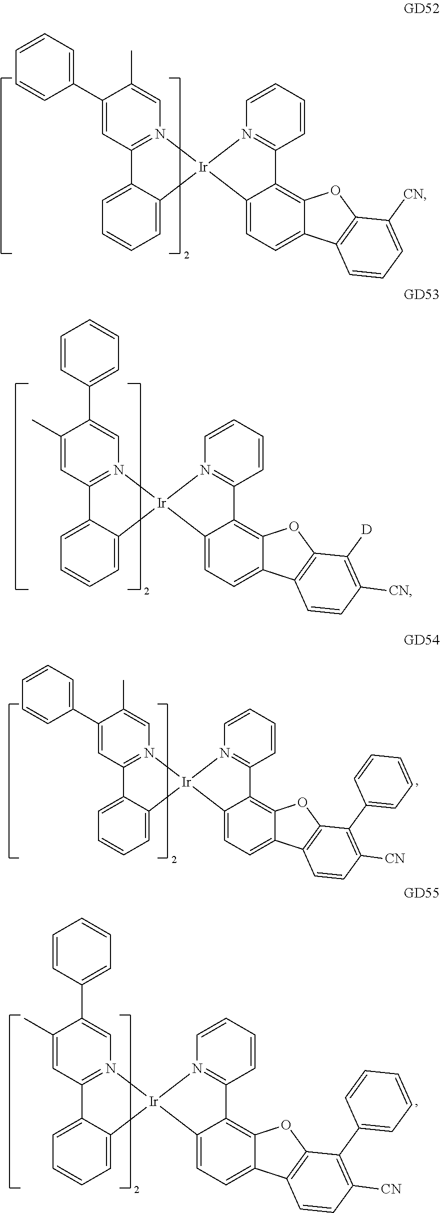 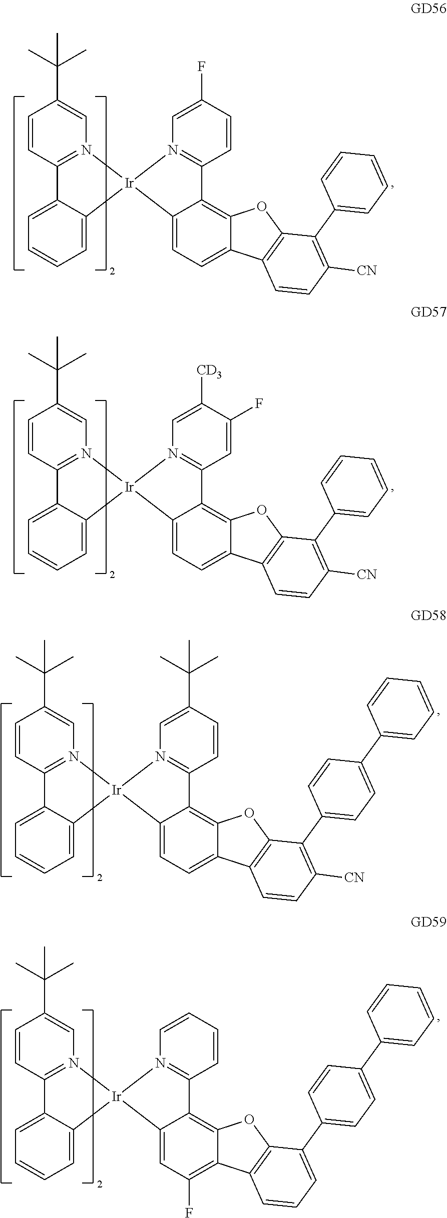 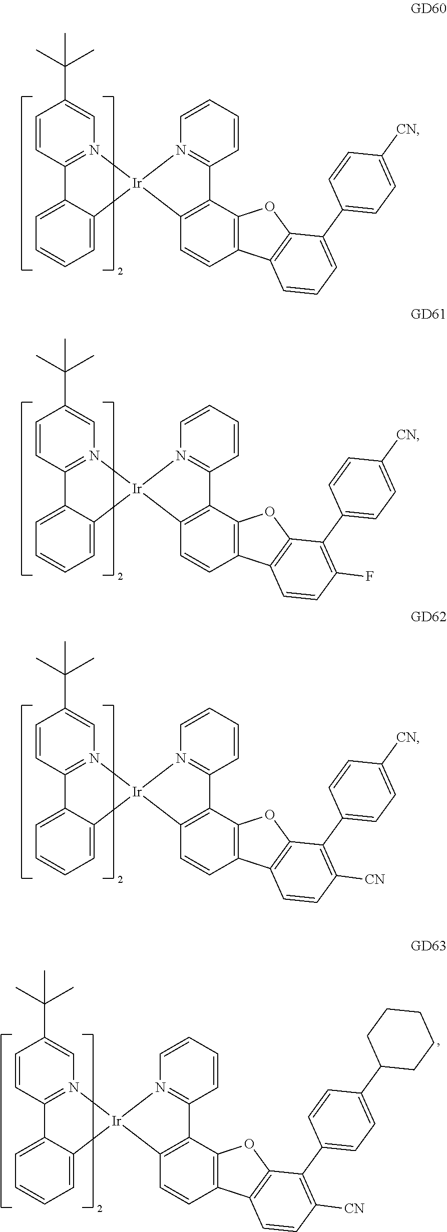 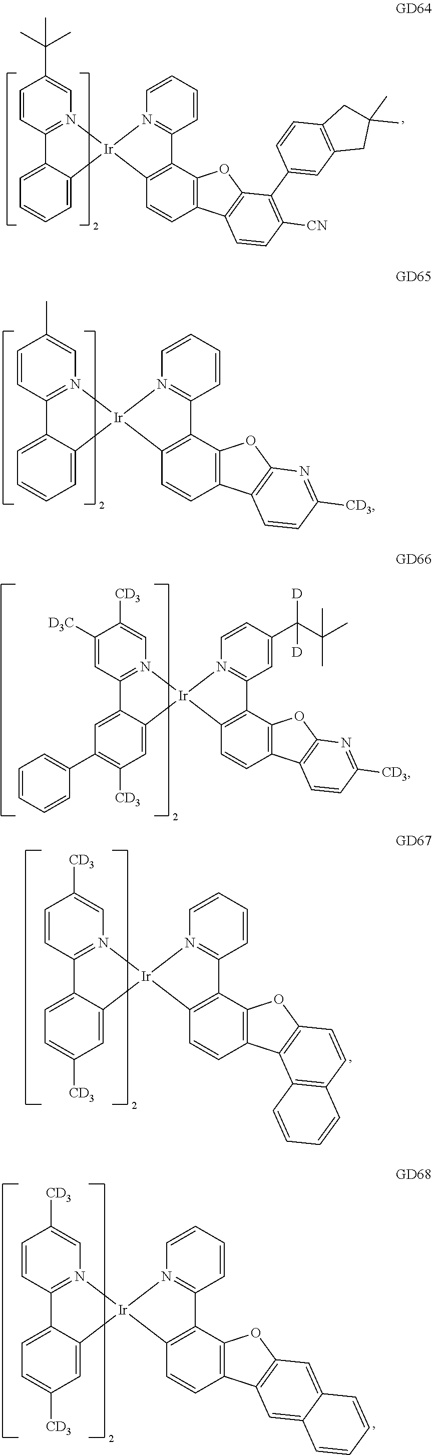 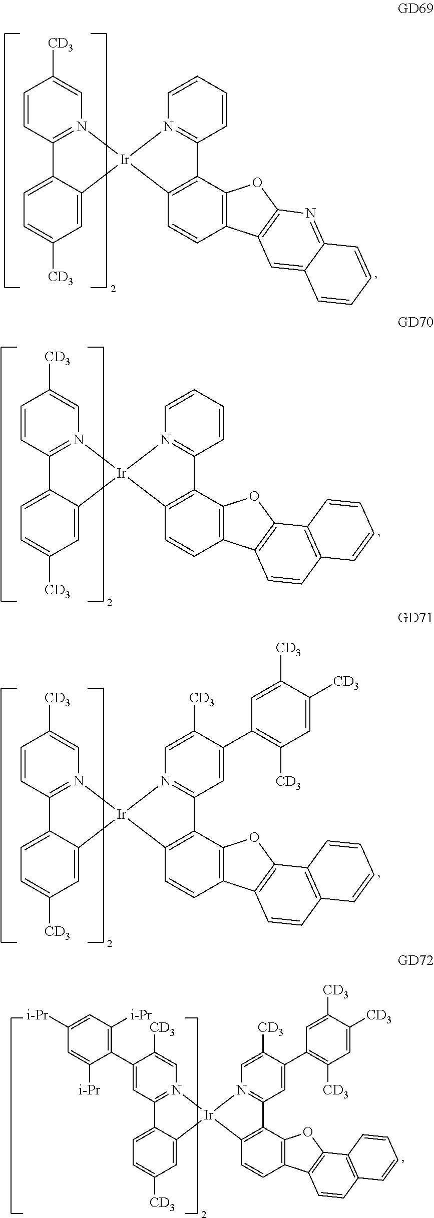 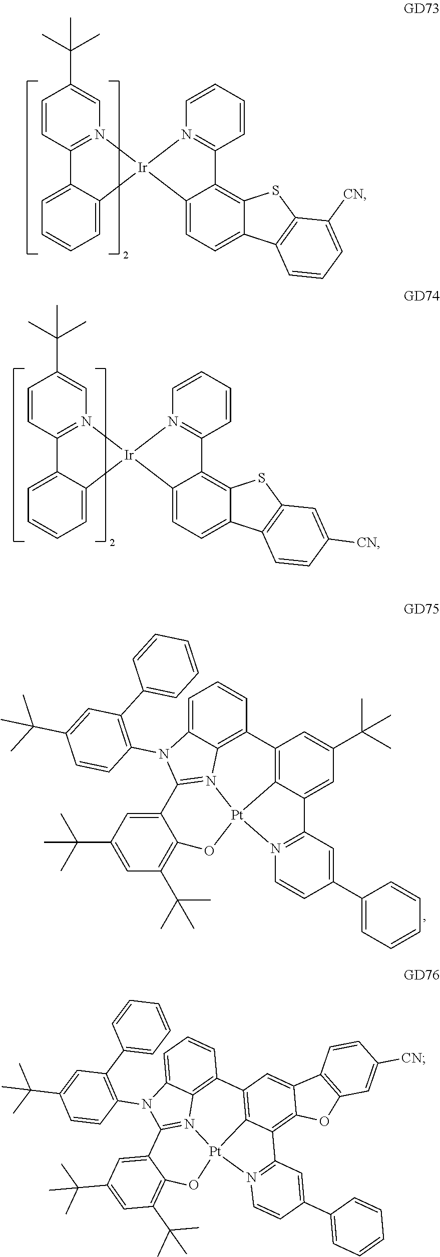
wherein optionally, hydrogens in the above Compound GD1 to Compound GD76 can be partially or fully substituted with deuterium.

14. The organic electroluminescent device according to claim 11, wherein the organic layer is the light-emitting layer, the compound is the host compound, and the light-emitting layer further comprises a second host compound, wherein the second host compound comprises at least one chemical group selected from the group consisting of: benzene, pyridine, pyrimidine, triazine, carbazole, azacarbazole, indolocarbazole, dibenzothiophene, aza-dibenzothiophene, dibenzofuran, azadibenzofuran, dibenzoselenophene, triphenylene, azatriphenylene, fluorene, silafluorene, naphthalene, quinoline, isoquinoline, quinazoline, quinoxaline, phenanthrene, azaphenanthrene and combinations thereof; and
preferably, the second host compound comprises at least one chemical group selected from the group consisting of: benzene, carbazole, indolocarbazole, fluorene, silafluorene and combinations thereof.

15. The organic electroluminescent device according to claim 14, wherein the second host compound has a structure represented by Formula 3 or Formula 4: 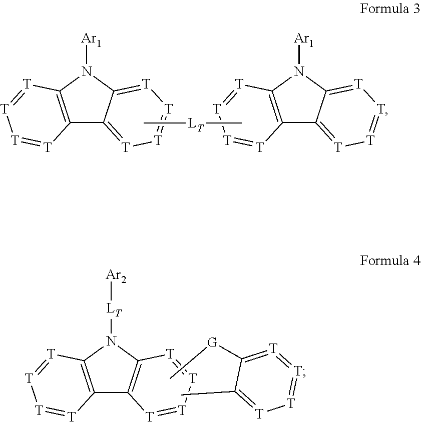
wherein
G is, at each occurrence identically or differently, selected from C(R_{g})₂, NR_{g}, O or S;
L₁ is, at each occurrence identically or differently, selected from a single bond, substituted or unsubstituted alkylene having 1 to 20 carbon atoms, substituted or unsubstituted cycloalkylene having 3 to 20 carbon atoms, substituted or unsubstituted arylene having 6 to 20 carbon atoms, substituted or unsubstituted heteroarylene having 3 to 20 carbon atoms or a combination thereof;
T is, at each occurrence identically or differently, selected from C, CRₜ or N;
Rₜ and R_{g} are, at each occurrence identically or differently, selected from the group consisting of: hydrogen, deuterium, halogen, substituted or unsubstituted alkyl having 1 to 20 carbon atoms, substituted or unsubstituted cycloalkyl having 3 to 20 ring carbon atoms, substituted or unsubstituted heteroalkyl having 1 to 20 carbon atoms, a substituted or unsubstituted heterocyclic group having 3 to 20 ring atoms, substituted or unsubstituted arylalkyl having 7 to 30 carbon atoms, substituted or unsubstituted alkoxy having 1 to 20 carbon atoms, substituted or unsubstituted aryloxy having 6 to 30 carbon atoms, substituted or unsubstituted alkenyl having 2 to 20 carbon atoms, substituted or unsubstituted alkynyl having 2 to 20 carbon atoms, substituted or unsubstituted aryl having 6 to 30 carbon atoms, substituted or unsubstituted heteroaryl having 3 to 30 carbon atoms, substituted or unsubstituted alkylsilyl having 3 to 20 carbon atoms, substituted or unsubstituted arylsilyl having 6 to 20 carbon atoms, substituted or unsubstituted alkylgermanyl having 3 to 20 carbon atoms, substituted or unsubstituted arylgermanyl having 6 to 20 carbon atoms, substituted or unsubstituted amino having 0 to 20 carbon atoms, an acyl group, a carbonyl group, a carboxylic acid group, an ester group, a cyano group, an isocyano group, a hydroxyl group, a sulfanyl group, a sulfinyl group, a sulfonyl group, a phosphino group and combinations thereof;
Ar₁ and Ar₂ are, at each occurrence identically or differently, selected from substituted or unsubstituted aryl having 6 to 30 carbon atoms, substituted or unsubstituted heteroaryl having 3 to 30 carbon atoms and combinations thereof; and
adjacent substituents Rₜ and R_{g} can be optionally joined to form a ring.

16. The organic electroluminescent device according to claim 14, wherein the second host compound is selected from the group consisting of the following structures:

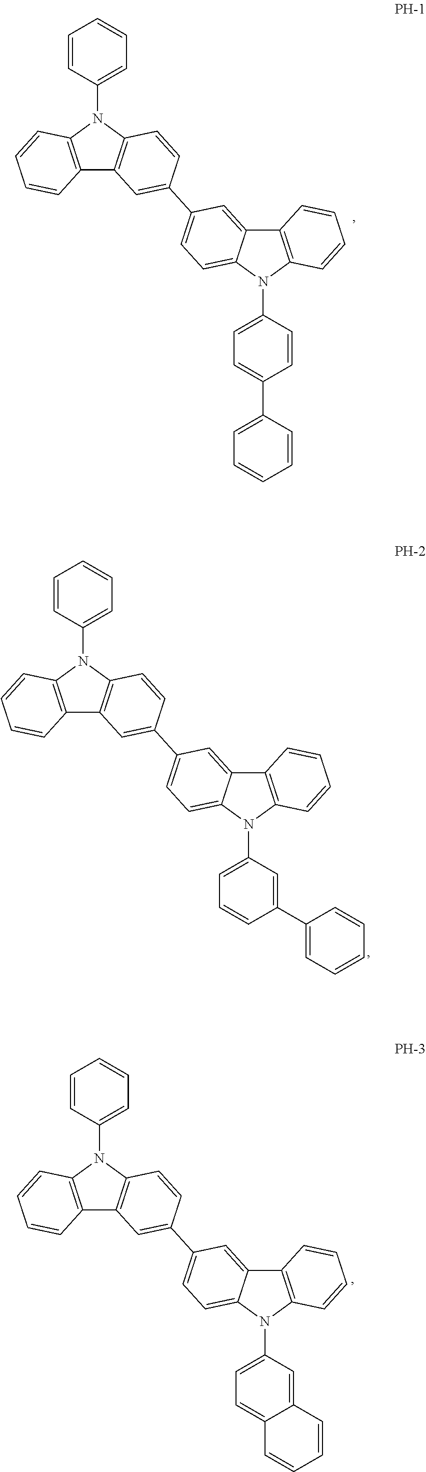 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cccc(-n3c4ccccc4c4cc(-c5ccc6c(c5)c5ccccc5n6-c5ccccc5)ccc43)c2)cc1 ; c1ccc(-n2c3ccccc3c3cc(-c4ccc5c(c4)c4ccccc4n5-c4ccc5ccccc5c4)ccc32)cc1]

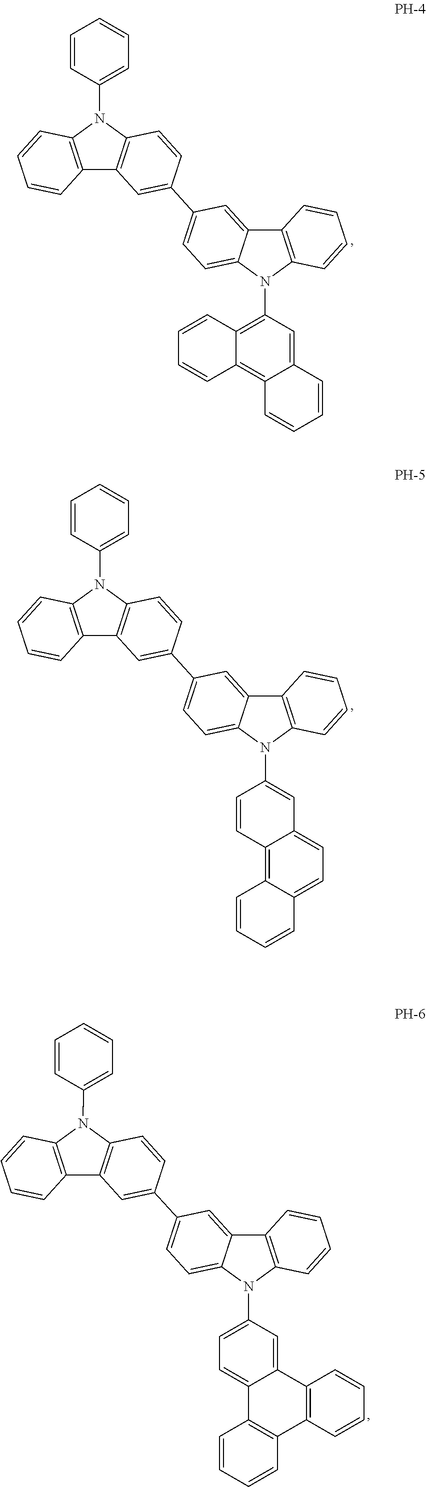 [SMILES: c1ccc(-n2c3ccccc3c3cc(-c4ccc5c(c4)c4ccccc4n5-c4cc5ccccc5c5ccccc45)ccc32)cc1 ; c1ccc(-n2c3ccccc3c3cc(-c4ccc5c(c4)c4ccccc4n5-c4ccc5c(ccc6ccccc65)c4)ccc32)cc1 ; c1ccc(-n2c3ccccc3c3cc(-c4ccc5c(c4)c4ccccc4n5-c4ccc5c6ccccc6c6ccccc6c5c4)ccc32)cc1]

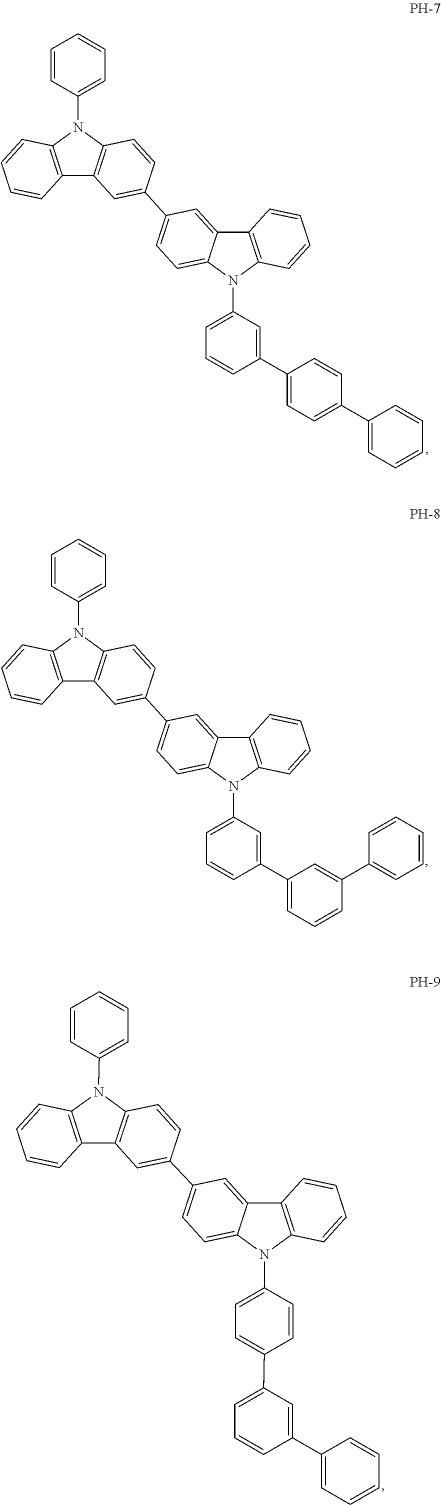 [SMILES: c1ccc(-c2ccc(-c3cccc(-n4c5ccccc5c5cc(-c6ccc7c(c6)c6ccccc6n7-c6ccccc6)ccc54)c3)cc2)cc1 ; c1ccc(-c2cccc(-c3cccc(-n4c5ccccc5c5cc(-c6ccc7c(c6)c6ccccc6n7-c6ccccc6)ccc54)c3)c2)cc1 ; c1ccc(-c2cccc(-c3ccc(-n4c5ccccc5c5cc(-c6ccc7c(c6)c6ccccc6n7-c6ccccc6)ccc54)cc3)c2)cc1]

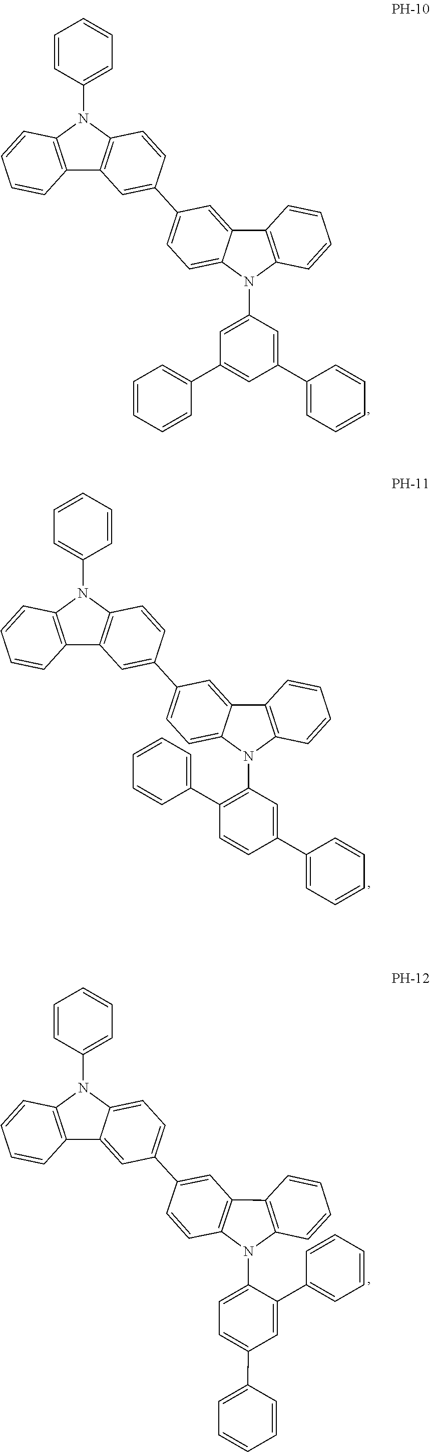 [SMILES: c1ccc(-c2cc(-c3ccccc3)cc(-n3c4ccccc4c4cc(-c5ccc6c(c5)c5ccccc5n6-c5ccccc5)ccc43)c2)cc1 ; c1ccc(-c2ccc(-c3ccccc3)c(-n3c4ccccc4c4cc(-c5ccc6c(c5)c5ccccc5n6-c5ccccc5)ccc43)c2)cc1 ; c1ccc(-c2ccc(-n3c4ccccc4c4cc(-c5ccc6c(c5)c5ccccc5n6-c5ccccc5)ccc43)c(-c3ccccc3)c2)cc1]

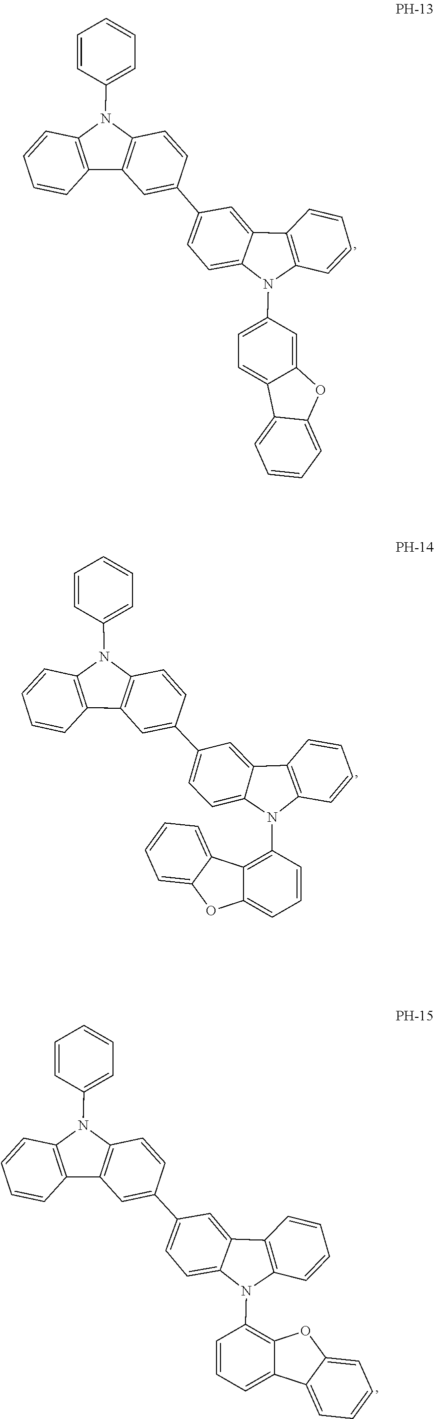 [SMILES: c1ccc(-n2c3ccccc3c3cc(-c4ccc5c(c4)c4ccccc4n5-c4ccc5c(c4)oc4ccccc45)ccc32)cc1 ; c1ccc(-n2c3ccccc3c3cc(-c4ccc5c(c4)c4ccccc4n5-c4cccc5oc6ccccc6c45)ccc32)cc1 ; c1ccc(-n2c3ccccc3c3cc(-c4ccc5c(c4)c4ccccc4n5-c4cccc5c4oc4ccccc45)ccc32)cc1]

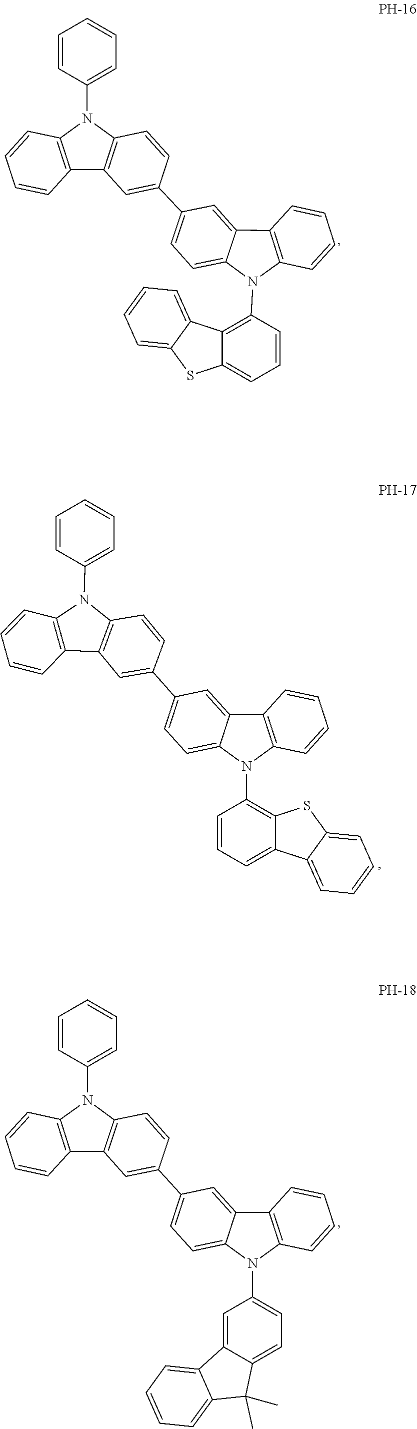 [SMILES: c1ccc(-n2c3ccccc3c3cc(-c4ccc5c(c4)c4ccccc4n5-c4cccc5sc6ccccc6c45)ccc32)cc1 ; c1ccc(-n2c3ccccc3c3cc(-c4ccc5c(c4)c4ccccc4n5-c4cccc5c4sc4ccccc45)ccc32)cc1 ; CC1(C)c2ccccc2-c2cc(-n3c4ccccc4c4cc(-c5ccc6c(c5)c5ccccc5n6-c5ccccc5)ccc43)ccc21]

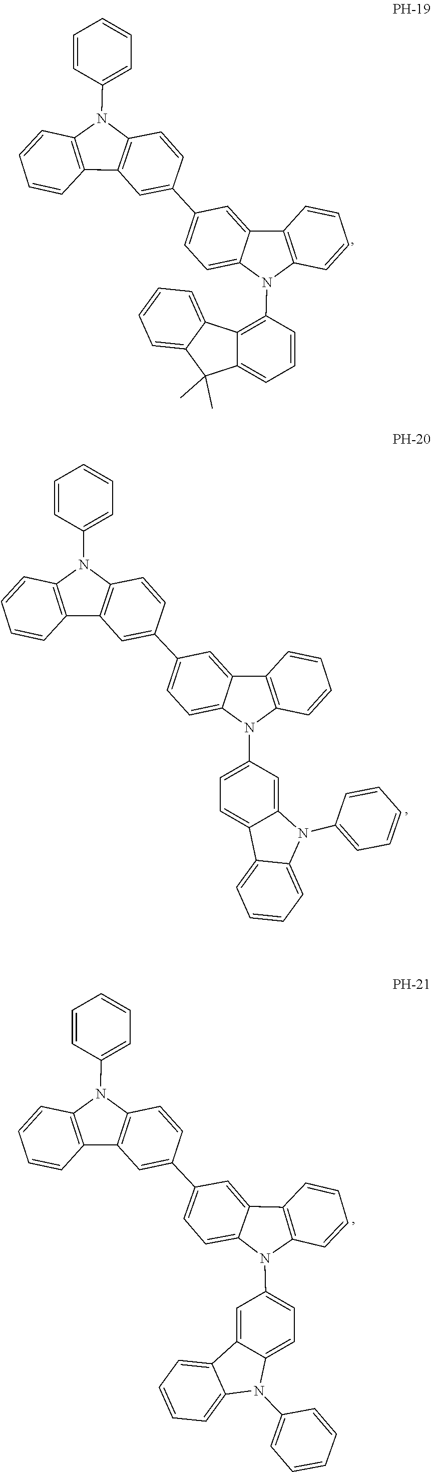 [SMILES: CC1(C)c2ccccc2-c2c(-n3c4ccccc4c4cc(-c5ccc6c(c5)c5ccccc5n6-c5ccccc5)ccc43)cccc21 ; c1ccc(-n2c3ccccc3c3cc(-c4ccc5c(c4)c4ccccc4n5-c4ccc5c6ccccc6n(-c6ccccc6)c5c4)ccc32)cc1 ; c1ccc(-n2c3ccccc3c3cc(-c4ccc5c(c4)c4ccccc4n5-c4ccc5c(c4)c4ccccc4n5-c4ccccc4)ccc32)cc1]

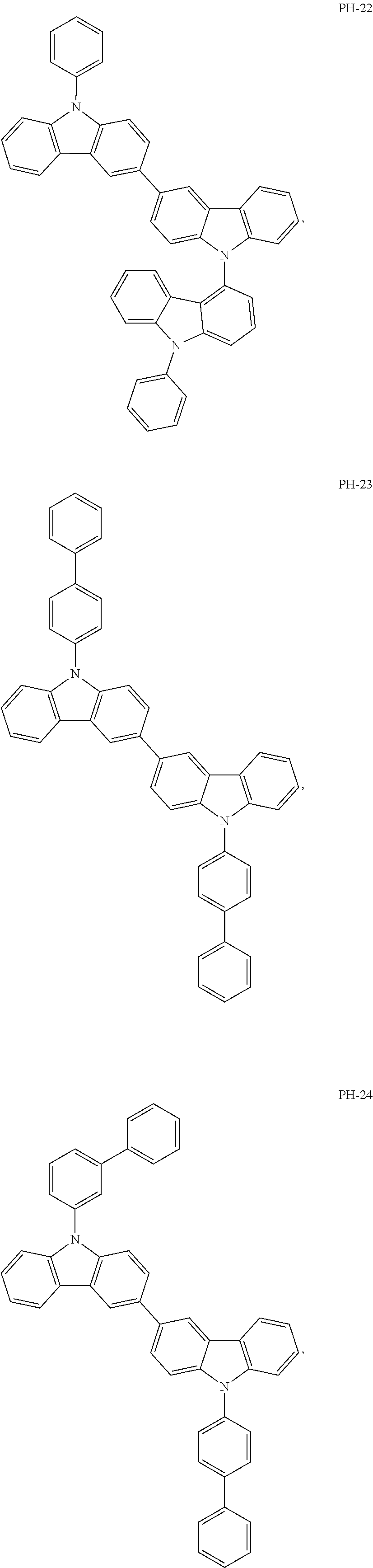 [SMILES: c1ccc(-n2c3ccccc3c3cc(-c4ccc5c(c4)c4ccccc4n5-c4cccc5c4c4ccccc4n5-c4ccccc4)ccc32)cc1 ; c1ccc(-c2ccc(-n3c4ccccc4c4cc(-c5ccc6c(c5)c5ccccc5n6-c5ccc(-c6ccccc6)cc5)ccc43)cc2)cc1 ; c1ccc(-c2ccc(-n3c4ccccc4c4cc(-c5ccc6c(c5)c5ccccc5n6-c5cccc(-c6ccccc6)c5)ccc43)cc2)cc1]

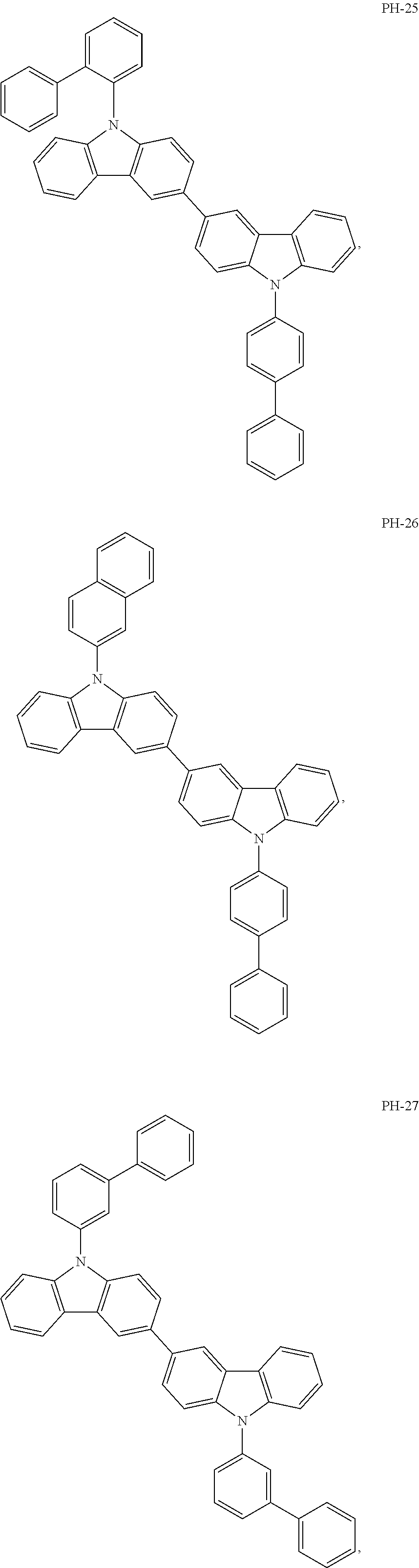 [SMILES: c1ccc(-c2ccc(-n3c4ccccc4c4cc(-c5ccc6c(c5)c5ccccc5n6-c5ccccc5-c5ccccc5)ccc43)cc2)cc1 ; c1ccc(-c2ccc(-n3c4ccccc4c4cc(-c5ccc6c(c5)c5ccccc5n6-c5ccc6ccccc6c5)ccc43)cc2)cc1 ; c1ccc(-c2cccc(-n3c4ccccc4c4cc(-c5ccc6c(c5)c5ccccc5n6-c5cccc(-c6ccccc6)c5)ccc43)c2)cc1]

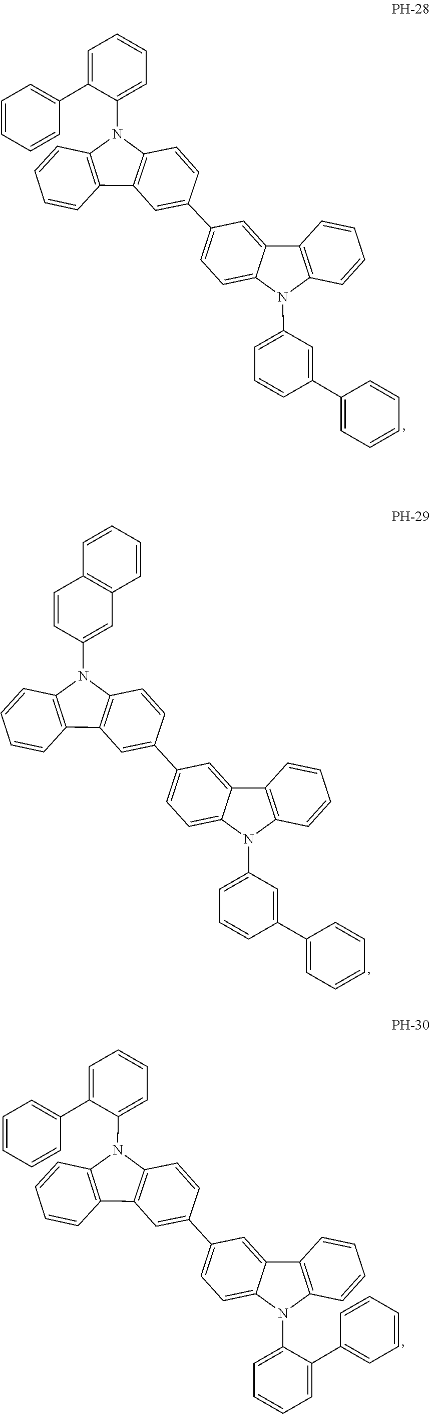 [SMILES: c1ccc(-c2cccc(-n3c4ccccc4c4cc(-c5ccc6c(c5)c5ccccc5n6-c5ccccc5-c5ccccc5)ccc43)c2)cc1 ; c1ccc(-c2cccc(-n3c4ccccc4c4cc(-c5ccc6c(c5)c5ccccc5n6-c5ccc6ccccc6c5)ccc43)c2)cc1 ; c1ccc(-c2ccccc2-n2c3ccccc3c3cc(-c4ccc5c(c4)c4ccccc4n5-c4ccccc4-c4ccccc4)ccc32)cc1]

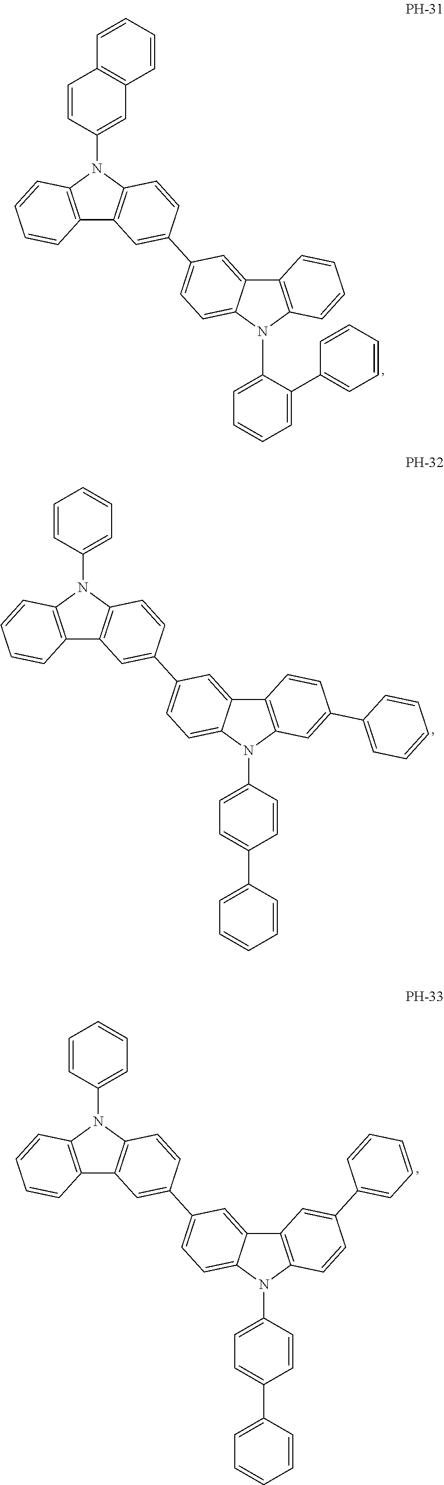 [SMILES: c1ccc(-c2ccccc2-n2c3ccccc3c3cc(-c4ccc5c(c4)c4ccccc4n5-c4ccc5ccccc5c4)ccc32)cc1 ; c1ccc(-c2ccc(-n3c4ccc(-c5ccc6c(c5)c5ccccc5n6-c5ccccc5)cc4c4ccc(-c5ccccc5)cc43)cc2)cc1 ; c1ccc(-c2ccc(-n3c4ccc(-c5ccccc5)cc4c4cc(-c5ccc6c(c5)c5ccccc5n6-c5ccccc5)ccc43)cc2)cc1]

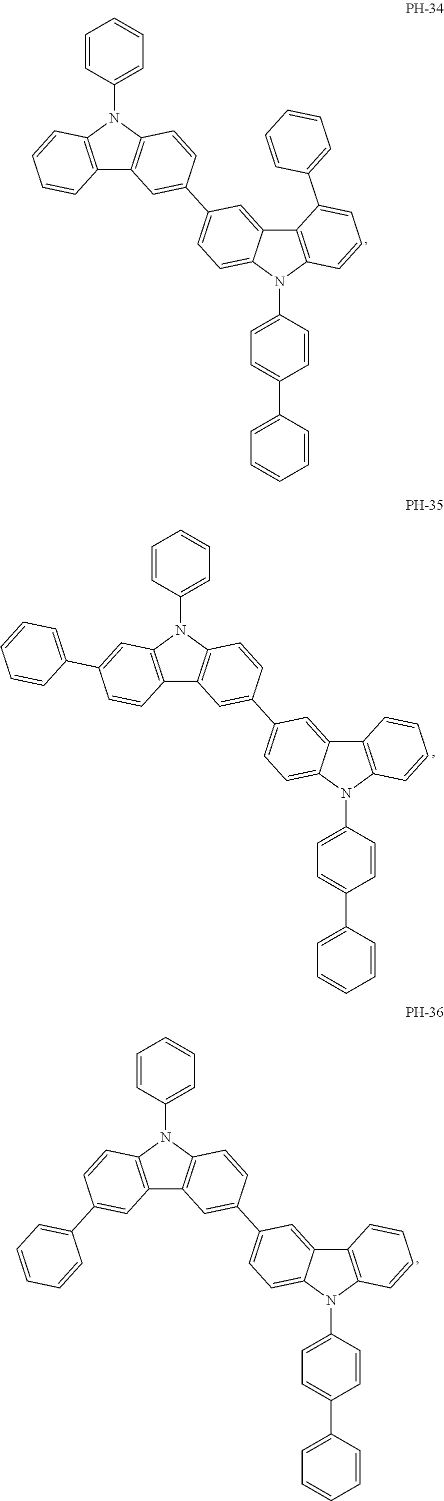 [SMILES: c1ccc(-c2ccc(-n3c4ccc(-c5ccc6c(c5)c5ccccc5n6-c5ccccc5)cc4c4c(-c5ccccc5)cccc43)cc2)cc1 ; c1ccc(-c2ccc(-n3c4ccccc4c4cc(-c5ccc6c(c5)c5ccc(-c7ccccc7)cc5n6-c5ccccc5)ccc43)cc2)cc1 ; c1ccc(-c2ccc(-n3c4ccccc4c4cc(-c5ccc6c(c5)c5cc(-c7ccccc7)ccc5n6-c5ccccc5)ccc43)cc2)cc1]

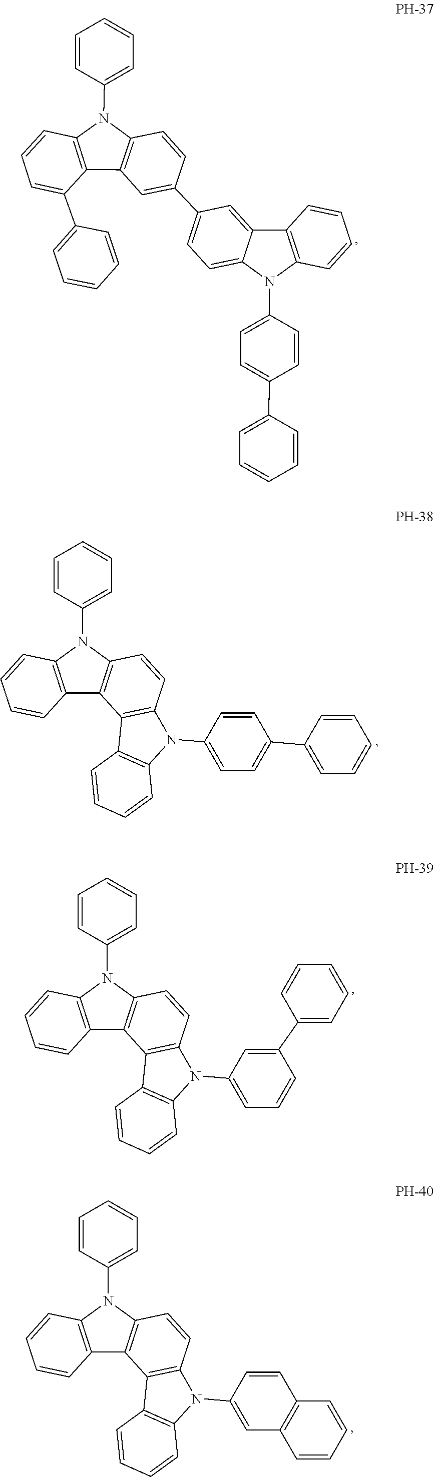 [SMILES: c1ccc(-c2ccc(-n3c4ccccc4c4cc(-c5ccc6c(c5)c5c(-c7ccccc7)cccc5n6-c5ccccc5)ccc43)cc2)cc1 ; c1ccc(-c2ccc(-n3c4ccccc4c4c5c6ccccc6n(-c6ccccc6)c5ccc43)cc2)cc1 ; c1ccc(-c2cccc(-n3c4ccccc4c4c5c6ccccc6n(-c6ccccc6)c5ccc43)c2)cc1 ; c1ccc(-n2c3ccccc3c3c4c5ccccc5n(-c5ccc6ccccc6c5)c4ccc32)cc1]

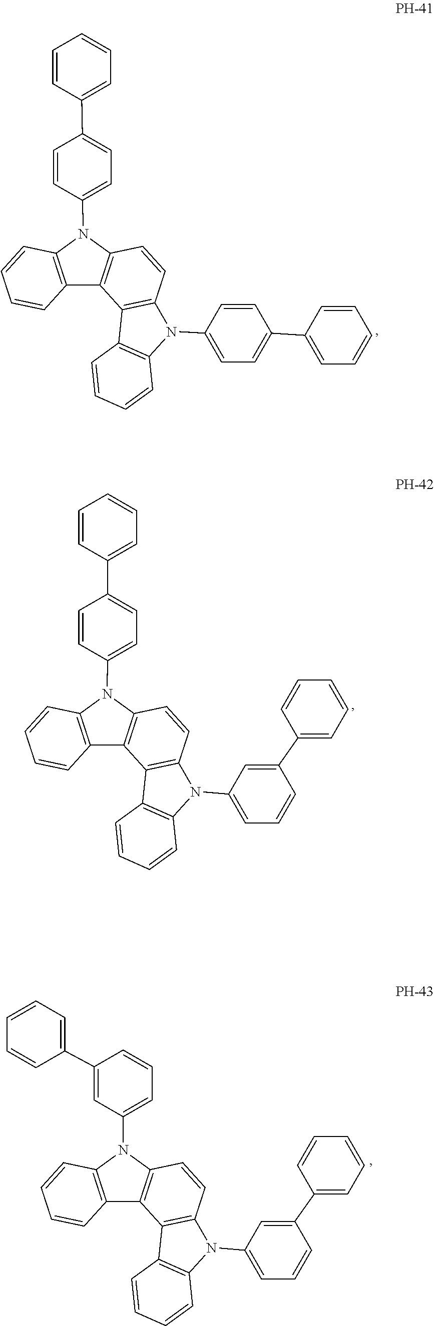 [SMILES: c1ccc(-c2ccc(-n3c4ccccc4c4c5c6ccccc6n(-c6ccc(-c7ccccc7)cc6)c5ccc43)cc2)cc1 ; c1ccc(-c2ccc(-n3c4ccccc4c4c5c6ccccc6n(-c6cccc(-c7ccccc7)c6)c5ccc43)cc2)cc1 ; c1ccc(-c2cccc(-n3c4ccccc4c4c5c6ccccc6n(-c6cccc(-c7ccccc7)c6)c5ccc43)c2)cc1]

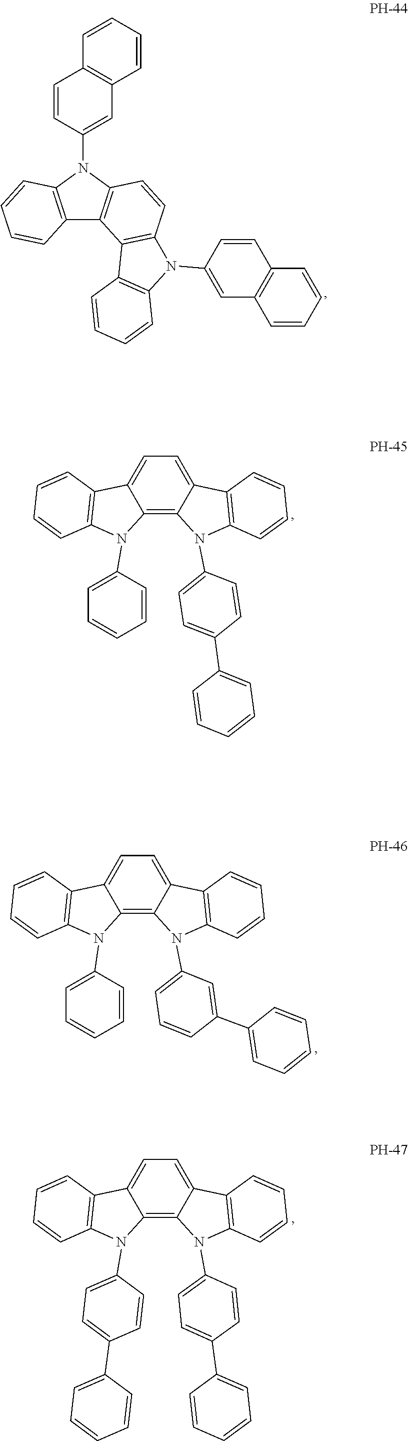 [SMILES: c1ccc2cc(-n3c4ccccc4c4c5c6ccccc6n(-c6ccc7ccccc7c6)c5ccc43)ccc2c1 ; c1ccc(-c2ccc(-n3c4ccccc4c4ccc5c6ccccc6n(-c6ccccc6)c5c43)cc2)cc1 ; c1ccc(-c2cccc(-n3c4ccccc4c4ccc5c6ccccc6n(-c6ccccc6)c5c43)c2)cc1 ; c1ccc(-c2ccc(-n3c4ccccc4c4ccc5c6ccccc6n(-c6ccc(-c7ccccc7)cc6)c5c43)cc2)cc1]

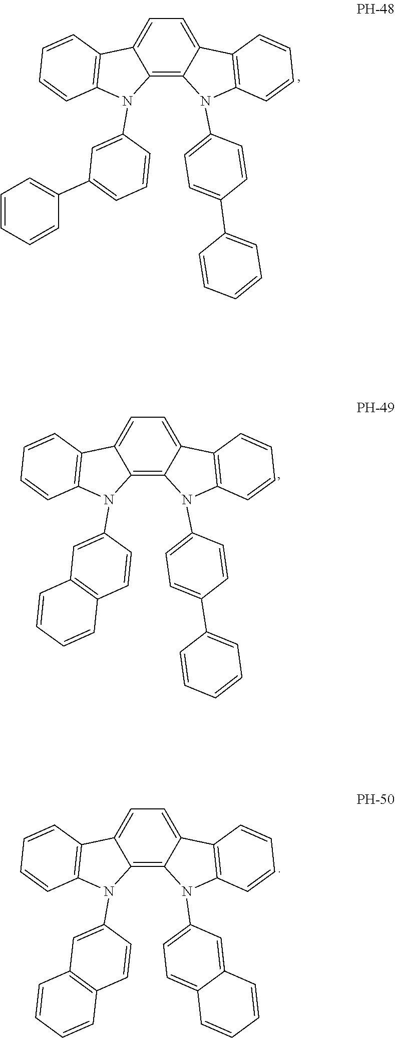 [SMILES: c1ccc(-c2ccc(-n3c4ccccc4c4ccc5c6ccccc6n(-c6cccc(-c7ccccc7)c6)c5c43)cc2)cc1 ; c1ccc(-c2ccc(-n3c4ccccc4c4ccc5c6ccccc6n(-c6ccc7ccccc7c6)c5c43)cc2)cc1 ; c1ccc2cc(-n3c4ccccc4c4ccc5c6ccccc6n(-c6ccc7ccccc7c6)c5c43)ccc2c1]

17. A compound composition, comprising the compound according to claim 1.
